SEQUENCE LISTING (11 sequences)

SEQ ID NO: 1 - protein, 707 aa, Homo sapiens [sequence omitted]

SEQ ID NO: 2 - protein, 427 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGP

SEQ ID NO: 3 - protein, 417 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYG

SEQ ID NO: 4 - protein, 242 aa, Homo sapiens
MVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 5 - protein, 204 aa, Homo sapiens
MLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 6 - protein, 251 aa, Homo sapiens
MAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGQGYSLFLVAAHEFGHALGLDHSSVPEALMYPMYRFTEGPPLHKDDVNGIRHLYG

SEQ ID NO: 7 - protein, 268 aa, Homo sapiens
MHPGVLAAFLFLSWTHCRALPLPSGGDEDDLSEEDLQFAERYLRSYYHPTNLAGILKENAASSMTERLREMQSFFGLEVTGKLDDNTLDVMKKPRCGVPDVGEYNVFPRTLKWSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGP

SEQ ID NO: 8 - protein, 167 aa, Homo sapiens
MFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGP

SEQ ID NO: 9 - protein, 7 aa, Homo sapiens
FQTFEGD

SEQ ID NO: 10 - protein, 15 aa, Homo sapiens
CGVPDLGRFQTFEGD

SEQ ID NO: 11 - protein, 708 aa, Rattus norvegicus
MSPWQPLLLVLLALGYSFAAPHQRQPTYVVFPRDLKTSNLTDTQLAEDYLYRYGYTRAAQMMGEKQSLRPALLMLQKQLSLPQTGELDSETLKAIRSPRCGVPDVGKFQTFDGDLKWHHHNITYWIQSYTEDLPRDVIDDSFARAFAVWSAVTPLTFTRVYGLEADIVIQFGVAEHGDGYPFDGKDGLLAHAFPPGPGIQGDAHFDDDELWSLGKGAVVPTYFGNANGAPCHFPFTFEGRSYLSCTTDGRNDGKPWCGTTADYDTDRKYGFCPSENLYTEHGNGDGKPCVFPFIFEGHSYSACTTKGRSDGYRWCATTANYDQDKADGFCPTRADVTVTGGNSAGEMCVFPFVFLGKQYSTCTSEGRSDGRLWCATTSNFDADKKWGFCPDQGYSLFLVAAHEFGHALGLDHSSVPEALMYPMYHYHEDSPLHEDDIKGIHHLYGRGSKPDPRPPATTAAEPQPTAPPTMCSTAPPMAYPTGGPTVAPTGAPSPGPTGPPTAGPSEAPTESSTPDDNPCNVDVFDAIADIQGALHFFKDGRYWKFSNHGGNQLQGPFLIARTWPAFPSKLNSAFEDPQPKKIFFFLWAQMWVYTGQSVLGPRSLDKLGLGSEVTLVTGLLPRRGGKALLISRERIWKFDLKSQKVDPQSVTRLDNEFSGVPWNSHNVFQYQDKAYFCHDKYFWRVSFHNRVNQVDHVAYVTYDLLQCP

(19) United States
(12) Patent Application Publication
(10) Pub. No.: US 2012/0129843 A1
(43) Pub. Date: May 24, 2012

(54) PYRIDYL-THIAZOLYL INHIBITORS OF PRO-MATRIX METALLOPROTEINASE ACTIVATION

(76) Inventors: Yan ZHANG, Fort Washington, PA (US); Brett Andrew Tounge, Blue Bell, PA (US); Aihua Wang, Jamison, PA (US); Michael Hawkins, Ambler, PA (US); Kristi Anne Leonard, Lansdale, PA (US); Joseph Kent Barbay, Flourtown, PA (US); Umar S.M. Maharoof, North Wales, PA (US)

(21) Appl. No.: 13/297,412

(22) Filed: Nov. 16, 2011

Related U.S. Application Data
(60) Provisional application No. 61/414,970, filed on Nov. 18, 2010.

(51) Int. Cl. A61K 31/4439; C07D 417/14; A61K 31/496; A61K 31/5377; A61K 31/551; A61P 35/00; A61P 19/02; A61P 29/00; A61P 9/00; A61P 1/04; A61P 9/12; A61P 11/00; A61P 1/00; A61P 1/02; A61P 17/02; A61P 1/16; A61P 19/04; A61P 25/00; A61P 11/06; A61P 13/12; A61P 25/06; A61P 9/10; C07D 417/04

(52) U.S. Cl. 514/218; 546/270.4; 514/342; 544/364; 514/253.1; 544/131; 514/236.8; 540/575

(57) ABSTRACT

This invention relates to thiazole I and its therapeutic and prophylactic uses, wherein the variables R_{z}, Q, J, R¹, R³, R⁵, and R⁶ are defined in the specification. Disorders treated and/or prevented include rheumatoid arthritis.

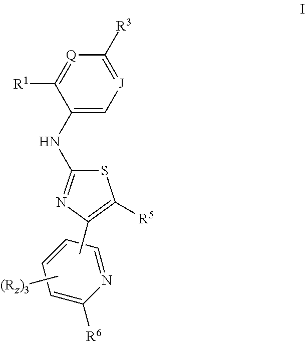

### CROSS-REFERENCE TO RELATED APPLICATIONS

The present application claims the benefits of the filing of U.S. Provisional Application No. 61/414,970 filed Nov. 18, 2010. The complete disclosures of the aforementioned related patent applications are hereby incorporated herein by reference for all purposes.

### TECHNICAL FIELD

The present invention relates to novel pyridyl-thiazolyl compounds and their therapeutic and prophylactic uses. Disorders treated and/or prevented include inflammation related disorders and disorders ameliorated by inhibiting the proteolytic activation of pro-matrix metalloproteinases.

### BACKGROUND OF THE INVENTION

Matrix metalloproteinases (MMPs) are a family of structurally related zinc-dependent proteolytic enzymes that digest extracellular matrix proteins such as collagen, elastin, laminin and fibronectin. Currently, at least 28 different mammalian MMP proteins have been identified and they are grouped based on substrate specificity and domain structure. Enzymatic activities of the MMPs are precisely controlled, not only by their gene expression in various cell types, but also by activation of their inactive zymogen precursors (proMMPs) and inhibition by endogenous inhibitors and tissue inhibitors of metalloproteinases (TIMPs). The enzymes play a key role in normal homeostatic tissue remodeling events, but are also considered to play a key role in pathological destruction of the matrix in many connective tissue diseases such as arthritis, periodontitis, and tissue ulceration and also in cancer cell invasion and metastasis.

A role for MMPs in oncology is well established, as up-regulation of any number of MMPs are one mechanism by which malignant cells can overcome connective tissue barriers and metastasize (Curr Cancer Drug Targets 5(3): 203-20, 2005). MMPs also appear to have a direct role in angiogenesis, which is another reason they have been an important target for oncology indications (Int J Cancer 115(6): 849-60, 2005; J Cell Mol Med 9(2): 267-85, 2005). Several different classes of MMPs are involved in these processes, including MMP9. Other MMP mediated indications include the cartilage and bone degeneration that results in osteoarthritis and rheumatoid arthritis. The degeneration is due primarily to MMP digestion of the extracellular matrix (ECM) in bone and joints (Aging Clin Exp Res 15(5): 364-72, 2003). Various MMPs, including MMP9 and MMP13 have been found to be elevated in the tissues and body fluids surrounding the damaged areas.

Elevated MMP levels, including MMP9 and MMP13 are also believed to be involved in atherosclerotic plaque rupture, aneurysm and vascular and myocardial tissue morphogenesis (Expert Opin Investig Drugs 9(5): 993-1007, 2000; Curr Med Chem 12(8): 917-25, 2005). Elevated levels of MMPs, including MMP9 and MMP13, have often been associated with these conditions. Several other pathologies such as gastric ulcers, pulmonary hypertension, chronic obstructive pulmonary disease, inflammatory bowel disease, periodontal disease, skin ulcers, liver fibrosis, emphysema, and Marfan syndrome all appear to have an MMP component as well (Expert Opinion on Therapeutic Patents 12(5): 665-707, 2002).

Within the central nervous system, altered MMP expression has been linked to several neurodegenerative disease states (Expert Opin Investig Drugs 8(3): 255-68, 1999), most notably in stroke (Glia 50(4): 329-39, 2005). MMPs, including MMP9, have been shown to have an impact in propagating the brain tissue damage that occurs following an ischemic or hemorrhagic insult. Studies in human stroke patients and in animal stroke models have demonstrated that expression levels and activity of MMPs, including MMP9, increase sharply over a 24 hour period following an ischemic event. Administration of MMP inhibitors has been shown to be protective in animal models of stroke (Expert Opin Investig Drugs 8(3): 255-68, 1999; J Neurosci 25(27): 6401-8, 2005). In addition, MMP9 knockout animals also demonstrate significant neuroprotection in similar stroke models (J Cereb Blood Flow Metab 20(12): 1681-9, 2000). In the US, stroke is the third leading cause of mortality, and the leading cause of disability. Thus stroke comprises a large unmet medical need for acute interventional therapy that could potentially be addressed with MMP inhibitors.

It has also been suggested that MMP9 may play a role in the progression of multiple sclerosis (MS). Studies have indicated that serum levels of MMP9 are elevated in active patients, and are concentrated around MS lesions (Lancet Neurol 2(12): 747-56, 2003). Increased serum MMP9 activity would promote infiltration of leukocytes into the CNS, a causal factor and one of the hallmarks of the disease. MMPs may also contribute to severity and prolongation of migraines. In animal models of migraine (cortical spreading depression), MMP9 is rapidly upregulated and activated leading to a breakdown in the BBB, which results in mild to moderate edema (J Clin Invest 113(10): 1447-55, 2004). It is this brain swelling and subsequent vasoconstriction which causes the debilitating headaches and other symptoms associated with migraine. In the cortical spreading depression model, MMP inhibitors have been shown to prevent the opening of the BBB (J Clin Invest 113(10): 1447-55, 2004). Related research has shown that MMP9 is specifically upregulated in damaged brain tissues following traumatic brain injury (J Neurotrauma 19(5): 615-25, 2002), which would be predicted to lead to further brain damage due to edema and immune cell infiltration. MMPs may also have additional roles in additional chronic CNS disorders. In an animal model of Parkinson's disease, MMP9 was found to be rapidly upregulated after striatal injection of a dopaminergic neuron poison (MPTP).

With regard to structure and activation of the inactive zymogen form, a prototypical MMP is matrix metalloproteinase 9 (MMP9). MMP9 is also known as macrophage gelatinase, gelatinase B, 92 kDa gelatinase, 92 kDa type IV collagenase, and type V collagenase. The inactive form of MMP9, proMMP9, is expressed with several different domains including a signal sequence for secretion, a propeptide domain which inhibits activity of proMMP9, a catalytic domain for protein cleavage, a fibronectin type-II (FnII) domain consisting of three fibronectin-type II repeats, and a hemopexin-like domain thought to assist in substrate docking. The hemopexin-like domain also serves as a binding domain for interaction with tissue inhibitors of metalloproteinases (TIMPs). The inactive zymogen form of MMP9, proMMP9, is maintained through a cysteine-switch mechanism, in which a Cys in the propeptide forms a complex with the catalytic zinc in the catalytic domain and occludes the active site (Proc Natl Acad Sci USA 87(14): 5578-82, 1990). Activation of proMMP9 occurs in a two-step process. A protease cleaves an initial site after Met60, disrupting the zinc coordination and destabilizing the propeptide interaction with the catalytic domain. This initial cleavage allows access to the second cleavage site at Phe107, after which the propeptide is removed and the mature active form of the enzyme is released (Biol Chem 378(3-4): 151-60, 1997). The identity of the proMMP9 activating proteases is unknown in vivo, although there is evidence that activation can occur through the actions of MMP3, chymase and trypsin (J Biol Chem 267(6): 3581-4, 1992; J Biol Chem 272(41): 25628-35, 1997; J Biol Chem 280(10): 9291-6, 2005).

Based on the demonstrated involvement in numerous pathological conditions, inhibitors of matrix metalloproteases (MMPs) have therapeutic potential in a range of disease states. However, non-selective active site MMP inhibitors have performed poorly in clinical trials. The failures have often been caused by dose-limiting toxicity and the manifestation of significant side effects, including the development of musculoskeletal syndrome (MSS). It has been suggested that development of more selective MMP inhibitors might help to overcome some of the problems that hindered clinical success in the past, but there are a number of obstacles to developing more selective MMP active site inhibitors. MMPs share a catalytically important Zn2+ ion in the active site and a highly conserved zinc-binding motif. In addition, there is considerable sequence conservation across the entire catalytic domain for members of the MMP family.

A novel approach to developing more selective MMP inhibitors is to target the pro domain of the inactive zymogens, proMMPs, with allosteric small-molecule inhibitors that bind and stabilize the inactive pro form of the protein and inhibit processing to the active enzyme. There is significantly less sequence identity within the pro domains of MMP proteins, no catalytically important Zn2+ ion, and no highly conserved zinc-binding motif. Thus targeting the pro domain of proMMPs is an attractive mechanism of action for inhibiting the activity of the MMP proteins Inhibition of proMMP9 activation has been observed with a specific monoclonal antibody (Hybridoma 12(4): 349-63, 1993). The activation of proMMP9 by trypsin has also been shown to be inhibited by Bowman-Birk inhibitor proteins and derived peptide inhibitors (Biotechnol Lett 26(11): 901-5, 2004). There are no reports, however, of allosteric small-molecule inhibitors that bind the pro domain and inhibit activation of proMMP9, proMMP13, or any other proMMP. The present invention provides tricyclic compounds as allosteric small-molecule inhibitors of the proteolytic activation of proMMP9, proMMP13, and methods of treatment using such inhibitors.

### SUMMARY OF THE INVENTION

The invention comprises the compounds of Formula I

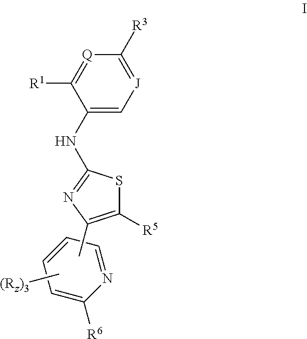

Wherein:

R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### Q is N or C—R²;

R² is H, or CH₃; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is Cl, SO₂NH₂, SO₂CH₃, CO₂H, CONH₂, NO₂, —CN, CH₃, CF₃, or H;

### J is N, or C—R⁴;

R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C₍₁₋₄₎alkyl, F, Cl, Br, —CN, or OC₍₁₋₄₎alkyl;

### R⁵ is H, F, Cl, Br, CF₃, or CH₃;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, or CF₃; or if R_{z} is H, C₍₁₋₆₎alkyl, OCH₃, F, Cl, Br, —CN, or CF₃ then R⁶ may also be

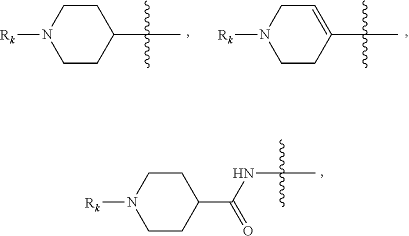

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂C₍₁₋₄₎alkylNA¹A², SOC₍₁₋₄₎alkylNA¹A², pyridinyl, pyrimidinyl, pyrazinyl, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R⁶ may be optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, CH₂C₍₃₋₆₎cycloalkyl, C₍₃₋₆₎cycloalkyl,

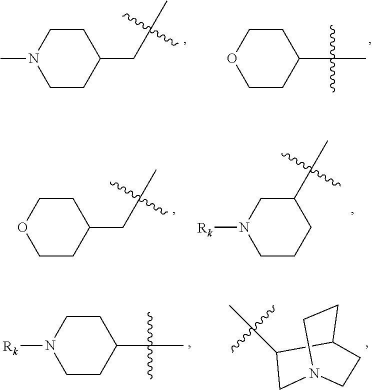

C₍₂₋₆₎alkylOH, C₍₂₋₆₎alkylOCH₃, C₍₂₋₆₎alkylCO₂C₍₁₋₄₎alkyl, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

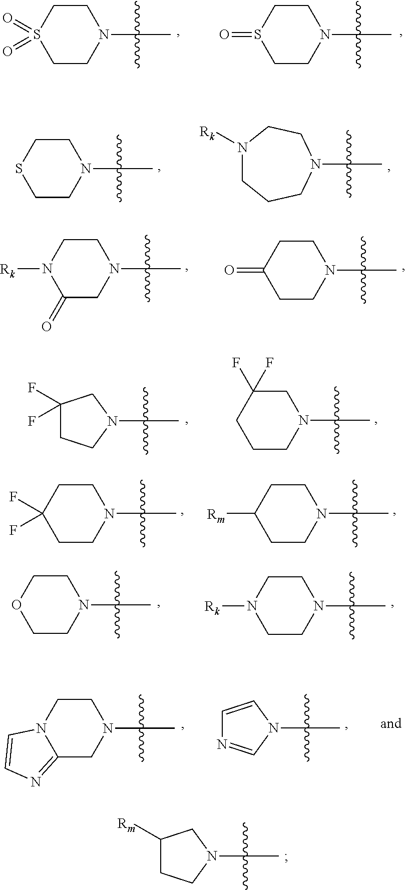

wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₆₎alkyl, COC₍₁₋₄₎alkyl, SO₂C₍₁₋₄₎alkyl, trifluoromethylpyridyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and
R_{z} is independently selected from the group consisting of H, C₍₁₋₆₎alkyl, OCH₃, F, Cl, Br, —CN, and CF₃; or if R⁶ is H, C₍₁₋₆₎alkyl, OCH₃, F, Cl, Br, —CN, or CF₃ then R_{z} may be selected from the group consisting of:

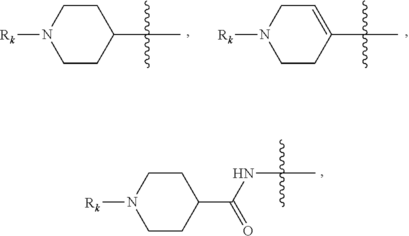

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R_{z} may be optionally substituted with up to four methyl groups on two or more ring carbon atoms or optionally substituted with up to two CF₃ groups on any two ring carbon atoms;
and solvates, hydrates, tautomers, and pharmaceutically acceptable salts thereof.

### BRIEF DESCRIPTION OF THE DRAWINGS

Embodiments of the present invention will now be described, by way of an example only, with reference to the accompanying drawings wherein:

FIG. 1: Shown are western blots with two different antibodies illustrating the effects of a small molecule allosteric processing inhibitor, Compound α, on the activation of proMMP9 in synoviocytes harvested from female Lewis rats after inducing arthritis with i.p. administration of Streptococcal cell wall peptidoglycan polysaccharides. A mouse monoclonal antibody, mAb L51/82, detected pro and processed forms of MMP9. The mouse monoclonal antibody showed that Compound α caused a dose-dependent reduction in the appearance of the 80 kD active form of MMP9 and the appearance of an 86 kD form of the protein (FIG. 1A, lanes 3-6). A rabbit polyclonal antibody, pAb-1246, detected the 80 kD active form of MMP9, but did not recognize the 100 kD form of proMMP9. The rabbit polyclonal antibody showed that the small molecule allosteric processing inhibitor caused a dose-dependent reduction in the appearance of the 80 kD active form of MMP9 (FIG. 1B, lanes 2-6).

FIG. 2: Shown are western blots illustrating increased proMMP9 and increased active MMP9 in tibia-tarsus joints (ankles) from female Lewis rats after inducing arthritis with i.p. administration of Streptococcal cell wall peptidoglycan polysaccharides (SCW). In healthy ankles of rats administered saline, mAb-L51/82 detected small amounts of an approximately 100 kD proMMP9 and an approximately 80 kD form of active MMP9 (FIG. 2A, lanes 1 and 2). The amount of proMMP9 increased markedly in ankle homogenates 5 and 18 days after SCW-administration (FIG. 2A, lanes 3-5 and 6-8, respectively). The amount of active 80 kD MMP9 increased mildly 5 days after SCW-administration (FIG. 2A, lanes 3-5) and increased markedly 18 days after SCW-administration (FIG. 2A, lanes 6-8). In healthy ankles of rats administered saline, mAb-1246 detected small amounts active 80 kD MMP9 (FIG. 2B, lanes 1 and 2). The 80 kD active MMP9 increased mildly 5 days after SCW-administration (FIG. 2A, lanes 3-5) and increased markedly 18 days after SCW-administration (FIG. 2A, lanes 6-8).

FIG. 3: Shown are western blots with two different antibodies illustrating the effects of a small molecule allosteric processing inhibitor, Compound α, on the activation of proMMP9 in tibia-tarsus joints (ankles) from female Lewis rats after inducing arthritis with i.p. administration of Streptococcal cell wall peptidoglycan polysaccharides (SCW). Both proMMP9 and active MMP9 were abundantly present in ankles of SCW-induced vehicle-treated rats (FIGS. 3A and 3B, lanes 1-3). Treatment of rats with Compound α did not reduce the abundance of proMMP-9 (FIG. 3A, lanes 4-9). However, treatment of rats with Compound α resulted in a notable reduction in the active 80 kD form of MMP9 detected with pAb-1246 (FIG. 3B, lanes 4-9) and also with mAb-L51/82 (FIG. 3A, lanes 4-9).

### DETAILED DESCRIPTION OF THE INVENTION

The invention comprises the compounds of Formula I

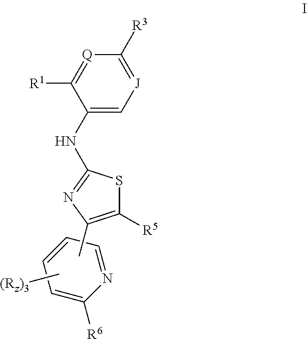

wherein:
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### Q is N or C—R²;

R² is H, or CH₃; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is Cl, SO₂NH₂, SO₂CH₃, CO₂H, CONH₂, NO₂, —CN, CH₃, CF₃, or H;

### J is N, or C—R⁴;

R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C₍₁₋₄₎alkyl, F, Cl, Br, —CN, or OC₍₁₋₄₎alkyl;

### R⁵ is H, F, Cl, Br, CF₃, or CH₃;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, or CF₃; or if R_{z} is H, C₍₁₋₆₎alkyl, OCH₃, F, Cl, Br, —CN, or CF₃ then R⁶ may also be

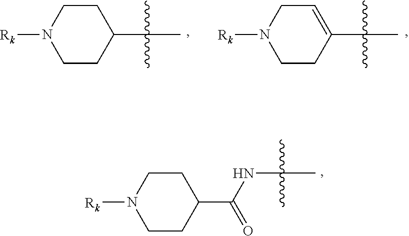

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂C₍₁₋₄₎alkylNA¹A², SOC₍₁₋₄₎alkylNA¹A², pyridinyl, pyrimidinyl, pyrazinyl, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R⁶ may be optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, CH₂C₍₃₋₆₎cycloalkyl, C₍₃₋₆₎cycloalkyl,

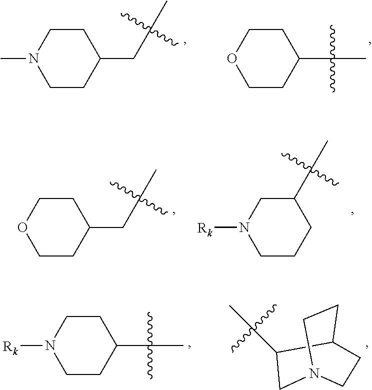

C₍₂₋₆₎alkylOH, C₍₂₋₆₎alkylOCH₃, C₍₂₋₆₎alkylCO₂C₍₁₋₄₎alkyl, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

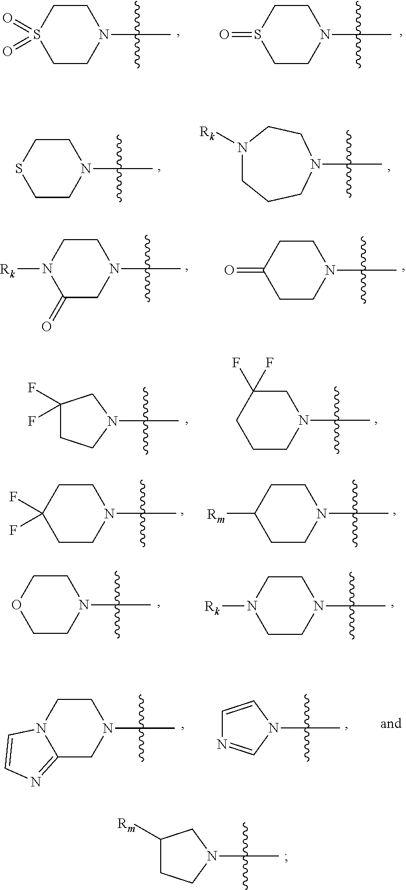

wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₆₎alkyl, COC₍₁₋₄₎alkyl, SO₂C₍₁₋₄₎alkyl, trifluoromethylpyridyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and
R_{z} is independently selected from the group consisting of H, C₍₁₋₆₎alkyl, OCH₃, F, Cl, Br, —CN, and CF₃; or if R⁶ is H, C₍₁₋₆₎alkyl, OCH₃, F, Cl, Br, —CN, or CF₃ then R_{z} may be selected from the group consisting of:

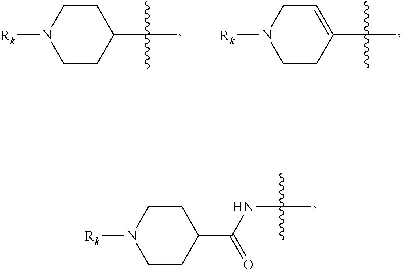

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R_{z} may be optionally substituted with up to four methyl groups on two or more ring carbon atoms or optionally substituted with up to two CF₃ groups on any two ring carbon atoms;
and solvates, hydrates, tautomers, and pharmaceutically acceptable salts thereof.

In another embodiment of the invention:

R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### Q is N or C—R²;

R² is H, or CH₃; or R² and R¹ may be taken together with the ring to which they are attached, to form a fused ring system selected from the group consisting of: quinolinyl, isoquinolinyl, quinazolinyl, quinoxalinyl, benzimidazolyl, benzofuranyl, 2,3-dihydro-benzofuranyl, benzothiophenyl, benzothiazolyl, and indazolyl, wherein said quinolinyl, isoquinolinyl, quinazolinyl, quinoxalinyl, benzimidazolyl, benzothiazolyl, benzofuranyl, 2,3-dihydro-benzofuranyl, benzothiophenyl, and indazolyl are optionally substituted with one methyl group or up to two fluorine atoms;
R³ is C₁, SO₂NH₂, SO₂CH₃, CO₂H, CONH₂, NO₂, —CN, CH₃, CF₃, or H;

### J is N, or C—R⁴;

R⁴ is CH₃, —CN, —CONH₂, —CO₂H, —NO₂, —CONHC₍₁₋₄₎alkyl, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), or R⁴ is selected from the group consisting of: pyrazolyl, imidazolyl, oxazolyl, isoxazolyl, thiazolyl, furyl, and thiophenyl wherein said pyrazolyl, imidazolyl, oxazolyl, isoxazolyl, thiazolyl, furyl, and thiophenyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;

### R^{d} is CH₃, F, Cl, Br, —CN, or OCH₃;

### R⁵ is H, F, Cl, Br, CF₃, or CH₃;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, CF₃,

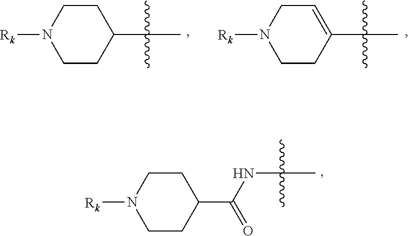

CO₂H, CO₂C₍₁₋₄₎alkyl, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², OC₍₂₋₆₎alkyl, OC₍₂₋₆₎alkylNA¹A², or CH₂NA¹A²;
A¹ is H, or C₍₁₋₃₎alkyl;
A² is H, C₍₁₋₆₎alkyl, CH₂C₍₃₋₆₎cycloalkyl, C₍₃₋₆₎cycloalkyl,

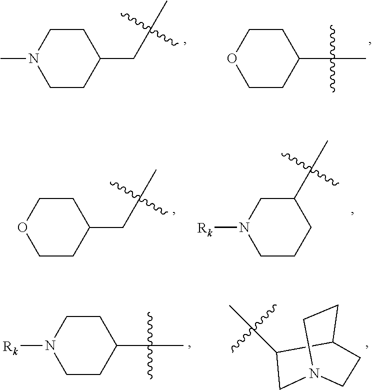

C₍₂₋₆₎alkylOH, C₍₂₋₆₎alkylOCH₃, C₍₂₋₆₎alkylCO₂C₍₁₋₃₎alkyl, SO₂C₍₁₋₄₎alkyl, C(O)Ph, C(O)C₍₁₋₄₎alkyl, pyrazinyl, or pyridyl;
or A¹ and A² are taken together with their attached nitrogen to form a ring selected from the group consisting of:

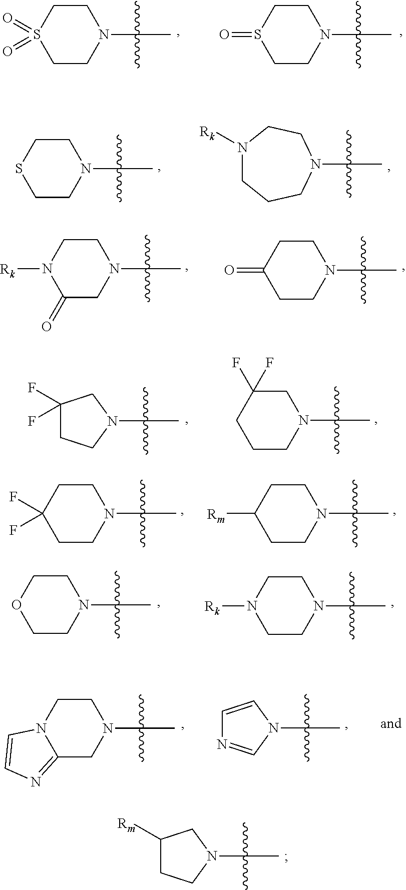

wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₅₎alkyl, COC₍₁₋₄₎alkyl, SO₂C₍₁₋₄₎alkyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH; and
R_{z} is independently selected from the group consisting of H, C₍₁₋₆₎alkyl, OCH₃, F, Cl, Br, —CN, and CF₃;
and solvates, hydrates, tautomers, and pharmaceutically acceptable salts thereof.

In another embodiment of the invention:

R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;

### Q is N or C—R²;

R² is H, or CH₃; or R² and R¹ may be taken together with the ring to which they are attached, to form a fused ring system selected from the group consisting of: quinolinyl, benzofuranyl, and 2,3-dihydro-benzofuranyl, wherein said quinolinyl, benzofuranyl, and 2,3-dihydro-benzofuranyl are optionally substituted with one methyl group or up to two fluorine atoms;
R³ is C₁, SO₂NH₂, SO₂CH₃, CO₂H, CONH₂, NO₂, —CN, CH₃, CF₃, or H;

### J is N, or C—R⁴;

R⁴ is —CN, —CONH₂, —CO₂H, —NO₂, —CO₂C₍₁₋₄₎alkyl, SO₂C₍₁₋₃₎alkyl, —SO₂NH₂, CH₂CONH₂, or R⁴ is selected from the group consisting of: pyrazolyl, and oxazolyl, wherein said pyrazolyl, and oxazolyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;

### R^{d} is CH₃, F, or Cl;

### R⁵ is H, F, Cl, Br, or CH₃;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN,

### CH₂OH, CF₃,

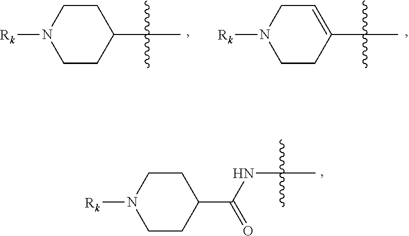

CO₂H, CO₂C₍₁₋₄₎alkyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², OC₍₂₋₃₎alkyl, OC₍₂₋₃₎alkylNA¹A², or CH₂NA¹A²;
A¹ is H, or C₍₁₋₃₎alkyl;
A² is H, C₍₁₋₅₎alkyl, CH₂-cyclopropyl, C₍₂₋₆₎alkylOCH₃, CH₂CH₂CO₂CH₂CH₃, C₍₂₋₆₎alkylOH,

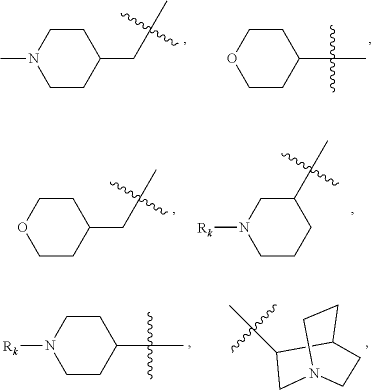

C(O)C₍₁₋₄₎alkyl;
or A¹ and A² are taken together with their attached nitrogen to form a ring selected from the group consisting of:

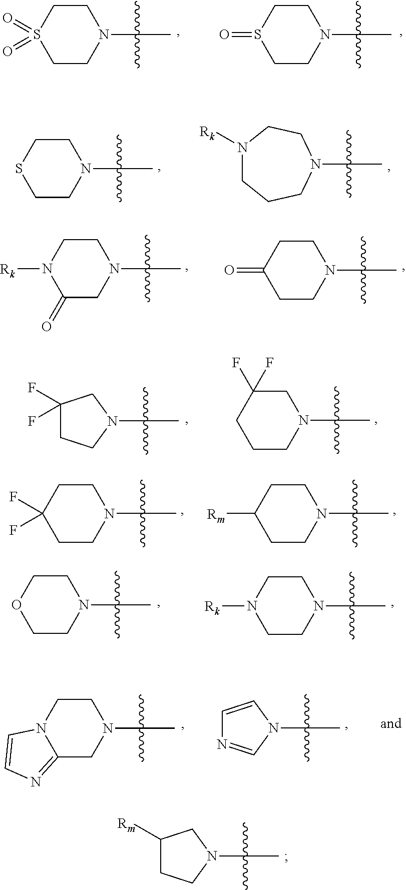

Rₖ is selected from the group consisting of H, CO₂C(CH₃)₃, C₍₁₋₃₎alkyl, COC₍₁₋₄₎alkyl, SO₂C₍₁₋₄₎alkyl, CH₂CH₂CF₃, CH₂-cyclopropyl, and C₍₃₋₆₎cycloalkyl;

### Rₘ is H, OCH₃, CH₂OH, NH(CH₃), N(CH₃)₂, NH₂, CH₃, F, or OH; and

R_{z} is independently selected from the group consisting of H, C₍₁₋₄₎alkyl, and Br;
and solvates, hydrates, tautomers, and pharmaceutically acceptable salts thereof.

In another embodiment of the invention:

R¹ is OC₍₁₋₄₎alkyl, SC₍₁₋₄₎alkyl, C₍₁₋₄₎alkyl, OCH₂C₍₃₋₅₎cycloalkyl, OC₍₃₋₅₎cycloalkyl, or OCF₃;

### Q is N or C—R²;

R² is H; or R¹ and R² may be taken together with their attached ring to form the fused bicycle 2-methyl benzofuran-7-yl;

### R³ is SO₂NH₂, SO₂CH₃, CO₂H, CONH₂, CH₃, —CN, or H;

### J is N, or C—R⁴;

R⁴ is —CN, —CONH₂, —CO₂H, SO₂C₍₁₋₃₎alkyl, —SO₂NH₂, —NO₂, CH₂CONH₂, or R⁴ is selected from the group consisting of: pyrazolyl, and oxazolyl, wherein said pyrazolyl, and oxazolyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;

### R^{d} is CH₃, F, or Cl;

### R⁵ is H;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN,

### CH₂OH, CF₃,

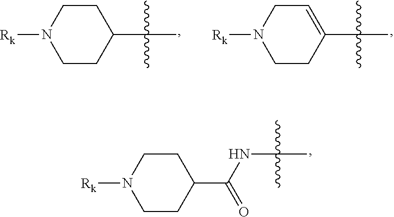

CO₂H, CO₂C₍₁₋₄₎alkyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², OC₍₂₋₃₎alkyl, OC₍₂₋₃₎alkylNA¹A², or CH₂NA¹A²;
A¹ is H, or C₍₁₋₃₎alkyl;
A² is H, C₍₁₋₅₎alkyl, CH₂-cyclopropyl, C₍₂₋₃₎alkylOCH₃, CH₂CH₂CO₂CH₂CH₃, CH₂CH₂OH,

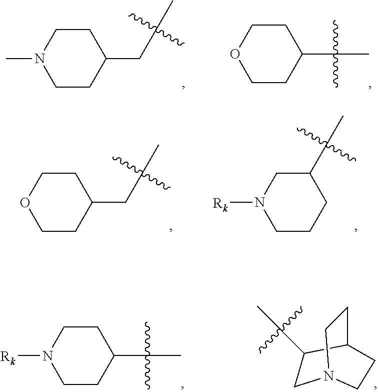

C(O)C₍₁₋₄₎alkyl;
or A¹ and A² are taken together with their attached nitrogen to form a ring selected from the group consisting of:

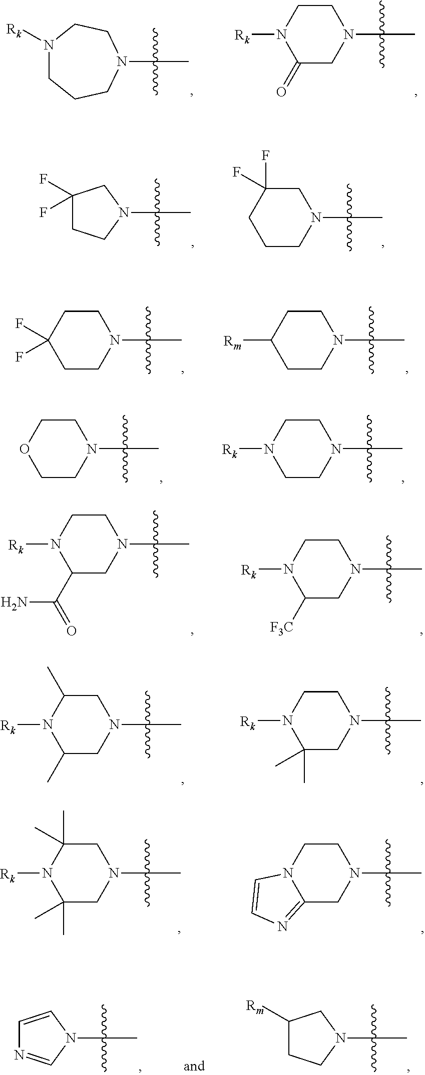

Rₖ is selected from the group consisting of H, CO₂C(CH₃)₃, C₍₁₋₃₎alkyl, COC₍₁₋₄₎alkyl, SO₂C₍₁₋₄₎alkyl, CH₂CH₂CF₃, CH₂-cyclopropyl, and C₍₃₋₆₎cycloalkyl;

### Rₘ is H, OCH₃, CH₂OH, NH(CH₃), N(CH₃)₂, NH₂, CH₃, F, or OH; and

R_{z} is independently selected from the group consisting of H, C₍₁₋₄₎alkyl, and Br;
and solvates, hydrates, tautomers, and pharmaceutically acceptable salts thereof.

In another embodiment of the invention:

R¹ is OC₍₁₋₃₎alkyl, isobutyl, or OCF₃;

### Q is C—R²;

R² is H; or R¹ and R² may be taken together with their attached ring to form 2-methyl benzofuran-7-yl;

### R³ is H, CH₃, or CONH₂;

### J is C—R⁴;

R⁴ is CONH₂, SO₂CH₃, SO₂CH₂CH₃, NO₂, oxazolyl, CO₂H, CH₂CONH₂, —CN, or SO₂NH₂; or R₄ is H, provided that R₃ is CONH₂; or R⁴ may also be H provided that R¹ and R² are taken together with the ring to which they are attached, to form a fused ring system;

### R⁵ is H;

R⁶ is H, —CH═CH—CH₂OCH₃, CH₂CH₂CH₂OCH₃, NA¹A², Cl, C(O)NA¹A², CO₂H, N(CH₃)CH₂CH₂N(CH₃)₂, C(O)N(CH₃)CH₂CH₂N(CH₃)₂, NHC₍₂₋₃₎alkylNA¹A², C(O)NHCH₂CH₂N(CH₃)₂, CH₂OH, CH₃, Br, OCH₃,

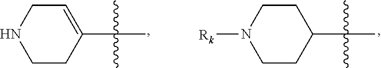

### or CF₃;

### A¹ is CH₃, or H;

A² is H, CH₂-cyclopropyl, C₍₂₋₃₎alkylOCH₃, CH₂CH₂CO₂CH₂CH₃, CH₂CH₂OH, C₍₁₋₅₎alkyl,

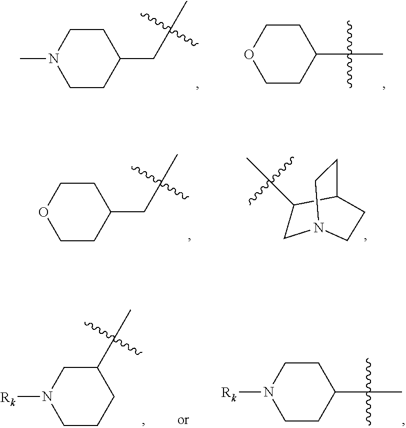

or A¹ and A² may be taken together with their attached nitrogen to form a ring selected from the group consisting of:

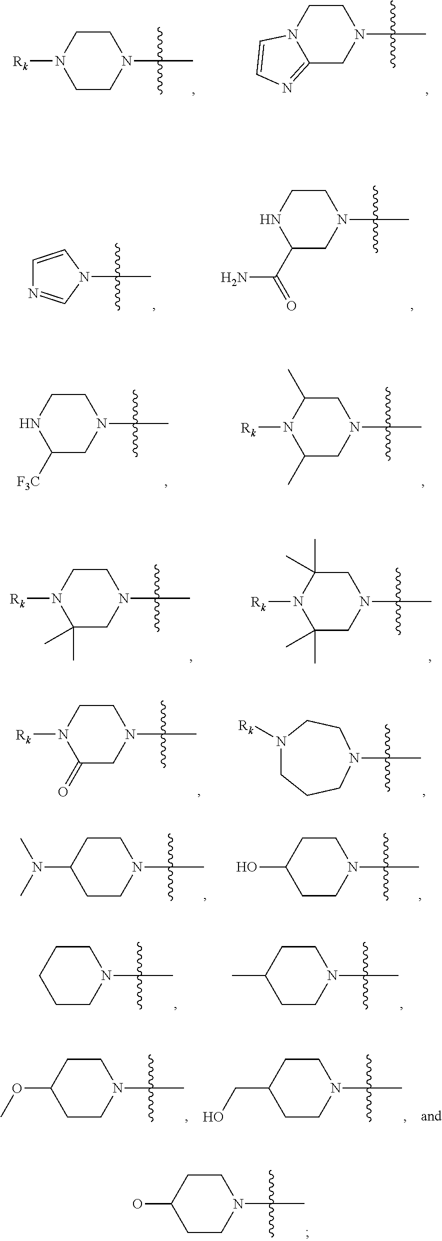

Rₖ is H, CO₂C(CH₃)₃, COCH₃, cyclopropyl, CH₂CH₂CF₃, CH₂-cyclopropyl, SO₂CH₃, or C₍₁₋₃₎alkyl; and
R_{z} is independently selected from the group consisting of H, CH₃, isopropyl, and Br;
and solvates, hydrates, tautomers, and pharmaceutically acceptable salts thereof.

Another embodiment of the invention is a compound selected from the group consisting of:

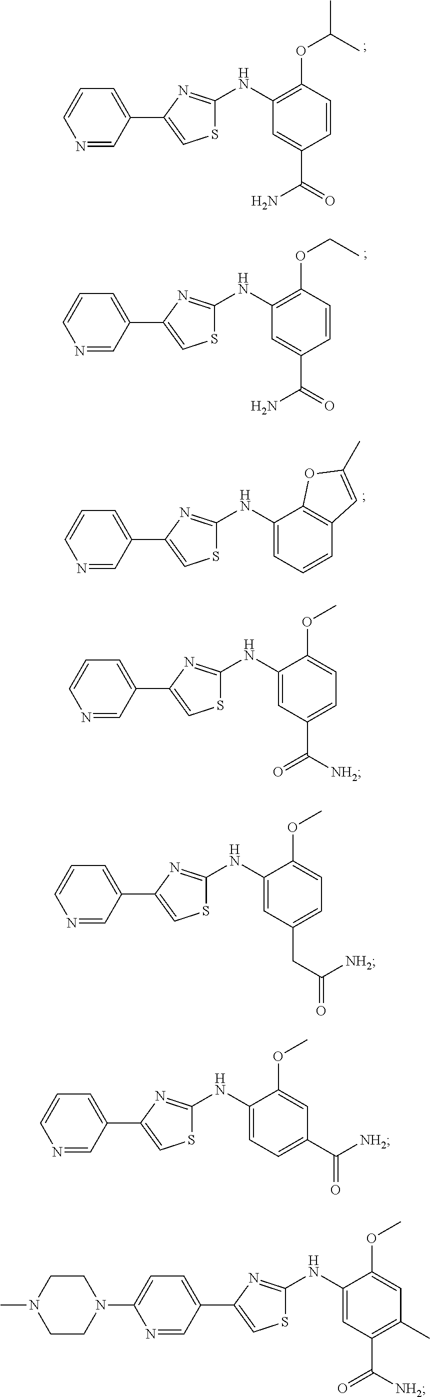

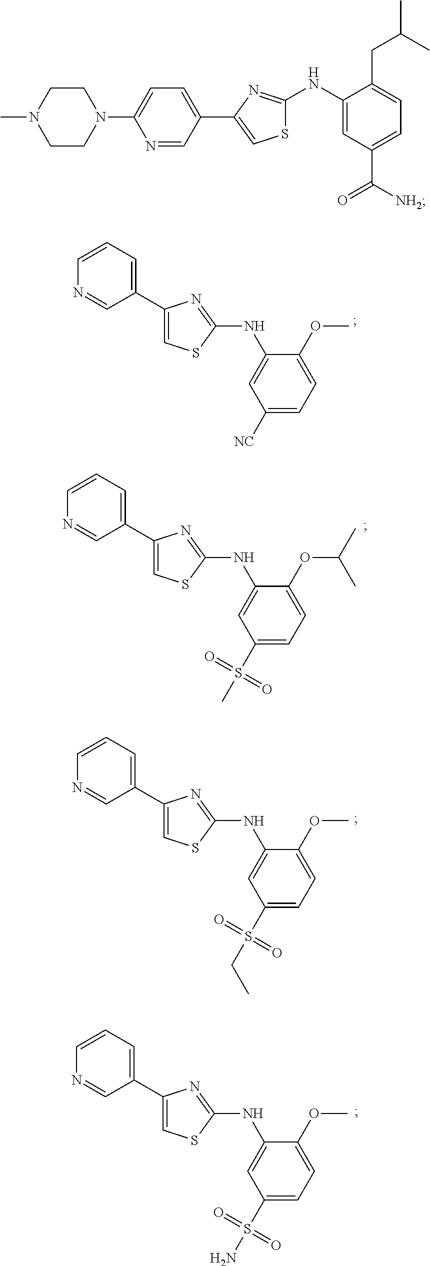

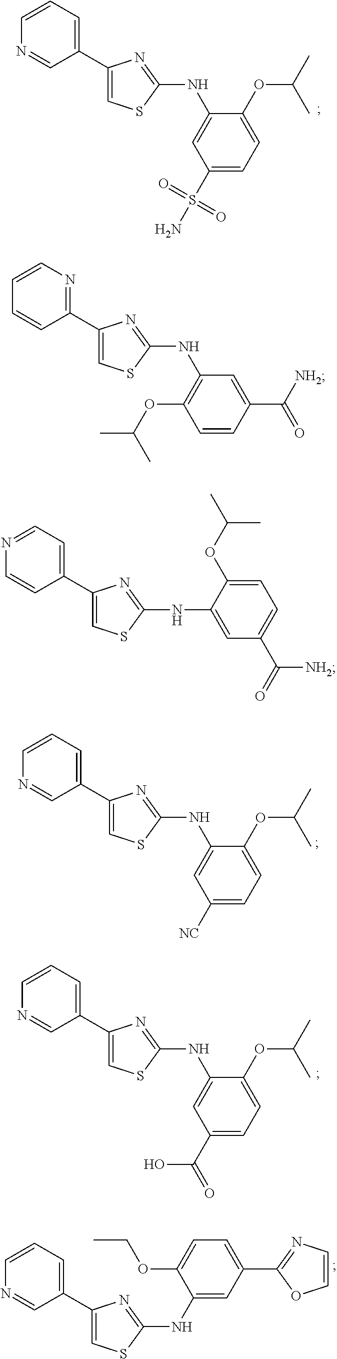

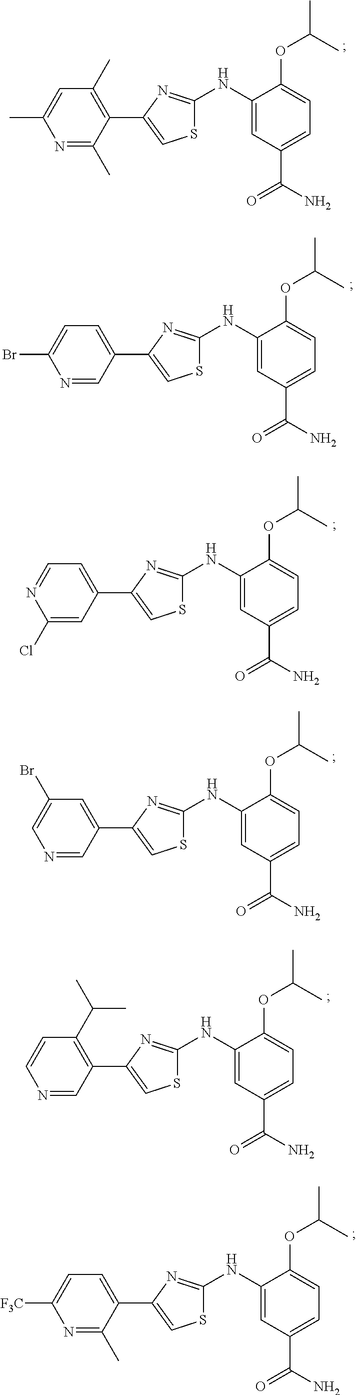

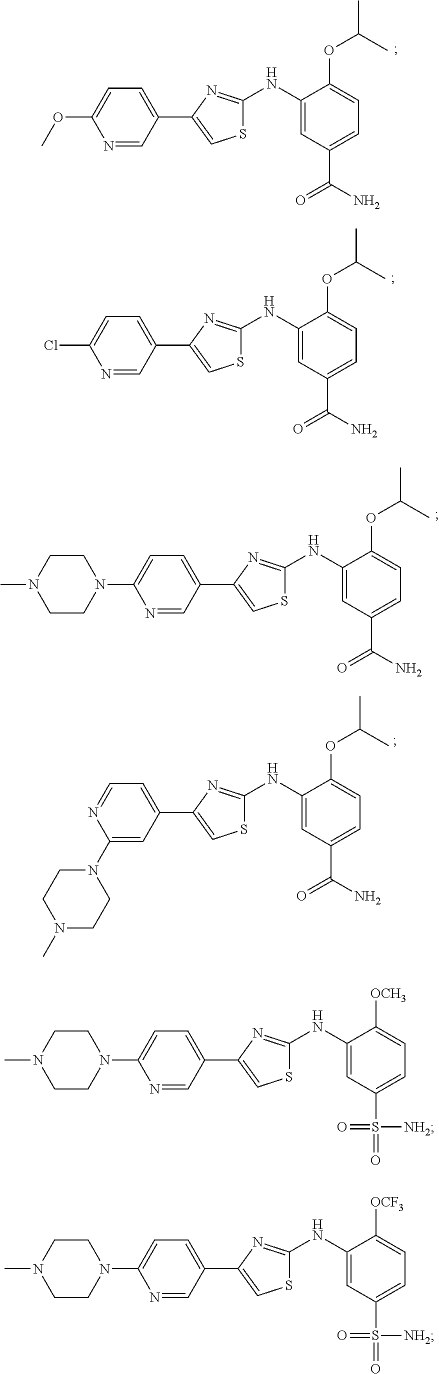

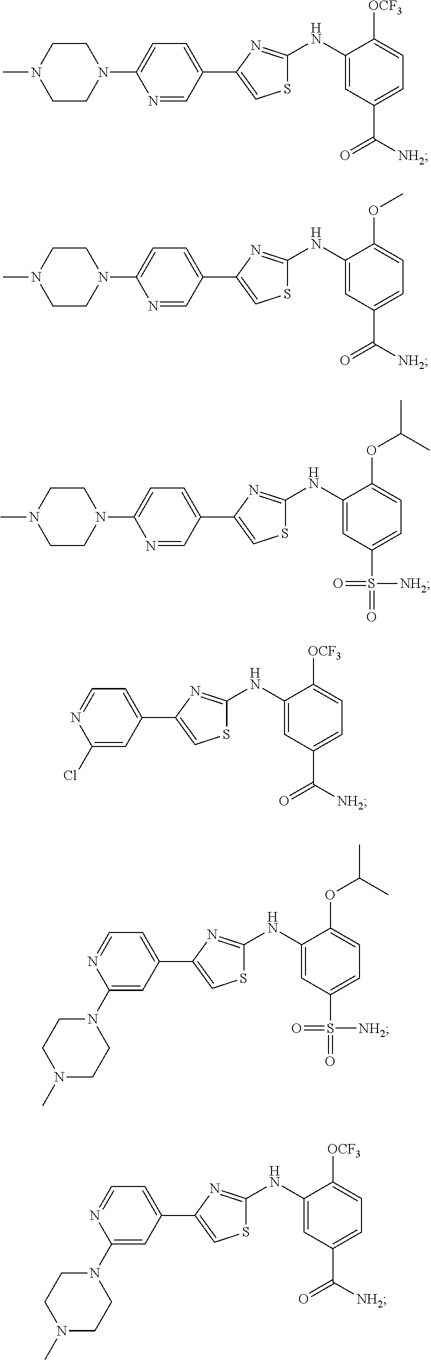

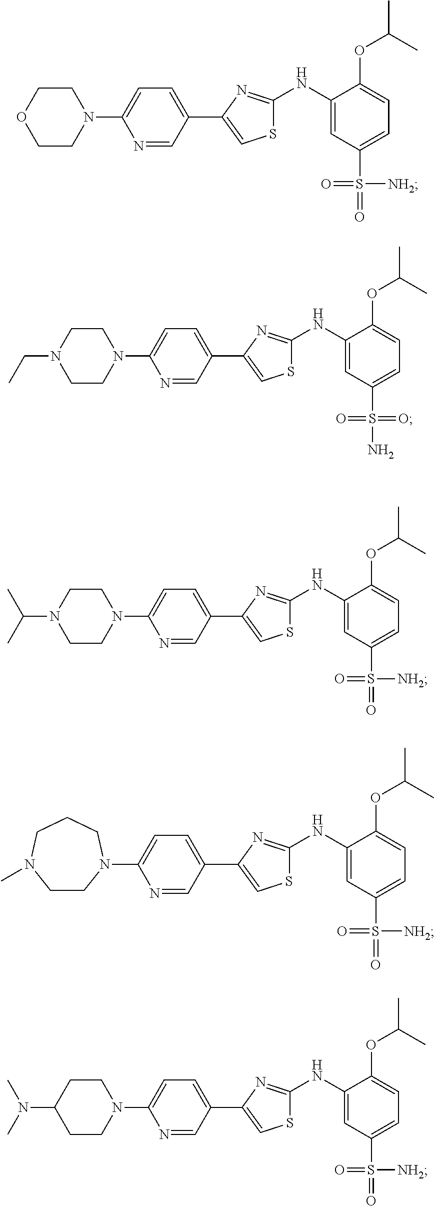

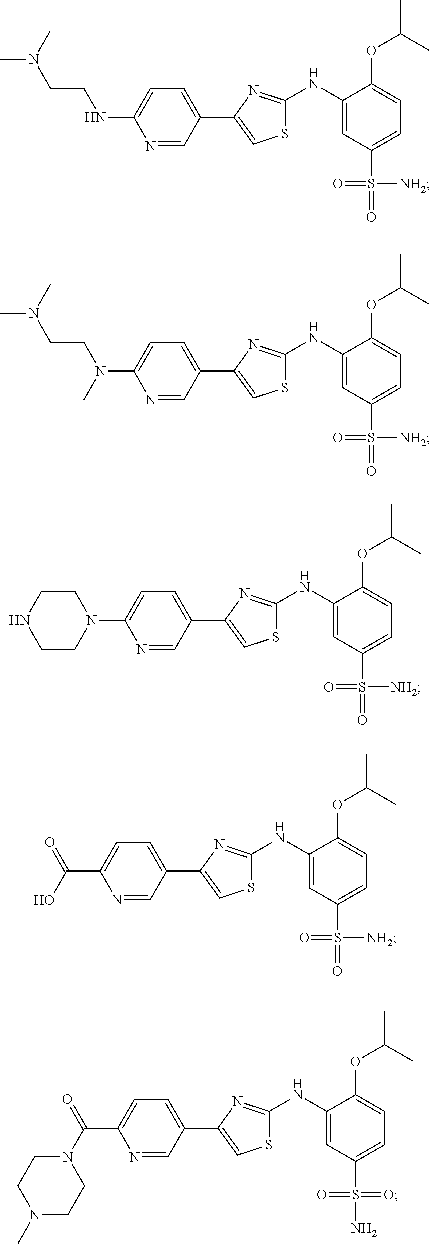

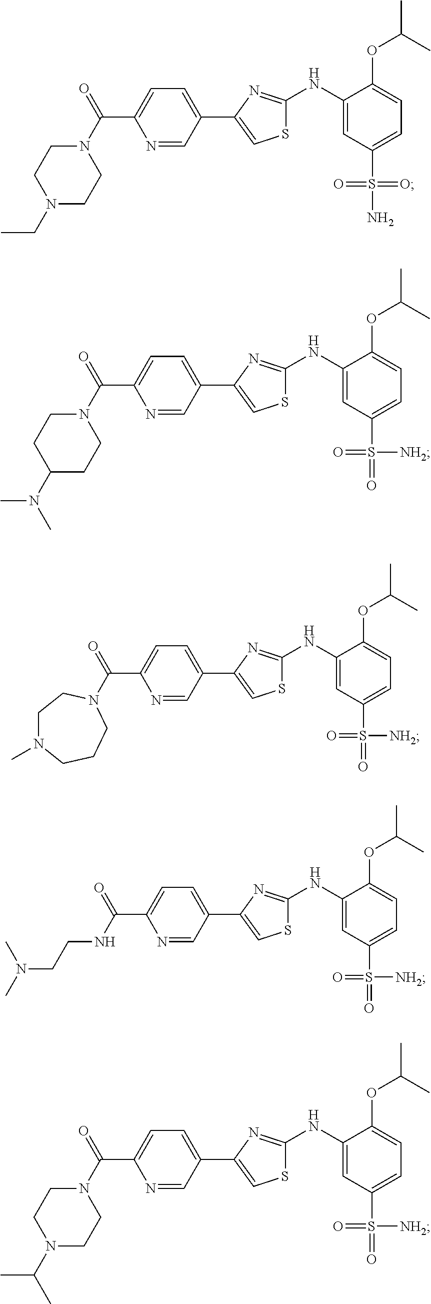

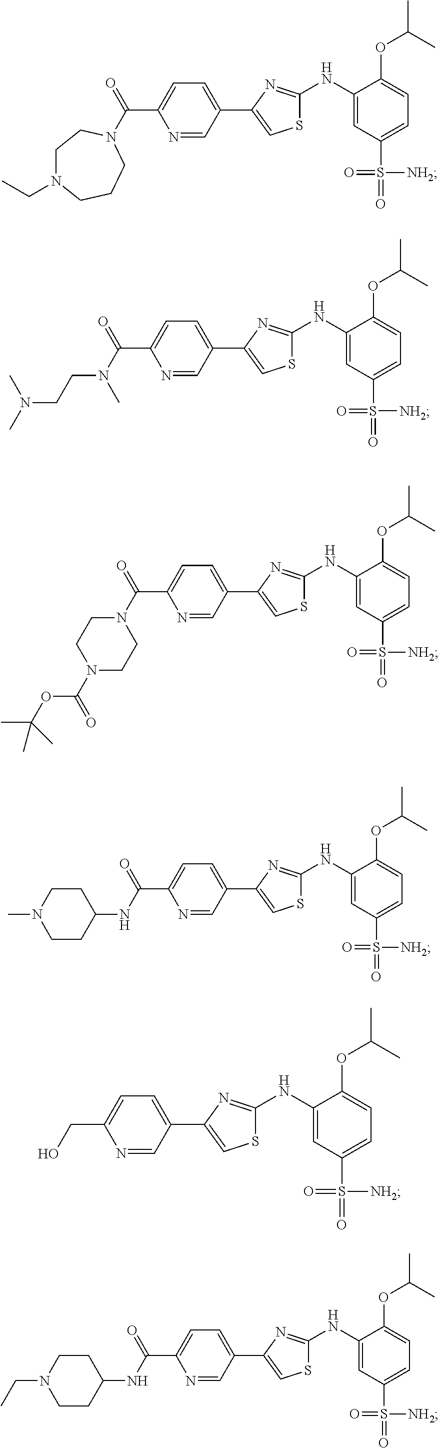

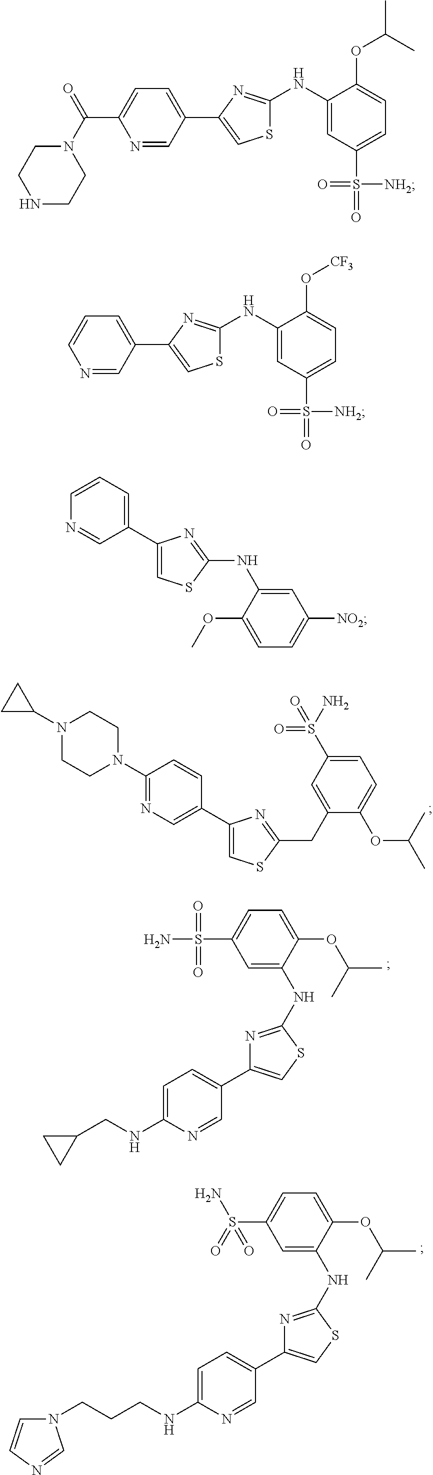

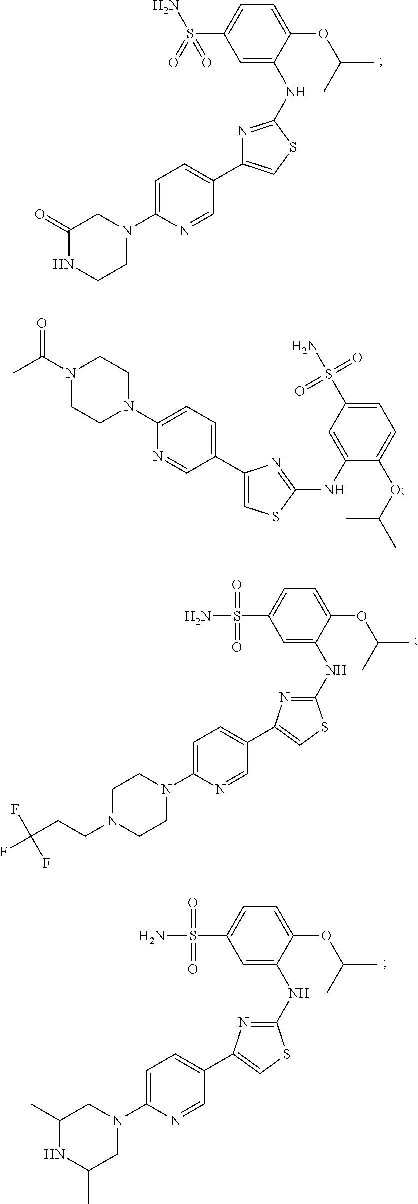

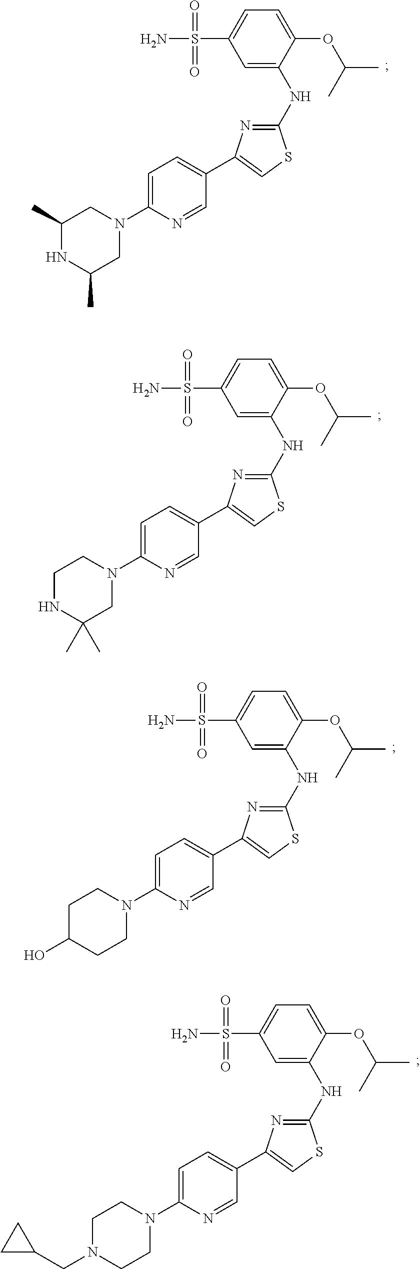

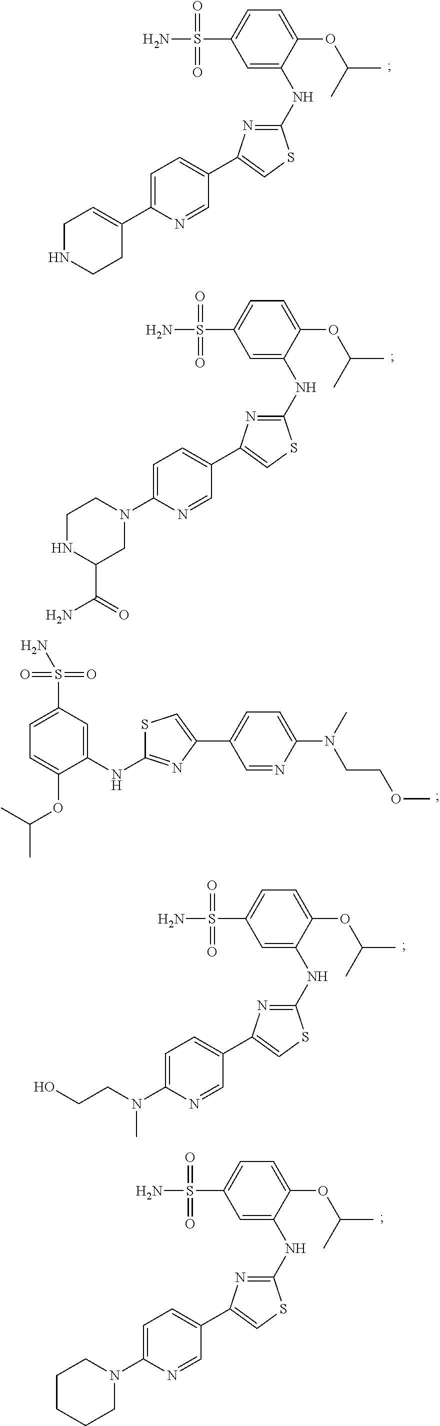

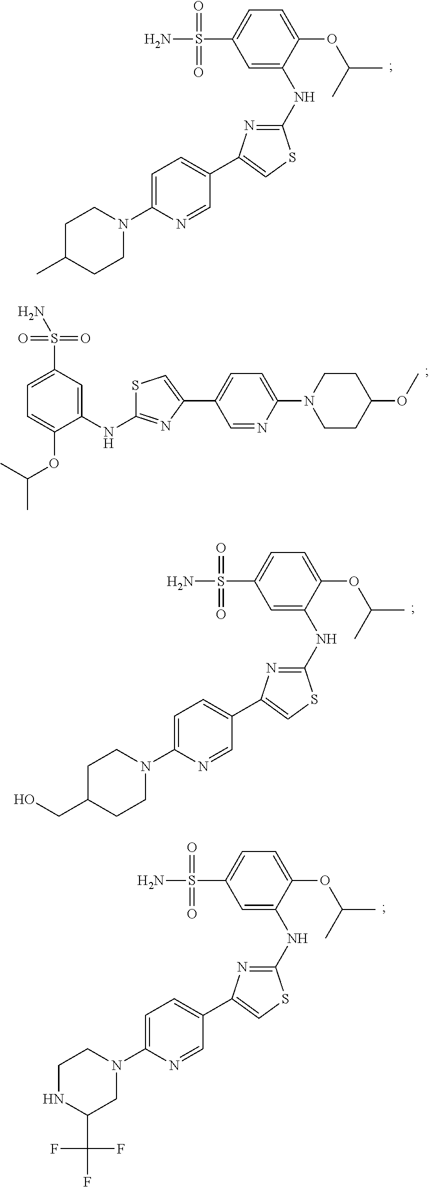

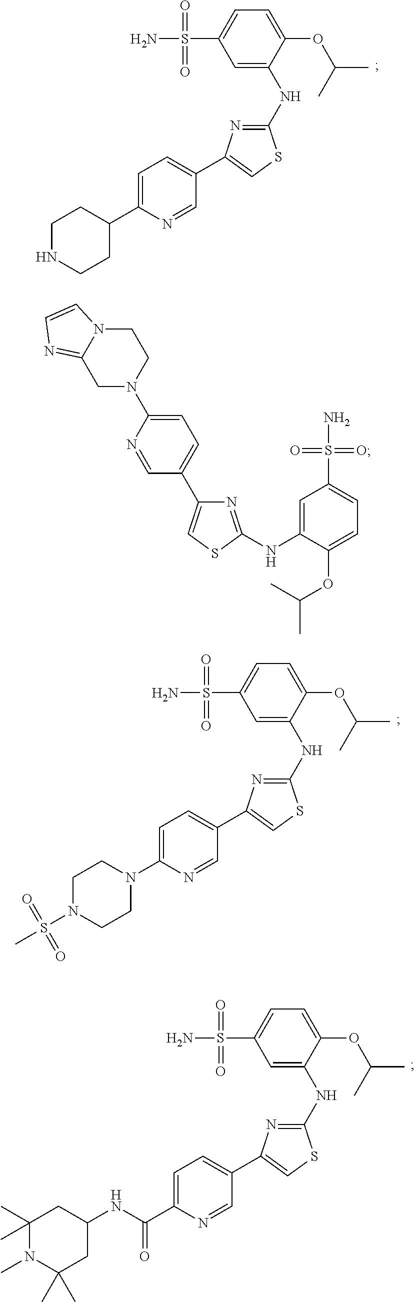

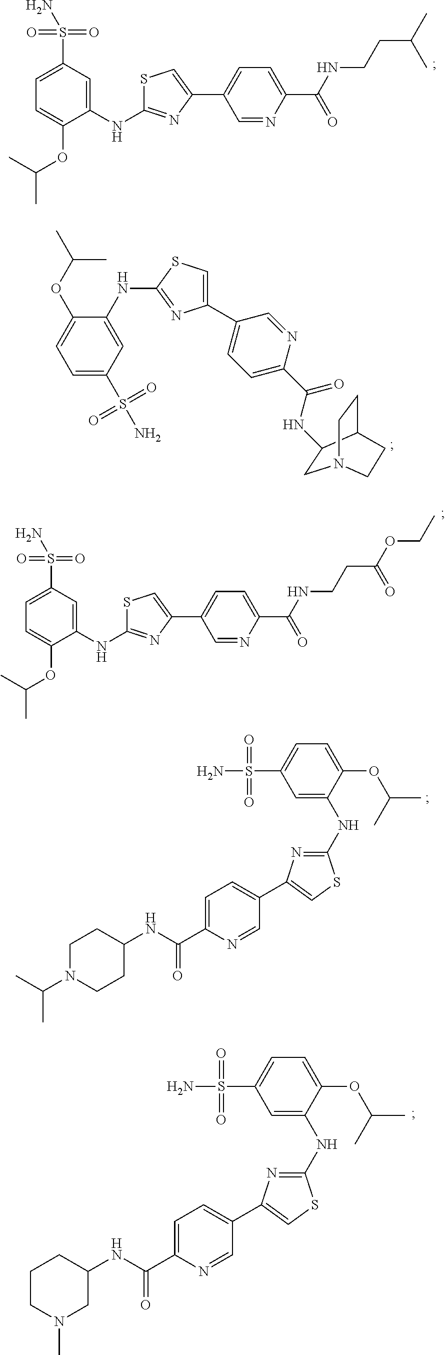

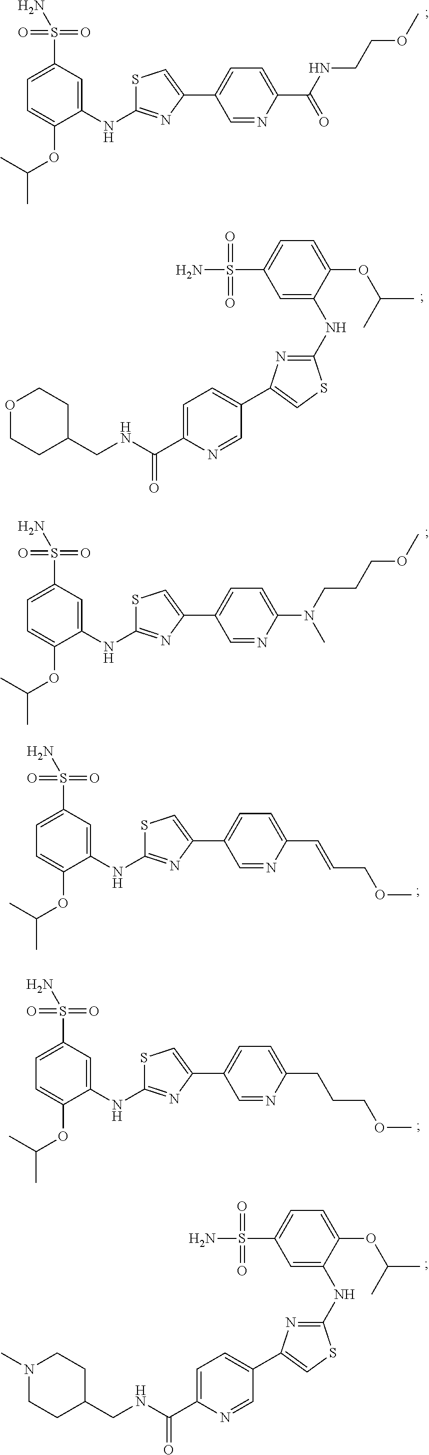

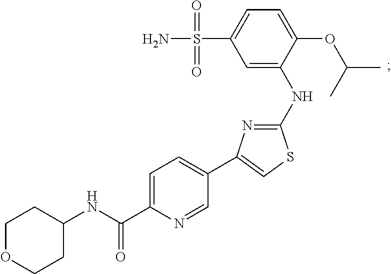

and solvates, hydrates, tautomers, and pharmaceutically acceptable salts thereof.

In another embodiment of the invention:

### R¹ is OCH(CH₃)₂;

### Q is C—R²;

### R² is H;

### R³ is H;

### J is C—R⁴;

### R⁴ is —CONH₂, —CO₂H, or —SO₂NH₂;

### R⁵ is H;

R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, or CF₃; or if R_{z} is H, C₍₁₋₆₎alkyl, OCH₃, F, Cl, Br, —CN, or CF₃ then R⁶ may also be

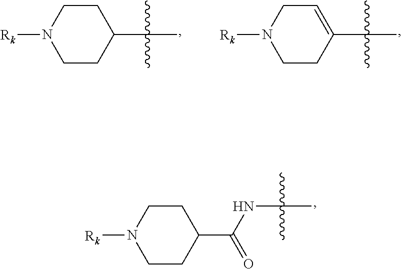

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂C₍₁₋₄₎alkylNA¹A², SOC₍₁₋₄₎alkylNA¹A², pyridinyl, pyrimidinyl, pyrazinyl, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R⁶ may be optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, CH₂C₍₃₋₆₎cycloalkyl, C₍₃₋₆₎cycloalkyl

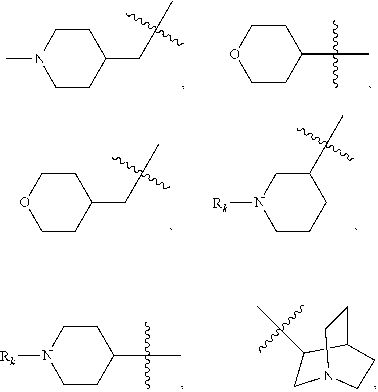

C₍₂₋₆₎alkylOH, C₍₂₋₆₎alkylOCH₃, C₍₂₋₆₎alkylCO₂C₍₁₋₄₎alkyl, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of:

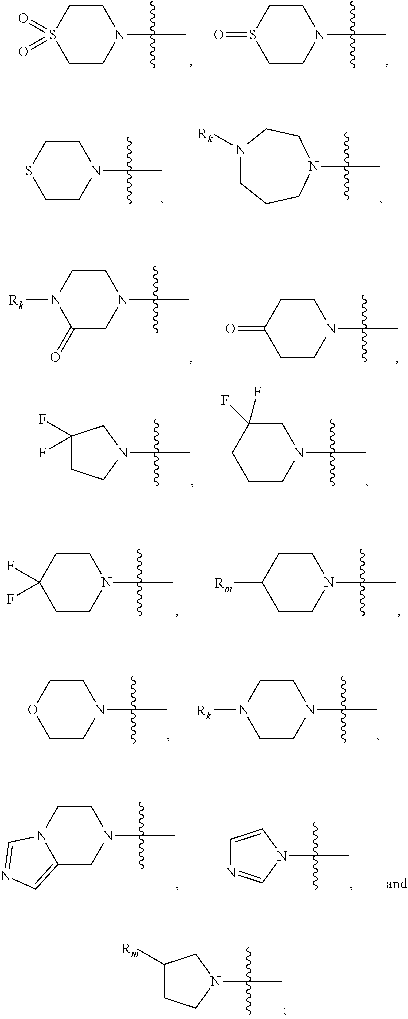

wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₆₎alkyl, COC₍₁₋₄₎alkyl, SO₂C₍₁₋₄₎alkyl, trifluoromethylpyridyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and
R_{z} is independently selected from the group consisting of H, C₍₁₋₆₎alkyl, OCH₃, F, Cl, Br, —CN, and CF₃; or if R⁶ is H, C₍₁₋₆₎alkyl, OCH₃, F, Cl, Br, —CN, or CF₃ then R_{z} may be selected from the group consisting of:

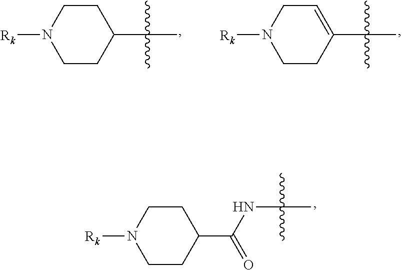

CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², CO₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R_{z}, may be optionally substituted with up to four methyl groups on two or more ring carbon atoms or optionally substituted with up to two CF₃ groups on any two ring carbon atoms;
and solvates, hydrates, tautomers, and pharmaceutically acceptable salts thereof.

Another embodiment of the invention is a pharmaceutical composition, comprising a compound of Formula I and a pharmaceutically acceptable carrier.

Another embodiment of the invention is a pharmaceutical composition, comprising a compound listed in the Examples section of this specification and a pharmaceutically acceptable carrier.

The present invention also provides a method for preventing, treating or ameliorating an MMP9 mediated syndrome, disorder or disease comprising administering to a subject in need thereof an effective amount of a compound of Formula I or a form, composition or medicament thereof.

The present invention also provides a method for preventing, treating or ameliorating an MMP13 mediated syndrome, disorder or disease comprising administering to a subject in need thereof an effective amount of a compound of Formula I or a form, composition or medicament thereof.

The present invention also provides a method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

The present invention also provides a method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

The present invention provides a method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

The present invention provides a method of preventing, treating or ameliorating a neoplastic disorder, wherein said neoplastic disorder is ovarian cancer, comprising administering to a subject in need thereof an effective amount of a compound of Formula I or a form, composition or medicament thereof.

The present invention provides a method of preventing, treating or ameliorating a cardiovascular disease, wherein said cardiovascular disease is selected from the group consisting of: atherosclerotic plaque rupture, aneurysm, vascular tissue morphogenesis, coronary artery disease, and myocardial tissue morphogenesis, comprising administering to a subject in need thereof an effective amount of a compound of Formula I or a form, composition or medicament thereof.

The present invention provides a method of preventing, treating or ameliorating atherosclerotic plaque rupture, comprising administering to a subject in need thereof an effective amount of a compound of Formula I or a form, composition or medicament thereof.

The present invention provides a method of preventing, treating or ameliorating rheumatoid arthritis, comprising administering to a subject in need thereof an effective amount of a compound of Formula I or a form, composition or medicament thereof.

The present invention provides a method of preventing, treating or ameliorating asthma, comprising administering to a subject in need thereof an effective amount of a compound of Formula I or a form, composition or medicament thereof.

The present invention provides a method of preventing, treating or ameliorating chronic obstructive pulmonary disease, comprising administering to a subject in need thereof an effective amount of a compound of Formula I or a form, composition or medicament thereof.

The present invention provides a method of preventing, treating or ameliorating inflammatory bowel syndrome, comprising administering to a subject in need thereof an effective amount of a compound of Formula I or a form, composition or medicament thereof.

The present invention provides a method of preventing, treating or ameliorating abdominal aortic aneurism, comprising administering to a subject in need thereof an effective amount of a compound of Formula I or a form, composition or medicament thereof.

The present invention provides a method of preventing, treating or ameliorating osteoarthritis, comprising administering to a subject in need thereof an effective amount of a compound of Formula I or a form, composition or medicament thereof.

The present invention provides a method of preventing, treating or ameliorating idiopathic pulmonary fibrosis, comprising administering to a subject in need thereof an effective amount of a compound of Formula I or a form, composition or medicament thereof.

The invention also relates to methods of inhibiting MMP9 activity in a mammal by administration of an effective amount of at least one compound of Formula I.

The invention also relates to methods of inhibiting MMP13 activity in a mammal by administration of an effective amount of at least one compound of Formula I.

In another embodiment, the invention relates to a compound as described in the Examples section for use as a medicament, in particular, for use as a medicament for treating a MMP9 mediated syndrome, disorder or disease.

In another embodiment, the invention relates to the use of a compound as described in the Examples section for the preparation of a medicament for the treatment of a disease associated with an elevated or inappropriate MMP9 activity.

In another embodiment, the invention relates to a compound as described in the Examples section for use as a medicament, in particular, for use as a medicament for treating a MMP13 mediated syndrome, disorder or disease.

In another embodiment, the invention relates to the use of a compound as described in the Examples section for the preparation of a medicament for the treatment of a disease associated with an elevated or inappropriate MMP13 activity.

### DEFINITIONS

The term “alkyl” refers to both linear and branched chain radicals of up to 12 carbon atoms, preferably up to 6 carbon atoms, unless otherwise indicated, and includes, but is not limited to, methyl, ethyl, propyl, isopropyl, butyl, isobutyl, sec-butyl, tert-butyl, pentyl, isopentyl, hexyl, isohexyl, heptyl, octyl, 2,2,4-trimethylpentyl, nonyl, decyl, undecyl and dodecyl. Any alkyl group may be optionally substituted with one OCH₃, one OH, or up to two fluorine atoms.

The term “alkenyl,” whether used alone or as part of a substituent group, for example, “C₍₁₋₄₎alkenyl(aryl),” refers to a partially unsaturated branched or straight chain monovalent hydrocarbon radical having at least one carbon-carbon double bond, whereby the double bond is derived by the removal of one hydrogen atom from each of two adjacent carbon atoms of a parent alkyl molecule and the radical is derived by the removal of one hydrogen atom from a single carbon atom. Atoms may be oriented about the double bond in either the cis (Z) or trans (E) conformation. Typical alkenyl radicals include, but are not limited to, ethenyl, propenyl, allyl (2-propenyl), butenyl and the like. Examples include C₍₂₋₈₎alkenyl or C₍₂₋₄₎alkenyl groups.

The term “alkoxy” refers to a saturated branched or straight chain monovalent hydrocarbon alcohol radical derived by the removal of the hydrogen atom from the hydroxide oxygen substituent on a parent alkane. Examples include C₍₁₋₆₎alkoxy or C₍₁₋₄₎alkoxy groups. Any alkoxy group may be optionally substituted with one OCH₃, one OH, or up to two fluorine atoms.

The term “C_{(a-b)}” (where a and b are integers referring to a designated number of carbon atoms) refers to an alkyl, alkenyl, alkynyl, alkoxy or cycloalkyl radical or to the alkyl portion of a radical in which alkyl appears as the prefix root containing from a to b carbon atoms inclusive. For example, C₍₁₋₄₎ denotes a radical containing 1, 2, 3 or 4 carbon atoms.

The term “cycloalkyl” refers to a saturated or partially unsaturated monocyclic or bicyclic hydrocarbon ring radical derived by the removal of one hydrogen atom from a single ring carbon atom. Typical cycloalkyl radicals include cyclopropyl, cyclobutyl, cyclopentyl, cyclopentenyl, cyclohexyl, cyclohexenyl, cycloheptyl and cyclooctyl. Additional examples include C₍₃₋₆₎cycloalkyl, C₍₅₋₈₎cycloalkyl, decahydronaphthalenyl, and 2,3,4,5,6,7-hexahydro-1H-indenyl. Any cycloalkyl group may be optionally substituted with one OCH₃, one OH, or up to two fluorine atoms.

### Abbreviations

Herein and throughout this application, the following abbreviations may be used.
Ac —C(O)CH₃
aq. aqueous
Bu butyl
[bmim][BF₄] 1-butyl-3-methylimidazolium tetrafluoroborate
conc. concentrated
d days
DCM dichloromethane
DMSO dimethylsulfoxide
DPPP 1,3-bis(diphenylphosphino)propane
eq equivalents
Et ethyl
g gram
h hours
HATU 2-(7-aza-1H-benzotriazole-1-yl)-1,1,3,3-tetramethyluronium hexafluorophosphate
hept heptanes
HPLC high pressure liquid chromatography
KHMDS ((CH₃)₃Si)₂NK
M molar
Me methyl
mL milliliter
mmol millimole
mg milligram
min minutes
N normal
NMR nuclear magnetic resonance
Pd(dppf)Cl_{2 [}1,1′-bis(diphenylphosphino)ferrocene]dichloropalladium(II)
Ph phenyl
iPr isopropyl
RP reverse phase
sat. saturated
TFA trifluoroacetic acid
THF tetrahydrofuran
OTs tosylate
TLC thin layer chromatography
TsOH para-toluenesulfonic acid
v volume

Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate (SEH), sodium hydroxide, triethanolamine or zinc.

### Methods of Use

The present invention is directed to a method for preventing, treating or ameliorating a MMP9 and/or MMP13 mediated syndrome, disorder or disease comprising administering to a subject in need thereof an effective amount of a compound of Formula I or a form, composition or medicament thereof.

Examples of a MMP9 and/or MMP13 mediated syndrome, disorder or disease for which the compounds of Formula I are useful include angiogenesis, osteoarthritis, rheumatoid arthritis, gastric ulcers, pulmonary hypertension, chronic obstructive pulmonary disorder, inflammatory bowel syndrome, periodontal disease, skin ulcers, liver fibrosis, emphysema, Marfan syndrome, stroke, multiple sclerosis, abdominal aortic aneurysm, coronary artery disease, idiopathic pulmonary fibrosis, renal fibrosis, migraine, and cardiovascular disorders including: atherosclerotic plaque, ruptive aneurysm, vascular tissue morphogenesis, and myocardial tissue morphogenesis.

The term “administering” with respect to the methods of the invention, means a method for therapeutically or prophylactically preventing, treating or ameliorating a syndrome, disorder or disease as described herein by using a compound of Formula I or a form, composition or medicament thereof. Such methods include administering an effective amount of said compound, compound form, composition or medicament at different times during the course of a therapy or concurrently in a combination form. The methods of the invention are to be understood as embracing all known therapeutic treatment regimens.

The term “subject” refers to a patient, which may be animal, typically a mammal, typically a human, which has been the object of treatment, observation or experiment. In one aspect of the invention, the subject is at risk of (or susceptible to) developing a syndrome, disorder or disease that is associated with elevated MMP9 and/or MMP13 expression or MMP9 and/or MMP13 overexpression, or a patient with an inflammatory condition that accompanies syndromes, disorders or diseases associated with elevated MMP9 and/or MMP13 expression or MMP9 and/or MMP13 overexpression.

The term “therapeutically effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating the symptoms of a syndrome, disorder or disease being treated.

When employed as inhibitors of pro-matrix metalloproteinase activation, the compounds of the invention may be administered in an effective amount within the dosage range of about 0.5 mg to about 10 g, preferably between about 0.5 mg to about 5 g, in single or divided daily doses. The dosage administered will be affected by factors such as the route of administration, the health, weight and age of the recipient, the frequency of the treatment and the presence of concurrent and unrelated treatments.

It is also apparent to one skilled in the art that the therapeutically effective dose for compounds of the present invention or a pharmaceutical composition thereof will vary according to the desired effect. Therefore, optimal dosages to be administered may be readily determined by one skilled in the art and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

The compounds of Formula I may be formulated into pharmaceutical compositions comprising any known pharmaceutically acceptable carriers. Exemplary carriers include, but are not limited to, any suitable solvents, dispersion media, coatings, antibacterial and antifungal agents and isotonic agents. Exemplary excipients that may also be components of the formulation include fillers, binders, disintegrating agents and lubricants.

The pharmaceutically-acceptable salts of the compounds of Formula I include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups may be quaternized with, for example, alkyl halides.

The pharmaceutical compositions of the invention may be administered by any means that accomplish their intended purpose. Examples include administration by parenteral, subcutaneous, intravenous, intramuscular, intraperitoneal, transdermal, buccal or ocular routes. Alternatively or concurrently, administration may be by the oral route. Suitable formulations for parenteral administration include aqueous solutions of the active compounds in water-soluble form, for example, water-soluble salts, acidic solutions, alkaline solutions, dextrose-water solutions, isotonic carbohydrate solutions and cyclodextrin inclusion complexes.

The present invention also encompasses a method of making a pharmaceutical composition comprising mixing a pharmaceutically acceptable carrier with any of the compounds of the present invention. Additionally, the present invention includes pharmaceutical compositions made by mixing a pharmaceutically acceptable carrier with any of the compounds of the present invention. As used herein, the term “composition” is intended to encompass a product comprising the specified ingredients in the specified amounts, as well as any product which results, directly or indirectly, from combinations of the specified ingredients in the specified amounts.

### Polymorphs and Solvates

Furthermore, the compounds of the present invention may have one or more polymorph or amorphous crystalline forms and as such are intended to be included in the scope of the invention. In addition, the compounds may form solvates, for example with water (i.e., hydrates) or common organic solvents. As used herein, the term “solvate” means a physical association of the compounds of the present invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include ethanolates, methanolates, and the like.

It is intended that the present invention include within its scope polymorphs and solvates of the compounds of the present invention. Thus, in the methods of treatment of the present invention, the term “administering” shall encompass the means for treating, ameliorating or preventing a syndrome, disorder or disease described herein with the compounds of the present invention or a polymorph or solvate thereof, which would obviously be included within the scope of the invention albeit not specifically disclosed.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

### General Schemes:

Compounds of formula I, can be prepared by methods known to those who are skilled in the art. The following reaction schemes are only meant to represent examples of the invention and are in no way meant to be a limit of the invention.

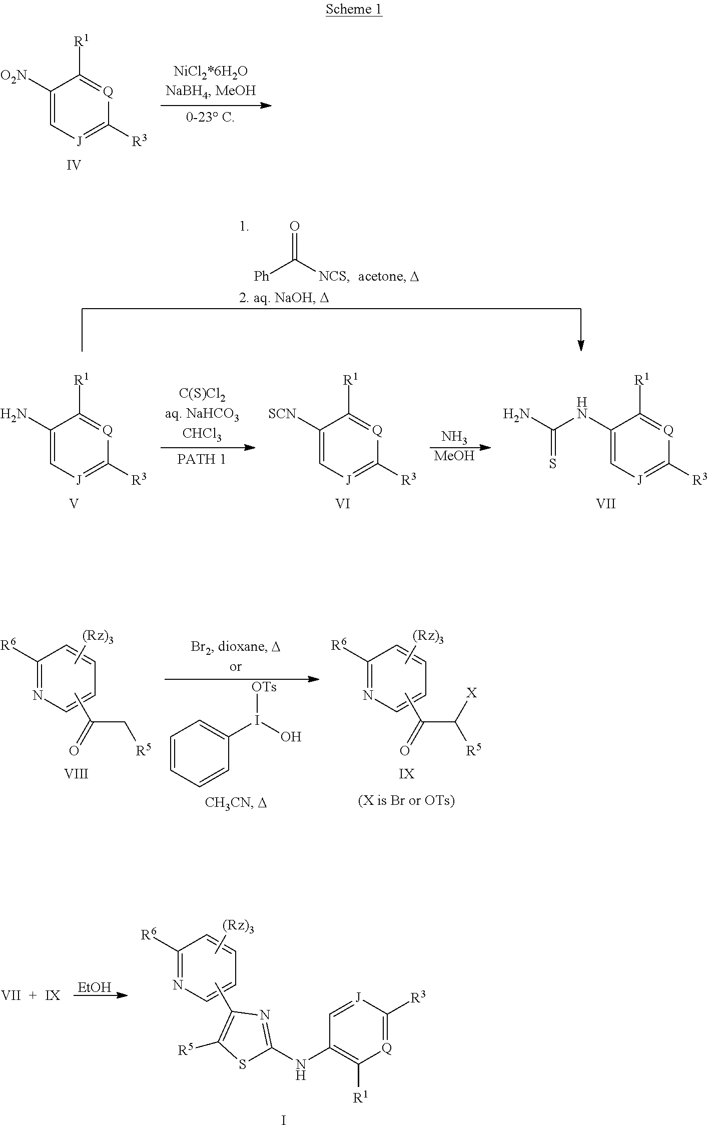

Scheme 1 illustrates synthetic routes leading to compounds of formula I. Thiourea VII may be prepared starting with nitro aromatic IV by reduction to the corresponding aniline V. The aniline V is converted to an isothiocyanate by reaction with thiophosgene and a base, and the isothiocyanate VI is treated with ammonia to provide thiourea VII (path 1). Alternatively, following path 2, aniline V can be converted to thiourea VII by reaction with benzoyl isothiocyanate, typically by heating to reflux in acetone, followed by hydrolysis under basic aqueous conditions. Pyridinyl ketone VIII is converted to IX (X is Br) by heating with bromine, or is reacted with hydroxyl(tosyloxy)-iodobenzene to form IX (X is OTs). Intermediate IX undergoes condensation with thiourea VII to afford compounds of formula I.

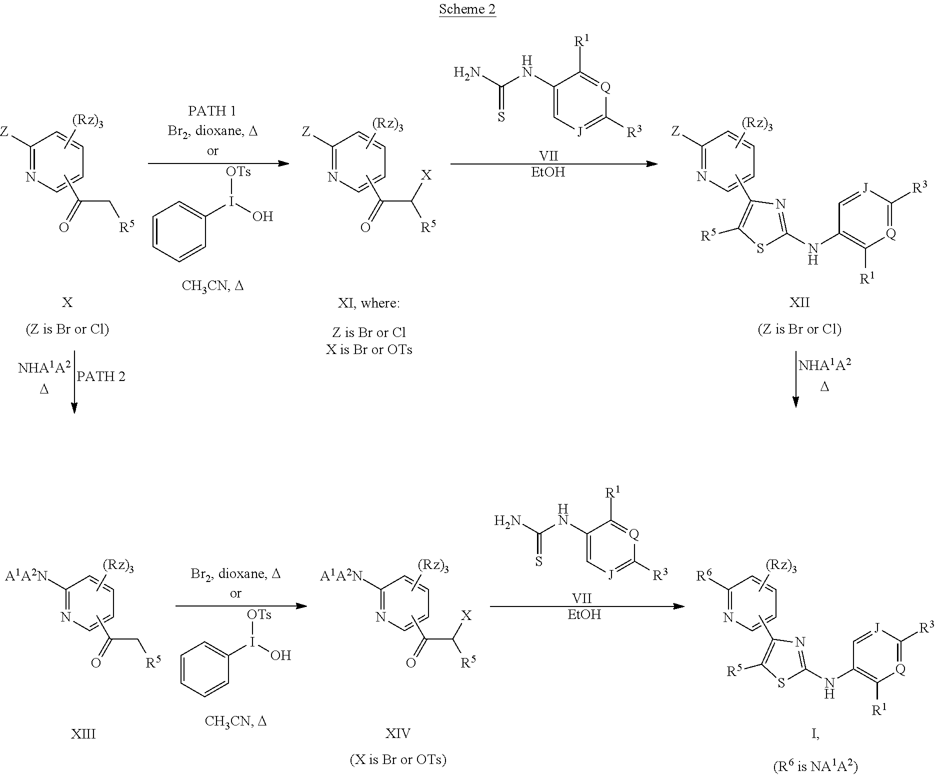

Synthetic routes to compounds of formula I where R⁶ is NA¹A² are illustrated in Scheme 2. Following path 1,2-bromo or 2-chloro pyridine X is converted to XI, which is reacted with thiourea VII to provide XII. Heating XII with an amine NHA¹A² yields compounds of formula I, where R⁶ is NA¹A². Alternatively, according to the route shown in path 2, 2-bromo or 2-chloro pyridine X is heated with an amine NHA¹A² to provide 2-amino pyridine XIII, which is converted to bromide or tosylate XIV and condensed with thiourea VII in analogy to the route shown in Scheme 1, to yield compounds of formula I, where R⁶ is NA¹A².

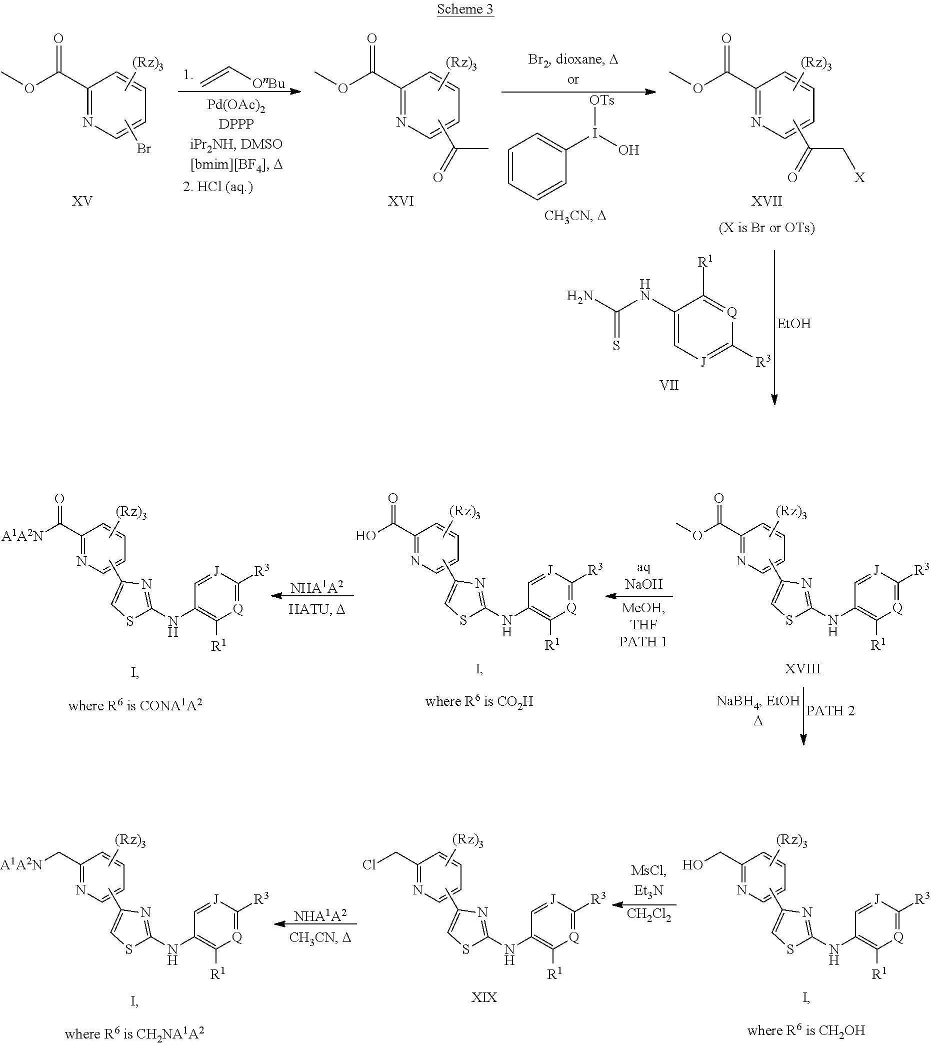

Scheme 3 illustrates methods of synthesis of compounds of formula I where R⁶ is CO₂H, CONA¹A², CH₂OH, or CH₂NA¹A² and R⁵ is H. Bromo-pyridine XV undergoes a Heck reaction with n-butoxy-ethene, yielding an enol ether intermediate, which provides ketone XVI on acidic aqueous hydrolysis (Tetrahedron 2005, 61, 9902). Treatment with bromine or hydroxyl(tosyloxy)-iodobenzene affords XVII, which is condensed with thiourea VII to afford thiazole XVIII. According to path 1, this intermediate may be hydrolyzed under basic aqueous conditions to afford compounds of formula I, where R⁶ is CO₂H. These compounds may be converted to compounds of formula I, where R⁶ is CONA¹A², by treatment with NHA¹A² and a peptide coupling reagent such as HATU. Alternatively, according to path 2, upon treatment with a reducing agent, such as NaBH₄, XVIII undergoes reduction to compounds of formula I, where R⁶ is CH₂OH and R⁵ is H. These compounds may be converted to chloride XIX, for instance by reaction with mesyl chloride and triethylamine in dichloromethane. Heating with amine NHA¹A² then affords compounds of formula I, where R⁶ is CH₂NA¹A².

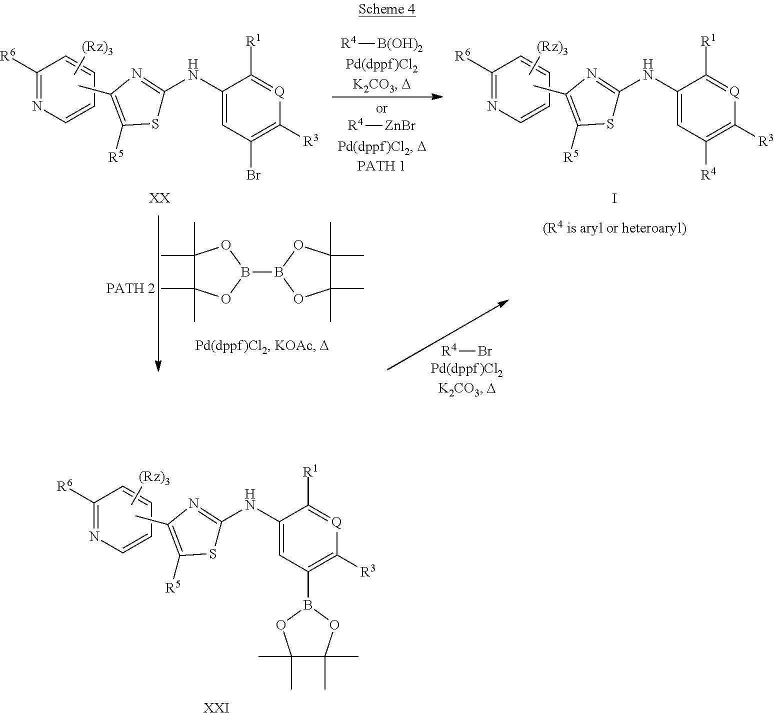

Additional routes to compounds of formula I where R⁴ is aryl or heteroaryl, are depicted in Scheme 4. An aryl bromide XX, prepared as described in Scheme 1, can react with a boronic acid (or ester), or a zinc reagent, in the presence of a palladium catalyst to yield compounds of formula I where R⁴ is aryl or heteroaryl, as shown in path 1. Alternatively, as path 2 illustrates, XX can be converted to the corresponding boronate ester XXI by treatment with bis(pinacolato)diboron and a palladium catalyst. The boronate ester XXI may be converted to compounds of formula I where R⁴ is aryl or heteroaryl, by reaction with an aryl or heteroaryl bromide under palladium catalyzed conditions.

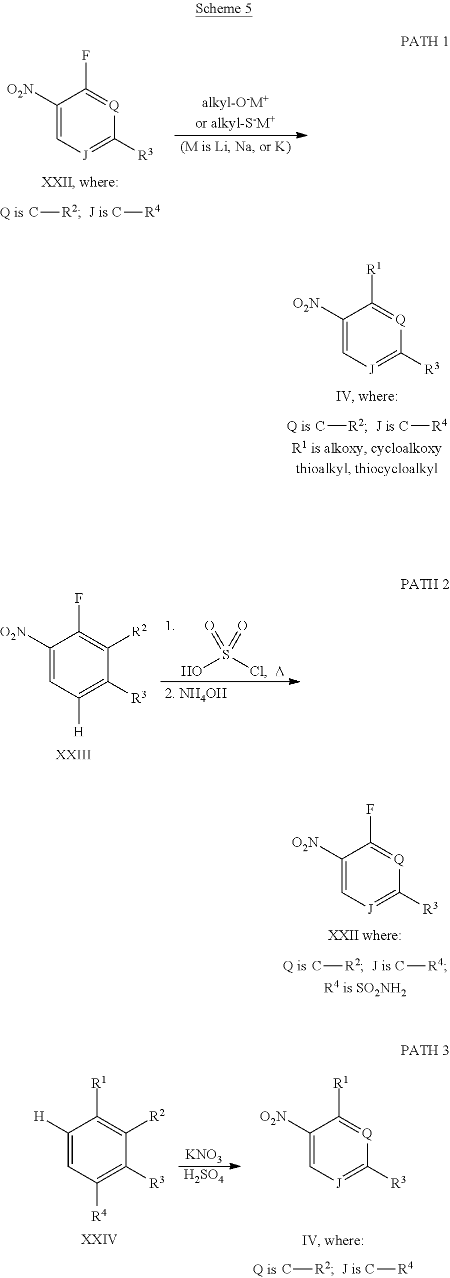

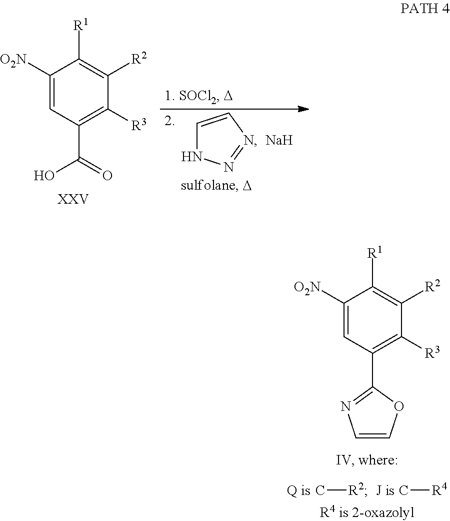

Many intermediates of formulae IV, V, VI, and VII (as used in Scheme 1) are commercially available. Scheme 5 illustrates synthetic routes (paths 1 to 4) to aryl nitro compounds of formula IV, which may be converted to compounds of formula I as described in Scheme 1. A 2-nitrofluoro benzene XXII can be reacted with a metal alkoxide or thiolate to yield IV, where R¹ is alkoxy, cycloalkoxy, thioalkyl, or thiocycloalkyl (path 1). As shown in path 2, in the case where R⁴ is SO₂NH₂, the required starting material XXII may be obtained by heating 2-fluoronitro benzene XXIII (unsubstituted para to the fluorine) in neat chlorosulfonic acid, typically at reflux, followed by treatment of the aryl sulfonyl chloride intermediate with ammonium hydroxide solution. Additional aryl nitro compounds IV may be obtained by treatment of substituted aryls XXIV with a nitrating reagent, such as KNO₃/H₂SO₄, HNO₃/H₂SO₄, or HNO₃/Ac₂O (path 3). Those skilled in the art will recognize that path 3 is preferably employed when nitration is desired to occur at a position ortho or para to electron-donating substituents, such as alkoxy or alkyl, and meta to electron-withdrawing substituents, such as CONH₂. As depicted in path 4, nitro compounds of formula IV, where R⁴ is 2-oxazolyl, may be prepared by conversion of aryl carboxylic acid XXV to the corresponding acid chloride, for instance by heating in thionyl chloride, followed by reaction with a metal salt of 1,2,3-1H-triazole.

### EXAMPLES

### Intermediate 1

### 2-Bromo-1-(2,4,6-trimethyl-pyridin-3-yl)-ethanone.HBr

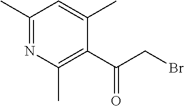

A suspension of bromine (0.04 mL, 0.778 mmol) in 1,4-dioxane (1 mL) was added to a stirred solution of commercially available 1-(2,4,6-trimethyl-pyridin-3-yl)-ethanone (134 mg, 0.819 mmol) in 1,4-dioxane (1 mL). The mixture was stirred at 50° C. for 24 h and the resulting cream-colored suspension was allowed to cool to room temperature and was filtered, washed with 2:1 heptane:EtOAc (v/v) and air dried to give the title compound.

### Intermediate 2

### 2-Bromo-1-(6-bromo-pyridin-3-yl)-ethanone.HBr

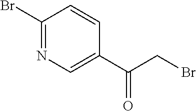

The title compound was prepared using 1-(6-bromo-pyridin-3-yl)-ethanone in place of 1-(2,4,6-trimethyl-pyridin-3-yl)-ethanone according to the procedure described in intermediate 1.

### Intermediate 3

### 2-Bromo-1-(5-bromo-pyridin-3-yl)-ethanone.HBr

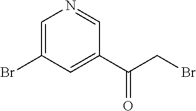

The title compound was prepared using 1-(5-bromo-pyridin-3-yl)-ethanone in place of 1-(2,4,6-trimethyl-pyridin-3-yl)-ethanone according to the procedure described in intermediate 1.

### Intermediate 4

### 2-Bromo-1-(4-isopropyl-pyridin-3-yl)-ethanone.HBr

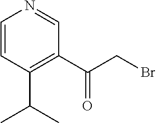

The title compound was prepared using 1-(4-isopropyl-pyridin-3-yl)-ethanone in place of 1-(2,4,6-trimethyl-pyridin-3-yl)-ethanone according to the procedure described in intermediate 1.

### Intermediate 5

### 2-Bromo-1-(2-methyl-6-trifluoromethyl-pyridin-3-yl)-ethanone.HBr

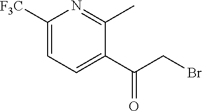

The title compound was prepared using 1-(2-methyl-6-trifluoromethyl-pyridin-3-yl)-ethanone in place of 1-(2,4,6-trimethyl-pyridin-3-yl)-ethanone according to the procedure described in intermediate 1.

### Intermediate 6

### 2-Bromo-1-(6-methoxy-pyridin-3-yl)-ethanone.HBr

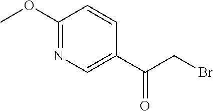

The title compound was prepared using 1-(6-methoxy-pyridin-3-yl)-ethanone in place of 1-(2,4,6-trimethyl-pyridin-3-yl)-ethanone according to the procedure described in intermediate 1.

### Intermediate 7

Toluene-4-sulfonic acid 2-(6-chloro-pyridin-3-yl)-2-oxo-ethyl ester

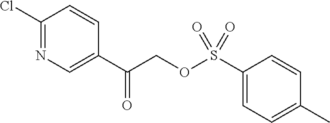

A mixture of 1-(6-chloro-pyridin-3-yl)-ethanone (300 mg, 1.93 mmol) and hydroxyl(tosyloxy)-iodobenzene (833 mg, 2.12 mmol) in CH₃CN was heated at 85° C. for 5 h and cooled. To the reaction mixture was added silica gel (300 mesh, ˜5 g), and the suspension was concentrated and purified through solid loading on column chromatography (30%-50% EtOAc/heptane) to yield the title compound as a white solid.

### Intermediate 8: Step a

### 1-[6-(4-Methyl-piperazin-1-yl)-pyridin-3-yl]-ethanone

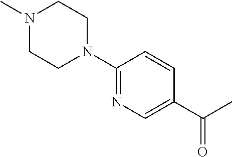

A mixture of 1-(6-chloro-pyridin-3-yl)-ethanone (515 mg, 3.31 mmol) and 1-methyl piperazine (10 mL) was heated at 100° C. for 4 h. The residue was partitioned between water and CH₂Cl₂, the organic layer was dried (Na₂SO₄), filtered, concentrated, and purified through column chromatography (3-10% MeOH/DCM) to yield the title compound as a white solid.

### Intermediate 8: Step b

### 2-Bromo-1-[6-(4-methyl-piperazin-1-yl)-pyridin-3-yl]-ethanone.HBr

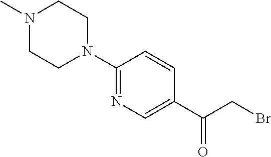

To 1-[6-(4-methyl-piperazin-1-yl)-pyridin-3-yl]-ethanone (610 mg, 2.78 mmol, intermediate 8, step a) in 48% HBr (6 mL) at 60° C. was added 0.778 M Br₂ in 1,4-dioxane (3.22 mL, 2.504 mmol). The mixture was stirred for 18 h at 60° C. and the resulting cream-colored suspension was allowed to cool to room temperature and was filtered, washed with 2:1 heptane:EtOAc (v/v) and air dried to give the title compound.

### Intermediate 9

### 3-[4-(2-Chloro-pyridin-4-yl)-thiazol-2-ylamino]-4-isopropoxy-benzenesulfonamide.TFA

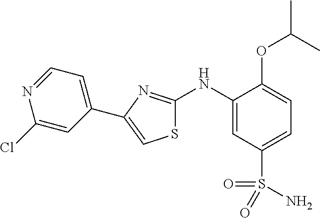

The title compound was prepared using commercially available 2-bromo-1-(2-chloro-pyridin-4-yl)-ethanone in place of 2-bromo-1-(2,4,6-trimethyl-pyridin-3-yl)-ethanone.HBr and 4-isopropoxy-3-thioureido-benzenesulfonamide (intermediate 26, step e) in place of 4-isopropoxy-3-thioureido-benzamide according to the procedure described in example 19.

### Intermediate 10

### 3-[4-(6-Chloro-pyridin-3-yl)-thiazol-2-ylamino]-4-isopropoxy-benzenesulfonamide.TsOH

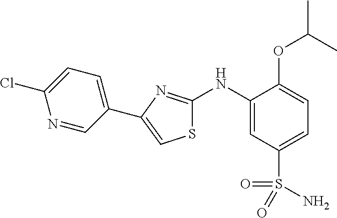

A mixture of toluene-4-sulfonic acid 2-(6-chloro-pyridin-3-yl)-2-oxo-ethyl ester (400 mg, 1.23 mmol, intermediate 7) and 4-isopropoxy-3-thioureido-benzenesulfonamide (355 mg, 1.23 mmol, intermediate 26, step e) in EtOH (10 mL) was stirred at room temperature for 48 h. The mixture was filtered and the solid was washed with EtOAc and air dried to afford the title compound as a white solid.

### Intermediate 11: Step a

### 5-Acetyl-pyridine-2-carboxylic acid methyl ester

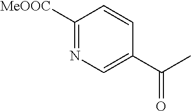

Following the method described in Tetrahedron 2005, 61, 9902, a mixture of 5-bromo-pyridine-2-carboxylic acid methyl ester (3 g, 13.9 mmol), butoxy-ethene (3.65 mL, 27.8 mmol), Pd(OAc)₂ (374 mg, 0.555 mmol), DPPP (458 mg, 1.11 mmol), and iPr₂NH (2.36 mL, 16.7 mmol) in 1-butyl-3-methylimidazolium tetrafluoroborate (1.5 mL) and DMSO (30 mL) was degassed under N₂ for 10 min and then heated at 117° C. for 6 h. After cooling, 2 N HCl (30 mL) was added and the mixture was stirred for 20 h. The reaction mixture was basified with 2 N Na₂CO₃ and extracted with dichloromethane. The organic layer was dried (Na₂SO₄), filtered, concentrated and purified through column chromatography to afford the title compound as a thick oil.

### Intermediate 11: Step b

### 5-[2-(Toluene-4-sulfonyloxy)-acetyl]-pyridine-2-carboxylic acid methyl ester

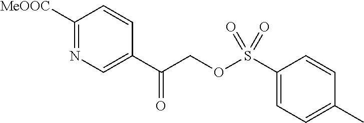

The title compound was prepared using 5-acetyl-pyridine-2-carboxylic acid methyl ester (intermediate 11, step a) in place of 1-(6-chloro-pyridin-3-yl)-ethanone according to the procedure described in intermediate 7.

### Intermediate 12

### 5-[2-(2-Isopropoxy-5-sulfamoyl-phenylamino)-thiazol-4-yl]-pyridine-2-carboxylic acid methyl ester.TsOH

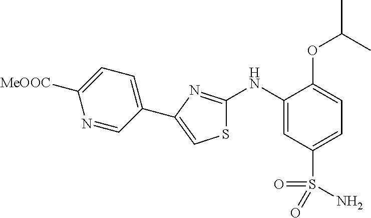

The title compound was prepared using 5-[2-(toluene-4-sulfonyloxy)-acetyl]-pyridine-2-carboxylic acid methyl ester (intermediate 11, step b) in place of toluene-4-sulfonic acid 246-chloro-pyridin-3-yl)-2-oxo-ethyl ester according to the procedure described in intermediate 10.

### Intermediate 13: Step a

### 4-Fluoro-3-nitro-benzamide

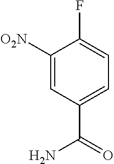

A round bottom flask fitted with a reflux condenser vented through an aqueous sodium hydroxide solution was charged with 4-fluoro-3-nitro-benzoic acid (5.0 g, 27.0 mmol, Aldrich). Thionyl chloride (20 mL) was added and the resulting suspension was heated in an 80° C. oil bath for 3 h. The mixture was concentrated and the residual oil was dissolved in THF (20 mL) and added slowly via pipette to an ice-cold solution of concentrated aqueous NH₄OH (20 mL). The resulting bright yellow mixture was stirred at 0° C. for 35 min. The mixture was partially concentrated to remove THF and the residual solution was extracted with EtOAc. The organic phase was dried (Na₂SO₄), filtered, and concentrated. The residue was purified by flash column chromatography (Silica gel, 1-3% EtOH—CH₂Cl₂) to afford the title compound as a white solid.

### Intermediate 13: Step b

### 4-Isopropoxy-3-nitro-benzamide

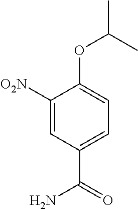

To a solution of iPrOH (0.619 mL, 8.09 mmol) in THF (25 mL) at 0° C., added a 0.5 M solution of KHMDS in toluene (16.2 mL, 8.09 mmol) followed by 4-fluoro-3-nitro-benzamide (993 mg, 5.39 mmol, intermediate 13, step a). The resulting brown suspension was stirred at 0° C. for 1 h, then was allowed to warm to 23° C. and was stirred for an additional 4 h. The mixture was partially concentrated to remove THF and was diluted with water and extracted with EtOAc. The organic phase was dried (Na₂SO₄), filtered, and concentrated, affording the crude title compound as an orange solid which was used without further purification in the next reaction.

### Intermediate 13: Step c

### 3-Amino-4-isopropoxy-benzamide

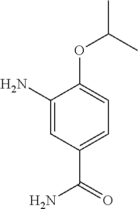

Sodium borohydride (250 mg, 6.60 mmol) was added slowly to a solution of nickel (II) chloride hexahydrate (567 mg, 2.20 mmol) in MeOH (30 mL) at 0° C. and the resulting black suspension was stirred for 30 min at 23° C. The mixture was cooled to 0° C. and to it was added a suspension of crude 4-isopropoxy-3-nitro-benzamide (0.987 g, 4.40 mmol, intermediate 13, step b) in MeOH (20 mL), followed by sodium borohydride (583 mg, 15.4 mmol). The mixture was stirred for 1 hour at 23° C. The mixture was partially concentrated to remove most of the MeOH, water was added to quench excess NaBH₄, and the mixture was partitioned between EtOAc and water. The aqueous phase was extracted with EtOAc. The organic phase was dried (Na₂SO₄), filtered, and concentrated. The residue was purified by flash column chromatography (Silica gel, 1-6% MeOH—CH₂Cl₂), yielding the title compound as a white powder.

### Intermediate 13: Step d

### 4-Isopropoxy-3-isothiocyanato-benzamide

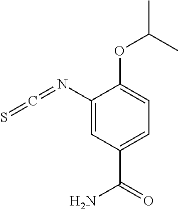

A solution of sodium bicarbonate (645 mg, 7.68 mmol) in water (15 mL) was added to 3-amino-4-isopropoxy-benzamide (497 mg, 2.56 mmol, intermediate 13, step c) in a mixture of chloroform (15 mL) and water (15 mL). Thiophosgene (0.206 mL, 2.69 mmol) was then added. The biphasic solution was stirred at room temperature for 2.5 h. TLC analysis indicated slight remaining starting material, so an additional 0.030 mL portion of thiophosgene was added and the mixture was stirred for 40 min. The phases were separated and the aqueous phase was extracted with CH₂Cl₂. The organic phase was dried (Na₂SO₄), filtered, and concentrated, yielding the crude title compound as an off-white solid.

### Intermediate 13: Step e

### 4-Isopropoxy-3-thioureido-benzamide

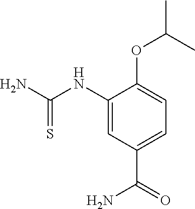

Crude 4-isopropoxy-3-isothiocyanato-benzamide (608 mg, intermediate 13, step d) was suspended in MeOH (2 mL). A 2 M solution of ammonia in MeOH (2 mL) was added and the resulting yellow solution was stirred at room temperature for 16 h. The reaction mixture was concentrated and the residue was purified by flash column chromatography (3-8% MeOH—CH₂Cl₂), affording the title compound as a white powder.

### Intermediate 14

### 4-Methoxy-3-thioureido-benzamide

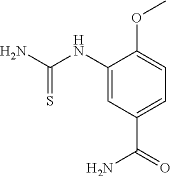

To a solution of 3-amino-4-methoxybenzamide (2.49 g, 15.0 mmol, Alfa) in acetone (30 mL) at reflux was added benzoyl isothiocyanate (2.22 mL, 16.5 mmol) and the mixture was stirred at reflux for 30 min, then was poured into water. The precipitate was collected by vacuum filtration and was treated with 10% aq. NaOH (15 mL). The mixture was refluxed for 40 min, was cooled to room temperature, and was poured into a mixture of ice and 6 N aq. HCl. The mixture was basified to pH 10 with conc. aq. NH₄OH and the resulting white solid precipitate was collected by vacuum filtration, affording the crude title compound, which was used without further purification.

### Intermediate 15: Step a

### 4-Ethoxy-3-nitro-benzamide

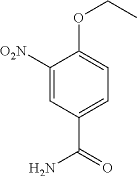

A mixture of 4-fluoro-3-nitro-benzamide (1.18 g, 6.39 mmol, intermediate 13, step a) and EtOH (5 mL) was treated with a solution of sodium ethoxide in EtOH (21 wt. %, 4.77 mL, 12.78 mmol, Aldrich) and the resulting brown suspension was stirred at room temperature for 15 min. The mixture was partitioned between EtOAc and water and the aqueous phase was extracted with EtOAc. The organic phase was dried (Na₂SO₄), filtered, and concentrated and the residue was purified by flash column chromatography (Silica gel, 60-100% EtOAc-Hept) affording the title compound as a yellow powder.

### Intermediate 15: Step b

### 3-Amino-4-ethoxy-benzamide

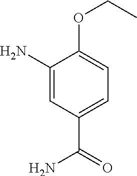

The title compound was prepared using 4-ethoxy-3-nitro-benzamide (intermediate 15, step a) in place of 4-isopropoxy-3-nitro-benzamide using the procedure described for intermediate 13, step c.

### Intermediate 15: Step c

### 4-Ethoxy-3-thioureido-benzamide

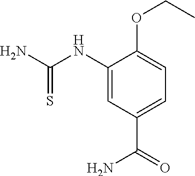

To a suspension of 3-amino-4-ethoxy-benzamide (597 mg, 3.31 mmol, intermediate 15, step b) in acetone (10 mL), was added benzoyl isothiocyanate (0.490 mL, 3.64 mmol) and the resulting white suspension was heated to reflux for 20 min. The mixture was poured into water and the white solid precipitate was collected by vacuum filtration. The solid was heated with 10% aq. NaOH solution (15 mL) in a 90° C. oil bath for 25 min. The reaction mixture was diluted with water and was extracted with EtOAc. The organic phase was dried (Na₂SO₄), filtered, and concentrated. The residue was purified by flash column chromatography (Silica gel, 1-6% MeOH—CH₂Cl₂), yielding the title compound as a white powder.

### Intermediate 16

### (2-Methyl-benzofuran-7-yl)-thiourea

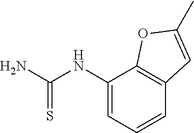

2-Methyl-benzofuran-7-ylamine.HCl (Chembridge) was partitioned between sat. aq. NaHCO₃ and CH₂Cl₂ to convert it to the corresponding free base, and the organic phase was dried (Na₂SO₄), filtered, and concentrated. The resulting brown oil (768 mg, 5.22 mmol) was heated to reflux in acetone (30 mL) and benzoyl isothiocyanate (0.772 mL, 5.74 mmol) was slowly added. The mixture was stirred at reflux for 30 min, was poured into ice water, and the precipitated solid was collected by vacuum filtration, and washed with water. The solid was stirred at reflux in 10% aq. NaOH (15 mL) for 40 min. The reaction mixture was poured into a mixture of ice and 6 N aq. HCl, resulting in formation of a white precipitate. The mixture was basified to pH 10 by addition of conc. aq. NH₄OH and was filtered. The title compound was isolated as a white solid by column chromatography (Silica gel, 20-60% EtOAc-Hept).

### Intermediate 17: Step a

### 3-Nitro-4-trifluoromethoxy-benzamide

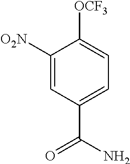

To concentrated aqueous sulfuric acid (3 mL) was slowly added 90% aqueous nitric acid (3 mL) and the resulting solution was cooled in an ice-bath. Solid 4-trifluoromethoxy-benzamide (1.0 g, 4.88 mmol, Alfa) was slowly added and the reaction mixture was stirred at room temperature for 10 min, then was poured into a stirred ice/water mixture. The white precipitate was collected by vacuum filtration and washed with water, affording the crude title compound, which was used without further purification.

### Intermediate 17: Step b

### 3-Amino-4-trifluoromethoxy-benzamide

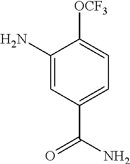

To a solution of nickel(II) chloride hexahydrate (470 mg, 1.98 mmol) in MeOH (10 mL) at 0° C. was slowly added sodium borohydride (225 mg, 5.94 mmol) (caution: gas evolution). The resulting black suspension was stirred at room temperature for 30 min, then was cooled to 0° C. before addition of crude 3-nitro-4-trifluoromethoxy-benzamide (0.99 g, 3.96 mmol, intermediate 17, step a) and a second portion of sodium borohydride (524 mg, 13.9 mmol). The resulting black suspension was stirred at room temperature for 0.5 h before addition of a small amount of water to quench remaining borohydride. The mixture was diluted with sat. aq. NaHCO₃ and extracted with CH₂Cl₂. To facilitate extraction of the polar product, the aqueous phase was saturated with NaCl, then was further extracted with CH₂Cl₂. The organic phase was washed with saturated aqueous NaCl and was dried (Na₂SO₄), filtered, and concentrated. The residual white solid was purified by flash column chromatography (Silica gel, 20-100% EtOAc-Hept), affording the title compound as an off-white solid.

### Intermediate 17: Step c

### 3-Isothiocyanato-4-trifluoromethoxy-benzamide

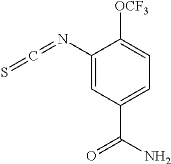

The title compound was prepared using 3-amino-4-trifluoromethoxy-benzamide (intermediate 17, step b) in place of 3-amino-4-isopropoxy-benzamide by the procedure described for intermediate 13, step d, affording the crude title compound as a white solid which was used without purification.

### Intermediate 17: Step d

### 3-Thioureido-4-trifluoromethoxy-benzamide

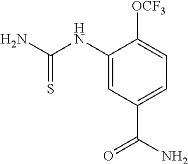

Crude 3-isothiocyanato-4-trifluoromethoxy-benzamide (444 mg, 1.69 mmol, intermediate 17, step c) was treated with a solution of ammonia in methanol (2 M, 10 mL, 20 mmol) and the resulting yellow solution was stirred at 40° C. for 30 min. The reaction mixture was concentrated onto Silica gel for purification by column chromatography (Silica gel, 20-100% EtOAc-Hept), which afforded the title compound as a white solid.

### Intermediate 18: Step a

### 2-(4-Methoxy-3-nitro-phenyl)-acetamide

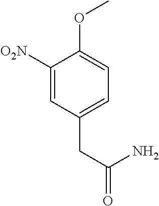

Nitric acid (90%, 6 mL) was added to an ice-cold suspension of (4-methoxy-phenyl)-acetic acid (3.02 g, 18.2 mmol, Aldrich) in acetic anhydride (20 mL). The resulting brown solution was stirred at 0° C. for 15 min, then at room temperature for 15 min, then was poured into ice. When melted, the mixture was extracted with CH₂Cl₂. The organic phase was dried (Na₂SO₄), filtered, and concentrated and the residue was re-concentrated from toluene, affording a brown oil. The crude oil was heated at 80° C. in thionyl chloride (15 mL) for 1 h. The reaction mixture was concentrated, and the residue was dissolved in THF (15 mL) and added to ice-cold concentrated aq. NH₄OH (15 mL). The resulting mixture was stirred at 0° C. for 30 min, was partially concentrated to remove THF, was diluted with water, and was extracted with EtOAc. The organic phase was dried (Na₂SO₄), filtered, and concentrated. The residue was purified by flash column chromatography (Silica gel, 75-100% EtOAc-Hept; then 0-3% MeOH-EtOAc), affording the title compound as a yellow powder.

### Intermediate 18: Step b

### 2-(3-Amino-4-methoxy-phenyl)-acetamide

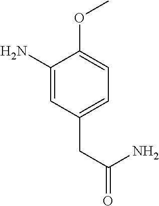

The title compound was prepared using 2-(4-methoxy-3-nitro-phenyl)-acetamide (intermediate 18, step a) in place of 4-isopropoxy-3-nitro-benzamide according to the procedure described for intermediate 13, step c (reaction time 15 min after addition of 2-(4-methoxy-3-nitro-phenyl)-acetamide), except that the crude product was used in the next reaction.

### Intermediate 18: Step c

### 2-(3-Isothiocyanato-4-methoxy-phenyl)-acetamide

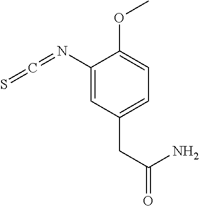

The title compound was prepared using 2-(3-amino-4-methoxy-phenyl)-acetamide (intermediate 18, step b) in place of 3-amino-4-isopropoxy-benzamide according to the procedure described for intermediate 13, step d.

### Intermediate 18: Step d

### 2-(4-Methoxy-3-thioureido-phenyl)-acetamide

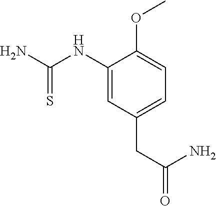

Crude 2-(3-isothiocyanato-4-methoxy-phenyl)-acetamide (369 mg, 1.66 mmol, intermediate 18, step c) was treated with a solution of ammonia in methanol (2 M, 10 mL, 20 mmol) and the resulting mixture was stirred at 40° C. for 40 min. The reaction mixture was allowed to cool to 23° C. and was filtered to collect the title compound as an off-white solid.

### Intermediate 19: Step a

### 3-Methoxy-4-nitro-benzamide

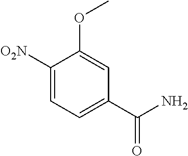

A round bottom flask fitted with a reflux condenser vented through an aqueous sodium hydroxide solution was charged with 3-methoxy-4-nitro-benzoic acid (3.07 g, 15.6 mmol, Aldrich). Thionyl chloride (10 mL) was added and the resulting suspension was heated in an 80° C. oil bath for 30 min. The mixture was concentrated and the residue was dissolved in THF (10 mL) and added slowly via pipette to an ice-cold solution of concentrated aqueous NH₄OH (10 mL). The resulting yellow suspension was stirred at 0° C. for 30 min. The suspension was partially concentrated to remove THF and was filtered to afford the crude title compound as a light yellow solid.

### Intermediate 19: Step b

### 4-Amino-3-methoxy-benzamide

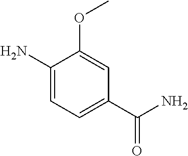

Sodium borohydride (0.764 g, 20.2 mmol) was added slowly to a solution of nickel (II) chloride hexahydrate (1.60 g, 6.73 mmol) in MeOH (25 mL) at 0° C. and the resulting black suspension was stirred for 20 min at 23° C. The mixture was cooled to 0° C. and to it was added crude 3-methoxy-4-nitro-benzamide (2.64 g, 13.5 mmol, intermediate 19, step a) followed by sodium borohydride (1.78 g, 47.1 mmol). The mixture was stirred for 30 min at 23° C. A small amount of water was added to quench excess NaBH₄ and the mixture was diluted with sat. aq. NaHCO₃ and was filtered through Celite. The filtrate was extracted with EtOAc. The organic phase was dried (Na₂SO₄), filtered, and concentrated, affording the crude title compound as an off-white solid.

### Intermediate 19: Step c

### 4-Isothiocyanato-3-methoxy-benzamide

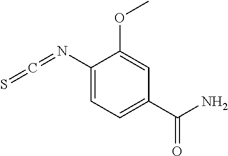

The title compound was prepared using 4-amino-3-methoxy-benzamide (intermediate 19, step b) in place of 3-amino-4-isopropoxy-benzamide according to the procedure described for intermediate 13, step d.

### Intermediate 19: Step d

### 3-Methoxy-4-thioureido-benzamide

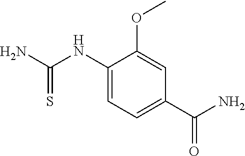

The title compound was prepared using 4-isothiocyanato-3-methoxy-benzamide (intermediate 19, step c) in place of 4-isopropoxy-3-isothiocyanato-benzamide according to the procedure described for intermediate 13, step e. The title compound was obtained as a white solid by filtration of the reaction mixture.

### Intermediate 20: Step a

### 4-Methoxy-2-methyl-benzamide

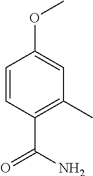

The title compound was prepared as a white powder using 4-methoxy-2-methyl-benzoic acid (Aldrich) in place of 3-methoxy-4-nitro-benzoic acid according to the procedure of intermediate 19, step a.

### Intermediate 20: Step b

### 4-Methoxy-2-methyl-5-nitro-benzamide

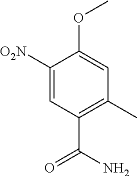

4-Methoxy-2-methyl-benzamide (7.75 g, 46.9 mmol, intermediate 20, step a) was cooled to 0° C. and concentrated sulfuric acid (40 mL) was added followed by potassium nitrate (4.74 g, 46.9 mmol) and the resulting brown suspension was stirred at room temperature for 40 min, then was slowly added to ice. The cream-colored precipitate was collected by vacuum filtration. The solid was dissolved in a mixture of THF and CH₂Cl₂ and was dried over Na₂SO₄, filtered, and concentrated, affording the crude title compound as a white solid.

### Intermediate 20: Step c

### 5-Amino-4-methoxy-2-methyl-benzamide

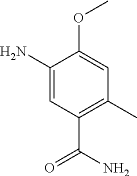

The title compound was prepared using 4-methoxy-2-methyl-5-nitro-benzamide (intermediate 20, step b) in place of 3-methoxy-4-nitro-benzamide according to the procedure of intermediate 19, step b. The crude product was purified by column chromatography (Silica gel, 0-7.5% MeOH—CH₂Cl₂), affording the title compound as a cream-colored solid.

### Intermediate 20: Step d

### 5-Isothiocyanato-4-methoxy-2-methyl-benzamide

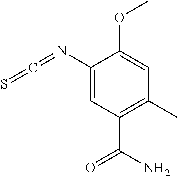

The title compound was prepared using 5-amino-4-methoxy-2-methyl-benzamide (intermediate 20, step c) in place of 3-amino-4-isopropoxy-benzamide according to the procedure described for intermediate 13, step d. During the extraction, precipitated solid made separation of the phases difficult; the solid was collected by vacuum filtration and was combined with the organic extracts. The crude title compound was obtained as a cream colored solid.

### Intermediate 20: Step e

### 4-Methoxy-2-methyl-5-thioureido-benzamide

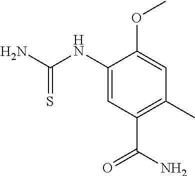

The title compound was prepared using 5-isothiocyanato-4-methoxy-2-methyl-benzamide (intermediate 20, step d) in place of 4-isopropoxy-3-isothiocyanato-benzamide according to the procedure described for intermediate 13, step e. The reaction mixture was concentrated to approximately half its original volume and cooled to 0° C., causing precipitation. The precipitated tan crystalline solid was collected by vacuum filtration and washed with MeOH to afford the title compound.

### Intermediate 21: Step a

### 4-Isobutyl-benzamide

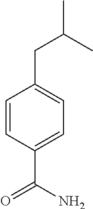

The title compound was prepared as a white powder using 4-isobutyl-benzoic acid (TCI) in place of 3-methoxy-4-nitro-benzoic acid according to the procedure of intermediate 19, step a.

### Intermediate 21: Step b

### 4-Isobutyl-3-nitro-benzamide

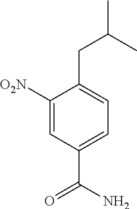

The title compound was prepared using 4-isobutyl-benzamide (intermediate 21, step a) in place of 4-methoxy-2-methyl-benzamide according to the procedure of intermediate 20, step b.

### Intermediate 21: Step c

### 3-Amino-4-isobutyl-benzamide

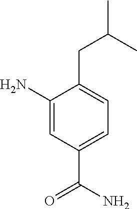

The title compound was prepared using 4-isobutyl-3-nitro-benzamide (intermediate 21, step b) in place of 3-methoxy-4-nitro-benzamide according to the procedure of intermediate 19, step b.

### Intermediate 21: Step d

### 4-Isobutyl-3-isothiocyanato-benzamide

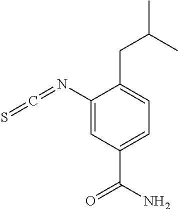

The title compound was prepared using 3-amino-4-isobutyl-benzamide (intermediate 21, step c) in place of 3-amino-4-isopropoxy-benzamide according to the procedure described for intermediate 13, step d. (The reaction was monitored by TLC and several additional portions of thiophosgene were added until the reaction approached complete conversion).

### Intermediate 21: Step e

### 4-Isobutyl-3-thioureido-benzamide

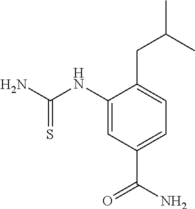

The title compound was prepared using 4-isobutyl-3-isothiocyanato-benzamide (intermediate 21, step d) in place of 4-isopropoxy-3-isothiocyanato-benzamide according to the procedure described for intermediate 13, step e. The reaction mixture was concentrated to approximately half its original volume and cooled to 0° C., causing precipitation. The precipitated white solid was collected by vacuum filtration and washed with MeOH to afford the title compound.

### Intermediate 22: Step a

### 3-Amino-4-methoxy-benzonitrile

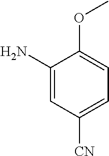

Sodium borohydride (0.159 g, 4.21 mmol) was added slowly to a solution of nickel (II) chloride hexahydrate (0.361 g, 1.40 mmol) in methanol (20 mL) at room temperature and stirred for 0.5 hours. To the resulting solution was added commercially available 4-methoxy-3-nitro-benzonitrile (0.500 g, 2.81 mmol) in methanol (10 mL), followed by sodium borohydride (0.372 g, 9.80 mmol) and stirred for 0.5 hours. The solution was then filtered through celite, and evaporated. Water was added and the crude product was extracted with ethyl acetate, dried with sodium sulfate and purified via column chromatography with heptanes: ethyl acetate to give the title compound.

### Intermediate 22: Step b

### 3-Isothiocyanato-4-methoxy-benzonitrile

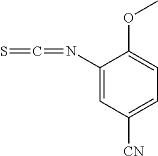

Thiophosgene (0.112 mL, 1.46 mmol) was added to a solution of 3-amino-4-methoxy-benzonitrile (0.216 g, 1.46 mmol, intermediate 22, step a) and sodium bicarbonate (0.368 g, 4.38 mmol) in chloroform (1 mL) and water (10 mL) and stirred at room temperature overnight. An additional 0.1 eq of thiophosgene was added to the reaction mixture and stirred for several hours. Excess ethyl acetate was added and the product was extracted, dried with sodium sulfate and purified via column chromatography with heptanes: ethyl acetate to give the title compound.

### Intermediate 22: Step c

### (5-Cyano-2-methoxy-phenyl)-thiourea

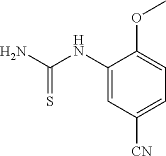

3-Isothiocyanato-4-methoxy-benzonitrile (0.140 g, 0.737 mmol, intermediate 22, step b) was dissolved in a solution of ammonia (2.0 M in methanol, 0.737 mL) and the reaction was heated in a sealed tube at 50° C. for 2 hours. The reaction mixture was then cooled to room temperature and filtered to give the title compound as a solid.

### Intermediate 23: Step a

### 1-Isopropoxy-4-methanesulfonyl-2-nitro-benzene

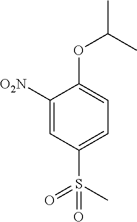

A solution of KHMDS (0.5 M in toluene, 13.68 mL, 6.84 mmol) was added dropwise to a solution of isopropanol (0.52 mL, 6.84 mmol) and THF (30 mL) at 0° C. and stirred for 10 minutes. The resulting solution was added to a solution of commercially available 4-methylsulfonyl-2-nitrofluorobenzene (1.00 g, 4.56 mmol) in THF (10 mL) at 0° C. and stirred for 0.5 hours. Water was added and the product was extracted with ethyl acetate, dried with sodium sulfate and evaporated to give the title compound.

### Intermediate 23: Step b

### 2-Isopropoxy-5-methanesulfonyl-phenylamine

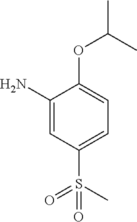

The title compound was prepared using 1-isopropoxy-4-methanesulfonyl-2-nitro-benzene (intermediate 23, step a) in place of 4-methoxy-3-nitro-benzonitrile, according to the procedure described in intermediate 22, step a, except that column chromatography was not done.

### Intermediate 23: Step c

### 1-Isopropoxy-2-isothiocyanato-4-methanesulfonyl-benzene

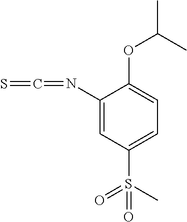

The title compound was prepared using 2-isopropoxy-5-methanesulfonyl-phenylamine (intermediate 23, step b) in place of 3-amino-4-methoxy-benzonitrile, according to the procedure described for intermediate 22, step b, except that column chromatography was not done.

### Intermediate 23: Step d

### (2-Isopropoxy-5-methanesulfonyl-phenyl)-thiourea

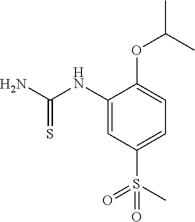

The title compound was prepared using 1-isopropoxy-2-isothiocyanato-4-methanesulfonyl-benzene (intermediate 23, step c) in place of 3-isothiocyanato-4-methoxy-benzonitrile, according to the procedure described for intermediate 22, step c, except that the reaction was done at room temperature.

### Intermediate 24: Step a

### 4-Ethanesulfonyl-2-isothiocyanato-1-methoxy-benzene

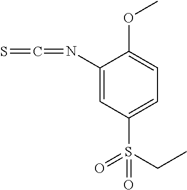

The title compound was prepared using commercially available 5-ethanesulfonyl-2-methoxy-phenylamine in place of 3-amino-4-methoxy-benzonitrile, according to the procedure described for intermediate 22, step b, except that column chromatography was not done.

### Intermediate 24: Step b

### (5-Ethanesulfonyl-2-methoxy-phenyl)-thiourea

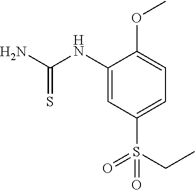

The title compound was prepared using 4-ethanesulfonyl-2-isothiocyanato-1-methoxy-benzene (intermediate 24, step a) in place of 3-isothiocyanato-4-methoxy-benzonitrile, according to the procedure described for intermediate 22, step c, except that ammonia (0.5 M in dioxane) was used in place of ammonia (2.0 M in methanol) and column chromatography was not done.

### Intermediate 25: Step a

### 3-Amino-4-methoxy-benzenesulfonamide

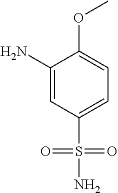

A solution of 30% aqueous ammonium hydroxide (3 mL) was added dropwise to a solution of commercially available 3-amino-4-methoxy-benzenesulfonyl fluoride (0.500 g, 2.44 mmol) at 0° C., then stirred at room temperature overnight. An additional aliquot of 30% aqueous ammonium hydroxide (5 mL) was added and the mixture was heated to 50° C. for several hours. Water was added and the product was extracted with ethyl acetate, dried with sodium sulfate and evaporated to give the title compound.

### Intermediate 25: Step b

### 3-Isothiocyanato-4-methoxy-benzenesulfonamide

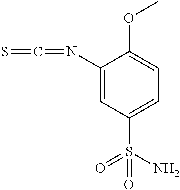

The title compound was prepared using 3-amino-4-methoxy-benzenesulfonamide (intermediate 25, step a) in place of 3-amino-4-methoxy-benzonitrile, according to the procedure described for intermediate 22, step b, except that column chromatography was not done.

### Intermediate 25: Step c

### 4-Methoxy-3-thioureido-benzenesulfonamide

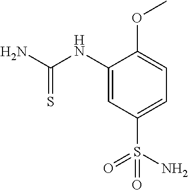

The title compound was prepared using 3-isothiocyanato-4-methoxy-benzenesulfonamide (intermediate 25, step b) in place of 3-isothiocyanato-4-methoxy-benzonitrile, according to the procedure described for intermediate 22, step c, except that ammonia (0.5 M in dioxane) was used in place of ammonia (2.0 M in methanol) and the reaction was concentrated slightly and filtered to give the product as a solid instead of using column chromatography.

### Intermediate 26: Step a

### 4-Fluoro-3-nitro-benzenesulfonamide

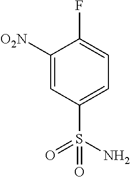

Following the procedure of J. Med. Chem. 2006, 49, 1173, a solution of commercially available 2-fluoronitrobenzene (10.00 g, 70.87 mmol) and chlorosulfonic acid (21 mL) was heated to reflux for 18 hours at 95° C. and then cooled to room temperature. The solution was then added dropwise over a 1 hour period to a solution of iPrOH (225 mL) and concentrated aqueous NH₄OH (54 mL) at −35° C. and stirred for 0.5 hours. The solution was maintained at −35° C. while concentrated aqueous HCl was added until the pH was acidic. The solution was then evaporated to remove some iPrOH, water was added and the solution was evaporated again to remove most of the iPrOH. More water was added, the solution was filtered and the solid was washed with 1 N aqueous HCl and water to give the title compound.

### Intermediate 26: Step b

### 4-Isopropoxy-3-nitro-benzenesulfonamide

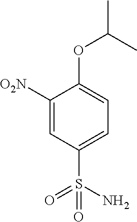

A solution of isopropanol (225 mL) and small chunks of sodium metal (1.92 g, 83.6 mmol) were heated to reflux for 2.5 hours, until the sodium was consumed. The resulting solution was added while still hot to a solution of 4-fluoro-3-nitro-benzenesulfonamide (8.37 g, 38.0 mmol, intermediate 26, step a) in THF/iPrOH (1/1, v/v, 150 mL) over a 10 minute period and stirred at room temperature for 3.5 hours. The reaction mixture was partitioned between EtOAc and brine and 1 N aqueous HCl. The organic phase was then washed with brine, dried with Na₂SO₄ and evaporated to give the title compound.

### Intermediate 26: Step c

### 3-Amino-4-isopropoxy-benzenesulfonamide

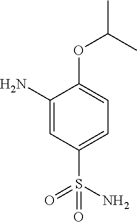

Sodium borohydride (1.88 g, 49.6 mmol) was added slowly to a solution of nickel (II) chloride hexahydrate (3.93 g, 16.5 mmol) in methanol (60 mL) at 0° C. and the resulting black suspension was stirred for 30 min at 23° C. The mixture was cooled to 0° C. and 4-isopropoxy-3-nitro-benzenesulfonamide (8.6 g, 33.0 mmol, intermediate 26, step b) was added followed by sodium borohydride (4.38 g, 115.6 mmol). The resulting black suspension was stirred for 30 min at 23° C. Water was added to the reaction mixture to quench excess NaBH₄, followed by addition of saturated aqueous NaHCO₃. The product was extracted with dichloromethane and the organic phase was washed with brine, dried with Na₂SO₄ and evaporated to give the title compound.

### Intermediate 26: Step d

### 4-Isopropoxy-3-isothiocyanato-benzenesulfonamide

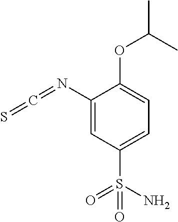

A solution of sodium bicarbonate (16.8 g, 199.5 mmol) in water (400 mL) was added to 3-amino-4-isopropoxy-benzenesulfonamide (15.3 g, 66.5 mmol, intermediate 26, step c) in a mixture of chloroform (200 mL) and water (200 mL). Thiophosgene (6.37 mL, 83.1 mmol) was then added. The biphasic solution was stirred at room temperature for 1.5 h. The phases were separated and the aqueous phase was extracted with CH₂Cl₂. The organic phase was washed with water, dried (Na₂SO₄), filtered, and concentrated, yielding the crude title compound as a tan solid.

### Intermediate 26: Step e

### 4-Isopropoxy-3-thioureido-benzenesulfonamide

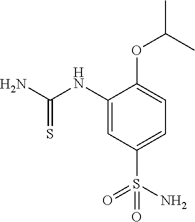

Crude 4-isopropoxy-3-isothiocyanato-benzenesulfonamide (17.8 g, 65.2 mmol, intermediate 26, step d) was treated with a 2 M solution of ammonia in MeOH (250 mL) and the resulting solution was stirred at room temperature for 18 h. The reaction mixture was then concentrated to about half the volume until a large amount of tan solid precipitated. The solution was cooled to 0° C. for 30 minutes and was filtered. The solid was washed with methanol and ether to give the title compound as a cream colored solid.

### Intermediate 27: Step a

### 4-Isopropoxy-3-nitro-benzoic acid isopropyl ester

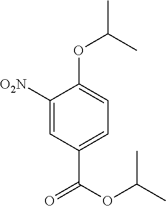

The title compound was prepared using commercially available 4-fluoro-3-nitro-benzoic acid methyl ester in place of 4-methylsulfonyl-2-nitrofluorobenzene, according to the procedure described for intermediate 23, step a, except that 3 equivalents of KHMDS and 3 equivalents of isopropanol were used, and the compound was purified via column chromatography with heptanes: ethyl acetate to give the title compound.

### Intermediate 27: Step b

### 3-Amino-4-isopropoxy-benzoic acid isopropyl ester

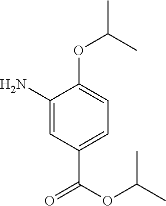

The title compound was prepared using 4-isopropoxy-3-nitro-benzoic acid isopropyl ester (intermediate 27, step a) in place of 4-methoxy-3-nitro-benzonitrile, according to the procedure described in intermediate 22, step a, except that column chromatography was not done.

### Intermediate 27: Step c

### 4-Isopropoxy-3-isothiocyanato-benzoic acid isopropyl ester

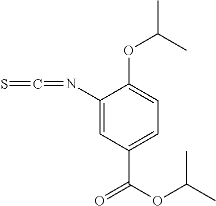

The title compound was prepared using 3-amino-4-isopropoxy-benzoic acid isopropyl ester (intermediate 27, step b) in place of 3-amino-4-methoxy-benzonitrile, according to the procedure described for intermediate 22, step b, except that column chromatography was not done.

### Intermediate 27: Step d

### 4-Isopropoxy-3-thioureido-benzoic acid isopropyl ester

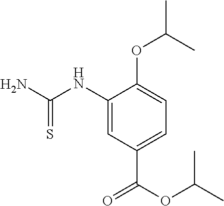

The title compound was prepared using 4-isopropoxy-3-isothiocyanato-benzoic acid isopropyl ester (intermediate 27, step c) in place of 3-isothiocyanato-4-methoxy-benzonitrile, according to the procedure described for intermediate 22, step c, except that the reaction was run at room temperature, ammonia (0.5 M in dioxane) was used in place of ammonia (2.0 M in methanol), and column chromatography was not done.

### Intermediate 27: Step e

### 4-Isopropoxy-3-thioureido-benzoic acid

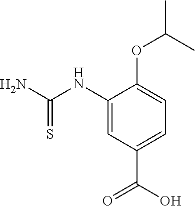

A solution of 2 N aqueous sodium hydroxide (1.78 mL, 3.56 mmol) was added to a solution of 4-isopropoxy-3-thioureido-benzoic acid isopropyl ester (0.353 g, 1.19 mmol, intermediate 27, step d) in methanol (5 mL) and stirred at room temperature overnight. The resulting solution was evaporated to remove methanol. A solution of 1 N aqueous HCl was added to adjust the pH to 4 and the product was extracted with ethyl acetate, dried with sodium sulfate and evaporated to give the title compound.

### Intermediate 28: Step a

### 4-Isopropoxy-3-nitro-benzonitrile

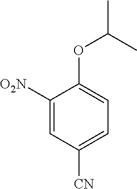

The title compound was prepared using commercially available 4-fluoro-3-nitro-benzonitrile in place of 4-methylsulfonyl-2-nitrofluorobenzene, according to the procedure described in intermediate 23, step a.

### Intermediate 28: Step b

### 3-Amino-4-isopropoxy-benzonitrile

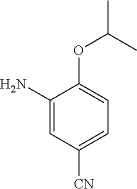

The title compound was prepared using 4-isopropoxy-3-nitro-benzonitrile (intermediate 28, step a) in place of 4-methoxy-3-nitro-benzonitrile, according to the procedure described in intermediate 22, step a.

### Intermediate 28: Step c

### 4-Isopropoxy-3-isothiocyanato-benzonitrile

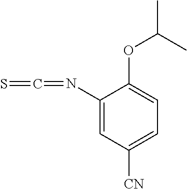

The title compound was prepared using 3-amino-4-isopropoxy-benzonitrile (intermediate 28, step b) in place of 3-amino-4-methoxy-benzonitrile, according to the procedure described in intermediate 22, step b, except column chromatography was not done.

### Intermediate 28: Step d

### (5-Cyano-2-isopropoxy-phenyl)-thiourea

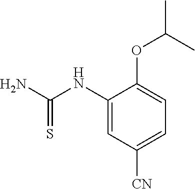

The title compound was prepared using 4-isopropoxy-3-isothiocyanato-benzonitrile (intermediate 28, step c) in place of 3-isothiocyanato-4-methoxy-benzonitrile, according to the procedure described for intermediate 22, step c, except that the reaction was run at room temperature, ammonia (0.5 M in dioxane) was used in place of ammonia (2.0 M in methanol), and column chromatography was not done.

### Intermediate 29: Step a

### 4-Ethoxy-3-nitro-benzoic acid ethyl ester

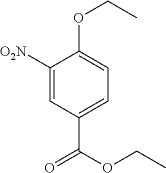

A solution of sodium ethoxide (21% in ethanol, 12.37 mL) was added slowly to a solution of commercially available 4-fluoro-3-nitro-benzoic acid methyl ester (3.00 g, 15.06 mmol) in ethanol (50 mL) at room temperature and stirred for 10 minutes. The reaction mixture was then evaporated, water was added and the product was extracted with ethyl acetate, dried with sodium sulfate and evaporated to give the title compound.

### Intermediate 29: Step b

### 4-Ethoxy-3-nitro-benzoic acid

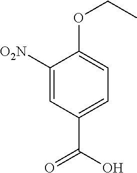

A solution of 2 N aqueous sodium hydroxide (8.36 mL) was added to a solution of 4-ethoxy-3-nitro-benzoic acid ethyl ester (2.00 g, 8.36 mmol, intermediate 29, step a) in ethanol (20 mL) and stirred at room temperature for 0.5 hours. An additional solution of 2 N aqueous sodium hydroxide (8.36 mL) was added and stirred for 0.5 hours. The reaction mixture was then evaporated to remove ethanol and 1 N aqueous HCl was added and the product was extracted with ethyl acetate, dried with sodium sulfate, and evaporated. Dichloromethane was added and the solution was filtered to give the title compound as a solid.

### Intermediate 29: Step c

### 2-(4-Ethoxy-3-nitro-phenyl)-oxazole

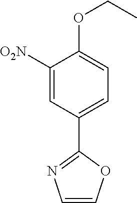

A solution of 4-ethoxy-3-nitro-benzoic acid (0.686 g, 3.48 mmol, intermediate 29, step b) and thionyl chloride (10 mL) was heated to 75° C. for 3 hours and then evaporated to dryness. In a separate flask, NaH (60% in mineral oil) (0.157 g, 3.92 mmol) was added to sulfolane (4 mL) and heated to 50° C. for several minutes. 1,2,3-1H-triazole (0.270 g, 3.92 mmol) was added and the reaction was stirred at 50° C. for 1.5 hours. The acid chloride was then dissolved in sulfolane (4 mL) and heated to 95° C. To this solution was added the triazine solution and the resulting solution was maintained at 95° C. for 1 hour. The reaction was cooled to room temperature, water was added and the crude product was extracted with ethyl acetate, dried with sodium sulfate and evaporated. The product was purified via column chromatography with heptanes: ethyl acetate to give the title compound.

### Intermediate 29: Step d

### 2-Ethoxy-5-oxazol-2-yl-phenylamine

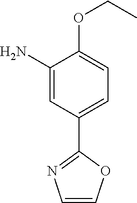

The title compound was prepared using 2-(4-ethoxy-3-nitro-phenyl)-oxazole (intermediate 29, step c) in place of 4-methoxy-3-nitro-benzonitrile, according to the procedure described in intermediate 22, step a.

### Intermediate 29: Step e

### 2-(4-Ethoxy-3-isothiocyanato-phenyl)-oxazole

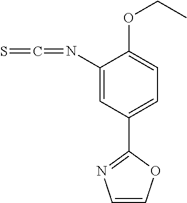

The title compound was prepared using 2-ethoxy-5-oxazol-2-yl-phenylamine (intermediate 29, step d) in place of 3-amino-4-methoxy-benzonitrile, according to the procedure described in intermediate 22, step b, except column chromatography was not done.

### Intermediate 29: Step f

### (2-Ethoxy-5-oxazol-2-yl-phenyl)-thiourea

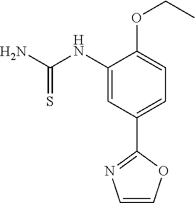

The title compound was prepared using 2-(4-ethoxy-3-isothiocyanato-phenyl)-oxazole (intermediate 29, step e) in place of 3-isothiocyanato-4-methoxy-benzonitrile, according to the procedure described for intermediate 22, step c, except that the reaction was run at room temperature, ammonia (0.5 M in dioxane) was used in place of ammonia (2.0 M in methanol), and the reaction mixture was concentrated instead of filtering.

### Intermediate 30: Step a

### 3-Nitro-4-trifluoromethoxy-benzenesulfonamide

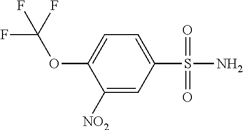

To chlorosulfonic acid (11.3 mL, 170 mmol) was slowly added commercially available 2-trifluoromethoxy-nitrobenzene (8 g, 38.6 mmol). The reaction mixture was heated at 120° C. for 4 h and then cooled down. The above crude mixture was added to a stirred solution of conc. aq. NH₄OH (34.7 mL, 514 mmol, 14.8 M) in iPrOH (100 mL) at −45° C. dropwise over 30 min. The reaction mixture was stirred at −45° C. for 1 h, and 2 N HCl was added to acidify the mixture. Concentration to remove iPrOH was followed by suspension in water, and filtration of the solid. The solid was washed successively with 1 N HCl and water, then air dried to yield the title compound as a white solid.

### Intermediate 30: Step b

### 3-Amino-4-trifluoromethoxy-benzenesulfonamide

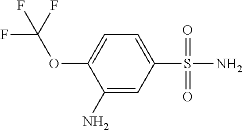

The title compound was prepared using 3-nitro-4-trifluoromethoxy-benzenesulfonamide (intermediate 30, step a) in place of 4-isopropoxy-3-nitro-benzamide according to the procedure of intermediate 13, step c.

### Intermediate 30: Step c

### 3-Isothiocyanato-4-trifluoromethoxy-benzenesulfonamide

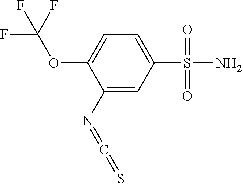

A solution of sodium bicarbonate (3.8 g, 45.2 mmol) in water (50 mL) was added to 3-amino-4-trifluoromethoxy-benzenesulfonamide (3.84 g, 15.0 mmol, intermediate 30, step b) in chloroform (100 mL). Thiophosgene (1.44 mL, 18.7 mmol) was then added. The biphasic solution was stirred at room temperature for 2 h. TLC analysis indicated slight remaining starting material, so an additional 0.5 mL portion of thiophosgene was added and the mixture was stirred for 40 min. The reaction mixture was partially concentrated to get rid of most chloroform. The precipitated solid was filtered, washed with water, and air dried, yielding the crude title compound as an off-white solid.

### Intermediate 30: Step d

### 3-Thioureido-4-trifluoromethoxy-benzenesulfonamide

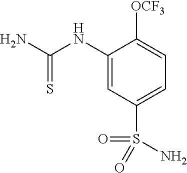

Crude 3-isothiocyanato-4-trifluoromethoxy-benzenesulfonamide (2.2 g, intermediate 30, step c) was dissolved in a 2 M solution of ammonia in MeOH (29.5 mL). The resulting yellow solution was stirred at room temperature for 16 h. The reaction mixture was concentrated and dried under vacuum to afford the title compound as a foamy solid.

### Intermediate 31

### tert-Butyl 5-(2-((2-isopropoxy-5-sulfamoylphenyl)amino)thiazol-4-yl)-5′,6′-dihydro-[2,4′-bipyridine]-1′(2′H)-carboxylate

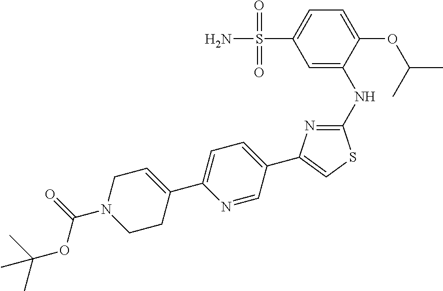

A solution of 3-((4-(6-chloropyridin-3-yl)thiazol-2-yl)amino)-4-isopropoxybenzenesulfonamide. TsOH (0.300 g, 0.502 mmol, intermediate 10), tert-butyl 4-(4,4,5,5-tetramethyl-1,3,2-dioxaborolan-2-yl)-5,6-dihydropyridine-1(2H)-carboxylate (0.186 g, 0.603 mmol), Pd(dppf)Cl₂.DCM (0.082 g, 0.100 mmol), and K₂CO₃ (0.278 g, 2.01 mmol) in dioxane:water (10 mL) was heated to 96° C. for 6 h. The reaction was then cooled to room temperature, water was added and the crude product was extracted with ethyl acetate, dried with sodium sulfate and purified via column chromatography to give the title compound.

### Intermediate 32

### tert-Butyl 4-(5-(2-((2-isopropoxy-5-sulfamoylphenyl)amino)thiazol-4-yl)pyridin-2-yl)piperidine-1-carboxylate

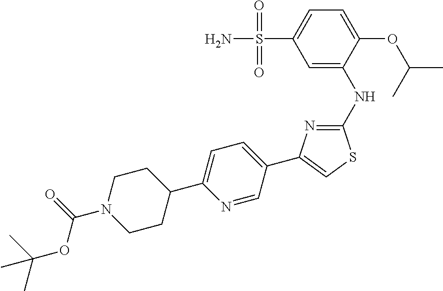

A solution of tert-butyl 5-(2-((2-isopropoxy-5-sulfamoylphenyl)amino)thiazol-4-yl)-5′,6′-dihydro-[2,4′-bipyridine]-1′(2′H)-carboxylate (0.200 g, 0.350 mmol, intermediate 31) and 10% Pd—C (30 mg) in methanol (6 mL) was subjected to a parr-shaker with 40 psi of hydrogen for 18 hours. The reaction was then filtered through celite and concentrated to give the title compound.

### Intermediate 33: Step a

### 1-(6-(4-Cyclopropylpiperazin-1-yl)pyridin-3-yl)ethanone

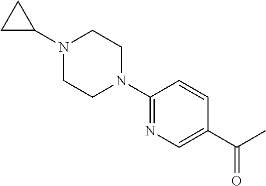

A solution of 1-cyclopropylpiperazine (0.162 g, 1.29 mmol), 1-(6-chloropyridin-3-yl)ethanone (0.200 g, 1.29 mmol) and DMSO (0.2 mL) was heated to 100° C. overnight. The reaction was cooled to room temperature, ethyl acetate was added and the reaction mixture was filtered to give the title compound as a solid.

### Intermediate 33: Step b

### 2-Bromo-1-(6-(4-cyclopropylpiperazin-1-yl)pyridin-3-yl)ethanone.HBr

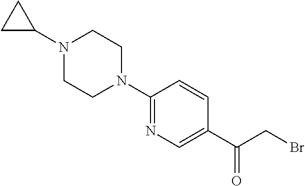

Bromine (0.020 mL, 0.393 mmol) was added to a solution of 1-(6-(4-cyclopropylpiperazin-1-yl)pyridin-3-yl)ethanone (0.107 g, 0.436 mmol) in 48% aqueous HBr (6 mL) at 70° C. and heated at that temperature overnight. The reaction was then cooled to room temperature and was evaporated several times in the presence of toluene to give the title compound.

### Example 1

### 4-Isopropoxy-3-(4-pyridin-3-yl-thiazol-2-ylamino)-benzamide.HBr

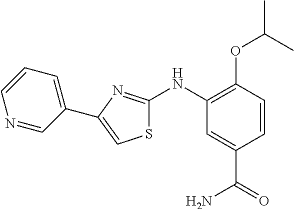

A mixture of 2-bromo-1-pyridin-3-yl-ethanone.HBr (Oakwood Products, 56.2 mg, 0.20 mmol), 4-isopropoxy-3-thioureido-benzamide (50.7 mg, 0.20 mmol, intermediate 13, step e) and EtOH (1 mL) was stirred at 23° C. for 2 d. The mixture was filtered and the collected yellow solid was suspended in EtOH (1 mL) and was heated at 100° C. for 10 min (microwave). The mixture was cooled to 0° C. and was filtered, washing with EtOH and heptane, and air-dried. The title compound was obtained as a yellow powder. ¹H NMR (300 MHz, DMSO-d₆) δ ppm 9.65 (s, 1H), 9.26-9.31 (m, 1H), 9.03 (d, J=1.9 Hz, 1H), 8.90 (d, J=7.9 Hz, 1H), 8.80 (d, J=5.3 Hz, 1H), 8.03 (dd, J=8.3, 5.7 Hz, 1H), 7.86 (s, 2H), 7.57 (dd, J=8.5, 2.1 Hz, 1H), 7.07-7.21 (m, 2H), 4.79 (sept, J=5.9 Hz, 1H), 1.35 (d, J=6.0 Hz, 6H). MS m/e 355.0.

### Example 2

### 4-Ethoxy-3-(4-pyridin-3-yl-thiazol-2-ylamino)-benzamide.TFA

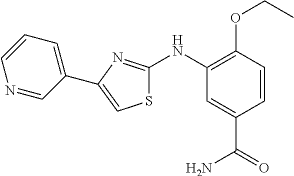

A mixture of 4-ethoxy-3-thioureido-benzamide (25.0 mg, 0.104 mmol, intermediate 15, step c), 2-bromo-1-pyridin-3-yl-ethanone.HBr (29.4 mg, 0.104 mmol), and EtOH (1 mL) was stirred at room temperature for 3 d. The mixture was filtered and the collected solid was purified by RP-HPLC (10-90% CH₃CN—H₂O, 0.1% TFA), affording the title compound. ¹H NMR (400 MHz, DMSO-d₆) δ ppm 9.70 (s, 1H), 9.23 (d, J=2.0 Hz, 1H), 9.05 (d, J=2.0 Hz, 1H), 8.59-8.69 (m, 2H), 7.85 (br. s., 1H), 7.77 (dd, J=7.9, 5.3 Hz, 1H), 7.71 (s, 1H), 7.56 (dd, J=8.4, 2.1 Hz, 1H), 7.15 (br. s., 1H), 7.08 (d, J=8.6 Hz, 1H), 4.20 (q, J=7.1 Hz, 2H), 1.42 (t, J=7.0 Hz, 3H). MS m/e 341.0 (M+H).

### Example 3

### (2-Methyl-benzofuran-7-yl)-(4-pyridin-3-yl-thiazol-2-yl)-amine

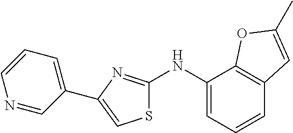

A mixture of (2-methyl-benzofuran-7-yl)-thiourea (103.1 mg, 0.500 mmol, intermediate 16) and 2-bromo-1-pyridin-3-yl-ethanone.HBr (140.5 mg, 0.500 mmol) was heated by microwave irradiation (100° C., 10 min, 300 W). The reaction mixture was partitioned between CH₂Cl₂ and sat. aq. NaHCO₃. The separated aq. phase was extracted twice with CH₂Cl₂. The organic phase was dried (Na₂SO₄), filtered, and concentrated and the residue was purified by flash column chromatography (Silica gel, 20-70% EtOAc-Hept) to afford the title compound. ¹H NMR (400 MHz, DMSO-d₆) δ 10.44 (s, 1H), 9.14 (d, J=1.71 Hz, 1H), 8.52 (dd, J=1.47, 4.65 Hz, 1H), 8.21-8.29 (m, 2H), 7.54 (s, 1H), 7.46 (dd, J=4.77, 7.95 Hz, 1H), 7.15-7.25 (m, 2H), 6.62 (d, J=0.98 Hz, 1H). MS m/e 308.1 (M+H).

### Example 4

### 4-Methoxy-3-(4-pyridin-3-yl-thiazol-2-ylamino)-benzamide.HBr

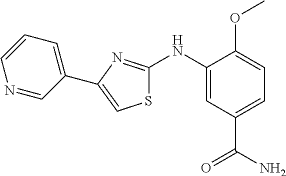

A mixture of 4-methoxy-3-thioureido-benzamide (112.6 mg, 0.500 mmol, intermediate 14) and 2-bromo-1-pyridin-3-yl-ethanone.HBr (140.5 mg, 0.500 mmol) was heated by microwave irradiation (100° C., 10 min, 300 W). The precipitated solid was collected by vacuum filtration and was washed with EtOH, affording the title compound. ¹H NMR (300 MHz, DMSO-d₆) δ 9.96 (s, 1H), 9.29 (d, J=1.51 Hz, 1H), 9.07 (d, J=2.26 Hz, 1H), 8.93 (d, J=8.29 Hz, 1H), 8.82 (d, J=4.90 Hz, 1H), 8.06 (dd, J=5.46, 8.10 Hz, 1H), 7.85-7.93 (m, 2H), 7.61 (dd, J=2.07, 8.48 Hz, 1H), 7.17 (br. s., 1H), 7.11 (d, J=8.29 Hz, 1H), 3.94 (s, 3H). MS m/e 327.1 (M+H).

### Example 5

### 2-[4-Methoxy-3-(4-pyridin-3-yl-thiazol-2-ylamino)-phenyl]-acetamide

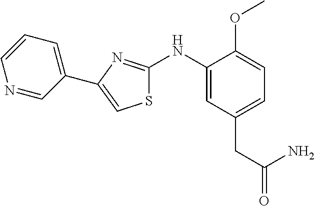

A mixture of 2-bromo-1-pyridin-3-yl-ethanone.HBr (70.2 mg, 0.25 mmol), 2-(4-methoxy-3-thioureido-phenyl)-acetamide (59.8 mg, 0.25 mmol, intermediate 18, step d), and EtOH (1 mL) was stirred at room temperature for 1 d. The reaction mixture was filtered to collect precipitated solid and the solid was washed with EtOH. The solid was vigorously stirred in a mixture of sat. aq. NaHCO₃ and CH₂Cl₂ until it dissolved (10 min). The phases were separated, the aqueous phase was further extracted with CH₂Cl₂, and the combined organic extracts were dried (Na₂SO₄), filtered, and concentrated, yielding the title compound as an off-white solid. ¹H NMR (400 MHz, DMSO-d₆) δ 9.65 (s, 1H), 9.15 (d, J=1.71 Hz, 1H), 8.45-8.53 (m, 2H), 8.30 (dt, J=1.96, 8.07 Hz, 1H), 7.43-7.53 (m, 3H), 6.93-7.00 (m, 1H), 6.83-6.93 (m, 2H), 3.85 (s, 3H), 3.35 (s, 2H). MS m/e 341.0 (M+H).

### Example 6

### 3-Methoxy-4-(4-pyridin-3-yl-thiazol-2-ylamino)-benzamide

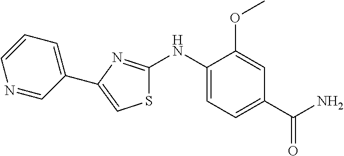

A mixture of 2-bromo-1-pyridin-3-yl-ethanone.HBr (112.4 mg, 0.40 mmol) and 3-methoxy-4-thioureido-benzamide (90.1 mg, 0.40 mmol, intermediate 19, step d) was heated by microwave irradiation (100° C., 10 min, 300 W). The reaction mixture was filtered and the solid was stirred vigorously in a mixture of EtOAc and sat. aq. NaHCO₃. The separated aq. phase was extracted twice with EtOAc. The organic phase was dried (Na₂SO₄), filtered, and concentrated. The crude product was purified by column chromatography (Silica gel, 0-2% MeOH—CH₂Cl₂) affording the title compound as a white powder. ¹H NMR (400 MHz, DMSO-d₆) δ 9.99 (s, 1H), 9.15 (s, 1H), 8.68 (d, J=8.31 Hz, 1H), 8.52 (d, J=4.40 Hz, 1H), 8.26 (d, J=8.07 Hz, 1H), 7.87 (br. s., 1H), 7.52-7.63 (m, 3H), 7.47 (dd, J=4.77, 7.95 Hz, 1H), 7.24 (br. s., 1H), 3.94 (s, 3H). MS m/e 327.1 (M+H).

### Example 7

### 4-Methoxy-2-methyl-5-{4-[6-(4-methyl-piperazin-1-yl)-pyridin-3-yl]-thiazol-2-ylamino}-benzamide.TFA

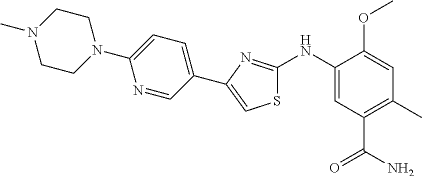

A mixture of 4-methoxy-2-methyl-5-thioureido-benzamide (30 mg, 0.125 mmol, intermediate 20, step e), 2-bromo-1-[6-(4-methyl-piperazin-1-yl)-pyridin-3-yl]-ethanone.HBr (47.5 mg, 0.125 mmol, intermediate 8, step b), and EtOH (0.3 mL) was stirred at room temperature for 4 d. The mixture was partitioned between sat. aq. NaHCO₃ and EtOAc. The aq. phase was extracted with EtOAc. The organic phase was dried (Na₂SO₄), filtered, and concentrated and the residue was purified by reverse phase HPLC (5-50% CH₃CN—H₂O, 0.1% TFA) affording the title compound as a yellow powder. ¹H NMR (400 MHz, DMSO-d₆) δ 9.82 (br. s., 1H), 9.61 (s, 1H), 8.71 (d, J=2.20 Hz, 1H), 8.62 (s, 1H), 8.09 (dd, J=2.45, 8.80 Hz, 1H), 7.59 (br. s., 1H), 7.16-7.21 (m, 2H), 7.02 (d, J=8.80 Hz, 1H), 6.89 (s, 1H), 4.48 (d, J=12.96 Hz, 2H), 3.88 (s, 3H), 3.47-3.55 (m, 2H), 3.01-3.22 (m, 4H), 2.85 (d, J=2.93 Hz, 3H), 2.37 (s, 3H). MS m/e 439.2 (M+H).

### Example 8

### 4-Isobutyl-3-{4-[6-(4-methyl-piperazin-1-yl)-pyridin-3-yl]-thiazol-2-ylamino}-benzamide.TFA

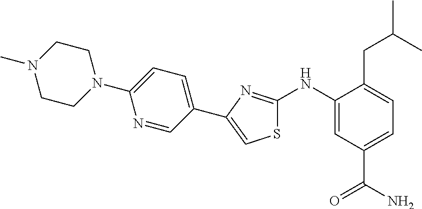

A mixture of 2-bromo-1-[6-(4-methyl-piperazin-1-yl)-pyridin-3-yl]-ethanone.HBr (50 mg, 0.132 mmol, intermediate 8, step b) and 4-isobutyl-3-thioureido-benzamide (33.2 mg, 0.132 mmol, intermediate 21, step e) in EtOH (0.5 mL) was heated by microwave irradiation (100° C., 10 min, 300 W). The reaction mixture was partitioned between EtOAc and sat. aq. NaHCO₃. The separated aq. phase was extracted twice with EtOAc. The organic phase was dried (Na₂SO₄), filtered, and concentrated and the residue was purified by reverse phase HPLC (4-50% CH₃CN—H₂O, 0.1% TFA) affording the title compound as a light yellow powder. ¹H NMR (400 MHz, DMSO-d₆) δ 9.87 (br. s., 1H), 9.44 (br. s., 1H), 8.68 (d, J=2.45 Hz, 1H), 8.52 (s, 1H), 8.07 (dd, J=2.32, 8.93 Hz, 1H), 7.92 (br. s., 1H), 7.54-7.59 (m, 1H), 7.23-7.30 (m, 2H), 7.16 (s, 1H), 7.02 (d, J=9.05 Hz, 1H), 4.45 (d, J=12.47 Hz, 2H), 3.53 (d, J=10.27 Hz, 2H), 3.01-3.22 (m, 4H), 2.86 (s, 3H), 2.62 (d, J=6.85 Hz, 2H), 1.82-1.94 (m, 1H), 0.86 (d, J=6.36 Hz, 6H). MS m/e 451.2 (M+H).

### Example 9

### 4-Methoxy-3-(4-pyridin-3-yl-thiazol-2-ylamino)-benzonitrile.HBr

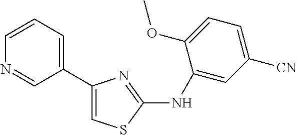

A solution of 2-bromo-1-pyridin-3-yl-ethanone.HBr (0.034 g, 0.121 mmol) and (5-cyano-2-methoxy-phenyl)-thiourea (0.025 g, 0.121 mmol, intermediate 22, step c) in ethanol (3 mL) was stirred at room temperature overnight. The reaction mixture was then filtered and washed with ethanol and dried to give the title compound. ¹H NMR (300 MHz, DMSO-d₆) δ 10.22 (s, 1H), 9.25 (s, 1H), 8.97 (d, J=1.88 Hz, 1H), 8.68-8.83 (m, 2H), 7.95 (dd, J=5.46, 7.72 Hz, 1H), 7.85 (s, 1H), 7.51 (dd, J=2.07, 8.48 Hz, 1H), 7.24 (d, J=8.67 Hz, 1H), 3.99 (s, 3H); MS m/e 309.1 (M+H).

### Example 10

### (2-Isopropoxy-5-methanesulfonyl-phenyl)-(4-pyridin-3-yl-thiazol-2-yl)-amine.TFA

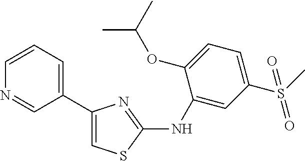

A solution of 2-bromo-1-pyridin-3-yl-ethanone.HBr (0.024 g, 0.087 mmol) and (2-isopropoxy-5-methanesulfonyl-phenyl)-thiourea (0.025 g, 0.087 mmol, intermediate 23, step d) in ethanol (3 mL) was stirred at room temperature overnight. The reaction mixture was then concentrated and the residue was purified via reverse phase HPLC with water/acetonitrile/0.1% TFA to give the title compound. ¹H NMR (300 MHz, MeOD) δ 9.50 (d, J=2.26 Hz, 1H), 9.30 (br. s., 1H), 8.96 (d, J=7.91 Hz, 1H), 8.69 (br. s., 1H), 7.93-8.05 (m, 1H), 7.69 (s, 1H), 7.57 (dd, J=2.26, 8.67 Hz, 1H), 7.24 (d, J=8.67 Hz, 1H), 4.92-4.95 (m, 1H), 3.08-3.20 (m, 3H), 1.46 (d, J=6.03 Hz, 6H); MS m/e 390.0 (M+H).

### Example 11

### (5-Ethanesulfonyl-2-methoxy-phenyl)-(4-pyridin-3-yl-thiazol-2-yl)-amine.TFA

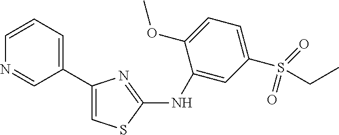

The title compound was prepared using (5-ethanesulfonyl-2-methoxy-phenyl)-thiourea (intermediate 24, step b) in place of (2-isopropoxy-5-methanesulfonyl-phenyl)-thiourea according to the procedure described in example 10. ¹H NMR (300 MHz, DMSO-d₆) δ 10.26 (s, 1H), 9.32 (d, J=1.88 Hz, 1H), 9.22 (br. s., 1H), 8.51-8.82 (m, 2H), 7.72-7.94 (m, 2H), 7.51 (dd, J=2.26, 8.67 Hz, 1H), 7.29 (d, J=8.67 Hz, 1H), 4.00 (s, 3H), 3.26 (q, J=7.16 Hz, 2H), 1.15 (t, J=7.35 Hz, 3H); MS m/e 376.0 (M+H).

### Example 12

### 4-Methoxy-3-(4-pyridin-3-yl-thiazol-2-ylamino)-benzenesulfonamide.TFA

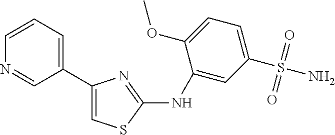

The title compound was prepared using 4-methoxy-3-thioureido-benzenesulfonamide (intermediate 25, step c) in place of (2-isopropoxy-5-methanesulfonyl-phenyl)-thiourea according to the procedure described in example 10. ¹H NMR (300 MHz, DMSO-d₆) δ 10.00 (s, 1H), 9.07-9.26 (m, 2H), 8.39-8.60 (m, 2H), 7.54-7.71 (m, 2H), 7.38 (dd, J=2.45, 8.48 Hz, 1H), 7.04-7.22 (m, 3H), 3.88 (s, 3H); MS ES+ 363.1 (M+H).

### Example 13

### 4-Isopropoxy-3-(4-pyridin-3-yl-thiazol-2-ylamino)-benzenesulfonamide.TFA

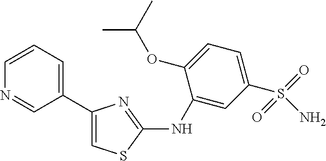

The title compound was prepared using 4-isopropoxy-3-thioureido-benzenesulfonamide (intermediate 26, step e) in place of (2-isopropoxy-5-methanesulfonyl-phenyl)-thiourea according to the procedure described in example 10. ¹H NMR (300 MHz, DMSO-d₆) δ 9.77 (s, 1H), 9.25 (dd, J=1.88, 18.84 Hz, 2H), 8.61 (d, J=4.90 Hz, 2H), 7.72 (s, 1H), 7.60-7.70 (m, 1H), 7.44 (dd, J=2.26, 8.67 Hz, 1H), 7.24 (s, 1H), 7.21 (br. s., 2H), 4.76-4.87 (m, 1H), 1.37 (d, J=6.03 Hz, 6H); MS m/e 391.0 (M+H).

### Example 14

### 4-Isopropoxy-3-(4-pyridin-2-yl-thiazol-2-ylamino)-benzamide

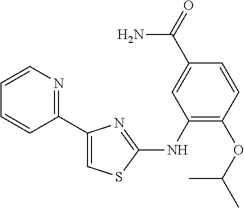

A solution of commercially available 2-bromo-1-pyridin-2-yl-ethanone (0.028 g, 0.139 mmol) and 4-isopropoxy-3-thioureido-benzamide (0.035 g, 0.139 mmol, intermediate 13, step e) in ethanol (5 mL) was stirred at room temperature for 18 hours and then the reaction mixture was filtered. To the collected solid was added saturated aq. NaHCO₃ and the product was extracted with ethyl acetate, dried with sodium sulfate and evaporated to give the title compound. ¹H NMR (400 MHz, DMSO-d₆) δ 9.56 (s, 1H), 9.24 (d, J=2.20 Hz, 1H), 9.13 (d, J=2.20 Hz, 1H), 8.57 (dd, J=1.59, 4.77 Hz, 1H), 8.35 (dt, J=1.90, 7.95 Hz, 1H), 7.88 (br. s., 1H), 7.56-7.64 (m, 2H), 7.51 (dd, J=4.52, 7.70 Hz, 1H), 7.22 (br. s., 1H), 7.16 (d, J=8.56 Hz, 1H), 4.77-4.91 (m, 1H), 1.42 (d, J=5.87 Hz, 6H); MS m/e 355.2 (M+H).

### Example 15

### 4-Isopropoxy-3-(4-pyridin-4-yl-thiazol-2-ylamino)-benzamide.HBr

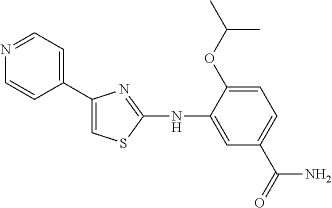

The title compound was prepared using commercially available 2-bromo-1-(4-pyridinyl)-1-ethanone hydrobromide.HBr and 4-isopropoxy-3-thioureido-benzamide (intermediate 13, step e) in place of 2-bromo-1-pyridin-3-yl-ethanone.HBr and (5-cyano-2-methoxy-phenyl)-thiourea, respectively, according to the procedure described in example 9. ¹H NMR (400 MHz, DMSO-d₆) δ 9.83 (s, 1H), 9.11 (d, J=2.20 Hz, 1H), 9.00 (d, J=6.85 Hz, 2H), 8.48 (d, J=6.60 Hz, 2H), 8.38 (br. s., 1H), 7.95 (br. s., 1H), 7.67 (dd, J=2.08, 8.44 Hz, 1H), 7.13-7.30 (m, 2H), 4.88 (sept, J=5.99, 1H), 1.35 (d, J=6.11 Hz, 6H); MS m/e 355.0 (M+H).

### Example 16

### 4-Isopropoxy-3-(4-pyridin-3-yl-thiazol-2-ylamino)-benzonitrile.HBr

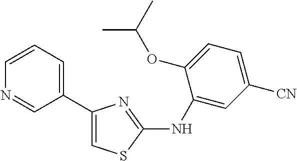

The title compound was prepared using (5-cyano-2-isopropoxy-phenyl)-thiourea (intermediate 28, step d) in place of (5-cyano-2-methoxy-phenyl)-thiourea according to the procedure described in example 9. ¹H NMR (300 MHz, DMSO-d₆) δ 9.91 (s, 1H), 9.25 (s, 1H), 8.95 (d, J=1.88 Hz, 1H), 8.71-8.82 (m, 2H), 7.96 (dd, J=5.65, 8.29 Hz, 1H), 7.86 (s, 1H), 7.47 (dd, J=2.07, 8.48 Hz, 1H), 7.25 (d, J=8.67 Hz, 1H), 4.87 (sept, J=5.84 Hz, 1H), 1.39 (d, J=6.03 Hz, 6H); MS m/e 337.0 (M+H).

### Example 17

### 4-Isopropoxy-3-(4-pyridin-3-yl-thiazol-2-ylamino)-benzoic acid.TFA

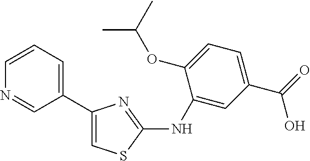

The title compound was prepared using 4-isopropoxy-3-thioureido-benzoic acid (intermediate 27, step e) in place of (2-isopropoxy-5-methanesulfonyl-phenyl)-thiourea according to the procedure described in example 10. ¹H NMR (300 MHz, DMSO-d₆) δ 12.51 (br. s., 1H), 9.60 (s, 1H), 9.07-9.32 (m, 2H), 8.28-8.64 (m, 2H), 7.47-7.76 (m, 3H), 7.10 (d, J=8.67 Hz, 1H), 4.56-4.87 (m, 1H), 1.33 (d, J=6.03 Hz, 6H); MS m/e 356.1 (M+H).

### Example 18

### (2-Ethoxy-5-oxazol-2-yl-phenyl)-(4-pyridin-3-yl-thiazol-2-yl)-amine.HBr

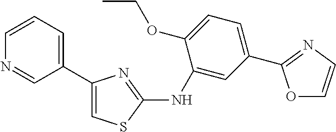

The title compound was prepared using (2-ethoxy-5-oxazol-2-yl-phenyl)-thiourea (intermediate 29, step f) in place of (5-cyano-2-methoxy-phenyl)-thiourea according to the procedure described in example 9. ¹H NMR (300 MHz, DMSO-d₆) δ 9.93 (s, 1H), 9.45 (d, J=2.26 Hz, 1H), 9.30 (d, J=1.88 Hz, 1H), 8.64-8.90 (m, 2H), 8.21 (s, 1H), 7.94-8.06 (m, 1H), 7.85 (s, 1H), 7.62 (dd, J=2.07, 8.48 Hz, 1H), 7.37 (s, 1H), 7.19 (d, J=8.67 Hz, 1H), 4.24 (q, J=6.78 Hz, 2H), 1.45 (t, J=6.97 Hz, 3H); MS m/e 365.1 (M+H).

### Example 19

### 4-Isopropoxy-3-[4-(2,4,6-trimethyl-pyridin-3-yl)-thiazol-2-ylamino]-benzamide.TFA

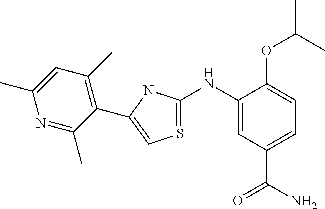

A mixture of 2-bromo-1-(2,4,6-trimethyl-pyridin-3-yl)-ethanone.HBr (31.9 mg, 0.099 mmol, intermediate 1) and 4-isopropoxy-3-thioureido-benzamide (20 mg, 0.079 mmol, intermediate 13, step e) in EtOH (1 mL) was stirred at room temperature for 24 h. The mixture was purified via reverse phase HPLC with water/acetonitrile/0.1% TFA to give the title compound. ¹H NMR (400 MHz, CHLOROFORM-d) δ 8.38 (s, 1H), 7.62 (d, J=8.31 Hz, 1H), 7.37 (s, 1H), 7.14 (br. s., 1H), 6.99 (d, J=8.80 Hz, 2H), 6.73 (s, 1H), 4.65-4.85 (m, 1H), 2.75 (s, 3H), 2.65 (s, 3H), 2.46 (s, 3H), 1.42 (d, J=6.11 Hz, 6H); MS m/e 397.2 (M+H).

### Example 20

### 4-Isopropoxy-3-[4-(6-bromo-pyridin-3-yl)-thiazol-2-ylamino]-benzamide.TFA

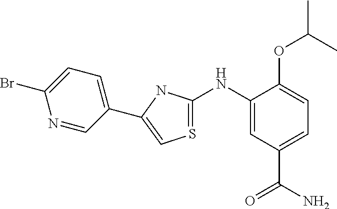

The title compound was prepared using 2-bromo-1-(6-bromo-pyridin-3-yl)-ethanone.HBr (intermediate 2) in place of 2-bromo-1-(2,4,6-trimethyl-pyridin-3-yl)-ethanone.HBr according to the procedure described in example 19. ¹H NMR (400 MHz, CHLOROFORM-d) δ 8.86 (d, J=2.20 Hz, 1H), 8.74 (d, J=1.47 Hz, 1H), 8.04 (dd, J=2.20, 8.31 Hz, 1H), 7.79 (s, 1H), 7.46-7.56 (m, 2H), 6.91-7.00 (m, 2H), 4.71-4.79 (m, 1H), 1.45 (d, J=6.11 Hz, 6H); MS m/e 433.0 (M+H).

### Example 21

### 3-[4-(2-Chloro-pyridin-4-yl)-thiazol-2-ylamino]-4-isopropoxy-benzamide.TFA

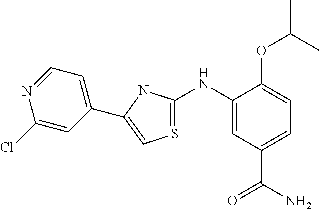

The title compound was prepared using commercially available 2-bromo-1-(2-chloro-pyridin-4-yl)-ethanone in place of 2-bromo-1-(2,4,6-trimethyl-pyridin-3-yl)-ethanone.HBr according to the procedure described in example 19. ¹H NMR (400 MHz, CHLOROFORM-d) δ 8.76 (d, J=1.96 Hz, 1H), 8.42 (d, J=5.14 Hz, 1H), 7.76-7.83 (m, 2H), 7.64-7.69 (m, 1H), 7.51 (dd, J=1.83, 8.44 Hz, 1H), 7.17 (s, 1H), 6.96 (d, J=8.56 Hz, 1H), 4.70-4.82 (m, 1H), 1.45 (d, J=6.11 Hz, 6H); MS m/e 389.1 (M+H).

### Example 22

### 3-[4-(5-Bromo-pyridin-3-yl)-thiazol-2-ylamino]-4-isopropoxy-benzamide.TFA

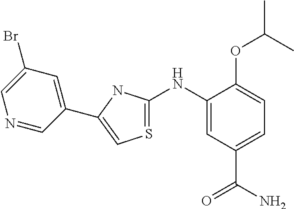

The title compound was prepared using 2-bromo-1-(5-bromo-pyridin-3-yl)-ethanone.HBr (intermediate 3) in place of 2-bromo-1-(2,4,6-trimethyl-pyridin-3-yl)-ethanone.HBr according to the procedure described in example 19. ¹H NMR (400 MHz, CHLOROFORM-d) δ 9.01 (d, J=1.96 Hz, 1H), 8.73 (d, J=1.96 Hz, 1H), 8.61 (d, J=2.20 Hz, 1H), 8.35 (t, J=2.08 Hz, 1H), 7.81 (s, 1H), 7.54 (dd, J=2.08, 8.44 Hz, 1H), 7.02 (s, 1H), 6.96 (d, J=8.31 Hz, 1H), 4.76 (sept, J=6.11 Hz, 1H), 1.45 (d, J=6.11 Hz, 6H); MS m/e 433.0 (M+H).

### Example 23

### 4-Isopropoxy-3-[4-(4-isopropyl-pyridin-3-yl)-thiazol-2-ylamino]-benzamide.TFA

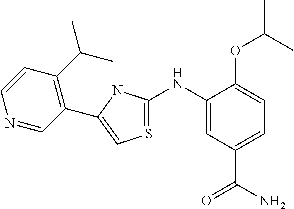

The title compound was prepared using 2-bromo-1-(4-isopropyl-pyridin-3-yl)-ethanone.HBr (intermediate 4) in place of 2-bromo-1-(2,4,6-trimethyl-pyridin-3-yl)-ethanone.HBr according to the procedure described in example 19. ¹H NMR (400 MHz, CHLOROFORM-d) δ 8.64 (d, J=2.20 Hz, 3H), 7.82 (s, 1H), 7.53 (dd, J=2.20, 8.56 Hz, 1H), 7.30 (s, 1H), 6.94 (d, J=8.80 Hz, 1H), 6.68 (s, 1H), 4.69-4.79 (m, 1H), 3.48-3.60 (m, 1H), 1.35-1.51 (m, 6H), 1.06-1.35 (m, 6H); MS m/e 397.2 (M+H).

### Example 24

### 4-Isopropoxy-3-[4-(2-methyl-6-trifluoromethyl-pyridin-3-yl)-thiazol-2-ylamino]-benzamide.TFA

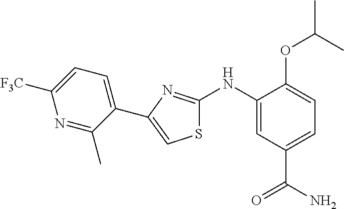

The title compound was prepared using 2-bromo-1-(2-methyl-6-trifluoromethyl-pyridin-3-yl)-ethanone.HBr (intermediate 5) in place of 2-bromo-1-(2,4,6-trimethyl-pyridin-3-yl)-ethanone.HBr according to the procedure described in example 19. ¹H NMR (400 MHz, CHLOROFORM-d) δ 8.71 (d, J=1.96 Hz, 1H), 8.16 (d, J=8.07 Hz, 1H), 7.78 (s, 1H), 7.60 (d, J=8.07 Hz, 1H), 7.50 (dd, J=1.96, 8.56 Hz, 1H), 6.95 (d, J=8.56 Hz, 1H), 6.85 (s, 1H), 4.72-4.79 (m, 1H), 2.85 (s, 3H), 1.45 (d, J=6.11 Hz, 6H); MS m/e 437.1 (M+H).

### Example 25

### 4-Isopropoxy-3-[4-(6-methoxy-pyridin-3-yl)-thiazol-2-ylamino]-benzamide.TFA

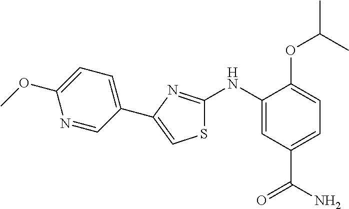

The title compound was prepared using 2-bromo-1-(6-methoxy-pyridin-3-yl)-ethanone.HBr (intermediate 6) in place of 2-bromo-1-(2,4,6-trimethyl-pyridin-3-yl)-ethanone.HBr according to the procedure described in example 19. ¹H NMR (400 MHz, CHLOROFORM-d) δ 8.76 (d, J=1.96 Hz, 1H), 8.68 (d, J=2.45 Hz, 1H), 8.06 (dd, J=2.45, 8.56 Hz, 1H), 7.76 (s, 1H), 7.52 (dd, J=2.20, 8.56 Hz, 1H), 6.94 (d, J=8.56 Hz, 1H), 6.79-6.83 (m, 2H), 4.70-4.82 (m, 1H), 3.98 (s, 3H), 1.44 (d, J=5.87 Hz, 6H); MS m/e 385.2 (M+H).

### Example 26

### 3-[4-(6-Chloro-pyridin-3-yl)-thiazol-2-ylamino]-4-isopropoxy-benzamide.TFA

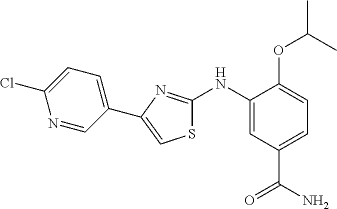

The title compound was prepared using toluene-4-sulfonic acid 2-(6-chloro-pyridin-3-yl)-2-oxo-ethyl ester (intermediate 7) in place of 2-bromo-1-(2,4,6-trimethyl-pyridin-3-yl)-ethanone.HBr according to the procedure described in example 19. ¹H NMR (400 MHz, CHLOROFORM-d) δ 8.88 (d, J=1.96 Hz, 1H), 8.74 (d, J=1.96 Hz, 1H), 8.14 (dd, J=2.45, 8.31 Hz, 1H), 7.79 (s, 1H), 7.51 (dd, J=2.20, 8.56 Hz, 1H), 7.39 (d, J=8.31 Hz, 1H), 6.92-7.00 (m, 2H), 4.75 (sept, J=6.02 Hz, 1H), 1.35-1.54 (m, 6H); MS m/e 389.1 (M+H).

### Example 27

### 4-Isopropoxy-3-{4-[6-(4-methyl-piperazin-1-yl)-pyridin-3-yl]-thiazol-2-ylamino}-benzamide.TFA

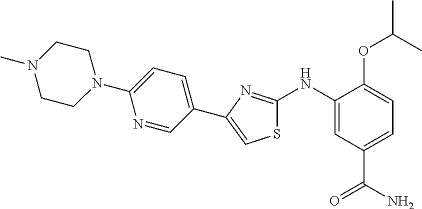

A mixture of 3-[4-(6-chloro-pyridin-3-yl)-thiazol-2-ylamino]-4-isopropoxy-benzamide (20 mg, 0.0514 mmol, example 26) and 1-methyl piperazine (515 mg, 5.14 mmol) was microwaved at 150° C. for 30 min. The mixture was purified via reverse phase HPLC with water/acetonitrile/0.1% TFA to give the title compound. ¹H NMR (400 MHz, MeOD) δ 9.02 (d, J=2.20 Hz, 1H), 8.68 (d, J=2.45 Hz, 1H), 8.35 (dd, J=2.45, 9.05 Hz, 1H), 7.56 (dd, J=2.20, 8.56 Hz, 1H), 7.23 (d, J=9.29 Hz, 1H), 7.17 (s, 1H), 7.09 (d, J=8.80 Hz, 1H), 4.81 (sept, J=6.02, 1H), 3.90 (br.s., 4H), 3.41-3.69 (m, 4H), 2.98 (s, 3H), 1.42 (d, J=6.11 Hz, 6H); MS m/e 453.2 (M+H).

### Example 28

### 4-Isopropoxy-3-{4-[2-(4-methyl-piperazin-1-yl)-pyridin-4-yl]-thiazol-2-ylamino}-benzamide.TFA

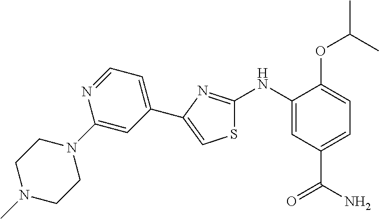

The title compound was prepared using 3-[4-(2-chloro-pyridin-4-yl)-thiazol-2-ylamino]-4-isopropoxy-benzamide.TFA (example 21) in place of 3-[4-(6-chloro-pyridin-3-yl)-thiazol-2-ylamino]-4-isopropoxy-benzamide according to the procedure described in example 27. ¹H NMR (400 MHz, MeOD) δ 9.56 (s, 1H), 8.04-8.09 (m, 2H), 7.87 (s, 1H), 7.53-7.60 (m, 2H), 7.09 (d, J=8.56 Hz, 1H), 4.82-4.90 (m, 1H), 3.59 (br. s., 8H), 3.05 (s, 3H), 1.47 (d, J=5.87 Hz, 6H); MS m/e 453.2 (M+H).

### Example 29

### 4-Methoxy-3-{4-[6-(4-methyl-piperazin-1-yl)-pyridin-3-yl]-thiazol-2-ylamino}-benzenesulfonamide

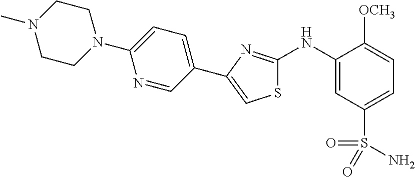

A mixture of 2-bromo-1-[6-(4-methyl-piperazin-1-yl)-pyridin-3-yl]-ethanone.HBr (50 mg, 0.132 mmol, intermediate 8, step b) and 4-methoxy-3-thioureido-benzenesulfonamide (31 mg, 0.119 mmol, intermediate 25, step c) in EtOH (1 mL) was stirred at room temperature for 24 h. A 2 N solution of NH₃ in MeOH (2 mL) was added dropwise; the solid initially dissolved to form a solution, then a precipitate formed. The mixture was filtered and the solid was washed with EtOAc and water and air dried to afford the title compound as a white solid. ¹H NMR (400 MHz, MeOD) δ 9.35 (s, 1H), 8.71-8.77 (m, 1H), 8.15 (br. s., 1H), 7.47-7.53 (m, 1H), 7.08 (s, 1H), 7.01 (s, 1H), 6.81-6.90 (m, 1H), 3.97 (s, 3H), 3.74 (br. s., 4H), 3.09 (br. s., 4H), 2.71 (s, 3H); MS m/e 461.2 (M+H).

### Example 30

### 3-{4-[6-(4-Methyl-piperazin-1-yl)-pyridin-3-yl]-thiazol-2-ylamino}-4-trifluoromethoxy-benzenesulfonamide

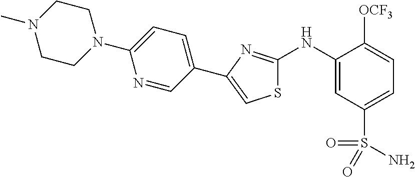

The title compound was prepared using 3-thioureido-4-trifluoromethoxy-benzenesulfonamide (intermediate 30, step d) in place of 4-methoxy-3-thioureido-benzenesulfonamide according to the procedure described in example 29. ¹H NMR (400 MHz, MeOD) δ 9.64 (d, J=1.96 Hz, 1H), 8.77 (d, J=1.96 Hz, 1H), 8.19 (dd, J=2.20, 8.80 Hz, 1H), 7.44-7.56 (m, 2H), 7.11 (s, 1H), 6.97 (d, J=8.80 Hz, 1H), 3.44 (brs, 8H), 2.91 (s, 3H); MS m/e 515.2 (M+H).

### Example 31

### 3-{4-[6-(4-Methyl-piperazin-1-yl)-pyridin-3-yl]-thiazol-2-ylamino}-4-trifluoromethoxy-benzamide

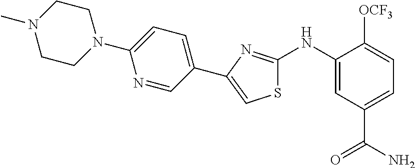

The title compound was prepared using 3-thioureido-4-trifluoromethoxy-benzamide (intermediate 17, step d) in place of 4-methoxy-3-thioureido-benzenesulfonamide according to the procedure described in example 29. ¹H NMR (400 MHz, MeOD) δ 9.50 (d, J=2.20 Hz, 1H), 8.63-8.69 (m, 2H), 7.54-7.60 (m, 2H), 7.42-7.47 (m, 2H), 4.48 (br. s., 2H), 3.76 (br. s., 4H), 3.43-3.55 (m, 2H), 3.04 (s, 3H); MS m/e 479.2 (M+H).

### Example 32

### 4-Methoxy-3-{4-[6-(4-methyl-piperazin-1-yl)-pyridin-3-yl]-thiazol-2-ylamino}-benzamide

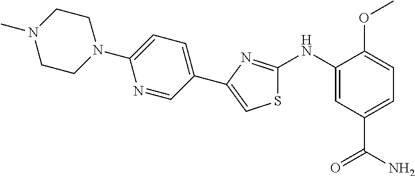

The title compound was prepared using 4-methoxy-3-thioureido-benzamide (intermediate 14) in place of 4-methoxy-3-thioureido-benzenesulfonamide according to the procedure described in example 29. ¹H NMR (400 MHz, DMSO-d₆) δ 9.70 (s, 1H), 9.11 (d, J=1.96 Hz, 1H), 8.78 (d, J=1.96 Hz, 1H), 8.13 (dd, J=2.20, 8.80 Hz, 1H), 7.85 (br. s., 1H), 7.51-7.56 (m, 1H), 7.23 (s, 1H), 6.98-7.17 (m, 3H), 4.00-4.50 (m, 4H), 3.82-3.98 (m, 3H), 3.10-3.70 (m, 4H), 2.85 (s, 3H); MS m/e 425.2 (M+H).

### Example 33

### 4-Isopropoxy-3-{4-[6-(4-methyl-piperazin-1-yl)-pyridin-3-yl]-thiazol-2-ylamino}-benzenesulfonamide.HCl

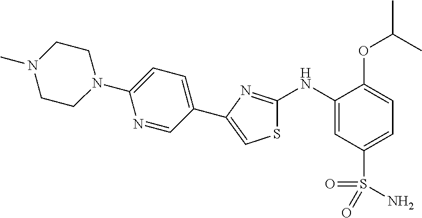

A mixture of 2-bromo-1-[6-(4-methyl-piperazin-1-yl)-pyridin-3-yl]-ethanone.HBr (200 mg, 0.528 mmol, intermediate 8, step b) and 4-isopropoxy-3-thioureido-benzenesulfonamide (137.4 mg, 0.475 mmol, intermediate 26, step e) in EtOH (3 mL) was stirred at room temperature for 24 h. A 2 N solution of NH₃ in MeOH (2 mL) was added dropwise; the solid initially dissolved to form a solution, then a precipitate formed. The mixture was filtered and the solid was washed with EtOAc, water and air dried. The neutral compound was suspended in MeOH (3 mL) and 1 N HCl/ether (10 mL) was added dropwise. The precipitate was filtered and washed with EtOAc and dried under vacuum to afford the title compound as a white solid. ¹H NMR (400 MHz, MeOD) δ 9.41 (s, 1H), 8.70 (s, 1H), 8.51 (d, J=9.78 Hz, 1H), 7.53-7.58 (m, 1H), 7.37 (d, J=9.29 Hz, 1H), 7.29 (s, 1H), 7.18 (d, J=8.56 Hz, 1H), 4.81-4.90 (m, 1H), 4.52 (br. s., 2H), 3.71 (br. s., 2H), 3.50 (br. s., 4H), 3.03 (s, 3H), 1.46 (d, J=5.87 Hz, 6H); MS m/e 489.3 (M+H).

### Example 34

### 3-[4-(2-Chloro-pyridin-4-yl)-thiazol-2-ylamino]-4-trifluoromethoxy-benzamide.TFA

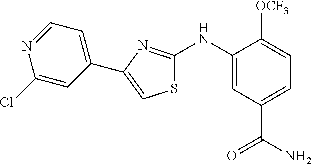

The title compound was prepared using commercially available 2-bromo-1-(2-chloro-pyridin-4-yl)-ethanone in place of 2-bromo-1-(2,4,6-trimethyl-pyridin-3-yl)-ethanone.HBr and 3-thioureido-4-trifluoromethoxy-benzamide (intermediate 17, step d) in place of 4-isopropoxy-3-thioureido-benzamide according to the procedure described in example 19. ¹H NMR (400 MHz, MeOD) δ 9.44 (d, J=1.96 Hz, 1H), 8.40 (d, J=5.38 Hz, 1H), 8.05 (s, 1H), 7.96 (dd, J=1.47, 5.38 Hz, 1H), 7.72 (s, 1H), 7.57 (dd, J=2.20, 8.56 Hz, 1H), 7.45 (dd, J=1.47, 8.31 Hz, 1H); MS m/e 415.1 (M+H).

### Example 35

### 4-Isopropoxy-3-{4-[2-(4-methyl-piperazin-1-yl)-pyridin-4-yl]-thiazol-2-ylamino}-benzenesulfonamide.TFA

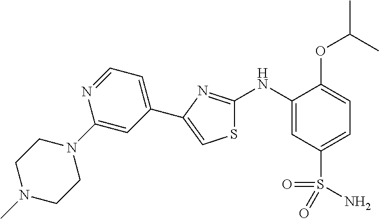

The title compound was prepared using 3-[4-(2-chloro-pyridin-4-yl)-thiazol-2-ylamino]-4-isopropoxy-benzenesulfonamide.TFA (intermediate 9) in place of 3-[4-(6-chloro-pyridin-3-yl)-thiazol-2-ylamino]-4-isopropoxy-benzamide according to the procedure described in example 27. ¹H NMR (400 MHz, MeOD) δ 9.39-9.58 (m, 1H), 7.84-8.04 (m, 2H), 7.80 (d, J=1.71 Hz, 1H), 7.50-7.59 (m, 1H), 7.42 (d, J=6.60 Hz, 1H), 7.13-7.22 (m, 1H), 4.77-4.95 (m, 1H), 3.40-4.50 (m, 8H), 3.05 (s, 3H), 1.48 (dd, J=1.71, 6.11 Hz, 6H); MS m/e 489.3 (M+H).

### Example 36

### 3-{4-[2-(4-Methyl-piperazin-1-yl)-pyridin-4-yl]-thiazol-2-ylamino}-4-trifluoromethoxy-benzamide.TFA

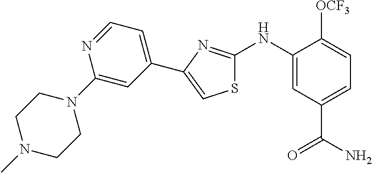

The title compound was prepared using 3-[4-(2-chloro-pyridin-4-yl)-thiazol-2-ylamino]-4-trifluoromethoxy-benzamide.TFA (example 34) in place of 3-[4-(6-chloro-pyridin-3-yl)-thiazol-2-ylamino]-4-isopropoxy-benzamide according to the procedure described in example 27. ¹H NMR (400 MHz, MeOD) δ 9.79 (d, J=2.20 Hz, 1H), 8.04-8.12 (m, 2H), 7.96 (s, 1H), 7.51-7.66 (m, 2H), 7.41-7.51 (m, 1H), 3.40-4.40 (m, 8H), 3.05 (s, 3H); MS m/e 479.2 (M+H).

### Example 37

### 4-Isopropoxy-3-[4-(6-morpholin-4-yl-pyridin-3-yl)-thiazol-2-ylamino]-benzenesulfonamide.TFA

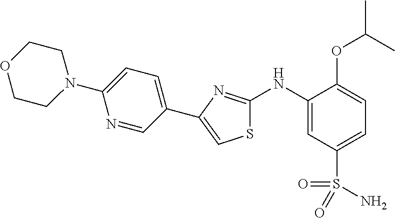

A mixture of 3-[4-(6-chloro-pyridin-3-yl)-thiazol-2-ylamino]-4-isopropoxy-benzenesulfonamide.TsOH (30 mg, 0.071 mmol, intermediate 10) and morpholine (0.6 mL) was microwaved at 150° C. for 30 min. The mixture was purified via reverse phase HPLC with water/acetonitrile/0.1% TFA to give the title compound. ¹H NMR (400 MHz, DMSO-d₆) δ 9.59 (s, 1H), 9.26 (d, J=2.20 Hz, 1H), 8.75 (d, J=2.45 Hz, 1H), 8.09 (dd, J=2.45, 8.80 Hz, 1H), 7.42 (dd, J=2.32, 8.68 Hz, 1H), 7.14-7.25 (m, 4H), 6.87 (d, J=9.05 Hz, 1H), 4.80 (sept, J=6.08 Hz, 1H), 3.65-3.77 (m, 4H), 3.41-3.55 (m, 4H), 1.37 (d, J=5.87 Hz, 6H); MS m/e 476.2 (M+H).

### Example 38

### 4-Isopropoxy-3-{4-[6-(4-ethyl-piperazin-1-yl)-pyridin-3-yl]-thiazol-2-ylamino}-benzenesulfonamide.TFA

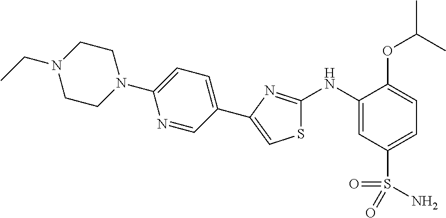

The title compound was prepared using 1-ethyl-piperazine in place of morpholine according to the procedure described in example 37. ¹H NMR (400 MHz, MeOD) δ 9.22 (d, J=2.20 Hz, 1H), 8.56 (d, J=2.20 Hz, 1H), 8.23 (dd, J=2.32, 9.17 Hz, 1H), 7.42 (dd, J=2.32, 8.68 Hz, 1H), 6.97-7.11 (m, 3H), 4.59-4.79 (m, 1H), 3.60-4.20 (m, 4H), 3.48 (br. s., 2H), 3.28-3.43 (m, 2H), 2.98-3.28 (m, 2H), 1.11-1.39 (m, 9H); MS m/e 503.3 (M+H).

### Example 39

### 4-Isopropoxy-3-{4-[6-(4-isopropyl-piperazin-1-yl)-pyridin-3-yl]-thiazol-2-ylamino}-benzenesulfonamide.TFA

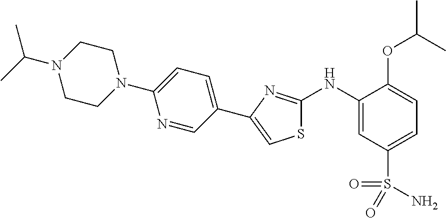

The title compound was prepared using 1-isopropyl-piperazine in place of morpholine according to the procedure described in example 37. ¹H NMR (400 MHz, MeOD) δ 9.23 (d, J=2.45 Hz, 1H), 8.57 (d, J=2.20 Hz, 1H), 8.24 (dd, J=2.20, 9.05 Hz, 1H), 7.42 (dd, J=2.45, 8.56 Hz, 1H), 7.00-7.11 (m, 3H), 4.65-4.80 (m, 1H), 3.70-4.50 (m, 4H), 3.30-3.60 (m, 5H), 1.24-1.50 (m, 12H); MS m/e 517.2 (M+H).

### Example 40

### 4-Isopropoxy-3-{4-[6-(4-methyl-[1,4]diazepan-1-yl)-pyridin-3-yl]-thiazol-2-ylamino}-benzenesulfonamideamide.TFA

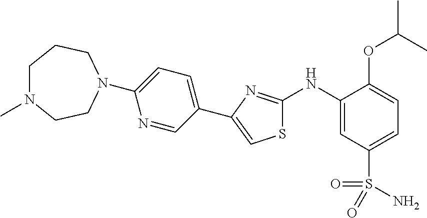

The title compound was prepared using 1-methyl-[1,4]diazepane in place of morpholine according to the procedure described in example 37. ¹H NMR (400 MHz, MeOD) δ 9.38 (d, J=2.20 Hz, 1H), 8.48-8.54 (m, 2H), 7.50-7.59 (m, 1H), 7.37 (d, J=10.03 Hz, 1H), 7.28 (s, 1H), 7.09-7.23 (m, 1H), 4.79-4.90 (m, 1H), 4.16 (br. s., 2H), 3.84 (br. s., 2H), 3.57-3.79 (m, 2H), 3.40-3.57 (m, 2H), 3.01 (s, 3H), 2.38-2.52 (m, 2H), 1.37-1.55 (m, 6H); MS m/e 503.3 (M+H).

### Example 41

### 3-[4-(4-Dimethylamino-3,4,5,6-tetrahydro-2H-[1,2′]bipyridinyl-5′-yl)-thiazol-2-ylamino]-4-isopropoxy-benzenesulfonamide.TFA

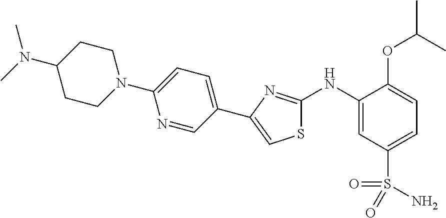

The title compound was prepared using dimethyl-piperidin-4-yl-amine in place of morpholine according to the procedure described in example 37. ¹H NMR (400 MHz, MeOD) δ 9.38 (d, J=2.20 Hz, 1H), 8.43-8.51 (m, 2H), 7.54 (dd, J=2.45, 8.56 Hz, 1H), 7.42 (d, J=10.27 Hz, 1H), 7.27 (s, 1H), 7.17 (d, J=8.56 Hz, 1H), 4.76-4.95 (m, 1H), 4.40 (d, J=13.94 Hz, 2H), 3.53-3.69 (m, 1H), 3.21-3.42 (m, 2H), 2.95 (s, 6H), 2.33 (d, J=11.49 Hz, 2H), 1.78-2.05 (m, 2H), 1.29-1.50 (m, 6H); MS m/e 517.2 (M+H).

### Example 42

### 3-(4-{6-[(2-Dimethylamino-ethyl)-amino]-pyridin-3-yl}-thiazol-2-ylamino)-4-isopropoxy-benzenesulfonamide.TFA

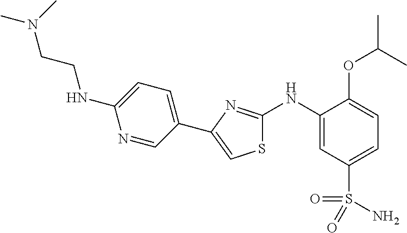

The title compound was prepared using N¹,N¹-dimethyl-ethane-1,2-diamine in place of morpholine according to the procedure described in example 37. ¹H NMR (400 MHz, MeOD) δ 9.47 (d, J=2.20 Hz, 1H), 8.61 (d, J=2.20 Hz, 1H), 8.45 (dd, J=2.20, 9.29 Hz, 1H), 7.55 (dd, J=2.45, 8.56 Hz, 1H), 7.26 (s, 1H), 7.14 (d, J=9.29 Hz, 1H), 7.17 (d, J=8.80 Hz, 1H), 4.80-4.91 (m, 1H), 3.87 (t, J=5.99 Hz, 2H), 3.46-3.54 (m, 2H), 3.05 (s, 6H), 1.43-1.50 (m, 6H); MS m/e 477.2 (M+H).

### Example 43

### 3-(4-{6-[(2-Dimethylamino-ethyl)-methyl-amino]-pyridin-3-yl}-thiazol-2-ylamino)-4-isopropoxy-benzenesulfonamide.TFA

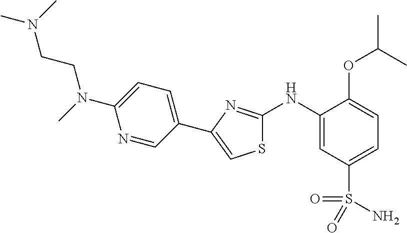

The title compound was prepared using N,N,N′-trimethyl-ethane-1,2-diamine in place of morpholine according to the procedure described in example 37. ¹H NMR (400 MHz, MeOD) δ 9.41 (d, J=2.20 Hz, 1H), 8.60-8.64 (m, 1H), 8.27 (dd, J=2.20, 9.29 Hz, 1H), 7.33-7.52 (m, 2H), 7.02-7.09 (m, 2H), 4.67-4.85 (m, 1H), 3.87-3.95 (m, 2H), 3.31-3.45 (m, 2H), 2.75-2.99 (m, 9H), 1.29-1.39 (m, 6H); MS m/e 491.2 (M+H).

### Example 44

### 4-Isopropoxy-3-[4-(6-piperazin-1-yl-pyridin-3-yl)-thiazol-2-ylamino]-benzenesulfonamide.TFA

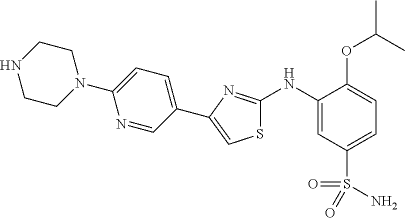

The title compound was prepared using piperazine in place of morpholine according to the procedure described in example 37. ¹H NMR (400 MHz, MeOD) δ 8.93 (br. s., 1H), 8.36-8.50 (m, 2H), 7.60 (dd, J=2.20, 8.56 Hz, 1H), 7.47 (d, J=10.27 Hz, 1H), 7.31 (s, 1H), 7.20 (d, J=8.80 Hz, 1H), 4.85 (sept, J=6.08, 1H), 3.98-4.24 (m, 4H), 3.43-3.65 (m, 4H), 1.43 (d, J=6.11 Hz, 6H); MS m/e 475.2 (M+H).

### Example 45

### 5-[2-(2-Isopropoxy-5-sulfamoyl-phenylamino)-thiazol-4-yl]-pyridine-2-carboxylic acid.HCl

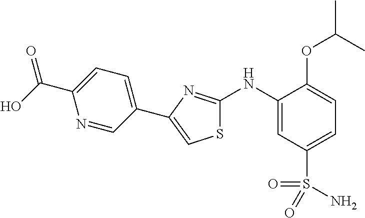

To a solution of 5-[2-(2-isopropoxy-5-sulfamoyl-phenylamino)-thiazol-4-yl]-pyridine-2-carboxylic acid methyl ester.TsOH (108 mg, 0.241 mmol, intermediate 12) in THF (0.5 mL) and MeOH (0.5 mL) was added 2 N NaOH (0.241 mL, 0.482 mmol). The reaction mixture was stirred for 1 h and concentrated. The residue was suspended in water (1 mL) and acidified with 1 N HCl. The mixture was filtered, washed with water and air-dried, affording the title compound as a white solid. ¹H NMR (400 MHz, MeOD) δ 9.46 (s, 1H), 9.35 (br. s., 1H), 9.00-9.11 (m, 1H), 8.49-8.53 (m, 1H), 7.84 (s, 1H), 7.60-7.50 (m, 1H), 7.17 (d, J=8.56 Hz, 1H), 4.84-4.75 (m, 1H), 1.45 (d, J=5.87 Hz, 6H); MS m/e 435.1 (M+H).

### Example 46

### 4-Isopropoxy-3-{4-[6-(4-methyl-piperazine-1-carbonyl)-pyridin-3-yl]-thiazol-2-ylamino}-benzenesulfonamide.TFA

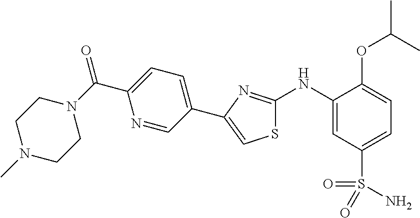

HATU (23 mg, 0.060 mmol) was added to a solution of 5-[2-(2-isopropoxy-5-sulfamoyl-phenylamino)-thiazol-4-yl]-pyridine-2-carboxylic acid.HCl (20 mg, 0.046 mmol, example 45) and 1-methyl-piperazine (36.8 mg, 0.368 mmol) in THF (1 mL) and the mixture was warmed to 70° C. After 4 h at 70° C., the mixture was purified via reverse phase HPLC with water/acetonitrile/0.1% TFA to give the title compound. ¹H NMR (400 MHz, MeOD) δ 9.43 (d, J=2.45 Hz, 1H), 9.16-9.25 (m, 1H), 8.48 (dd, J=2.20, 8.07 Hz, 1H), 7.78 (d, J=8.31 Hz, 1H), 7.53 (dd, J=2.45, 8.56 Hz, 1H), 7.45 (s, 1H), 7.14 (d, J=8.80 Hz, 1H), 4.62-4.92 (m, 1H), 4.43 (br. s., 2H), 3.42-3.72 (m, 3H), 3.12-3.42 (m, 3H), 2.98 (s, 3H), 1.43 (d, J=6.11 Hz, 6H); MS m/e 517.2 (M+H).

### Example 47

### 4-Isopropoxy-3-{4-[6-(4-ethyl-piperazine-1-carbonyl)-pyridin-3-yl]-thiazol-2-ylamino}-benzenesulfonamide.TFA

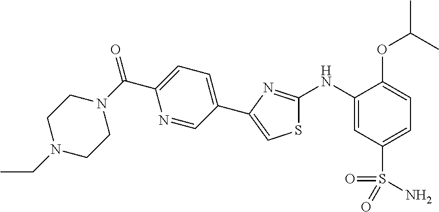

The title compound was prepared using 1-ethyl-piperazine in place of 1-methyl-piperazine according to the procedure described in example 46. ¹H NMR (400 MHz, MeOD) δ 9.43 (d, J=2.20 Hz, 1H), 9.21 (d, J=1.47 Hz, 1H), 8.49 (dd, J=2.20, 8.07 Hz, 1H), 7.79 (d, J=8.07 Hz, 1H), 7.50-7.57 (m, 1H), 7.46 (s, 1H), 7.14 (d, J=8.80 Hz, 1H), 4.66-4.92 (m, 1H), 4.36-4.59 (m, 1H), 3.50-3.80 (m, 3H), 3.10-3.47 (m, 6H), 1.24-1.50 (m, 9H); MS m/e 531.3 (M+H).

### Example 48

### 3-{4-[6-(4-Dimethylamino-piperidine-1-carbonyl)-pyridin-3-yl]-thiazol-2-ylamino}-4-isopropoxy-benzenesulfonamide.TFA

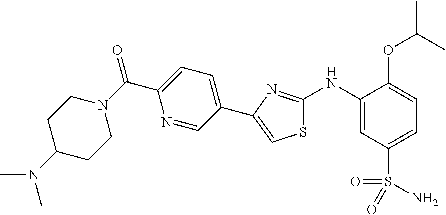

The title compound was prepared using dimethyl-piperidin-4-yl-amine in place of 1-methyl-piperazine according to the procedure described in example 46. ¹H NMR (400 MHz, MeOD) δ 9.43 (d, J=2.20 Hz, 1H), 9.20 (d, J=1.71 Hz, 1H), 8.50 (dd, J=2.20, 8.07 Hz, 1H), 7.69 (d, J=8.07 Hz, 1H), 7.44-7.57 (m, 2H), 7.14 (d, J=8.80 Hz, 1H), 4.76-4.93 (m, 1H), 4.06 (d, J=12.96 Hz, 1H), 3.53 (tt, J=3.70, 11.95 Hz, 1H), 3.09-3.25 (m, 1H), 2.89-2.95 (m, 8H), 2.21 (br. s., 1H), 2.03 (br. s., 1H), 1.70-1.91 (m, 2H), 1.43 (d, J=6.11 Hz, 6H); MS m/e 545.3 (M+H).

### Example 49

### 3-{4-[6-(4-Methyl-[1,4]diazepane-1-carbonyl)-pyridin-3-yl]-thiazol-2-ylamino}-4-isopropoxy-benzenesulfonamide.TFA

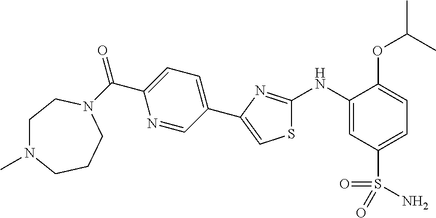

The title compound was prepared using 1-methyl-[1,4]diazepane in place of 1-methyl-piperazine according to the procedure described in example 46. ¹H NMR (400 MHz, MeOD) δ 9.41-9.47 (m, 1H), 9.24-9.19 (m, 1H), 8.41-8.53 (m, 1H), 7.71-7.82 (m, 1H), 7.53 (d, J=8.59 Hz, 1H), 7.45 (d, J=11.62 Hz, 1H), 7.14 (d, J=8.84 Hz, 1H), 4.83 (sept, J=5.87 Hz, 1H), 3.75 (br. s., 5H), 3.44 (br. s., 2H), 3.24-3.39 (m, 1H), 3.02 (d, J=13.89 Hz, 3H), 2.22 (br. s., 2H), 1.44 (d, J=6.06 Hz, 6H); MS m/e 531.3 (M+H).

### Example 50

### 5-[2-(2-Isopropoxy-5-sulfamoyl-phenylamino)-thiazol-4-yl]-pyridine-2-carboxylic acid (2-dimethylamino-ethyl)-amide.TFA

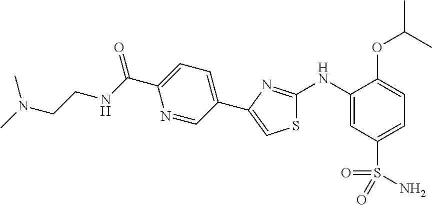

The title compound was prepared using N¹,N¹-dimethyl-ethane-1,2-diamine in place of 1-methyl-piperazine according to the procedure described in example 46. ¹H NMR (400 MHz, MeOD) δ 9.48 (s, 1H), 9.28 (s, 1H), 8.47 (d, J=7.83 Hz, 1H), 8.13 (d, J=8.34 Hz, 1H), 7.47-7.56 (m, 2H), 7.15 (d, J=8.59 Hz, 1H), 4.79-4.88 (m, 1H), 3.82 (t, J=5.56 Hz, 2H), 3.43 (t, J=5.43 Hz, 2H), 3.00 (s, 6H), 1.44 (d, J=5.81 Hz, 6H); MS m/e 505.2 (M+H).

### Example 51

### 4-Isopropoxy-3-{4-[6-(4-isopropyl-piperazine-1-carbonyl)-pyridin-3-yl]-thiazol-2-ylamino}-benzenesulfonamide.TFA

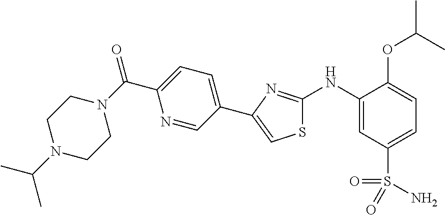

The title compound was prepared using 1-isopropyl-piperazine in place of 1-methyl-piperazine according to the procedure described in example 46. ¹H NMR (400 MHz, MeOD) δ 9.41 (d, J=2.20 Hz, 1H), 9.19 (br. s., 1H), 8.47 (dd, J=2.08, 8.19 Hz, 1H), 7.78 (d, J=8.07 Hz, 1H), 7.48-7.55 (m, 1H), 7.44 (s, 1H), 7.14 (d, J=8.80 Hz, 1H), 4.80-4.90 (m, 1H), 4.45 (br. s., 1H), 3.48-3.65 (m, 6H), 3.20-3.41 (m, 2H), 1.21-1.50 (m, 12H); MS m/e 545.3 (M+H).

### Example 52

### 3-{4-[6-(4-Ethyl-[1,4]diazepane-1-carbonyl)-pyridin-3-yl]-thiazol-2-ylamino}-4-isopropoxy-benzenesulfonamide.TFA

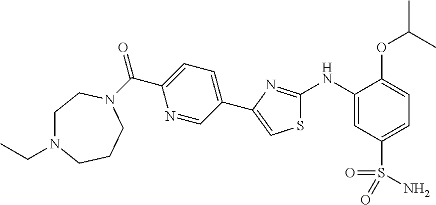

The title compound was prepared using 1-ethyl-[1,4]diazepane in place of 1-methyl-piperazine according to the procedure described in example 46. The compound was identified as a mixture of 2 rotamers in a 1:1 ratio. ¹H NMR (400 MHz, MeOD) δ 9.52 (d, J=2.20 Hz, 0.5H), 9.44 (d, J=2.20 Hz, 0.5H), 9.28 (s, 0.5H), 9.23 (br. s., 0.5H), 8.41-8.52 (m, 1H), 7.70-7.82 (m, 1H), 7.42-7.55 (m, 2H), 7.15 (d, J=8.80 Hz, 1H), 4.77-4.90 (m, 1H), 3.50-4.25 (m, 6H), 3.23-3.50 (m, 4H), 2.22 (br. s., 2H), 1.32-1.50 (m, 9H); MS m/e 545.3 (M+H).

### Example 53

### 5-[2-(2-Isopropoxy-5-sulfamoyl-phenylamino)-thiazol-4-yl]-pyridine-2-carboxylic acid (2-dimethylamino-ethyl)-methyl-amide.TFA

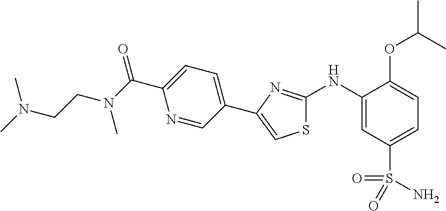

The title compound was prepared using N,N,N′-trimethyl-ethane-1,2-diamine in place of 1-methyl-piperazine according to the procedure described in example 46. ¹H NMR (400 MHz, MeOD) δ 9.61 (br. s., 1H), 9.28 (br. s., 1H), 8.44 (br. s., 1H), 7.89 (d, J=8.07 Hz, 1H), 7.40-7.55 (m, 2H), 7.15 (d, J=8.80 Hz, 1H), 4.76-4.90 (m, 1H), 3.98 (br. s., 1H), 3.89 (br. s., 1H), 3.64 (br. s., 1H), 3.45-3.58 (m, 1H), 3.02-3.22 (m, 9H), 1.45 (d, J=5.87 Hz, 6H); MS m/e 519.3 (M+H).

### Example 54

### 4-{5-[2-(2-Isopropoxy-5-sulfamoyl-phenylamino)-thiazol-4-yl]-pyridine-2-carbonyl}-piperazine-1-carboxylic acid tert-butyl ester.TFA

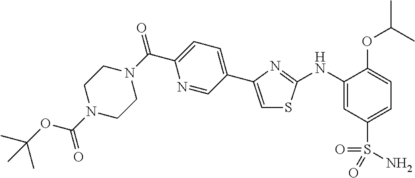

The title compound was prepared using piperazine-1-carboxylic acid tert-butyl ester in place of 1-methyl-piperazine according to the procedure described in example 46. ¹H NMR (400 MHz, MeOD) δ 9.37 (d, J=2.20 Hz, 1H), 9.18 (br. s., 1H), 8.54 (dd, J=1.83, 8.19 Hz, 1H), 7.74 (d, J=7.83 Hz, 1H), 7.44-7.55 (m, 2H), 7.13 (d, J=8.80 Hz, 1H), 4.82 (sept, J=6.08, 1H), 3.76 (br. s., 2H), 3.55 (br. s., 4H), 3.41-3.52 (m, 2H), 1.25-1.55 (m, 15H); MS m/e 603.3 (M+H).

### Example 55

### 5-[2-(2-Isopropoxy-5-sulfamoyl-phenylamino)-thiazol-4-yl]-pyridine-2-carboxylic acid (1-methyl-piperidin-4-yl)-amide.TFA

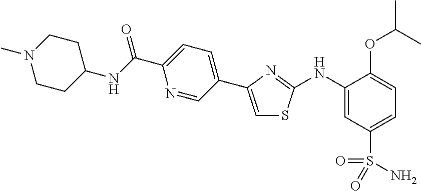

The title compound was prepared using 1-methyl-piperidin-4-ylamine in place of 1-methyl-piperazine according to the procedure described in example 46. ¹H NMR (400 MHz, MeOD) 9.48 (d, J=2.20 Hz, 1H), 9.30 (d, J=1.96 Hz, 1H), 8.47 (dd, J=2.08, 8.19 Hz, 1H), 8.12 (d, J=8.07 Hz, 1H), 7.53 (dd, J=2.20, 8.56 Hz, 1H), 7.48-7.50 (m, 1H), 7.10-7.18 (m, 1H), 4.81-4.90 (m, 1H), 4.10-4.25 (m, 1H), 3.58-3.65 (m, 2H), 3.10-3.31 (m, 2H), 2.91 (s, 3H), 2.22-2.32 (m, 2H), 1.90-2.05 (m, 2H), 1.45 (d, J=6.11 Hz, 6H); MS m/e 531.3 (M+H).

### Example 56

### 3-[4-(6-Hydroxymethyl-pyridin-3-yl)-thiazol-2-ylamino]-4-isopropoxy-benzenesulfonamide

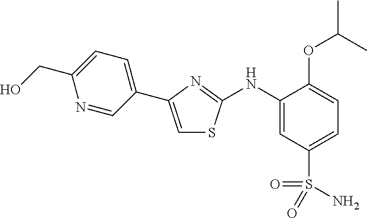

NaBH₄ (139 mg, 3.7 mmol) was added portion by portion to a suspension of 5-[2-(2-isopropoxy-5-sulfamoyl-phenylamino)-thiazol-4-yl]-pyridine-2-carboxylic acid methyl ester.TsOH (330 mg, 0.736 mmol, intermediate 12) in EtOH (10 mL) and THF (5 mL). The mixture was heated at 70° C. for 1 h and cooled down. To the reaction mixture was added silica gel (300 mesh, 4 g), and the resulting suspension was concentrated and purified through solid loading on column chromatography (1%-11% MeOH/DCM) to yield the title compound as a white solid. ¹H NMR (400 MHz, MeOD) δ 9.37 (d, J=2.45 Hz, 1H), 9.07 (d, J=2.20 Hz, 1H), 8.41 (dd, J=2.20, 8.07 Hz, 1H), 7.62 (d, J=8.31 Hz, 1H), 7.52 (dd, J=2.45, 8.56 Hz, 1H), 7.33 (s, 1H), 7.14 (d, J=9.05 Hz, 1H), 4.84 (m, 1H), 4.72 (s, 2H), 1.44 (d, J=5.87 Hz, 6H); MS m/e 421 (M+H).

### Example 57

### 5-[2-(2-Isopropoxy-5-sulfamoyl-phenylamino)-thiazol-4-yl]-pyridine-2-carboxylic acid (1-ethyl-piperidin-4-yl)-amide.TFA

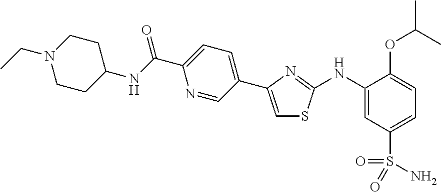

The title compound was prepared using 1-ethyl-piperidin-4-ylamine in place of 1-methyl-piperazine according to the procedure described in example 46. ¹H NMR (400 MHz, MeOD) δ 9.43 (d, J=2.20 Hz, 1H), 9.22 (d, J=1.96 Hz, 1H), 8.44 (dd, J=2.20, 8.07 Hz, 1H), 8.09 (d, J=8.31 Hz, 1H), 7.52 (dd, J=2.32, 8.68 Hz, 1H), 7.45 (s, 1H), 7.13 (d, J=9.05 Hz, 1H), 4.77-4.88 (m, 1H), 3.88-3.97 (m, 1H), 2.94-3.07 (m, 2H), 2.48 (q, J=7.17 Hz, 2H), 2.10-2.25 (m, 2H), 1.95-2.06 (m, 2H), 1.67-1.79 (m, 2H), 1.44 (d, J=6.11 Hz, 6H), 1.13 (t, J=7.21 Hz, 3H); MS m/e 545 (M+H).

### Example 58

### 4-Isopropoxy-3-{4-[6-(piperazine-1-carbonyl)-pyridin-3-yl]-thiazol-2-ylamino}-benzenesulfonamide.TFA

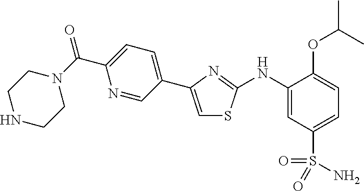

A mixture of 4-{5-[2-(2-isopropoxy-5-sulfamoyl-phenylamino)-thiazol-4-yl]-pyridine-2-carbonyl}-piperazine-1-carboxylic acid tert-butyl ester.TFA (22 mg, 0.0365 mmol, example 54) in TFA (0.3 mL) and dichloromethane (0.3 mL) was stirred for 18 h. The reaction mixture was purified via reverse phase HPLC with water/acetonitrile/0.1% TFA to give the title compound. ¹H NMR (400 MHz, MeOD) δ 9.43 (d, J=2.20 Hz, 1H), 9.22 (br. s., 1H), 8.50 (dd, J=1.83, 8.19 Hz, 1H), 7.79 (d, J=7.83 Hz, 1H), 7.44-7.55 (m, 2H), 7.14 (d, J=8.80 Hz, 1H), 4.83 (sept, J=6.11 Hz, 1H), 3.84-4.10 (m, 4H), 3.25-3.46 (m, 4H), 1.44 (d, J=6.11 Hz, 6H); MS m/e 503 (M+H).

### Example 59

### 3-(4-Pyridin-3-yl-thiazol-2-ylamino)-4-trifluoromethoxy-benzenesulfonamide

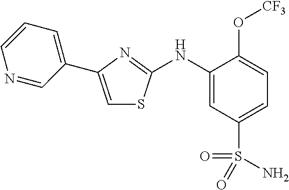

A mixture of 2-bromo-1-pyridin-3-yl-ethanone.HBr (0.030 g, 0.107 mmol) and 3-thioureido-4-trifluoromethoxy-benzenesulfonamide (0.034 g, 0.107 mmol, intermediate 30, step d) in ethanol (3 mL) was stirred at room temperature overnight. Triethylamine (0.050 mL) was added and the mixture was concentrated. The residue was purified by column chromatography to afford the title compound. ¹H NMR (400 MHz, DMSO-d₆) δ 10.49 (s, 1H), 9.55 (d, J=2.45 Hz, 1H), 9.20 (d, J=1.47 Hz, 1H), 8.52 (dd, J=1.59, 4.77 Hz, 1H), 8.31 (dt, J=1.83, 8.07 Hz, 1H), 7.71 (s, 1H), 7.63 (dd, J=1.47, 8.56 Hz, 1H), 7.52 (dd, J=2.20, 8.56 Hz, 1H), 7.49 (s, 2H), 7.45 (dd, J=4.77, 7.95 Hz, 1H). MS m/e 416.9 (M+H).

The following example was obtained from the Johnson and Johnson corporate compound collection:

| Example | Structure | Chemical name |
|---|---|---|
| 60 | 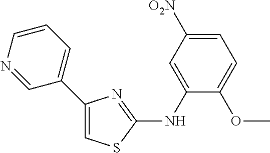 | (2-Methoxy-5- nitro-phenyl)- (4-pyridin-3- yl-thiazol-2-yl)- amine |

(2-Methoxy-5-nitro-phenyl)-(4-pyridin-3-yl-thiazol-2-yl)-amine (example 60) is synthesized by stirring roughly equimolar amounts of commercially available 2-bromo-1-pyridin-3-yl-ethanone.HBr and commercially available 1-(2-methoxy-5-nitrophenyl)-2-thiourea in ethanol at a temperature in the range 20-100° C. for a time period between 10 minutes and 3 days. The product is isolated by concentration of the reaction mixture and purification of the residue by reverse-phase HPLC.

### Example 61

### 3-((4-(6-(4-Cyclopropylpiperazin-1-yl)pyridin-3-yl)thiazol-2-yl)amino)-4-isopropoxybenzenesulfonamide.TFA

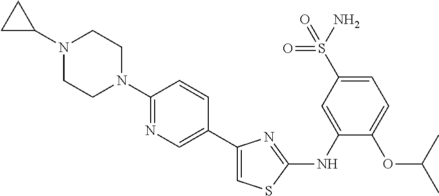

A solution of 2-bromo-1-(6-(4-cyclopropylpiperazin-1-yl)pyridin-3-yl)ethanone.HBr (0.025 g, 0.051 mmol, intermediate 33: Step b) and 4-isopropoxy-3-thioureidobenzenesulfonamide (0.015 g, 0.051 mmol, intermediate 26: Step e) in ethanol was stirred at room temperature overnight. The reaction mixture was then evaporated and purified via reverse phase HPLC eluting with water/acetonitrile/0.1% TFA to give the title compound. ¹H NMR (DMSO-d₆) δ: 9.62 (s, 1H), 9.30 (d, J=2.2 Hz, 1H), 8.79 (d, J=2.2 Hz, 1H), 8.16 (dd, J=8.9, 2.3 Hz, 1H), 7.42 (dd, J=8.4, 2.3 Hz, 1H), 7.29 (s, 1H), 7.21 (d, J=9.0 Hz, 1H), 7.16 (s, 2H), 7.04 (d, J=9.0 Hz, 1H), 4.81 (sept, J=6.1 Hz, 1H), 4.45-4.60 (m, 2H), 3.22-3.41 (m, 2H), 3.07-3.19 (m, 2H), 2.84-3.05 (m, 1H), 1.37 (d, J=5.9 Hz, 6H), 0.93-1.06 (m, 2H), 0.73-0.93 (m, 2H).

### Example 62

### 3-((4-(6-((Cyclopropylmethyl)amino)pyridin-3-yl)thiazol-2-yl)amino)-4-isopropoxybenzenesulfonamide.TFA

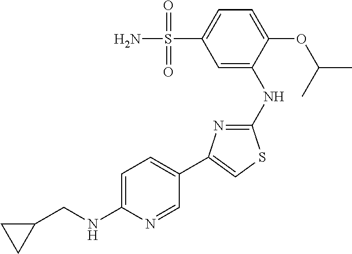

A solution of 3-((4-(6-chloropyridin-3-yl)thiazol-2-yl)amino)-4-isopropoxybenzenesulfonamide (0.050 g, 0.118 mmol, intermediate 10) and cyclopropylmethanamine (0.084 g, 1.18 mmol) in DMSO (0.5 mL) was heated in a sealed tube at 120° C. for 3 days. The reaction mixture was cooled to room temperature and purified via reverse phase HPLC eluting with water/acetonitrile/0.1% TFA to give the title compound. ¹H NMR (400 MHz, DMSO-d₆) δ 9.75 (s, 1H), 9.18 (d, J=2.20 Hz, 1H), 8.38 (d, J=10.51 Hz, 1H), 8.26 (br. s., 1H), 7.35-7.55 (m, 2H), 7.04-7.30 (m, 4H), 4.66-4.90 (m, 1H), 3.27 (d, J=6.85 Hz, 2H), 1.36 (d, J=6.11 Hz, 6H), 1.05-1.21 (m, 1H), 0.50-0.62 (m, 2H), 0.24-0.38 (m, 2H); MS m/e 460.1 (M+H).

### Example 63

### 3-((4-(6-((3-(1H-Imidazol-1-yl)propyl)amino)pyridin-3-yl)thiazol-2-yl)amino)-4-isopropoxybenzenesulfonamide.TFA

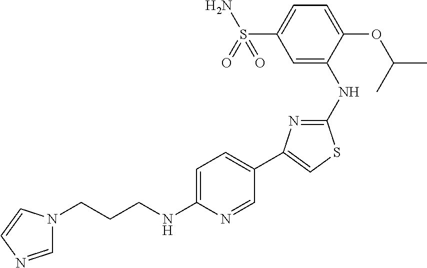

The title compound was prepared using 3-(1H-imidazol-1-yl)propan-1-amine in place of cyclopropylmethanamine according to the procedure for Example 62 to give the title compound. ¹H NMR (400 MHz, DMSO-d₆) δ 9.72 (s, 1H), 9.19 (d, J=2.20 Hz, 1H), 9.15 (s, 1H), 8.29-8.44 (m, 2H), 7.83 (t, J=1.59 Hz, 1H), 7.73 (s, 1H), 7.38-7.49 (m, 2H), 7.13-7.29 (m, 3H), 7.02 (d, 1H), 4.76-4.91 (m, 1H), 4.32 (t, J=6.97 Hz, 2H), 3.40 (t, J=6.60 Hz, 2H), 2.18 (quin, J=6.85 Hz, 2H), 1.37 (d, J=6.11 Hz, 6H); MS m/e 514.2 (M+H).

### Example 64

### 4-Isopropoxy-3-((4-(6-(3-oxopiperazin-1-yl)pyridin-3-yl)thiazol-2-yl)amino)benzenesulfonamide.TFA

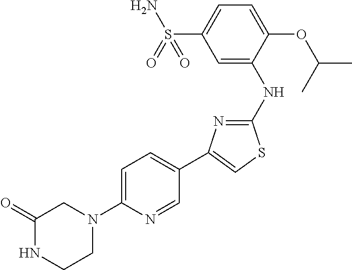

The title compound was prepared using piperazin-2-one in place of morpholine according to the procedure for Example 37 to give the title compound. ¹H NMR (MeOD) δ: 9.43 (d, J=2.2 Hz, 1H), 8.48-8.62 (m, 2H), 7.54 (dd, J=8.6, 2.4 Hz, 1H), 7.43 (d, J=9.5 Hz, 1H), 7.33 (s, 1H), 7.16 (d, J=8.8 Hz, 1H), 4.76-4.86 (m, 1H), 4.30 (s, 2H), 3.92 (dd, J=6.1, 4.4 Hz, 2H), 3.59 (dd, J=6.2, 4.5 Hz, 2H), 1.44 (d, J=6.1 Hz, 6H); MS m/e 489.0 (M+H).

### Example 65

### 3-((4-(6-(4-Acetylpiperazin-1-yl)pyridin-3-yl)thiazol-2-yl)amino)-4-isopropoxybenzenesulfonamide.TFA

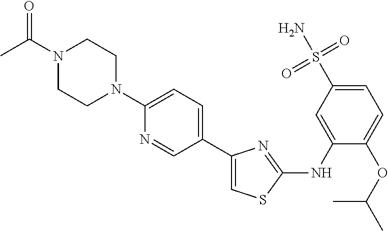

The title compound was prepared using 1-(piperazin-1-yl)ethanone in place of morpholine according to the procedure for Example 37 to give the title compound. ¹H NMR (MeOH) δ: 9.39 (d, J=2.4 Hz, 1H), 8.38-8.50 (m, 2H), 7.52 (dd, J=8.6, 2.2 Hz, 1H), 7.34 (d, J=9.5 Hz, 1H), 7.27 (s, 1H), 7.15 (d, J=8.8 Hz, 1H), 4.84 (sept, J=6.1 Hz, 1H), 3.78-3.85 (m, 6H), 3.70-3.78 (m, 2H), 2.17 (s, 3H), 1.44 (d, J=6.1 Hz, 6H).

### Example 66

### 4-Isopropoxy-3-((4-(6-(4-(3,3,3-trifluoropropyl)piperazin-1-yl)pyridin-3-yl)thiazol-2-yl)amino)benzenesulfonamide.TFA

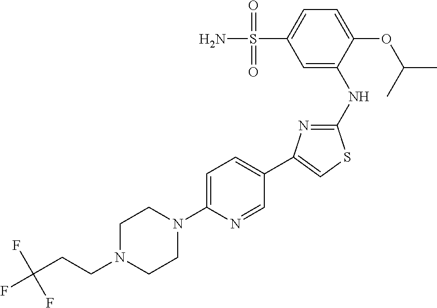

A solution of 1,1,1-trifluoro-3-iodopropane (0.04 mL, 0.32 mmol), 4-isopropoxy-3-((4-(6-(piperazin-1-yl)pyridin-3-yl)thiazol-2-yl)amino)benzenesulfonamide (0.030 g, 0.063 mmol, Example 44), Cs₂CO₃ (0.062 g, 0.190 mmol), acetonitrile (1 mL) and DMF (0.5 mL) was heated to 95° C. for 3 hours. The reaction mixture was concentrated and purified via reverse phase HPLC eluting with water/acetonitrile/0.1% TFA to give the title compound. ¹H NMR (MeOH) δ: 9.32 (d, J=2.2 Hz, 1H), 8.62 (d, J=2.2 Hz, 1H), 8.39 (dd, J=9.3, 2.2 Hz, 1H), 7.52 (dd, J=8.6, 2.2 Hz, 1H), 7.26 (d, J=9.3 Hz, 1H), 7.20 (s, 1H), 7.14 (d, J=9.0 Hz, 1H), 4.83 (sept, J=6.1 Hz, 1H), 3.90-4.05 (m, 4H), 3.43-3.55 (m, 6H), 2.74-2.94 (m, 2H), 1.44 (d, J=6.1 Hz, 6H).

### Example 67

### 3-((4-(6-(3,5-Dimethylpiperazin-1-yl)pyridin-3-yl)thiazol-2-yl)amino)-4-isopropoxybenzenesulfonamide.TFA

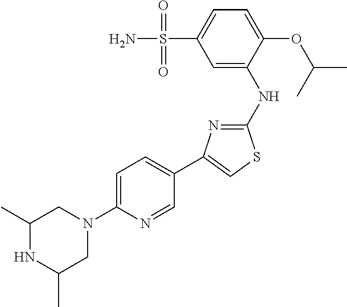

The title compound was prepared using 2,6-dimethylpiperazine in place of morpholine according to the procedure for Example 37 to give the title compound. ¹H NMR (MeOD) δ: 9.33 (d, J=2.2 Hz, 1H), 8.60 (d, J=2.2 Hz, 1H), 8.46 (dd, J=9.4, 2.3 Hz, 1H), 7.52 (dd, J=8.6, 2.4 Hz, 1H), 7.38 (d, J=9.5 Hz, 1H), 7.24 (s, 1H), 7.14 (d, J=9.0 Hz, 1H), 4.75-4.88 (m, 1H), 4.45 (dd, J=14.2, 2.0 Hz, 2H), 3.44-3.63 (m, 2H), 3.09-3.24 (m, 2H), 1.33-1.49 (m, 12H).

### Example 68

### 3-((4-(6-(cis-3,5-Dimethylpiperazin-1-yl)pyridin-3-yl)thiazol-2-yl)amino)-4-isopropoxybenzenesulfonamide.TFA

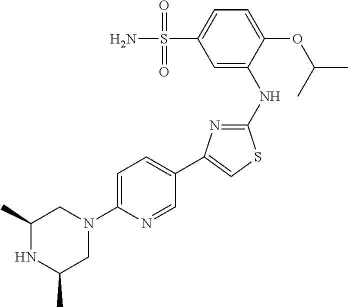

The title compound was prepared using cis-2,6-dimethylpiperazine in place of morpholine according to the procedure for Example 37 to give the title compound. ¹H NMR (MeOD) δ: 9.33 (d, J=2.4 Hz, 1H), 8.61 (d, J=2.2 Hz, 1H), 8.45 (dd, J=9.4, 2.3 Hz, 1H), 7.52 (dd, J=8.6, 2.4 Hz, 1H), 7.37 (d, J=9.3 Hz, 1H), 7.24 (s, 1H), 7.14 (d, J=8.8 Hz, 1H), 4.83 (sept, J=6.1 Hz, 1H), 4.46 (dd, J=14.3, 2.1 Hz, 2H), 3.44-3.65 (m, 2H), 3.17 (dd, J=14.3, 11.6 Hz, 2H), 1.34-1.51 (m, 12H).

### Example 69

### 3-((4-(6-(3,3-Dimethylpiperazin-1-yl)pyridin-3-yl)thiazol-2-yl)amino)-4-isopropoxybenzenesulfonamide.TFA

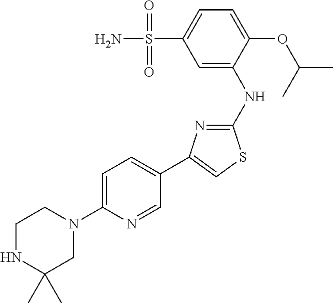

The title compound was prepared using 2,2-dimethylpiperazine in place of morpholine according to the procedure for Example 37 to give the title compound. ¹H NMR (MeOD) δ: 9.35 (d, J=2.2 Hz, 1H), 8.65 (d, J=2.2 Hz, 1H), 8.42 (dd, J=9.3, 2.2 Hz, 1H), 7.53 (dd, J=8.6, 2.2 Hz, 1H), 7.30 (d, J=9.3 Hz, 1H), 7.22 (s, 1H), 7.15 (d, J=8.8 Hz, 1H), 4.83 (sept, J=6.0 Hz, 1H), 3.86-3.98 (m, 2H), 3.79 (s, 2H), 3.40-3.54 (m, 2H), 1.49 (s, 6H), 1.44 (d, J=6.1 Hz, 6H).

### Example 70

### 3-((4-(6-(4-Hydroxypiperidin-1-yl)pyridin-3-yl)thiazol-2-yl)amino)-4-isopropoxybenzenesulfonamide.TFA

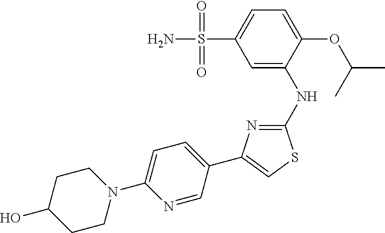

The title compound was prepared using piperidin-4-ol in place of morpholine according to the procedure for Example 37 to give the title compound. ¹H NMR (MeOD) δ: 9.29 (d, J=2.4 Hz, 1H), 8.30 (dd, J=9.8, 2.2 Hz, 1H), 8.25 (d, J=2.0 Hz, 1H), 7.43 (dd, J=8.6, 2.4 Hz, 1H), 7.25 (d, J=9.5 Hz, 1H), 7.14 (s, 1H), 7.05 (d, J=8.8 Hz, 1H), 4.67-4.78 (m, 1H), 3.91 (tt, J=7.5, 3.7 Hz, 1H), 3.83 (ddd, J=13.4, 7.1, 3.7 Hz, 2H), 3.36-3.50 (m, 2H), 1.85-1.97 (m, 2H), 1.53-1.64 (m, 2H), 1.35 (d, J=6.1 Hz, 6H).

### Example 71

### 3-((4-(6-(4-(Cyclopropylmethyl)piperazin-1-yl)pyridin-3-yl)thiazol-2-yl)amino)-4-isopropoxybenzenesulfonamide.TFA

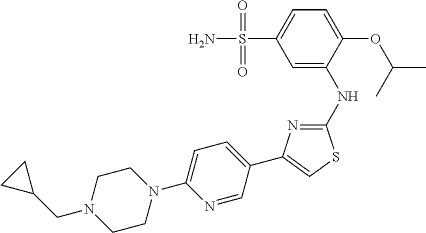

The title compound was prepared using 1-(cyclopropylmethyl)piperazine in place of morpholine according to the procedure for Example 37 to give the title compound. ¹H NMR (MeOH) δ: 9.36 (d, J=2.2 Hz, 1H), 8.66 (d, J=2.2 Hz, 1H), 8.44 (dd, J=9.3, 2.4 Hz, 1H), 7.54 (dd, J=8.6, 2.4 Hz, 1H), 7.30 (d, J=9.3 Hz, 1H), 7.24 (s, 1H), 7.17 (d, J=8.8 Hz, 1H), 4.75-4.90 (m, 1H), 3.42-4.00 (m, 4H), 3.33 (dt, J=3.2, 1.7 Hz, 1H), 3.16 (d, J=7.3 Hz, 2H), 2.17 (s, 4H), 1.46 (d, J=6.1 Hz, 6H), 0.77-0.86 (m, 2H), 0.44-0.54 (m, 2H).

### Example 72

### 4-Isopropoxy-3-((4-(1′,2′,3′,6′-tetrahydro-[2,4′-bipyridin]-5-yl)thiazol-2-yl)amino)benzenesulfonamide.TFA

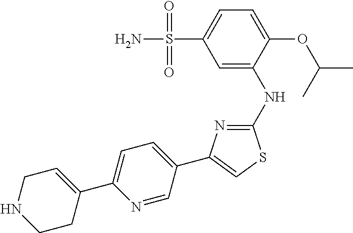

Trifluoroacetic acid (1 mL) was added to a solution of tert-butyl 5-(2-((2-isopropoxy-5-sulfamoylphenyl)amino)thiazol-4-yl)-5′,6′-dihydro-[2,4′-bipyridine]-1′(2′H)-carboxylate (0.040 g, 0.070 mmol, intermediate 31) in DCM (1 mL) at room temperature and stirred for 2 hours. The reaction mixture was concentrated and purified via reverse phase HPLC eluting with water/acetonitrile/0.1% TFA to give the title compound. ¹H NMR (MeOH) δ: 9.41 (d, J=2.2 Hz, 1H), 9.17 (d, J=1.7 Hz, 1H), 8.69 (dd, J=8.6, 2.2 Hz, 1H), 7.93 (d, J=8.3 Hz, 1H), 7.45-7.60 (m, 2H), 7.16 (d, J=8.8 Hz, 1H), 6.77 (br. s., 1H), 4.78-4.89 (m, 1H), 3.96-4.01 (m, 2H), 3.53 (t, J=6.0 Hz, 2H), 2.86-3.01 (m, 2H), 1.44 (d, J=6.1 Hz, 6H).

### Example 73

### 4-(5-(2-((2-Isopropoxy-5-sulfamoylphenyl)amino)thiazol-4-yl)pyridin-2-yl)piperazine-2-carboxamide.TFA

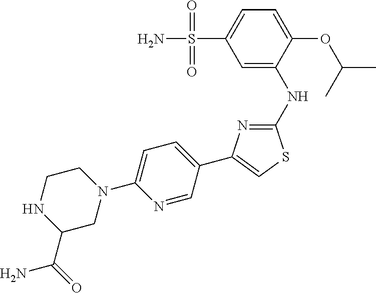

The title compound was prepared using piperazine-2-carboxamide in place of morpholine according to the procedure for Example 37 to give the title compound. ¹H NMR (MeOH) δ: 9.34 (d, J=2.2 Hz, 1H), 8.76 (d, J=2.2 Hz, 1H), 8.38 (dd, J=9.2, 2.3 Hz, 1H), 7.53 (dd, J=8.7, 2.3 Hz, 1H), 7.25 (d, J=9.0 Hz, 1H), 7.05-7.21 (m, 2H), 4.79-4.87 (m, 1H), 4.75 (dt, J=14.3, 1.8 Hz, 1H), 4.31-4.43 (m, 1H), 4.18 (dd, J=11.0, 3.7 Hz, 1H), 3.56 (dt, J=12.5, 2.6 Hz, 1H), 3.38-3.50 (m, 2H), 3.32-3.38 (m, 1H), 1.44 (d, J=6.1 Hz, 6H).

### Example 74

### 4-Isopropoxy-3-((4-(6-((2-methoxyethyl)(methyl)amino)pyridin-3-yl)thiazol-2-yl)amino)benzenesulfonamide.TFA

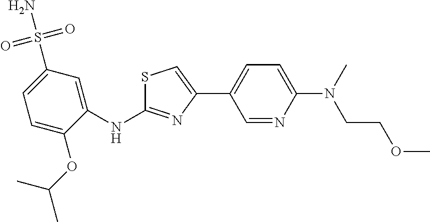

The title compound was prepared using 2-methoxy-N-methylethanamine in place of morpholine according to the procedure for Example 37 to give the title compound. ¹H NMR (MeOD) δ: 9.45 (d, J=2.2 Hz, 1H), 8.37-8.52 (m, 2H), 7.53 (dd, J=8.6, 2.2 Hz, 1H), 7.39 (d, J=9.5 Hz, 1H), 7.28 (s, 1H), 7.16 (d, J=8.6 Hz, 1H), 4.78-4.86 (m, 1H), 3.89 (t, J=4.9 Hz, 2H), 3.71 (t, J=4.9 Hz, 2H), 3.36-3.39 (m, 3H), 3.34 (s, 3H), 1.44 (d, J=6.1 Hz, 6H).

### Example 75

### 3-((4-(6-((2-Hydroxyethyl)(methyl)amino)pyridin-3-yl)thiazol-2-yl)amino)-4-isopropoxybenzenesulfonamide.TFA

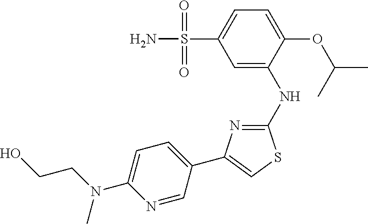

The title compound was prepared using 2-(methylamino)ethanol in place of morpholine according to the procedure for Example 37 to give the title compound. ¹H NMR (MeOD) δ: 9.44 (d, J=2.4 Hz, 1H), 8.34-8.50 (m, 2H), 7.53 (dd, J=8.6, 2.4 Hz, 1H), 7.38 (d, J=9.5 Hz, 1H), 7.27 (s, 1H), 7.16 (d, J=8.8 Hz, 1H), 4.76-4.88 (m, 1H), 3.84-3.91 (m, 2H), 3.78-3.84 (m, 2H), 3.34 (s, 3H), 1.44 (d, J=6.1 Hz, 6H).

### Example 76

### 4-Isopropoxy-3-((4-(6-(piperidin-1-yl)pyridin-3-yl)thiazol-2-yl)amino)benzenesulfonamide.TFA

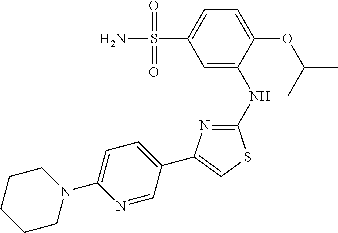

The title compound was prepared using piperidine in place of morpholine according to the procedure for Example 37 to give the title compound. ¹H NMR (MeOD) δ: 9.44 (d, J=2.2 Hz, 1H), 8.40-8.50 (m, 2H), 7.53 (dd, J=8.7, 2.3 Hz, 1H), 7.43 (d, J=9.5 Hz, 1H), 7.28 (s, 1H), 7.16 (d, J=8.8 Hz, 1H), 4.78-4.87 (m, 1H), 3.68-3.83 (m, 4H), 1.74-1.85 (m, 6H), 1.44 (d, J=6.1 Hz, 6H).

### Example 77

### 4-Isopropoxy-3-((4-(6-(4-methylpiperidin-1-yl)pyridin-3-yl)thiazol-2-yl)amino)benzenesulfonamide.TFA

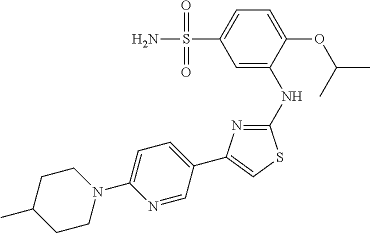

The title compound was prepared using 4-methylpiperidine in place of morpholine according to the procedure for Example 37 to give the title compound. ¹H NMR (MeOD) δ: 9.44 (d, J=2.4 Hz, 1H), 8.47 (dd, J=9.7, 2.3 Hz, 1H), 8.42 (d, J=2.2 Hz, 1H), 7.53 (dd, J=8.6, 2.4 Hz, 1H), 7.44 (d, J=9.5 Hz, 1H), 7.28 (s, 1H), 7.16 (d, J=9.0 Hz, 1H), 4.77-4.86 (m, 1H), 4.19 (d, J=13.7 Hz, 2H), 3.24-3.36 (m, 2H), 1.86-1.96 (m, 2H), 1.75-1.86 (m, 1H), 1.44 (d, J=6.1 Hz, 6H), 1.26-1.40 (m, 2H), 1.04 (d, J=6.6 Hz, 3H).

### Example 78

### 4-Isopropoxy-3-((4-(6-(4-methoxypiperidin-1-yl)pyridin-3-yl)thiazol-2-yl)amino)benzenesulfonamide.TFA

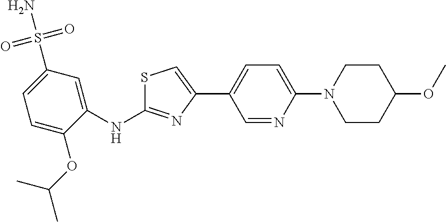

The title compound was prepared using 4-methoxypiperidine in place of morpholine according to the procedure for Example 37 to give the title compound. ¹H NMR (MeOD) δ: 9.43 (d, J=2.2 Hz, 1H), 8.46 (dd, J=9.5, 2.2 Hz, 1H), 8.41 (d, J=2.2 Hz, 1H), 7.53 (dd, J=8.7, 2.3 Hz, 1H), 7.42 (d, J=9.5 Hz, 1H), 7.28 (s, 1H), 7.16 (d, J=8.8 Hz, 1H), 4.77-4.88 (m, 1H), 3.77-3.93 (m, 2H), 3.55-3.73 (m, 3H), 3.41 (s, 3H), 1.97-2.10 (m, 2H), 1.67-1.89 (m, J=13.8, 7.1, 7.1, 3.7 Hz, 2H), 1.44 (d, J=5.9 Hz, 6H).

### Example 79

### 3-((4-(6-(4-(Hydroxymethyl)piperidin-1-yl)pyridin-3-yl)thiazol-2-yl)amino)-4-isopropoxybenzenesulfonamide.TFA

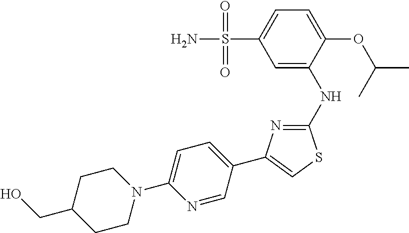

The title compound was prepared using piperidin-4-ylmethanol in place of morpholine according to the procedure for Example 37 to give the title compound. ¹H NMR (MeOD) δ: 9.42 (d, J=2.4 Hz, 1H), 8.43 (dd, J=9.5, 2.2 Hz, 1H), 8.38 (d, J=2.2 Hz, 1H), 7.53 (dd, J=8.6, 2.4 Hz, 1H), 7.39 (d, J=9.5 Hz, 1H), 7.23-7.29 (m, 1H), 7.16 (d, J=8.8 Hz, 1H), 4.77-4.88 (m, 1H), 4.19 (d, J=13.4 Hz, 2H), 3.48 (d, J=6.1 Hz, 2H), 3.23-3.30 (m, 2H), 1.80-2.03 (m, 3H), 1.44 (d, J=5.9 Hz, 6H), 1.33-1.43 (m, 2H).

### Example 80

### 4-Isopropoxy-3-((4-(6-(3-(trifluoromethyl)piperazin-1-yl)pyridin-3-yl)thiazol-2-yl)amino)benzenesulfonamide.TFA

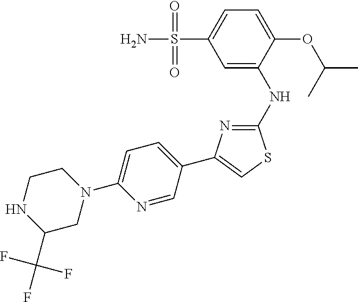

The title compound was prepared using 2-(trifluoromethyl)piperazine in place of morpholine according to the procedure for Example 37 to give the title compound. ¹H NMR (MeOD) δ: 9.37 (d, J=2.2 Hz, 1H), 8.68 (d, J=2.0 Hz, 1H), 8.41 (dd, J=9.2, 2.3 Hz, 1H), 7.53 (dd, J=8.6, 2.2 Hz, 1H), 7.29 (d, J=9.3 Hz, 1H), 7.19-7.23 (m, 1H), 7.15 (d, J=8.6 Hz, 1H), 4.77-4.87 (m, 1H), 4.60-4.69 (m, 1H), 4.29 (d, J=13.9 Hz, 1H), 4.17 (td, J=7.0, 3.4 Hz, 1H), 3.34-3.54 (m, 3H), 3.18-3.28 (m, 1H), 1.39-1.47 (m, 6H).

### Example 81

### 4-Isopropoxy-3-((4-(6-(piperidin-4-yl)pyridin-3-yl)thiazol-2-yl)amino)benzenesulfonamide.TFA

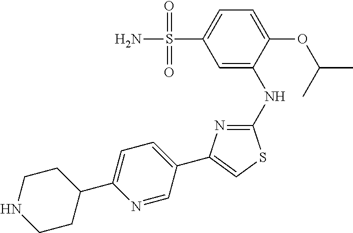

The title compound was prepared using tert-butyl 4-(5-(2-((2-isopropoxy-5-sulfamoylphenyl)amino)thiazol-4-yl)pyridin-2-yl)piperidine-1-carboxylate (intermediate 32) instead of tert-butyl 5-(2-((2-isopropoxy-5-sulfamoylphenyl)amino)thiazol-4-yl)-5′,6′-dihydro-[2,4′-bipyridine]-1′(2′H)-carboxylate according to the procedure for Example 72 to give the title compound. ¹H NMR (MeOD) δ: 9.38 (d, J=2.4 Hz, 1H), 9.18 (d, J=2.0 Hz, 1H), 8.83 (dd, J=8.6, 2.0 Hz, 1H), 7.87 (d, J=8.6 Hz, 1H), 7.57 (s, 1H), 7.50 (dd, J=8.6, 2.4 Hz, 1H), 7.08-7.17 (m, 1H), 4.83 (sept, J=6.1 Hz, 1H), 3.60 (d, J=12.7 Hz, 2H), 3.34-3.44 (m, 1H), 3.21 (td, J=12.8, 2.4 Hz, 2H), 2.28 (d, J=13.9 Hz, 2H), 2.04-2.19 (m, 2H), 1.44 (d, J=6.1 Hz, 6H).

### Example 82

### 3-((4-(6-(5,6-Dihydroimidazo[1,2-a]pyrazin-7(8H)-yl)pyridin-3-yl)thiazol-2-yl)amino)-4-isopropoxybenzenesulfonamide.TFA

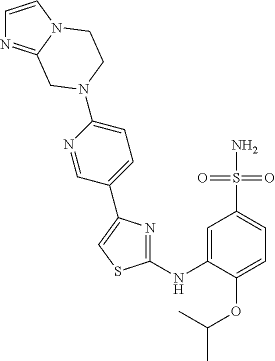

The title compound was prepared using 5,6,7,8-tetrahydroimidazo[1,2-a]pyrazine in place of morpholine according to the procedure for Example 37 to give the title compound. ¹H NMR (MeOD) δ: 9.29 (d, J=2.2 Hz, 1H), 8.72 (d, J=2.2 Hz, 1H), 8.30 (dd, J=8.9, 2.3 Hz, 1H), 7.38-7.63 (m, 3H), 7.04-7.28 (m, 3H), 5.10 (s, 2H), 4.74-4.87 (m, 1H), 4.37 (t, J=5.3 Hz, 2H), 4.20 (t, J=5.4 Hz, 2H), 1.43 (d, J=6.1 Hz, 6H).

### Example 83

### 4-Isopropoxy-3-((4-(6-(4-(methylsulfonyl)piperazin-1-yl)pyridin-3-yl)thiazol-2-yl)amino)benzenesulfonamide.TFA

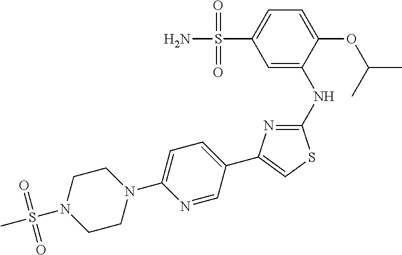

The title compound was prepared using 1-(methylsulfonyl)piperazine in place of morpholine according to the procedure for Example 37 to give the title compound. ¹H NMR (MeOD) δ: 9.43 (d, J=2.0 Hz, 1H), 8.52 (s, 2H), 7.43-7.60 (m, 2H), 7.34 (s, 1H), 7.16 (d, J=8.8 Hz, 1H), 4.75-4.86 (m, 1H), 3.78-3.96 (m, J=4.2 Hz, 4H), 3.42-3.54 (m, 4H), 1.44 (d, J=6.1 Hz, 6H).

### Example 84

### 5-(2-((2-Isopropoxy-5-sulfamoylphenyl)amino)thiazol-4-yl)-N-(1,2,2,6,6-pentamethylpiperidin-4-yl)picolinamide.TFA

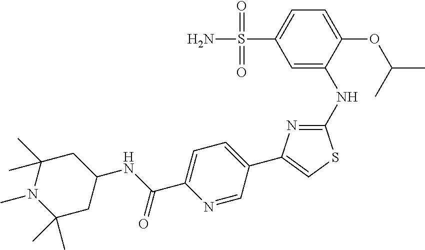

The title compound was prepared using 1,2,2,6,6-pentamethylpiperidin-4-amine in place of 1-methylpiperazine according to the procedure for Example 46 to give the title compound. ¹H NMR (MeOH) δ: 9.41-9.50 (m, 1H), 9.26 (d, J=1.7 Hz, 1H), 8.45 (dd, J=8.1, 2.2 Hz, 1H), 8.11 (d, J=8.1 Hz, 1H), 7.49-7.56 (m, 1H), 7.46 (s, 1H), 7.14 (d, J=8.8 Hz, 1H), 4.74-4.88 (m, 1H), 4.53 (tt, J=12.3, 3.7 Hz, 1H), 2.88 (s, 3H), 2.24 (dd, J=14.1, 3.5 Hz, 2H), 1.88-2.05 (m, 2H), 1.57 (s, 6H), 1.52 (s, 6H), 1.44 (d, J=6.1 Hz, 6H).

### Example 85

### N-Isopentyl-5-(2-((2-isopropoxy-5-sulfamoylphenyl)amino)thiazol-4-yl)picolinamide.TFA

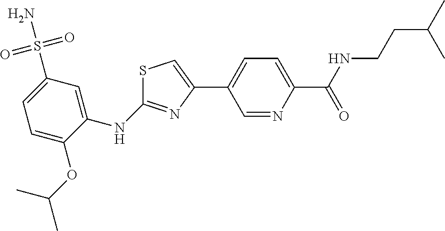

The title compound was prepared using 3-methylbutan-1-amine in place of 1-methylpiperazine according to the procedure for Example 46 to give the title compound. ¹H NMR (MeOH) δ: 9.42 (s, 1H), 9.26 (s, 1H), 8.52 (s, 1H), 8.15 (s, 1H), 7.49 (s, 2H), 7.16 (s, 1H), 3.41-3.52 (m, 2H), 1.62-1.76 (m, 1H), 1.50-1.62 (m, 2H), 1.44 (d, J=5.9 Hz, 6H), 0.98 (d, J=6.8 Hz, 6H).

### Example 86

### 5-(2-((2-Isopropoxy-5-sulfamoylphenyl)amino)thiazol-4-yl)-N-(1-azabicyclo[2.2.2]octan-3-yl)picolinamide.TFA

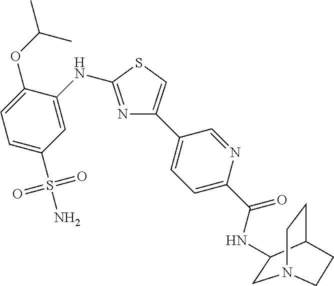

The title compound was prepared using 1-azabicyclo[2.2.2]octan-3-amine.2HCl in place of 1-methylpiperazine according to the procedure for Example 46 to give the title compound. ¹H NMR (MeOH) δ: 9.41-9.54 (m, 1H), 9.24 (br. s., 1H), 8.34-8.55 (m, 1H), 8.10 (t, J=7.6 Hz, 1H), 7.48-7.60 (m, 2H), 7.06-7.24 (m, 1H), 4.84 (sept, J=6.1 Hz, 1H), 4.50 (br. s., 1H), 3.76-3.96 (m, 1H), 3.43-3.51 (m, 1H), 3.33-3.43 (m, 4H), 2.38 (br. s., 1H), 2.17-2.34 (m, 1H), 2.03-2.17 (m, 2H), 1.85-2.03 (m, 1H), 1.39-1.50 (m, 6H).

### Example 87

### Ethyl 3-(5-(2-((2-isopropoxy-5-sulfamoylphenyl)amino)thiazol-4-yl)picolinamido)propanoate.TFA

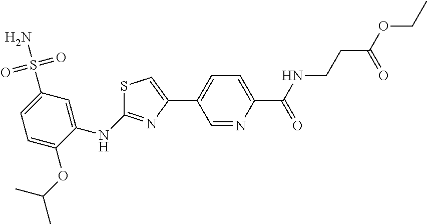

The title compound was prepared using ethyl 3-aminopropanoate.HCl in place of 1-methylpiperazine according to the procedure for Example 46 to give the title compound. ¹H NMR (MeOH) δ: 9.37 (d, J=2.4 Hz, 1H), 9.24 (d, J=2.0 Hz, 1H), 8.49 (dd, J=8.2, 2.1 Hz, 1H), 8.14 (d, J=8.3 Hz, 1H), 7.55 (dd, J=8.7, 2.3 Hz, 1H), 7.49 (s, 1H), 7.16 (d, J=8.8 Hz, 1H), 4.17 (q, J=7.1 Hz, 2H), 3.67-3.77 (m, 2H), 2.69 (t, J=6.7 Hz, 2H), 1.45 (d, J=5.9 Hz, 6H), 1.26 (t, J=7.2 Hz, 3H).

### Example 88

### 5-(2-((2-Isopropoxy-5-sulfamoylphenyl)amino)thiazol-4-yl)-N-(1-isopropylpiperidin-4-yl)picolinamide.TFA

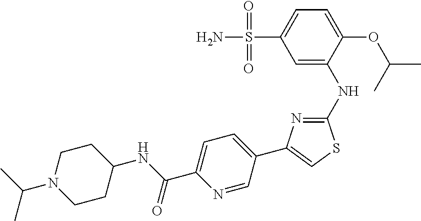

The title compound was prepared using ethyl 1-isopropylpiperidin-4-amine in place of 1-methylpiperazine according to the procedure for Example 46 to give the title compound. ¹H NMR (MeOH) δ: 9.46 (br. s., 1H), 9.24 (br. s., 1H), 8.45 (br. s., 1H), 7.96-8.20 (m, 1H), 7.54 (d, J=8.6 Hz, 1H), 7.45 (br. s., 1H), 7.14 (d, J=8.6 Hz, 1H), 4.75-4.88 (m, 1H), 4.17 (br. s., 1H), 3.49-3.61 (m, 3H), 3.13-3.26 (m, 2H), 2.22-2.37 (m, 2H), 1.90-2.06 (m, 2H), 1.44 (d, J=5.9 Hz, 6H), 1.37 (d, J=6.6 Hz, 6H).

### Example 89

### 5-(2-((2-Isopropoxy-5-sulfamoylphenyl)amino)thiazol-4-yl)-N-(1-methylpiperidin-3-yl)picolinamide.TFA

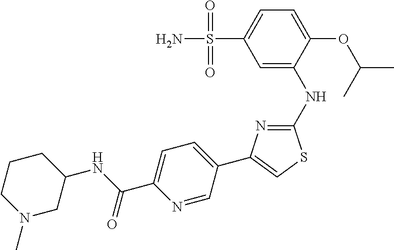

The title compound was prepared using 1-methylpiperidin-3-amine in place of 1-methylpiperazine according to the procedure for Example 46 to give the title compound. ¹H NMR (MeOH) δ: 9.38-9.47 (m, 1H), 9.24 (s, 1H), 8.44 (dd, J=8.3, 2.0 Hz, 1H), 8.09 (d, J=8.3 Hz, 1H), 7.53 (dd, J=8.7, 2.3 Hz, 1H), 7.45 (s, 1H), 7.14 (d, J=8.8 Hz, 1H), 4.75-4.89 (m, 1H), 4.23-4.38 (m, 1H), 3.67 (dd, J=11.7, 4.2 Hz, 1H), 3.54 (d, J=12.0 Hz, 1H), 2.90-2.97 (m, 4H), 2.12 (d, J=11.7 Hz, 2H), 1.82-1.99 (m, 2H), 1.63-1.82 (m, 1H), 1.44 (d, J=5.9 Hz, 6H).

### Example 90

### 5-(2-((2-Isopropoxy-5-sulfamoylphenyl)amino)thiazol-4-yl)-N-(2-methoxyethyl)picolinamide.TFA

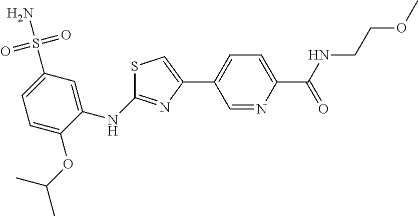

The title compound was prepared using 2-methoxyethanamine in place of 1-methylpiperazine according to the procedure for Example 46 to give the title compound. ¹H NMR (MeOH) δ: 9.40 (br. s., 1H), 9.24 (br. s., 1H), 8.50 (br. s., 1H), 8.14 (br. s., 1H), 7.53 (d, J=8.6 Hz, 1H), 7.47 (br. s., 1H), 7.15 (d, J=8.6 Hz, 1H), 4.76-4.85 (m, 1H), 3.55-3.67 (m, 4H), 3.40 (s, 3H), 1.44 (d, J=6.1 Hz, 6H).

### Example 91

### 5-(2-((2-Isopropoxy-5-sulfamoylphenyl)amino)thiazol-4-yl)-N-((tetrahydro-2H-pyran-4-yl)methyl)picolinamide.TFA

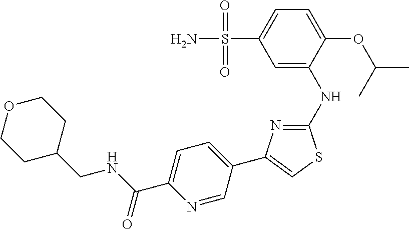

The title compound was prepared using (tetrahydro-2H-pyran-4-yl)methanamine in place of 1-methylpiperazine according to the procedure for Example 46 to give the title compound. ¹H NMR (MeOH) δ: 9.42 (d, J=2.2 Hz, 1H), 9.27 (d, J=2.2 Hz, 1H), 8.56 (d, J=8.6 Hz, 1H), 8.18 (d, J=8.1 Hz, 1H), 7.43-7.59 (m, 2H), 7.16 (d, J=8.8 Hz, 1H), 4.79-4.86 (m, 1H), 3.90-4.00 (m, J=13.0 Hz, 2H), 3.35-3.49 (m, 4H), 1.64-1.75 (m, 2H), 1.44 (d, J=6.1 Hz, 6H), 1.22-1.41 (m, 3H).

### Example 92

### 4-Isopropoxy-3-((4-(6-((3-methoxypropyl)(methyl)amino)pyridin-3-yl)thiazol-2-yl)amino)benzenesulfonamide.TFA

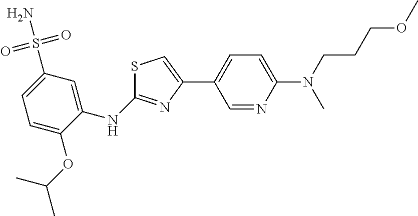

The title compound was prepared using 3-methoxy-N,N-dimethylpropan-1-amine in place of morpholine according to the procedure for Example 37 to give the title compound. ¹H NMR (MeOH) δ: 9.43 (d, J=2.4 Hz, 1H), 8.46 (dd, J=9.7, 2.1 Hz, 1H), 8.41 (d, J=2.0 Hz, 1H), 7.53 (dd, J=8.7, 2.3 Hz, 1H), 7.32 (d, J=9.8 Hz, 1H), 7.27 (s, 1H), 7.16 (d, J=8.8 Hz, 1H), 4.78-4.86 (m, 1H), 3.75 (t, J=6.8 Hz, 2H), 3.46 (t, J=5.6 Hz, 2H), 3.34 (s, 3H), 3.28 (s, 3H), 1.97 (quin, J=6.2 Hz, 2H), 1.44 (d, J=6.1 Hz, 6H).

### Example 93

### (E)-4-Isopropoxy-3-((4-(6-(3-methoxyprop-1-en-1-yl)pyridin-3-yl)thiazol-2-yl)amino)benzenesulfonamide.TFA

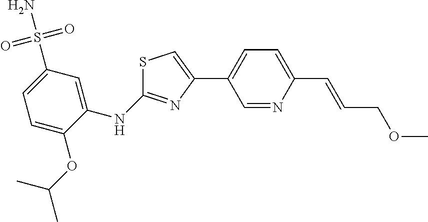

A solution of 3-((4-(6-chloropyridin-3-yl)thiazol-2-yl)amino)-4-isopropoxybenzenesulfonamide.TsOH (0.070 g, 0.117 mmol, intermediate 10), (E)-2-(3-methoxyprop-1-en-1-yl)-4,4,5,5-tetramethyl-1,3,2-dioxaborolane (0.035 g, 0.176 mmol), Pd(PPh₃)₄ (0.019 g, 0.023 mmol), and Na₂CO₃ (0.234 mL) in DME (1.5 mL) was heated to 96° C. for 4 hours. The reaction mixture was concentrated and purified via reverse phase HPLC eluting with water/acetonitrile/0.1% TFA to give the title compound. ¹H NMR (MeOD) δ: 9.38 (d, J=2.2 Hz, 1H), 9.01 (d, J=2.0 Hz, 1H), 8.81 (dd, J=8.8, 2.0 Hz, 1H), 8.11 (d, J=8.6 Hz, 1H), 7.61 (s, 1H), 7.53 (dd, J=8.6, 2.2 Hz, 1H), 6.96-7.20 (m, 2H), 6.82 (d, J=16.1 Hz, 1H), 4.73-4.91 (m, 1H), 4.23 (dd, J=4.0, 2.1 Hz, 2H), 3.46 (s, 3H), 1.45 (d, J=6.1 Hz, 6H).

### Example 94

### 4-Isopropoxy-3-((4-(6-(3-methoxypropyl)pyridin-3-yl)thiazol-2-yl)amino)benzenesulfonamide.TFA

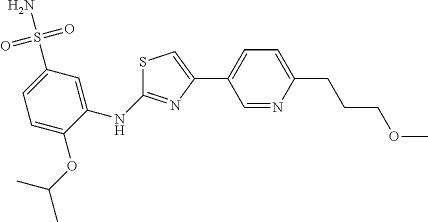

A solution of (E)-4-isopropoxy-3-((4-(6-(3-methoxyprop-1-en-1-yl)pyridin-3-yl)thiazol-2-yl)amino)benzenesulfonamide.TFA (0.026 g, 0.057 mmol, example 93) and 10% Pd—C in methanol (5 mL) was subjected to a parr-shaker with 40 psi of hydrogen for 4 hours. The reaction was then filtered through celite to give the title compound. ¹H NMR (MeOH) δ: 9.45 (d, J=2.4 Hz, 1H), 9.14 (d, J=1.7 Hz, 1H), 8.92 (dd, J=8.6, 2.0 Hz, 1H), 7.94 (d, J=8.3 Hz, 1H), 7.64 (s, 1H), 7.54 (dd, J=8.6, 2.2 Hz, 1H), 7.16 (d, J=8.8 Hz, 1H), 4.76-4.86 (m, 1H), 3.48 (t, J=5.9 Hz, 2H), 3.35 (s, 3H), 3.13 (t, J=7.5 Hz, 2H), 2.04-2.13 (m, 2H), 1.44 (d, J=6.1 Hz, 6H).

### Example 95

### 5-(2-((2-Isopropoxy-5-sulfamoylphenyl)amino)thiazol-4-yl)-N-((1-methylpiperidin-4-yl)methyl)picolinamide.TFA

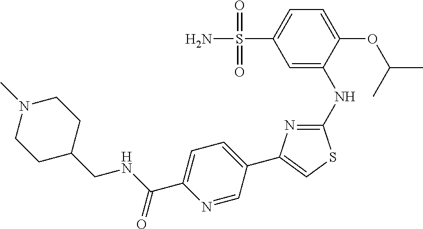

The title compound was prepared using (1-methylpiperidin-4-yl)methanamine in place of 1-methylpiperazine according to the procedure for Example 46 to give the title compound.

### Example 96

### 5-(2-((2-Isopropoxy-5-sulfamoylphenyl)amino)thiazol-4-yl)-N-(tetrahydro-2H-pyran-4-yl)picolinamide.TFA

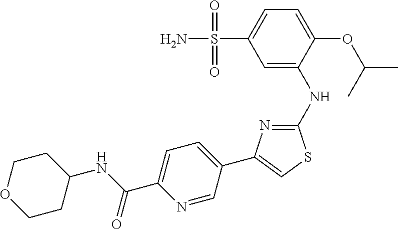

The title compound was prepared using tetrahydro-2H-pyran-4-amine in place of 1-methylpiperazine according to the procedure for Example 46 to give the title compound. MS m/e 518.1 (M+H).

### Compound α

### 3-(2′,4′-Dimethyl-[4,5′]bithiazolyl-2-ylamino)-4-isopropoxy-benzenesulfonamide.HBr

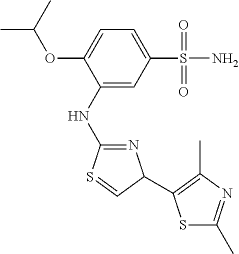

Compound α was tested in cell based and in-vitro assays (vide infra). The cell based and in-vivo activity of Compound α is provided as representative of the activity of the compounds of the present invention, but is not to be construed as limiting the invention in any way.

### Cloning, Expression and Purification

Cloning of Human proMMP9

Amino acid numbering for all human proMMP9 constructs was based on UniProtKB/Swiss-Prot P14780, full-length human matrix metalloproteinase-9 precursor, proMMP9(1-707) (SEQ ID NO:1). One construct, proMMP9(20-445) (SEQ ID NO:2), was based on the previously published crystal structure (Acta Crystallogr D Biol Crystallogr 58(Pt 7): 1182-92). The construct lacked the signal peptide at the N-terminus and also lacked the four hemopexin-like domains at the C-terminus. An N-terminal truncated construct was also designed with an N-terminus truncation after the first observable electron density in the previously published proMMP9 structure and a single amino acid was removed from the C-terminus to produce proMMP9(29-444) (SEQ ID NO:3). Other truncated constructs were also synthesized without the three fibronectin type-II domains (ΔFnII), amino acids 216-390. The ΔFnII constructs were proMMP9(29-444;ΔFnII) (SEQ ID NO:4), proMMP9(67-444;ΔFnII) (SEQ ID NO:5) and proMMP9(20-445;ΔFnII) (SEQ ID NO:6). Binding studies with the proMMP9 proteins without the FnII domains showed that compounds bound with similar affinity compared to the wild-type protein (data not shown).

In order to make the constructs with the FnII domains deleted, proMMP9(29-444;ΔFnII) (SEQ ID NO:4), proMMP9(67-444;ΔFnII) (SEQ ID NO:5) and proMMP9(20-445;ΔFnII) (SEQ ID NO:6), plasmids encoding the different proMMP9 truncations were used as templates for PCR to create two fragments of DNA corresponding to amino acid pairs including: 29-215/391-444, 67-215/391-444, and 20-215/391-445, respectively. Overlapping PCR was used to join the fragments. The 5′ primers had an Nde1 site and a start methionine and the 3′ primers had a stop codon and a Bgl2 site. The final PCR products were cloned into the TOPO TA cloning vector (Invitrogen) and the sequences were confirmed. Subsequently the vectors were digested with Nde1 and Bgl2 and the sequences were subcloned into Nde1 and BamH1 sites of the T7 expression vector pET11a (Novagen).

Expression of Truncated Forms of Human proMMP9

For expression in E. coli, all of the truncated proMMP9 constructs were transformed into BL21(DE3) RIL cells (Stratagene). Cells were initiated for an overnight culture from glycerol stocks in LB+Ampicillin (100 μg/ml) @ 37° C. shaking at 220 rpms. The overnight culture was subcultured 1:100 in LB+Ampicillin (100 ug/ml) and maintained at 37° C. shaking at 220 rpms. Samples were taken and A600 readings were monitored until an OD of 0.6 was achieved. The culture was induced with 1 mM IPTG and maintained under present growth conditions. Cultures were harvested 3 hours post induction at 6000×g for 10 min. Pellets were washed in 1×PBS with protease inhibitors and stored at −80° C.

Purification of truncated forms of human proMMP9

To purify the truncated proMMP9 proteins from E. coli, cell pellets were suspended in 25 mM Na₂HPO₄ pH 7, 150 mM NaCl, 10 mL/gram cell pellet. The cells were homogenized in a Dounce homogenizer, and then processed twice through a microfluidizer (Microfluidics International Corporation, model M-110Y). The lysate was centrifuged at 32,000×g for 45 minutes at 4° C. The supernatant was discarded. The pellet was suspended in 25 mM Na₂HPO₄ pH 7, 150 mM NaCl, 10 mM DTT, 1 mM EDTA, 10 mL/gram cell pellet. The pellet was homogenized in a Dounce homogenizer, and then centrifuged at 32,000×g for 45 minutes at 4° C. The supernatant was discarded. The pellet was suspended in 7 M urea, 25 mM Tris pH 7.5, 10 mM DTT, 1 mM EDTA, 6.5 mL/gram cell pellet, and then solubilized in a Dounce homogenizer and stirred for approximately 16 hours at ambient temperature. The solubilized protein solution was adjusted to pH 7.5, centrifuged at 45,000×g, 45 minutes at 4° C., and the supernatant, containing the denatured proMMP9, was filtered to 0.8 micron. A 5 mL HiTrap Q Sepharose HP column (GE Healthcare) was prepared according to manufacturer's instructions using Buffer A: 7 M urea, 25 mM Tris pH 7.5 and Buffer B: 7 M urea, 25 mM Tris pH 7.5, 1.0 M NaCl. The protein solution was applied to the HiTrap at 2.5 mL/minute. The column was washed to baseline absorbance with approximately 3.5 CV Buffer A. The proMMP9 was eluted in a 12CV linear gradient from 0% Buffer B to 12% Buffer B. Fractions were collected, analyzed on SDS-PAGE (Novex) and pooled based on purity. The pooled protein was re-natured by drop-wise addition to a solution, stirring and at ambient temperature, of 20 mM Tris pH 7.5, 200 mM NaCl, 5 mM CaCl₂, 1 mM ZnCl₂, 0.7 M L-arginine, 10 mM reduced and 1 mM oxidized glutathione, and was stirred for approximately 16 hours at 4° C. The refolded protein was concentrated to approximately 2.5 mg/mL in Jumbo Sep centrifugal concentrators (Pall) with 10,000 MWCO membranes. The concentrated protein solution was dialyzed at 4° C. for approximately 16 hours against 20 mM Tris pH 7.5, 150 mM NaCl. The dialyzed protein solution was clarified by filtration to 0.8 micron, concentrated to 2 mg/mL as before, centrifuged at 45,000×g for 15 minutes at 4° C. and filtered to 0.2 micron. It was purified on a HiLoad 26/60 Superdex 200 column (GE Healthcare) equilibrated in 20 mM Tris pH 7.5, 200 mM NaCl. Fractions were analyzed by SDS-PAGE and pooled based on purity. The pooled protein was concentrated in a Jumbo Sep concentrator as before and centrifuged at 16,000×g for 10 minutes at 4° C. The protein concentration was determined using Bio-Rad Protein Assay (Bio-Rad Laboratories, Inc.) with bovine serum albumin as a standard. The supernatant was aliquoted, frozen in liquid nitrogen and stored at −80° C.

Full-length human proMMP9

Full-length proMMP9(1-707) (SEQ ID NO:1) was expressed in HEK293 cells or in COS-1 cells as a secreted protein using a pcDNA3.1 expression vector. When expressed as a secreted protein in HEK293 cells or COS-1 cells, there is cotranslational removal of the signal peptide, amino acids 1-19 of full-length proMMP9(1-707) (SEQ ID NO:1). The final purified proMMP9(1-707) (SEQ ID NO:1) protein lacks the signal peptide.

Prior to transfection with the proMMP9(1-707) (SEQ ID NO:1) construct, the HEK293 cells were suspension adapted (shake flasks) in a serum free media (Freestyle 293) supplemented with pluronic acid (F-68) at a final concentration of 0.1%. Once cells reached a density of 1.2×10⁶/mL they were transiently transfected using standard methods. Transient transfection of COS-1 cells was done in flasks with adherent cell cultures and serum free media. For both HEK293 and COS-1 cells, the conditioned media was collected for purification of the proMMP9(1-707) (SEQ ID NO:1) protein. 1.0 M HEPES pH 7.5 was added to 9 L of conditioned media for a final concentration of 50 mM. The media was concentrated to 600 mL in a Kvicklab concentrator fitted with a hollow fiber cartridge of 10,000 MWCO (GE Healthcare). This was clarified by centrifugation at 6,000×g, 15 minutes, at 4° C. and then further concentrated to 400 mL in Jumbo Sep centrifugal concentrators (Pall) with 10,000 MWCO membranes. The concentrated protein was dialyzed against 50 mM HEPES pH 7.5, 10 mM CaCl₂, 0.05% Brij 35, overnight at 4° C. and then dialysis was continued for several hours at 4° C. in fresh dialysis buffer. The dialyzed protein was centrifuged at 6,000×g, 15 minutes, at 4° C., and filtered to 0.45 micron. 12 mL of Gelatin Sepharose 4B resin (GE Healthcare) was equilibrated in 50 mM HEPES pH 7.5, 10 mM CaCl₂, 0.05% Brij 35 in a 2.5 cm diameter Econo-Column (Bio-Rad Laboratories). The filtered protein solution was loaded onto the Gelatin Sepharose resin using gravity flow at approximately 3 mL/minute. The resin was washed with 10CV 50 mM HEPES pH 7.5, 10 mM CaCl₂, 0.05% Brij 35 and eluted with 30 mL 50 mM HEPES pH 7.5, 10 mM CaCl₂, 0.05% Brij 35, 10% DMSO, collected in 5 mL fractions. Fractions containing protein, confirmed by A280 absorbance, were dialyzed, in 500 times the volume of the fractions, against 50 mM HEPES pH 7.5, 10 mM CaCl₂, 0.05% Brij 35, overnight at 4° C. Dialysis was continued for an additional 24 hours in two fresh buffer changes. The dialyzed fractions were analyzed on SDS-PAGE and pooled based on purity. The pooled protein was concentrated to 1.2 mg/mL in Jumbo Sep centrifugal concentrators with 10,000 MWCO membranes. Protein concentration was determined with DC™ protein assay (Bio-Rad Laboratories, Inc.). The protein was aliquoted, frozen in liquid nitrogen and stored at −80° C.

Full-Length Rat proMMP9

Amino acid numbering for full-length rat proMMP9 was based on UniProtKB/Swiss-Prot P50282, full-length rat matrix metalloproteinase-9 precursor, proMMP9(1-708) (SEQ ID NO:11). The full-length rat proMMP9 was produced with the same methods as described for full-length human proMMP9. In brief, full-length rat proMMP9(1-708) (SEQ ID NO:11) was expressed in HEK293 cells as a secreted protein using a pcDNA3.1 expression vector. When expressed in HEK293 cells and secreted into the media, there is cotranslational removal of the signal peptide, so the final purified full-length rat proMMP9(1-708) (SEQ ID NO:11) protein lacks the signal peptide.

Human proMMP13

The sequence for proMMP13 was amino acids 1-268 from UniProtKB/Swiss-Prot P45452, proMMP13(1-268) (SEQ ID NO:7). The expression construct included a C-terminal Tev cleavage sequence flanking recombination sequences for use in the Invitrogen Gateway system. The construct was recombined into an entry vector using the Invitrogen Gateway recombination reagents. The resulting construct was transferred into a HEK293 expression vector containing a C-terminal 6X-histidine tag. Protein was expressed via transient transfection utilizing HEK293 cells and secreted into the media. When expressed in HEK293 cells and secreted into the media, there is cotranslational removal of the signal peptide, amino acids 1-19 of proMMP13(1-268) (SEQ ID NO:7). The final purified proMMP13(1-268) (SEQ ID NO:7) protein lacks the signal peptide. HEK293 media were harvested and centrifuged. Media were loaded on GE Healthcare His Trap FF columns, washed with buffer A (20 mM Tris pH 7.5, 200 mM NaCl, 2 mM CaCl₂, 10 mM imidazole), and eluted with buffer B (20 mM Tris pH 7.5, 200 mM NaCl, 2 mM CaCl₂ 200 mM imidazole). The eluted protein was loaded on a Superdex 200 column equilibrated with buffer C (20 mM HEPES pH 7.4, 100 mM NaCl, 0.5 mM CaCl₂). Fractions containing proMMP13(1-268) (SEQ ID NO:7) were pooled and concentrated to >2 mg/mL.

### Human Catalytic MMP3

Catalytic MMP3 was amino acids 100-265 of human MMP3 from UniProtKB/Swiss-Prot P08254, MMP3(100-265) (SEQ ID NO:8). The corresponding nucleotide sequence was subcloned into a pET28b vector to add a C-terminal 6X-Histidine tag and the construct was used for expression in E. coli. The protein was purified to >95% purity from 4.5 M urea solubilized inclusion bodies by standard techniques. Aliquots of purified protein were stored at −70° C. Purified recombinant human catalytic MMP3 is also available from commercial sources (e.g., Calbiochem®, 444217).

### Biological Assays

### ThermoFluor® Assays

### Generalized ThermoFluor® Methods

The ThermoFluor® (TF) assay is a 384-well plate-based binding assay that measures thermal stability of proteins (Biomol Screen 2001, 6, 429-40; Biochemistry 2005, 44, 5258-66). The experiments were carried out using instruments available from Johnson & Johnson Pharmaceutical Research & Development, LLC. TF dye used in all experiments was 1,8-anilinonaphthalene-8-sulfonic acid (1,8-ANS) (Invitrogen: A-47).

Compounds were arranged in a pre-dispensed plate (Greiner Bio-one: 781280), wherein compounds were serially diluted in 100% DMSO across 11 columns within a series. Columns 12 and 24 were used as DMSO reference and contained no compound. For multiple compound concentration-response experiments, the compound aliquots (50 mL) were robotically predispensed directly into black 384-well polypropylene PCR microplates (Abgene: TF-0384/k) using a Cartesian Hummingbird liquid handler (DigiLab, Holliston, Mass.). Following compound dispense, protein and dye solutions were added to achieve the final assay volume of 3 μL. The assay solutions were overlayed with 1 μL of silicone oil (Fluka, type DC 200: 85411) to prevent evaporation.

Assay plates were robotically loaded onto a thermostatically controlled PCR-type thermal block and then heated from 40 to 90° C. at a ramp-rate of 1° C./min for all experiments. Fluorescence was measured by continuous illumination with UV light (Hamamatsu LC6) supplied via fiber optics and filtered through a band-pass filter (380-400 nm; >60D cutoff). Fluorescence emission of the entire 384-well plate was detected by measuring light intensity using a CCD camera (Sensys, Roper Scientific) filtered to detect 500±25 nm, resulting in simultaneous and independent readings of all 384 wells. A single image with 20-sec exposure time was collected at each temperature, and the sum of the pixel intensity in a given area of the assay plate was recorded vs temperature and fit to standard equations to yield the Tₘ (J Biomol Screen 2001, 6, 429-40).

Thermodynamic parameters necessary for fitting compound binding for each proMMP were estimated by differential scanning calorimetry (DSC) and from ThermoFluor® data. The heat capacity of unfolding for each protein was estimated from the molecular weight and from ThermoFluor® dosing data. Unfolding curves were fit singly, then in groups of 12 ligand concentrations the data were fit to a single K_{D} for each compound.

ThermoFluor® with proMMP9(67-444;ΔFnII) (SEQ ID NO:5)

The protein sample preparations had to include a desalting buffer exchange step via a PD-10 gravity column (GE Healthcare). The desalting buffer exchange was performed prior to diluting the protein to the final assay concentration of 3.5 μM proMMP9(67-444;ΔFnII) (SEQ ID NO:5). The concentration of proMMP9(67-444;ΔFnII) (SEQ ID NO:5) was determined spectrophotometrically based on a calculated extinction coefficient of ε₂₈₀=33900 M⁻¹cm⁻¹, a calculated molecular weight of 22.6 kDa, and calculated pI of 5.20. ThermoFluor® reference conditions were defined as follows: 80 μg/mL (3.5 μM) proMMP9(67-444;ΔFnII) (SEQ ID NO:5), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(67-444;ΔFnII) (SEQ ID NO:5) are as follows: Tₘ (° C.)=63 (+/−0.1), Δ_{U}H_{(Tm)} (cal mol⁻¹)=105000(+/−5000), Δ_{U}S_{(Tm)} (cal mol⁻¹ K⁻¹)=450, Δ_{U}Cₚ (cal mol⁻¹ K⁻¹)=2000.

ThermoFluor® with proMMP9(20-445;ΔFnII) (SEQ ID NO:6)

The protein sample preparations included a desalting buffer exchange step via a PD-10 gravity column (GE Healthcare). The desalting buffer exchange was performed prior to diluting the protein to the final assay concentration of 2.8 μM proMMP9(20-445;ΔFnII) (SEQ ID NO:6). The concentration of proMMP9(20-445;ΔFnII) (SEQ ID NO:6) was determined spectrophotometrically based on a calculated extinction coefficient of ε₂₈₀=39880 M⁻¹cm⁻¹, a calculated molecular weight of 28.2 kDa, and calculated pI of 5.5. ThermoFluor® reference conditions were define as follows: 80 μg/mL (2.8 μM) proMMP9(20-445;ΔFnII) (SEQ ID NO:6), 50 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP9(20-445;ΔFnII) (SEQ ID NO:6) are as follows: Tₘ (° C.)=72 (+/−0.1), Δ_{U}H_{(Tm)} (cal mol⁻¹)=160000(+/−5000), Δ_{U}S_{(Tm)} (cal mol⁻¹ K⁻¹)=434, Δ_{U}Cₚ (cal mol⁻¹ K⁻¹)=2400.

ThermoFluor® with proMMP13(1-268) (SEQ ID NO: 7)

The proMMP13(1-268) (SEQ ID NO:7) protein sample preparations included a desalting buffer exchange step via a PD-10 gravity column (GE Healthcare). The desalting buffer exchange was performed prior to diluting the protein to the final assay concentration of 3.5 μM. The concentration of proMMP13(1-268) (SEQ ID NO:7) was estimated spectrophotometrically based on a calculated extinction coefficient of ε₂₈₀=37000 M⁻¹cm⁻¹, a calculated molecular weight of 30.8 kDa, and calculated pI of 5.33. ThermoFluor® reference conditions were defined as follows: 100 μg/mL proMMP13(1-268) (SEQ ID NO:7), 25 μM 1,8-ANS, pH 7.0 Buffer (50 mM HEPES pH 7.0, 100 mM NaCl, 0.001% Tween-20, 2.5 mM MgCl₂, 300 μM CaCl₂). The thermodynamic parameters for proMMP13(1-268) (SEQ ID NO:7) are as follows: Tₘ (° C.)=67 (+/−0.1), Δ_{U}H_{(Tm)} (cal mol⁻¹)=107000(+/−5000), Δ_{U}S_{(Tm)} (cal mol⁻¹ K⁻¹)=318, Δ_{U}Cₚ (cal mol⁻¹ K⁻¹)=2600.

Thermofluor data for representative compounds of Formula I is shown in Table 1.

TABLE 1

| | proMMP9(20-445; | proMMP9(67-444; | proMMP13 |
|---|---|---|---|
| | ΔFnII) | ΔFnII) | (1-268) |
| Exam- | (SEQ ID NO: 6) | (SEQ ID NO: 5) | (SEQ ID NO: 7) |
| ple | binding, Kd (μM) | binding, Kd (μM) | binding, Kd (μM) |
| 1 | 0.16 | 1.0 | 0.31 |
| 2 | 0.73 | 1.5 | 2.6 |
| 3 | 33 | 38 | >95 |
| 4 | 1.3 | 3.3 | 12 |
| 5 | 53 | 33 | >95 |
| 6 | 70 | 11 | ND |
| 7 | 1.2 | 3.9 | ND |
| 8 | 0.37 | 2.9 | ND |
| 9 | 7.4 | 43 | 18 |
| 10 | 0.66 | 3.5 | ND |
| 11 | 5.9 | 16 | ND |
| 12 | 17 | 29 | >95 |
| 13 | 2.3 | 10 | 1.2 |
| 14 | 0.21 | 1.9 | 0.93 |
| 15 | 0.25 | 0.47 | 0.73 |
| 16 | 20 | 2.9 | ND |
| 17 | 3.6 | 5.7 | 1.5 |
| 18 | 26 | 0.66 | 3.3 |
| 19 | 27 | 14 | 6.2 |
| 20 | 0.34 | 3.3 | 0.23 |
| 21 | 0.21 | 4.1 | 1.4 |
| 22 | 1.3 | 5.7 | 2.9 |
| 23 | 11 | 18 | 12 |
| 24 | 2.2 | >95 | 3.7 |
| 25 | 0.17 | 2.0 | 1.8 |
| 26 | 0.32 | 3.3 | 0.25 |
| 27 | 0.039 | 0.38 | 0.28 |
| 28 | 0.082 | 0.99 | ND |
| 29 | 0.93 | 1.6 | ND |
| 30 | 0.43 | 0.69 | ND |
| 31 | 0.11 | 0.61 | ND |
| 32 | 0.36 | 2.3 | ND |
| 33 | 0.046 | 0.12 | ND |
| 34 | 0.18 | 1.5 | ND |
| 35 | 0.19 | 0.46 | ND |
| 36 | 1.3 | 7.2 | ND |
| 37 | 1.3 | 2.4 | ND |
| 38 | 0.073 | 0.38 | ND |
| 39 | 0.070 | 0.17 | ND |
| 40 | 0.044 | 0.33 | ND |
| 41 | 0.051 | 0.22 | ND |
| 42 | 0.13 | 0.68 | ND |
| 43 | 0.12 | 1.7 | ND |
| 44 | 0.048 | 0.13 | ND |
| 45 | 0.47 | 3.7 | ND |
| 46 | 1.3 | 6.6 | ND |
| 47 | 0.90 | 1.5 | ND |
| 48 | 2.3 | 4.1 | ND |
| 49 | 0.83 | 2.0 | ND |
| 50 | 0.090 | 0.13 | ND |
| 51 | 1.2 | 2.7 | ND |
| 52 | 1.2 | 2.2 | ND |
| 53 | 0.89 | 2.3 | ND |
| 54 | 0.96 | 2.5 | ND |
| 55 | 0.81 | 2.6 | ND |
| 56 | 0.64 | 1.5 | ND |
| 57 | 0.11 | 192 | ND |
| 58 | 1.2 | 3.1 | ND |
| 59 | 36 | ND | >95 |
| 60 | >95 | >95 | 78 |
| 61 | ND | ND | ND |
| 62 | 0.51 | 2.2 | ND |
| 63 | 0.21 | 0.28 | ND |
| 64 | 0.26 | 2.5 | ND |
| 65 | 0.069 | ND | ND |
| 66 | 0.31 | ND | ND |
| 67 | 0.071 | ND | ND |
| 68 | 0.071 | ND | ND |
| 69 | 0.065 | ND | ND |
| 70 | 0.25 | ND | ND |
| 71 | 0.075 | ND | ND |
| 72 | 0.11 | ND | ND |
| 73 | 0.12 | ND | ND |
| 74 | 0.17 | ND | ND |
| 75 | 0.28 | ND | ND |
| 76 | 0.24 | ND | ND |
| 77 | 1.9 | ND | ND |
| 78 | 0.34 | ND | ND |
| 79 | 0.15 | ND | ND |
| 80 | 0.15 | ND | ND |
| 81 | 0.13 | ND | ND |
| 82 | 0.099 | ND | ND |
| 83 | 0.31 | ND | ND |
| 84 | 0.25 | 0.20 | ND |
| 85 | 4.6 | 32 | ND |
| 86 | 0.46 | ND | ND |
| 87 | 0.36 | ND | ND |
| 88 | 0.26 | ND | ND |
| 89 | 0.36 | ND | ND |
| 90 | 0.97 | ND | ND |
| 91 | 2.4 | ND | ND |
| 92 | 0.45 | ND | ND |
| 93 | 0.58 | 3.4 | ND |
| 94 | 0.43 | 1.3 | ND |
| 95 | ND | ND | ND |
| 96 | 1.9 | ND | ND |
| Interme- | 192 | 0.41 | ND |
| diate 12 | | | |
| Interme- | 96 | ND | ND |
| diate 9 | | | |

### Enzyme Assays

proMMP9/MMP3 P126 Activation Assay

Compounds were assessed for inhibition of proMMP9 activation by catalytic MMP3, MMP3(100-265) (SEQ ID NO:8) using full-length proMMP9(1-707) (SEQ ID NO:1) purified from HEK293 cells and a peptide (Mca-PLGL-Dpa-AR-NH₂, BioMol P-126) that fluoresces upon cleavage by catalytic MMP9. The assay buffer employed was 50 mM Hepes, pH 7.5, 10 mM CaCl₂, 0.05% Brij-35. DMSO was included at a final concentration of 2%, arising from the test compound addition. On the day of assay, proMMP9(1-707) (SEQ ID NO:1) purified from HEK293 cells and MMP3(100-265) (SEQ ID NO:8) were diluted to 400 nM in assay buffer. The reaction volume was 50 μL. In 96-well black plates (Costar 3915), 44 μL of assay buffer was mixed with 1.0 μL of test compound, 2.5 μL of 400 nM proMMP9(1-707) (SEQ ID NO:1) purified from HEK293 cells and the reaction was initiated with 2.5 μL of 400 nM MMP3(100-265) (SEQ ID NO:8).The plate was sealed and incubated for 80 min at 37° C. Final concentrations were 20 nM proMMP9(1-707) (SEQ ID NO:1) purified from HEK293 cells and 20 nM MMP3(100-265) (SEQ ID NO:8), and concentrations of test compounds were varied to fully bracket the IC₅₀. Immediately following the 80 min incubation, 50 μL of 40 μM P-126 substrate was added (freshly diluted in assay buffer), and the resulting activity associated with catalytic MMP9 was kinetically monitored at 328 nm excitation, 393 nm emission for 10-15 min at 37° C., using a Spectramax Gemini XPS reader (Molecular Devices). Reactivity of residual MMP3 towards P-126 substrate was minimal under these conditions. Initial velocities were plotted by use of a four-parameter logistics equation (GraphPad Prism® software) for determination of IC₅₀.

### ProMMP13/Plasmin P126 Activation Assay

Compounds were assessed for inhibition of proMMP13 activation by plasmin using a peptide (Mca-PLGL-Dpa-AR-NH₂, BioMol P-126) that fluoresces upon cleavage by catalytic MMP13. The assay buffer employed was 50 mM Hepes, pH 7.5, 10 mM CaCl₂, 0.05% Brij-35. DMSO was included at a final concentration of 2%, arising from the test compound addition. On the day of assay, proMMP13(1-268) (SEQ ID NO:7) purified from HEK293 cells and plasmin were diluted to 160 nM and 320 nM, respectively, in assay buffer. The reaction volume was 50 μL. In 96-well black plates (Costar 3915), 44 μL of assay buffer was mixed with 1.0 μL of test compound, 2.5 μL of 160 nM proMMP13(1-268) (SEQ ID NO:7), and the reaction was initiated with 2.5 μL of 320 nM plasmin. The plate was sealed and incubated for 40 min at 37° C. Final concentrations were 8 nM proMMP13(1-268) (SEQ ID NO:7) and 16 nM plasmin, and concentrations of test compounds were varied to fully bracket the IC₅₀. Immediately following the 40 min incubation, 50 μL of 40 μM P-126 substrate was added (freshly diluted in assay buffer), and the resulting activity associated with catalytic MMP13 was kinetically monitored at 328 nm excitation, 393 nm emission for 10-15 min at 37° C., using a Spectramax Gemini XPS reader (Molecular Devices). Plasmin was not reactive towards P-126 substrate under these conditions. Initial velocities were plotted by use of a four-parameter logistics equation (GraphPad Prism® software) for determination of IC₅₀.

### ProMMP9/MMP3 DQ Gelatin Activation Assay

Compounds were assessed for inhibition of proMMP9 activation by catalytic MMP3 using a quenched fluorescein gelatin substrate (DQ gelatin, Invitrogen D12054) that fluoresces upon cleavage by activated MMP9. The assay buffer employed was 50 mM Hepes, pH 7.5, 10 mM CaCl₂, 0.05% Brij-35. DMSO was included at a final concentration of 0.2%, arising from the test compound addition. On the day of assay, full-length proMMP9(1-707) (SEQ ID NO:1) from COS-1 cells and catalytic MMP3(100-265) (SEQ ID NO:8) were diluted to 60 nM and 30 nM, respectively, in assay buffer. Test compounds in DMSO were diluted 250-fold in assay buffer at 4× the final concentration. The reaction volume was 12 μL, and all reactions were conducted in triplicate. In 384-well half-volume plates (Perkin Elmer ProxiPlate 384 F Plus, 6008260), 4 μL of test compound in assay buffer was mixed with 4 μL of 60 nM full-length proMMP9(1-707) (SEQ ID NO:1) from COS-1 cells. The plate was sealed and incubated for 30 min at 37° C. Final concentrations were 20 nM full-length proMMP9(1-707) (SEQ ID NO:1) from COS-1 cells and 10 nM MMP3(100-265) (SEQ ID NO:8), and concentrations of test compounds were varied to fully bracket the IC₅₀. Immediately following the 30 min incubation, 4 μL of 40 μg/ml DQ gelatin substrate was added (freshly diluted in assay buffer), and incubated for 10 min at room temperature. The reaction was stopped by the addition of 4 μL of 50 mM EDTA, and the resulting activity associated with catalytic MMP9 was determined at 485 nm excitation, 535 nm emission using an Envision fluorescent reader (Perkin Elmer). Reactivity of residual MMP3 towards DQ gelatin was minimal under these conditions. Percent inhibition of test compounds were determined from suitable positive (DMSO only in assay buffer) and negative (EDTA added prior to reaction initiation) controls. Plots of % inhibition vs. test compound concentration were fit to a four-parameter logistics equation (GraphPad Prism® software) for determination of IC₅₀.

Enzyme assay data for representative compounds of Formula I is shown in Table 2.

TABLE 2

| | proMMP9/MMP3 | ProMMP9/MMP3 | ProMMP13/Plasmin |
|---|---|---|---|
| | P126 | DQ gel | P126 |
| Exam- | Activation Assay, | Activation Assay, | Activation Assay, |
| ple | IC₅₀ (μM) | IC₅₀ (μM) | IC₅₀ (μM) |
| 1 | 0.36 | ND | ND |
| 2 | 0.74 | ND | ND |
| 3 | ~5 | ND | ND |
| 4 | 5.0 | ND | ND |
| 5 | ~19 | ND | ND |
| 6 | 11 | ND | ND |
| 7 | 2.4 | ND | ND |
| 8 | 1.8 | ND | ND |
| 9 | 2.6 | ND | ND |
| 10 | 2.6 | ND | ND |
| 11 | ND | ND | ND |
| 12 | >20 | ND | ND |
| 13 | 2.4 | ND | ND |
| 14 | 0.50 | ND | ND |
| 15 | 0.28 | ND | ND |
| 16 | 3.2 | ND | ND |
| 17 | 4.3 | ND | ND |
| 18 | 0.67 | ND | ND |
| 19 | ~4 | ND | ND |
| 20 | 0.64 | ND | ND |
| 21 | 0.60 | ND | ND |
| 22 | 4.6 | ND | ND |
| 23 | ~14 | ND | ND |
| 24 | ~3 | ND | ND |
| 25 | 0.46 | ND | ND |
| 26 | 0.68 | ND | ND |
| 27 | 0.15 | ND | 0.90 |
| 28 | 0.21 | ND | ND |
| 29 | 1.4 | ND | ND |
| 30 | 0.75 | ND | ND |
| 31 | 0.64 | ND | ND |
| 32 | 0.84 | ND | ND |
| 33 | 0.14 | ND | ND |
| 34 | 1.1 | ND | ND |
| 35 | 0.11 | ND | ND |
| 36 | 2.3 | ND | ND |
| 37 | ~2 | ND | ND |
| 38 | 0.17 | ND | ND |
| 39 | 0.087 | ND | ND |
| 40 | 0.087 | ND | ND |
| 41 | 0.059 | ND | ND |
| 42 | 0.15 | ND | ND |
| 43 | 0.20 | ND | ND |
| 44 | 0.073 | ND | ND |
| 45 | 0.37 | ND | ND |
| 46 | 1.3 | ND | ND |
| 47 | 0.98 | ND | ND |
| 48 | 1.1 | ND | ND |
| 49 | 1.0 | ND | ND |
| 50 | 0.16 | ND | ND |
| 51 | 1.8 | ND | ND |
| 52 | 1.0 | ND | ND |
| 53 | 0.97 | ND | ND |
| 54 | 3.7 | ND | ND |
| 55 | 0.42 | ND | ND |
| 56 | 0.39 | ND | ND |
| 57 | 0.24 | ND | ND |
| 58 | 1.4 | ND | ND |
| 59 | >20 | ND | ND |
| 60 | ND | 5.5 | ND |
| 61 | 0.30 | ND | ND |
| 62 | 0.61 | ND | ND |
| 63 | 0.15 | ND | ND |
| 64 | 0.12 | ND | ND |
| 65 | 0.21 | ND | ND |
| 66 | >20 | ND | ND |
| 67 | 0.13 | ND | ND |
| 68 | 0.13 | ND | ND |
| 69 | 0.10 | ND | ND |
| 70 | 0.34 | ND | ND |
| 71 | 0.17 | ND | ND |
| 72 | 0.14 | ND | ND |
| 73 | 0.21 | ND | ND |
| 74 | 0.16 | ND | ND |
| 75 | 0.11 | ND | ND |
| 76 | >20 | ND | ND |
| 77 | >20 | ND | ND |
| 78 | ~0.3 | ND | ND |
| 79 | 0.34 | ND | ND |
| 80 | 0.36 | ND | ND |
| 81 | 0.13 | ND | ND |
| 82 | 0.37 | ND | ND |
| 83 | 0.14 | ND | ND |
| 84 | 0.26 | ND | ND |
| 85 | ~20 | ND | ND |
| 86 | 0.40 | ND | ND |
| 87 | ~2 | ND | ND |
| 88 | 0.33 | ND | ND |
| 89 | 0.35 | ND | ND |
| 90 | 1.7 | ND | ND |
| 91 | 0.64 | ND | ND |
| 92 | 0.23 | ND | ND |
| 93 | 0.52 | ND | ND |
| 94 | 0.52 | ND | ND |
| 95 | 0.31 | ND | ND |
| 96 | 0.59 | ND | ND |
| Interme- | >20 | ND | ND |
| diate 12 | | | |
| Interme- | ND | ND | ND |
| diate 9 | | | |

### Cell-based Assays

Activation of proMMP9 in Rat Synoviocyte Cultures

A primary synoviocytes line was derived from the periarticular tissue of arthritic rats. Arthritis was induced in female Lewis rats following an i.p. administration of streptococcal cell wall peptidoglycan polysaccharides (J Exp Med 1977; 146:1585-1602). Rats with established arthritis were sacrificed, and hind-limbs were severed, immersed briefly in 70% ethanol, and placed in a sterile hood. The skin was removed and the inflamed tissue surrounding the tibia-tarsal joint was harvested using a scalpel. Tissue from six rats was pooled, minced to approximately 8 mm³ pieces, and cultured in Dulbecco's Modified Eagle's Medium (DMEM) containing 15% fetal calf serum (FCS). In the following weeks, cells migrated out of the tissue piece, proliferated, and formed a monolayer of adherent cells. The synoviocytes were lifted from culture plates with 0.05% trypsin and passaged weekly at 1:4 ratios in DMEM containing 10% FCS. Synoviocytes were used at passage 9 to investigate the ability of Compound-α to inhibit the maturation of MMP9 to active form.

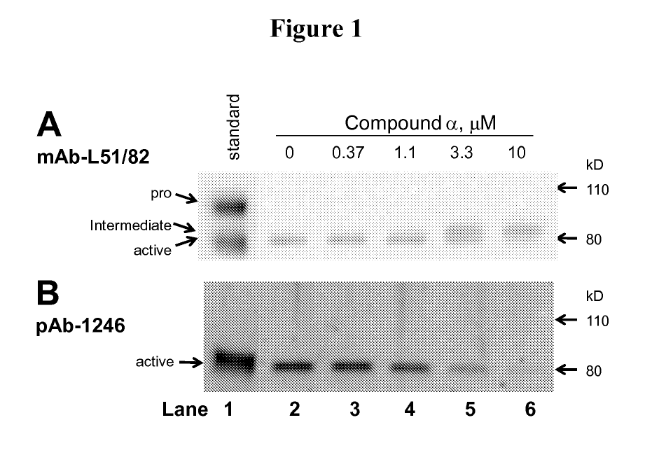

Rat synoviocytes spontaneously expressed and activated MMP9 when cultured in collagen gels and stimulated with tumor necrosis factor-alpha (TNFα) (FIG. 1 and Table 3). Eight volumes of an ice-cold solution of 3.8 mg/mL rat tail collagen (Sigma Cat #C3867-1VL) were mixed with 1 volume of 1 M sodium bicarbonate and 1 volume of 10× Roswell Park Memorial Institute medium. The pH of the mixture was adjusted to pH 7 with 1 N sodium hydroxide and equal volumes of the pH-adjusted collagen solution were mixed with DMEM containing 0.8 million synoviocytes per mL. One half mL volumes were dispensed into Costar 24-well culture dishes and placed for one hr at 37° C. and 5% CO₂, during which time the collagen solution formed a gel. Individual gels were dislodged into wells of 12-well Costar plates containing 1 mL/well of DMEM adjusted to contain 0.05% BSA and 100 ng/mL mouse TNFα (R&D Systems Cat #410-MT-010). The plates were agitated 10 seconds to ensure that the collagen gels did not adhere to the well bottoms. After overnight culture at 37° C. and 5% CO₂, wells were adjusted to contain an additional 0.5 mL of DMEM containing 0.05% BSA and Compound-α at 4× the final desired concentration (final culture volumes were 2 mL). The plates were cultured an additional 48 hrs, at which time 1 mL of conditioned media were harvested into fresh eppendorf tubes containing 40 μL/mL of a 50% slurry of gelatin-conjugated sepharose (GE Healthcare Cat #17-0956-01). Samples were rotated for 2 hrs at 4° C. before centrifugation 1 min×200 g. Supernatants were discarded. The gelatin-sepharose pellets were washed once with 1 mL of ice cold DMEM, resuspended in 50 μL of 2× reducing Leamli buffer and heated 5 min at 95° C. Fifteen μL of eluted proteins were resolved on 4-12% NuPAGE gels and transferred to 0.45 μm pore-sized nitrocellose blots. Next, blots were incubated in blocking buffer (5% milk in Tris-buffered saline containing 0.1% Tween-20) for 1 hr at RT and probed overnight (4° C.) with blocking buffer containing 1 μg/mL primary antibodies. Blots were next probed 1 hr at RT with 1/10,000 dilutions of goat anti-mouse IgG-HRP or goat anti-rabbit IgG-HRP (Santa Cruz) in blocking buffer and developed using SuperSignal® West Fempto Maximum Sensitivity Substrate. Chemiluminesence signal was analyzed using a ChemiDoc imaging system (BioRad Laboratories) and Quantity One® image software. Electrophoretic mobility was estimated based on the mobility of standards (Novex Sharp Pre-Stained Protein Standards P/N 57318).

Mouse mAb-L51/82 (UC Davis/NIH NeuroMab Facility, Antibody Incorporated) was used to detect pro and processed forms of MMP9. Synoviocyte-conditioned media contained an approximately 80 kD form of MMP9 (FIG. 1A, lane 2). In the presence of 0.37-10 μM Compound-α (FIG. 1A, lanes 3-6), the 80 kD active MMP9 form was reduced in a dose dependent fashion, and a form of approximately 86 kD appeared. The 86 kD form was predominant in the presence of 10 μM Compound-α (FIG. 1A, lane 6). Lane 1 was loaded with a standard containing 3 ng of full-length rat proMMP9(1-708) (SEQ ID NO:11) and 3 ng of full-length rat proMMP9(1-708) (SEQ ID NO:11) converted to catalytic rat MMP9 by catalytic MMP3. The electrophoretic mobility of the 80 kD form present in synoviocyte conditioned medium was the same as the active MMP9 standard. The 86 kD form produced by synoviocytes in the presence of Compound-α demonstrated greater mobility than the full-length rat proMMP9(1-708) (SEQ ID NO:11) standard which ran with a mobility of approximately 100 kD. The 86 kD form demonstrated a mobility similar to an incompletely processed intermediate form described previously that retains the cysteine switch and lacks catalytic activity (J Biol Chem; 1992; 267:3581-4).

ProMMP9 is activated when cleaved between R106 and F107 (J Biol Chem; 1992; 267:3581-4). A rabbit polyclonal antibody (pAb-1246) was generated to the active MMP9 N-terminal neoepitope using an approach similar to that reported previously (Eur J Biochem; 1998; 258:37-43). Rabbits were immunized and boosted with a peptide, human MMP9(107-113) (SEQ ID NO:9) conjugated to keyhole limpet hemocyanin, and antibodies were affinity purified from serum using FQTFEGD-conjugated agarose affinity resin and 100 mM glycine (pH 2.5) elution. To resolve N-terminal neoepitope antibodies from antibodies directed to other epitopes within the sequence, eluted antibody was dialyzed in PBS and cross-absorbed by mixing with a peptide, human proMMP9(99-113) (SEQ ID NO:10), that was conjugated to agarose. The unbound fraction containing N-terminal neoepitope antibodies was recovered and was designated pAb-1246.

FIG. 1B, lane 1 demonstrated that pAb-1246 bound the 80 kD active MMP9 standard, but did not recognize the 100 kD proMMP9 standard. pAb-1246 detected 80 kD active MMP9 in synoviocyte conditioned medium, and Compound-α caused a dose-dependent reduction in active MMP9 (FIG. 1B, lanes 2-6). Band chemiluminescence intensities were measured directly and reported in Table 3. The production of active MMP9 was inhibited by Compound-α with an IC₅₀ of approximately 1.1 μM. pAb-1246 did not recognize the 86 kD form, providing further evidence that this likely represented an intermediate form whose further maturation was blocked by Compound-α.

TABLE 3
Compound-α blocked production
of active MMP9 by rat synoviocytes ^{a}

| Compound-α, | | Signal of 80 kD band | |
|---|---|---|---|
| μM | | (INT*mm²) ^{b} | % Inhibition ^{c} |
| 0 | | 84384 | 0 |
| 0.37 | μM | 74381 | 12 |
| 1.1 | μM | 45381 | 46 |
| 3.3 | μM | 11554 | 86 |
| 10 | μM | 2578 | 97 |

^{a} Rat synoviocytes embedded in collagen gels were stimulated 72 hrs with TNFα. Cultures were supplemented with the indicated concentrations of Compound-α for the final 48 hrs and conditioned media were assessed for the 80 kD active form of MMP9 by Western blotting with pAb-1246 developed against the N-terminal activation neoepitope.
^{b} Chemiluminesence captured during a 30 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by synoviocytes cultured in the absence of Compound-α.

Activation of proMMP9 by Human Fetal Lung Fibroblast Cultures

Compound-α was assessed additionally for ability to block the maturation of proMMP9 to active MMP9 in cultures of human fetal lung fibroblasts (HFL-1, American Type Culture Collection #CCL-153). Unlike rat synoviocytes, HFL-1 cells were unable to process proMMP9 to the active form without addition of neutrophil elastase. Elastase did not directly cause processing of recombinant proMMP9 (data not shown). Rather, the function of elastase in this assay may be to inactivate tissue inhibitors of matrix metalloproteinases (TIMPs) that repress endogenous pathways of MMP9 activation (Am J Respir Crit. Care Med; 1999; 159:1138-46).

HLF-1 were maintained in monolayer culture in DMEM with 10% FCS and were used between passage numbers 5-15. HLF-1 were embedded in collagen gels as described for rat SCW synoviocytes (vida supra). Half mL gels containing 0.4 million cells were dislodged into wells of 12 well Costar plates containing 1 mL/well of DMEM adjusted to contain 0.05% BSA and 100 ng/mL human TNFα (R&D Systems Cat #210-TA/CF). After overnight culture (37° C. and 5% CO₂) wells were adjusted to contain an additional 0.5 mL of DMEM containing 0.05% BSA and with or without 13.2 μM Compound-α (final concentration was 3.3 μM Compound-α). Next, cultures were adjusted to contain 30 nM human elastase (Innovative Research). The plates were cultured an additional 72 hrs, at which time MMP9 secreted into the conditioned media was bound to gelatin-sepharose and evaluated by Western blot analysis as described for the rat synoviocyte cultures (vida supra). mAb-51/82 detected three forms of MMP9 in HFL-1 cultures.

These included a form of approximately 100 kD with mobility similar to recombinant rat proMMP9, an approximately 80 kD form with mobility similar to rat active MMP9, and an approximately 86 kD intermediate form. The band intensities are provided in Table 4. In the absence of Compound-α, most of the MMP9 was present as the 80 kD form. In the presence of Compound-α, the 80 kD form was a minor fraction of the total signal while nearly half of the signal were contributed each by the 100 kD and 86 kD forms. The total signal of the three bands was similar with or without Compound-α. These data indicate that the 100 kD and 86 kD forms of MMP9 were effectively stabilized by Compound-α and the formation of the 80 kD form was suppressed.

TABLE 4
Compound-α blocked processing of MMP9 by HFL-1 cells ^{a}

| Compound-α, | Signal (INT*mm²) ^{b} | | | | Percent of total signal | | |
|---|---|---|---|---|---|---|---|
| 3.3 μM | 100 kD | 86 kD | 80 kD | Total | 100 kD | 86 kD | 80 kD |
| − | 17190 | 24858 | 61925 | 103973 | 16 | 24 | 60 |
| + | 42107 | 43147 | 6092 | 91346 | 46 | 47 | 7 |

^{a} Human fetal lung fibroblasts (HFL-1) embedded in collagen gels were stimulated 90 hrs with TNFα. Cultures were supplemented with or without 3.3 μM Compound-α and with 30 nM elastase for the final 72 hrs and conditioned media were assessed for the MMP9 forms by Western blotting with mAb-L51/82.
^{b} Chemiluminesence captured during a 150 s exposure was analyzed using a ChemiDoc imaging system (BioRad Laboratories) and Quantity One ® image software. Signals were measured within uniform sized boxes drawn to circumscribe the bands and were the product of the average intensity (INT) and the box area (mm²). Values given have been corrected for background signal.

A second experiment was performed to determine if the 80 kD form was mature active MMP9 and to determine the potency of Compound-α as an inhibitor of MMP9 maturation in this assay. HFL-1 cells embedded in collagen gels were cultured as described above in the presence of TNFα overnight and the cultures were then adjusted to contain 30 nM elastase and graded concentrations of Compound-α for an additional 72 hrs at which time MMP9 secreted into the conditioned media was bound to gelatin-sepharose and evaluated by Western blot analysis for active MMP9 using pAb-1246 raised against the N-terminal neoepitope of active MMP9 (Table 5). In the absence of Compound-α, pAb-1246 readily detected MMP9 with an electrophoretic mobility of approximately 80 kD. Compound-α effectively inhibited the ability of HFL-1 cultures to process proMMP9 to active MMP9. Inhibition occurred over a dose range with an IC₅₀ of approximately 0.3 μM Compound-α.

TABLE 5
Compound-α blocked production of active
MMP9 by human fetal lung fibroblasts ^{a}

| Compound-α, | | Signal of 80 kD band | |
|---|---|---|---|
| μM | | (INT*mm²) ^{b} | % Inhibition ^{c} |
| 0 | | 168781 | 0 |
| 0.12 | μM | 168211 | 0 |
| 0.37 | μM | 45996 | 73 |
| 1.1 | μM | 1747 | 99 |
| 3.3 | μM | 152 | 100 |
| 10 | μM | 0 | 100 |

^{a} Human fetal lung fibroblasts (HFL-1) embedded in collagen gels were stimulated 90 hrs with TNFα. Cultures were supplemented with the indicated concentrations of Compound-α and 30 nM elastase for the final 72 hrs and conditioned media were assessed for active MMP9 by Western blotting with pAb-1246 developed against the N-terminal activation neoepitope.
^{b} Chemiluminesence captured during a 10 s exposure was analyzed using a ChemiDoc imaging system (BioRad Laboratories) and Quantity One ® image software. Signals were measured within uniform sized boxes drawn to circumscribe the 80 kD bands and were the product of the average intensity (INT) and the box area (mm²). Values given have been corrected for background signal.
^{c} Percent signal reduction relative to the signal generated by HFL-1 cells cultured in the absence of Compound-α.

### In Vivo Studies

Expression and activation of proMMP9 in vivo is associated with rat SCW-arthritis MMP9 protein expression was reportedly increased in the synovial fluid of patients with rheumatoid arthritis (Clinical Immunology and Immunopathology; 1996; 78:161-71). A preliminary study was performed to assess MMP9 expression and activation in a rat model of arthritis.

A polyarthritis can be induced in female Lewis rats following i.p. administration of streptococcal cell wall (SCW) proteoglycan-polysaccharides (PG-PS) (J Exp Med 1977; 146:1585-1602). The model has an acute phase (days 3-7) that is complement and neutrophil-dependent and that resolves. A chronic erosive phase begins at about day ten and is dependent on the development of specific T cell immunity to the PG-GS, which resists digestion and remains present in synovial macrophages for months. Like rheumatoid arthritis, SCW-induced arthritis is reduced by TNF inhibitors, and the dependence of SCW-induced arthritis on macrophages (Rheumatology; 2001; 40:978-987) and the strong association of rheumatoid arthritis severity with synovial-tissue macrophage counts (Ann Rheum Dis; 2005; 64:834-838) makes SCW-arthritis an attractive model for testing potential therapeutic agents. SCW PG-PS 10S (Beckton Dickinson Cat#210866) suspended in saline was vortexed for 30 seconds and sonicated for 3 min with a probe type sonicator prior to injection. Female Lewis (LEW/N) rats, 5-6 weeks of age (80-100 g) were injected (i.p.) with SCW PG-PS (15 μg of rhamnose/gram BW) in the lower left quadrant of the abdomen using a 1 mL syringe fitted with a 23-gauge needle. Control (disease-free) rats were treated in a similar manner with sterile saline. Control rats were sacrificed on day 5 and groups of SCW-injected rats were sacrificed on day 5 when acute inflammation was maximal or on day 18 when chronic inflammation was established.

Hind-limbs were skinned, severed just above the tibia-tarsus joint and below the metatarsals, and the tibia-tarsus joints (ankles) were weighed, snap frozen and pulverized on dry ice using a hammer and anvil. The pulverized tissue was suspended in 3 volumes (w:v) of ice-cold homogenization buffer containing 50 mM Tris pH 7.5, 150 mM NaCl, 5 mM EDTA, 1% Triton X100, 0.05% Brij 30, 10% dimethylsulfoxide and Complete EDTA-free Protease Inhibitor Cocktail (Roche Diagnostics). The suspended tissue was homogenized sequentially with a Kinematica AG Polytron and a Dounce homogenizer. Homogenates were centrifuged at 16,000×g for 10 min at 4° C. and the soluble fractions were saved. Dimethylsulfoxide was removed from a portion of each soluble fraction using PD MiniTrap™ G-25 desalting columns (GE Healthcare). Homogenates (0.25 mL), free of DMSO, were diluted with an equal volume of binding buffer (i.e., homogenization buffer without dimethylsufoxide) and adjusted to contain 50 μL of a 50% slurry of gelatin-conjugated sepharose. Following 2 hours of rotation at 4° C. the beads were washed twice in binding buffer and eluted in 100 μL 2×-reducing Laemmli buffer with heating to 95° C. for 5 minutes. Eluates (20 μL) were resolved on 4-12% NuPAGE gels, transferred to 0.45 μm pore-sized nitrocellose and immunoblotted for detection of proMMP9, active MMP9, and other processed forms using mAb-L51/82 and pAb-1246 as described above for detection of MMP9 forms in synoviocyte and HFL-1 cell conditioned media.

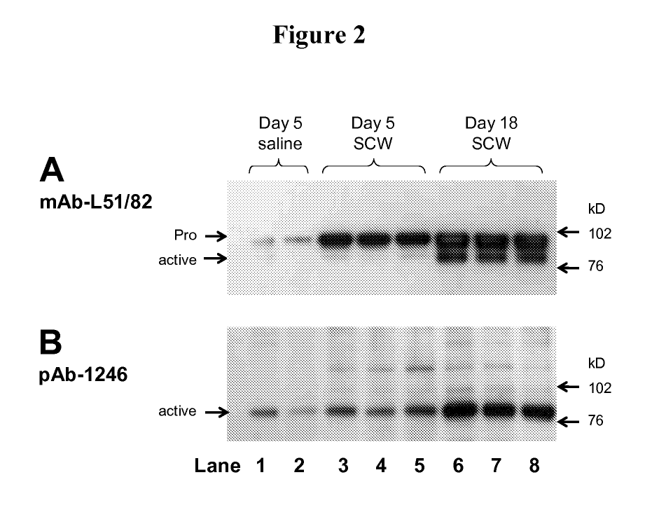

In healthy ankles of rats administered saline, mAb-L51/82 detected small amounts of an approximately 100 kD (proMMP9) and an approximately 80 kD form of MMP9 (FIG. 2A, lanes 1 and 2). proMMP9 was increased markedly in ankle homogenates 5 and 18 days after SCW-administration (FIG. 2A, lanes 3-5 and 6-8, respectively). The 80 kD MMP9 was increased mildly 5 days after SCW-administration (FIG. 2A, lanes 3-5) and was increased markedly 18 days after SCW-administration (FIG. 2A, lanes 6-8). In healthy ankles of rats administered saline, mAb-1246 detected small amounts active MMP9 at 80 kD (FIG. 2B, lanes 1 and 2). The 80 kD active MMP9 was increased mildly 5 days after SCW-administration (FIG. 2A, lanes 3-5) and was increased markedly 18 days after SCW-administration (FIG. 2A, lanes 6-8).

Efficacy of Compound-α in Rats with SCW Arthritis

Having shown that active MMP9 is increased in rats with SCW-induced arthritis, we next sought to determine the ability of Compound-α to reduce disease severity and to reduce active MMP9.

Compound-α Reduced Ankle Swelling of Rats with SCW-Induced Arthritis

To induce arthritis, Female Lewis (LEW/N) rats, 5-6 weeks of age (80-100 g) were injected (i.p.) with SCW PG-PS as described above. Eighteen days later, arthritis was well established. Calipers were used to measure the width (anterior to posterior surface) of the left and right hind ankles of each rat. Each ankle was measured 3 times and averaged, and treatment groups were randomized based on ankle thickness (Table 6). Commencing on day 18, randomized groups of arthritic rats (n=5 rats/group) received vehicle or 5, 20, or 50 mg/kg Compound-α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 26.

By day 18 mean ankle thickness was increased an average of >4.4 mm compared to disease free rats. Rats treated with vehicle alone continued to gradually develop a more severe arthritis based on ankle thickness measurements over the eight-day treatment period (Table 6). Treatment with Compound-α induced a dose-dependent decrease in ankle thickness measurements. By day 26, the disease associated increase in ankle thickness had been reduced 27, 37, and 46 percent by 5, 20, and 50 mg/kg Compound-α, respectively.

TABLE 6
Ankle thickness of rats with SCW-arthritis dosed with vehicle vs. Compound-α

| | | Ankle thickness (mm) ^{a} | | Day 26 Δ mm | |
|---|---|---|---|---|---|
| Treatment | | Day 18 | Day 26 | (vs. group 1) | % Inh |
| Group 1: | mean (n = 4) | 7.20 | 7.26 | 0 | 100 |
| Sterile Saline | SD | 0.043 | 0.012 | | |
| Vehicle | p-value ^{b} | 0.0000 | 0.0001 | | |
| Day 18-26 | | | | | |
| Group 2: | mean (n = 5) | 11.86 | 12.31 | 5.04 | 0 |
| PG-PS (15 μg/gramBW) | SD | 0.77 | 1.26 | | |
| Vehicle | p-value* | na | na | | |
| Day 18-26 | | | | | |
| Group 3: | mean (n = 5) | 11.79 | 10.93 | 3.67 | 27 |
| PG-PS (15 μg/gramBW) | SD | 0.56 | 0.21 | | |
| Compound-α (5 mg/kg) | p value* | 0.88 | 0.043 | | |
| Day 18-26 | | | | | |
| Group 4: | mean (n = 5) | 11.76 | 10.42 | 3.15 | 37 |
| PG-PS (15 μg/gramBW) | SD | 0.73 | 0.93 | | |
| Compound-α (20 mg/kg) | p-value* | 0.85 | 0.028 | | |
| Day 18-26 | | | | | |
| Group 5: | mean (n = 5) | 11.68 | 9.99 | 2.73 | 46 |
| PG-PS (15 μg/gramBW) | SD | 0.62 | 0.73 | | |
| Compound-α (50 mg/kg) | p-value* | 0.71 | 0.0075 | | |
| Day 18-26 | | | | | |

^{a} Calipers were used to measure the width (anterior to posterior surface) of the left and right hind ankles of each rat. Each ankle was measured 3 times and averaged.
^{b} Student's t-test vs. group 2

Hind paw inflammation clinical scores were assigned based on swelling and erythema. By day 18, nearly all rats induced with SCW PG-PS had a clinical score of 8 based on an 8-point scale (Table 7). Treatment with Compound-α induced a dose dependent decrease in clinical score measurements with significant effects emerging at the 20 mg/kg dose (Table 7).

TABLE 7
Clinical Scores of rats with SCW-arthritis
dosed with vehicle vs. Compound-α

| | | Clinical Scores | | |
|---|---|---|---|---|
| | | (0-8) ^{a} | | Δ Day 18 vs. |
| Treatment | | Day 18 | Day 26 | day 26 |
| Group 1: | mean (n = 4) | 0 | 0 | 0 |
| Sterile Saline | SD | 0 | 0 | |
| Vehicle | p-value ^{b} | | <0.0001 | |
| Day 18-26 | | | | |
| Group 2: | mean (n = 5) | 7.80 | 7.80 | 0 |
| PG-PS (15 μg/gramBW) | SD | 0.45 | 0.45 | |
| Vehicle | p-value | | na | |
| Day 18-26 | | | | |
| Group 3: | mean (n = 5) | 8.00 | 6.80 | −1.20 |
| PG-PS (15 μg/gramBW) | SD | 0.00 | 1.09 | |
| Compound-α (5 mg/kg) | p-value | | 0.095 | |
| Day 18-26 | | | | |
| Group 4: | mean (n = 5) | 8.00 | 5.20 | −2.80 |
| PG-PS (15 μg/gramBW) | SD | 0.00 | 1.79 | |
| Compound-α (20 mg/kg) | p-value | | 0.014 | |
| Day 18-26 | | | | |
| Group 5: | mean (n = 5) | 7.80 | 4.40 | −3.40 |
| PG-PS (15 μg/gramBW) | SD | 0.45 | 1.67 | |
| Compound-α (50 mg/kg) | p-value | | 0.0023 | |
| Day 18-26 | | | | |

^{a} Hind paw inflammation clinical scores were assigned based on swelling and erythema as follows: 1 = ankle involvement only; 2 = involvement of ankle and proximal ½ of tarsal joint; 3 = involvement of the ankle and entire tarsal joint down to the metatarsal joints; and 4 = involvement of the entire paw including the digits. Scores of both hind-paws were summed for a maximal score of 8.
^{b} Student's t-test vs. group 2

Compound-α Reduced Active MMP9 in Ankles of Rats with SCW-Induced Arthritis Demonstrated by Western Blot Analysis

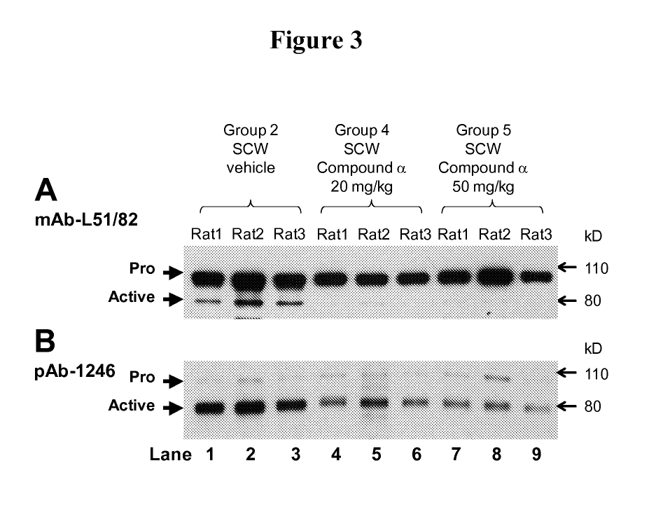

Rats in the study reported in Tables 4 and 5 were sacrificed on Day 26 four hours after the AM dose. Ankles harvested from the right-hind-limbs were processed by the method described above. Pro and active MMP9 were abundantly present in ankles of SCW-induced vehicle-treated rats (FIGS. 3A and 3B, lanes 1-3). Treatment of rats with Compound-α did not reduce the abundance of proMMP9 (FIG. 3A, lanes 4-9). However, treatment of rats with Compound-α resulted in a notable reduction in the active 80 kD form of MMP9 detected with pAb-1246 (FIG. 3B, lanes 4-9 vs. 1-3) and with mAb-L51/82 (FIG. 3A, lanes 4-9 vs. 1-3).

Compound-α Reduced MMP9 Mediated Gelatinase Activity in the Livers of Rats with SCW Arthritis In situ zymography provides an alternative approach to assess active MMP9 in tissues (Frederiks). Tissue sections are overlain with fluorescene-conjugated gelatin wherein the conjugation is sufficiently dense to cause the fluorescene to be dye-quenched (DQ). Proteolytic degradation of the DQ-gelatin releases the fluorescene from the quenching effect giving rise to bright green fluorescence at the site of degradation. Because in situ zymography requires the use of frozen sections, calcified tissues are problematic. However, an additional feature of the SCW arthritis model is the development of hepatic granulomatous disease (J Immunol; 1986; 137:2199-2209), and MMP9 reportedly plays a role in macrophage recruitment in the granulomas response to mycobacteria (Infect Immun; 2006; 74:6135-6144). Consequently, granulomatous livers from SCW-treated rats were assessed for active MMP9 by in situ zymography.

As described above, Female Lewis (LEW/N) rats, 5-6 weeks of age (80-100 g) were injected (i.p.) with saline or SCW PG-PS. On day 28, when the granulomatous response was well established, animals were sacrificed and livers were frozen in OCT cryo-sectioning medium and 10 μm sections were cut on a Cryome HM 500 M cryotome and mounted on glass microscope slides. Sections were air dried briefly. MMP9 was confirmed as the source of the gelatinase activity in the liver by treating liver sections with monoclonal antibodies directed against the active site of the two major gelatinases MMP9 and MMP2. Liver sections overlain with 50 μL of 100 μg/mL neutralizing mouse monoclonal antibodies directed against MMP9 (Calbiochem, clone 6-6B), or MMP2 (Millipore, clone CA-4001), or with PBS for 1 hr at room temperature. Tissues were rinsed once with PBS, blotted, and briefly air dried and then overlain with DQ-gelatin (Invitrogen) dissolved to 1 mg/mL in deionized water and then diluted 1:10 in 1% wt/vol low gelling point agarose type VII (Sigma) in PBS. The sections were covered with coverslips, incubated in the dark at room temperature for 20 min, and imaged on an Olympus IX80 inverted microscope fitted with fluorescence optics, using SlideBook™ imaging software (Intelligent Imaging Innovations, Inc., Philadelphia, Pa.; version 5.0). Fluorescence intensity was determined (Table 8). When compared to a saline-treated rat, gelatinase activity was abundantly expressed in granulomatous liver sections obtained from a rat with SCW arthritis. The activity in the granulomatous liver sections was almost completely inhibited by treatment with anti-MMP9 monoclonal antibody but not by treatment with anti-MMP2 monoclonal antibody.

TABLE 8
Indentification of MMP9 as the gelatinase responsible for signals
detected by in situ zymography in SCW-granulomatous livers

| | | |
|---|---|---|
| Disease | Section | Intensity (RLU × 10⁶) |

| induction | treatment | Mean | SD |
|---|---|---|---|
| Saline-healthy | PBS | 11.4 | 2.91 |
| SCW- | PBS | 109 | 19.3 |
| granulomatous | Anti-MMP9 | 1.02 | 0.17 |
| | Anti-MMP2 | 128 | 36.2 |

Key: RLU = relative light units; SCW = Streptococcal cell wall peptidoglycan-polysaccharide equivalent to 15 μg rhamnose/gram BW.

Next, liver in situ zymography was used to assess the relative presence of active MMP9 in rats dosed with vehicle vs. Compound-α. Female Lewis (LEW/N) rats, 5-6 weeks of age (80-100 g) were injected (i.p.) with saline or SCW PG-PS. Commencing on day 25, randomized groups of rats (n=3 rats/group) received vehicle or 20 or 50 mg/kg Compound-α BID by oral gavage. Vehicle consisted of an aqueous mixture containing 2% (v:v) N-methylpyrrolidone, 5% (v:v) glycerine, and 20% (w:v) captisol. Treatment continued daily through the morning of day 28. Four hrs after the AM dose on day 28, rats were sacrificed and livers assessed for active MMP9 by in situ zymography (Table 9). Gelatinase activity was increased markedly in SCW-induced rats, but activity was reduced by approximately 80% in animals treated with 50 mg/kg Compound-α.

TABLE 9
In situ zymography determination of gelatinase activity in livers of
SCW-induced rats dosed with vehicle vs. Compound-α

| | Intensity (RLU × 10⁶) | | | | | t-test vs. |
|---|---|---|---|---|---|---|
| Treatment | Rat 1 | Rat 2 | Rat 3 | Mean | SD | SCW-vehicle |
| Saline | 3.3 | 1.1 | 1.6 | 2.0 | 1.15 | 0.001 |
| Vehicle | | | | | | |
| Day 25-28 | | | | | | |
| SCW | 65.1 | 43.4 | 58.9 | 55.8 | 11.17 | 1 |
| Vehicle | | | | | | |
| Day 25-28 | | | | | | |
| SCW | 43.0 | 69.0 | 53.7 | 55.2 | 13.06 | 0.96 |
| Compound-α | | | | | | |
| (20 mg/kg) | | | | | | |
| Day 25-28 | | | | | | |
| SCW | 3.2 | 25.6 | 4.5 | 11.1 | 12.57 | 0.010 |
| Compound-α | | | | | | |
| (50 mg/kg) | | | | | | |
| Day 25-28 | | | | | | |

Key:
RLU = relative light units;
SCW = Streptococcal cell wall peptidoglycan-polysaccharide equivalent to 15 μg rhamnose/gram BW.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

All publications disclosed in the above specification are hereby incorporated by reference in full.

We claim:

1. The compounds of Formula I 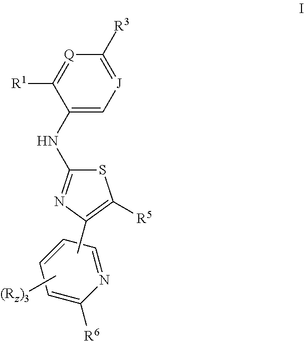
wherein:
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;
Q is N or C—R²;
R² is H, or CH₃; or R² and R¹ may be taken together with the ring to which they are attached, to form a fused ring system selected from the group consisting of: quinolinyl, isoquinolinyl, quinazolinyl, quinoxalinyl, benzimidazolyl, napthalyl, benzofuranyl, 2,3-dihydro-benzofuranyl, benzothiophenyl, benzothiazolyl, benzotriazolyl, indolyl, indolinyl, and indazolyl, wherein said quinolinyl, isoquinolinyl, quinazolinyl, quinoxalinyl, benzimidazolyl, benzothiazolyl, napthalyl, benzofuranyl, 2,3-dihydro-benzofuranyl, benzothiophenyl, benzotriazolyl, indolyl, indolinyl, and indazolyl are optionally substituted with one methyl group or up to two fluorine atoms;
R³ is Cl, SO₂NH₂, SO₂CH₃, CO₂H, CONH₂, NO₂, —CN, CH₃, CF₃, or H;
J is N, or C—R⁴;
R⁴ is NH₂, NHC₍₁₋₃₎alkyl, N(C₍₁₋₃₎alkyl)₂, C₍₁₋₃₎alkyl, —CN, —CH═CH₂, —CONH₂, —CO₂H, —NO₂, —CONHC₍₁₋₄₎alkyl, CON(C₍₁₋₄₎alkyl)₂, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), —SO₂N(C₍₁₋₄₎alkyl)₂, —CONHC₍₂₋₄₎alkyl-piperidinyl, —CONHC₍₂₋₄₎alkyl-pyrrolidinyl, —CONHC₍₂₋₄₎alkyl-piperazinyl, —CONHC₍₂₋₄₎alkyl-morpholinyl, —CONHCH₂Ph, or R⁴ is selected from the group consisting of: phenyl, pyridyl, pyrimidyl, pyrazyl, pyrazolyl, imidazolyl, oxazolyl, isoxazolyl, thiazolyl, furyl, and thiophenyl wherein said phenyl, pyridyl, pyrimidyl, pyrazyl, pyrazolyl, imidazolyl, oxazolyl, isoxazolyl, thiazolyl, furyl, and thiophenyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system; or R⁴ and R³ may be taken together with the ring to which they are attached, to form the fused ring system 2,3-dihydroisoindolin-1-one;
R^{d} is C₍₁₋₄₎alkyl, F, Cl, Br, —CN, or OC₍₁₋₄₎alkyl;
R⁵ is H, F, Cl, Br, CF₃, or CH₃;
R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, CF₃, 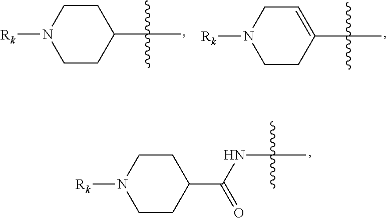
CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, SO₂C₍₁₋₄₎alkylNA¹A², SOC₍₁₋₄₎alkylNA¹A², pyridinyl, pyrimidinyl, pyrazinyl, NA¹A², C(O)NA¹A², SO₂NA¹A², SONA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R⁶ may be optionally substituted with up to four methyl groups on two or more ring carbon atoms or optionally substituted with up to two CF₃ groups on any two ring carbon atoms;
A¹ is H, or C₍₁₋₃₎alkyl;
A² is H, C₍₁₋₆₎alkyl, CH₂C₍₃₋₆₎cycloalkyl, C₍₃₋₆₎cycloalkyl, 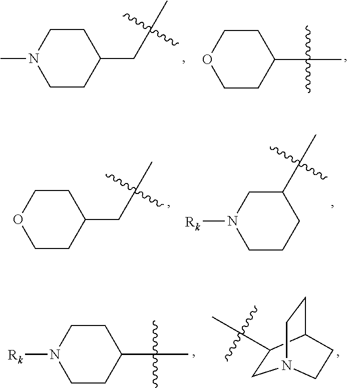
C₍₂₋₆₎alkylOH, C₍₂₋₆₎alkylOCH₃, C₍₂₋₆₎alkylCO₂C₍₁₋₄₎alkyl, SO₂C₍₁₋₄₎alkyl, C(O)Ph, C(O)C₍₁₋₄₎alkyl, pyrazinyl, or pyridyl, wherein said cycloalkyl, alkyl, pyrazinyl, pyridyl, or Ph groups may be optionally be substituted with two substituents selected from the group consisting of F, C₍₁₋₆₎alkyl, CF₃, pyrrolidinyl, CO₂H, C(O)NH₂, SO₂NH₂, OC₍₁₋₄₎alkyl, —CN, NO₂, OH, NH₂, NHC₍₁₋₄₎alkyl, N(C₍₁₋₄₎alkyl)₂; and said pyridyl, or Ph may be additionally be substituted with up to two halogens independently selected from the group consisting of: Cl, and Br; or A¹ and A² are taken together with their attached nitrogen to form a ring selected from the group consisting of: 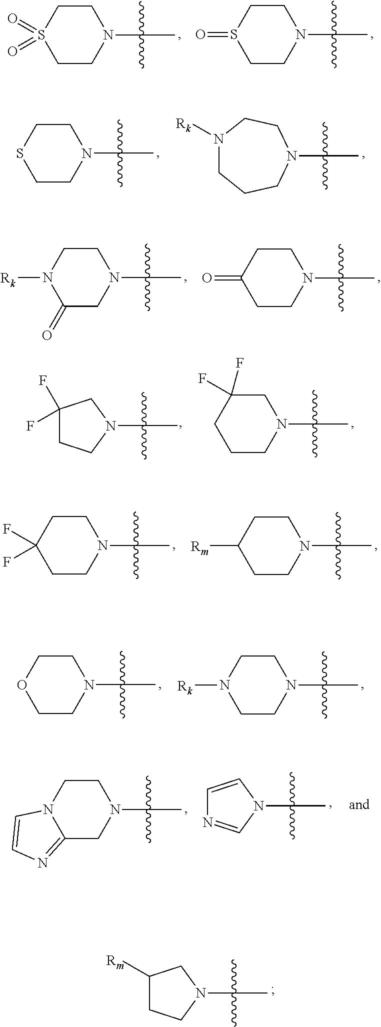
wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₆₎alkyl, COC₍₁₋₄₎alkyl, SO₂C₍₁₋₄₎alkyl, trifluoromethylpyridyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, C₍₁₋₆₎alkyl, F, or OH; and
R_{z} is independently selected from the group consisting of H, C₍₁₋₆₎alkyl, OCH₃, F, Cl, Br, —CN, and CF₃; or if R⁶ is H, C₍₁₋₆₎alkyl, OCH₃, F, Cl, Br, —CN, or CF₃ then R_{z} may be selected from the group consisting of: 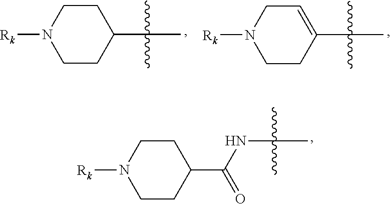
CO₂H, CO₂C₍₁₋₄₎alkyl, C(O)C₍₁₋₄₎alkyl, C(O)Ph, SO₂C₍₁₋₄₎alkyl, SOC₍₁₋₄₎alkyl, pyridinyl, pyrimidinyl, pyrazinyl, NA¹A², C(O)NA¹A², SO₂NA¹A², SONA¹A², SO₂C₍₁₋₄₎alkylNA¹A², SOC₍₁₋₄₎alkylNA¹A², C(O)N(C₍₁₋₃₎alkyl)C₍₂₋₆₎alkylNA¹A², C(O)NHC₍₂₋₆₎alkylNA¹A², NHC(O)C₍₁₋₆₎alkylNA¹A², N(C₍₁₋₃₎alkyl)C(O)C₍₁₋₆₎alkylNA¹A², C₍₁₋₆₎alkylOC₍₁₋₆₎alkyl, C₍₁₋₆₎alkylOC₍₃₋₆₎cycloalkyl, C₍₁₋₆₎alkylOC₍₂₋₆₎alkylNA¹A², C₍₁₋₆₎alkylNHC₍₂₋₆₎alkylNA¹A², C₍₁₋₆₎alkylN(C₍₁₋₃₎alkyl)C₍₂₋₆₎alkylNA¹A², NHC₍₂₋₆₎alkylNA¹A², N(C₍₁₋₃₎alkyl)C₍₂₋₆₎alkylNA¹A², OC₍₂₋₆₎alkyl, OC₍₃₋₆₎cycloalkyl, OC₍₂₋₆₎alkylNA¹A², or C₍₁₋₆₎alkylNA¹A²;
wherein any piperidinyl in R_{z} may be optionally substituted with up to four methyl groups on two or more ring carbon atoms or optionally substituted with up to two CF₃ groups on any two ring carbon atoms;
and solvates, hydrates, tautomers, and pharmaceutically acceptable salts thereof.

2. A compound of claim 1, wherein:
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;
R² is H, or CH₃; or R² and R¹ may be taken together with the ring to which they are attached, to form a fused ring system selected from the group consisting of: quinolinyl, isoquinolinyl, quinazolinyl, quinoxalinyl, benzimidazolyl, benzofuranyl, 2,3-dihydro-benzofuranyl, benzothiophenyl, benzothiazolyl, and indazolyl, wherein said quinolinyl, isoquinolinyl, quinazolinyl, quinoxalinyl, benzimidazolyl, benzothiazolyl, benzofuranyl, 2,3-dihydro-benzofuranyl, benzothiophenyl, and indazolyl are optionally substituted with one methyl group or up to two fluorine atoms;
R⁴ is CH₃, —CN, —CONH₂, —CO₂H, —NO₂, —CONHC₍₁₋₄₎alkyl, C₍₁₋₄₎alkylCONH₂, —NHCOC₍₁₋₄₎alkyl, —CO₂C₍₁₋₄₎alkyl, CF₃, SO₂C₍₁₋₄₎alkyl, —SO₂NH₂, —SO₂NH(C₍₁₋₄₎alkyl), or R⁴ is selected from the group consisting of: pyrazolyl, imidazolyl, oxazolyl, isoxazolyl, thiazolyl, furyl, and thiophenyl wherein said pyrazolyl, imidazolyl, oxazolyl, isoxazolyl, thiazolyl, furyl, and thiophenyl are optionally substituted with one Rd; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;
R^{d} is CH₃, F, Cl, Br, —CN, or OCH₃;
R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, CF₃, 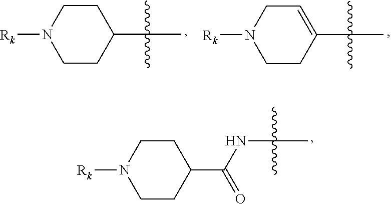
CO₂H, CO₂C₍₁₋₄₎alkyl, NA¹A², C(O)NA¹A², SO₂NA¹A², SONA¹A², C(O)N(CH₃)C₍₂₋₆₎alkylNA¹A², C(O)NHC₍₂₋₆₎alkylNA¹A², NHC(O)C₍₁₋₆₎alkylNA¹A², N(CH₃)C(O)C₍₁₋₆₎alkylNA¹A², CH₂OC₍₁₋₆₎alkyl, CH₂OC₍₃₋₆₎cycloalkyl, CH₂OC₍₂₋₆₎alkylNA¹A², CH₂NHC₍₂₋₆₎alkylNA¹A², CH₂N(CH₃)C₍₂₋₆₎alkylNA¹A², NHC₍₂₋₆₎alkylNA¹A², N(CH₃)C₍₂₋₆₎alkylNA¹A², OC₍₂₋₆₎alkyl, OC₍₂₋₆₎alkylNA¹A², or CH₂NA¹A²;
A¹ is H, or C₍₁₋₃₎alkyl;
A² is H, C₍₁₋₆₎alkyl, CH₂C₍₃₋₆₎cycloalkyl, C₍₃₋₆₎cycloalkyl, 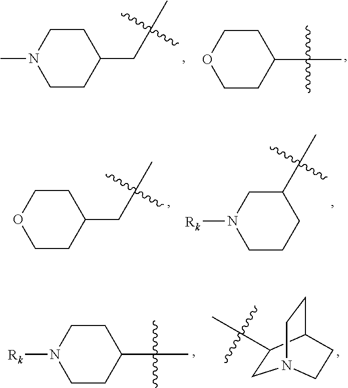
C₍₂₋₆₎alkylOH, C₍₂₋₆₎alkylOCH₃, C₍₂₋₆₎alkylCO₂C₍₁₋₃₎alkyl, SO₂C₍₁₋₄₎alkyl, C(O)Ph, C(O)C₍₁₋₄₎alkyl, pyrazinyl, or pyridyl;
or A¹ and A² are taken together with their attached nitrogen to form a ring selected from the group consisting of: 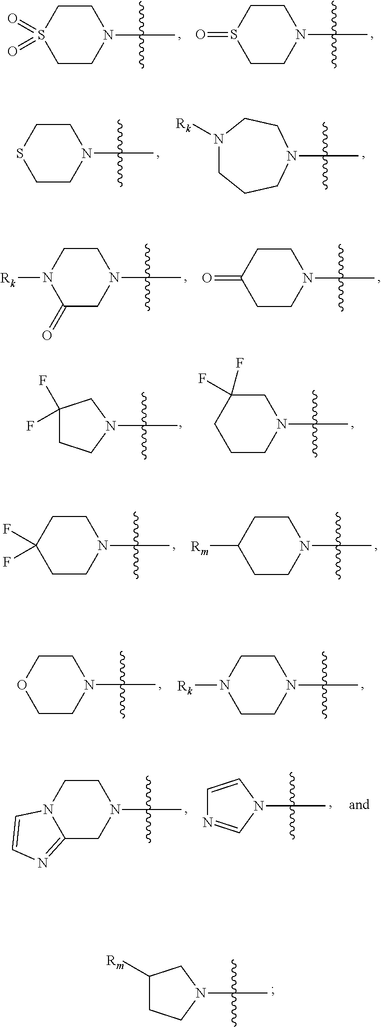
wherein any said A¹ and A² ring, except imidazolyl, may be optionally substituted with up to four methyl groups on two or more ring carbon atoms or optionally substituted with up to two CF₃ groups on any two ring carbon atoms, or optionally substituted with one —CONH₂ group on any one ring carbon atom;
Rₖ is selected from the group consisting of H, CO₂C(CH₃)₃, CH₂CF₃, CH₂CH₂CF₃, C₍₁₋₃₎alkyl, COC₍₁₋₄₎alkyl, SO₂C₍₁₋₄₎alkyl, CH₂C₍₃₋₆₎cycloalkyl, and C₍₃₋₆₎cycloalkyl;
Rₘ is H, OCH₃, CH₂OH, NH(C₍₁₋₄₎alkyl), N(C₍₁₋₄₎alkyl)₂, NH₂, CH₃, F, or OH; and
R_{z} is independently selected from the group consisting of H, C₍₁₋₆₎alkyl, OCH₃, F, Cl, Br, —CN, and CF₃;
and solvates, hydrates, tautomers, and pharmaceutically acceptable salts thereof.

3. A compound of claim 2, wherein:
R¹ is C₍₁₋₄₎alkoxy, C₍₁₋₄₎alkyl, SC₍₁₋₄₎alkyl, Cl, F, OCH₂C₍₃₋₆₎cycloalkyl, OC₍₃₋₆₎cycloalkyl, OCH₂CF₃, SCH₂C₍₃₋₆₎cycloalkyl, SC₍₃₋₆₎cycloalkyl, SCF₃, or OCF₃;
R² is H, or CH₃; or R² and R¹ may be taken together with the ring to which they are attached, to form a fused ring system selected from the group consisting of: quinolinyl, benzofuranyl, and 2,3-dihydro-benzofuranyl, wherein said quinolinyl, benzofuranyl, and 2,3-dihydro-benzofuranyl are optionally substituted with one methyl group or up to two fluorine atoms;
R⁴ is —CN, —CONH₂, —CO₂H, —NO₂, —CO₂C₍₁₋₄₎alkyl, SO₂C₍₁₋₃₎alkyl, —SO₂NH₂, CH₂CONH₂, or
R⁴ is selected from the group consisting of: pyrazolyl, and oxazolyl, wherein said pyrazolyl, and oxazolyl are optionally substituted with one R^{d}; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;
R^{d} is CH₃, F, or Cl;
R⁵ is H, F, Cl, Br, or CH₃;
R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, CF₃, 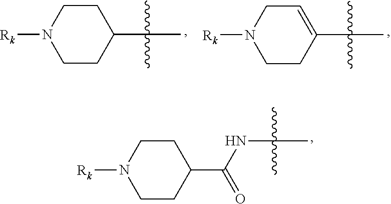
CO₂H, CO₂C₍₁₋₄₎alkyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², OC₍₂₋₃₎alkyl, OC₍₂₋₃₎alkylNA¹A², or CH₂NA¹A²;
A¹ is H, or C₍₁₋₃₎alkyl;
A² is H, C₍₁₋₅₎alkyl, CH₂-cyclopropyl, C₍₂₋₆₎alkylOCH₃, C₍₂₋₆₎alkylCO₂C₍₁₋₃₎alkyl, C₍₂₋₆₎alkylOH, 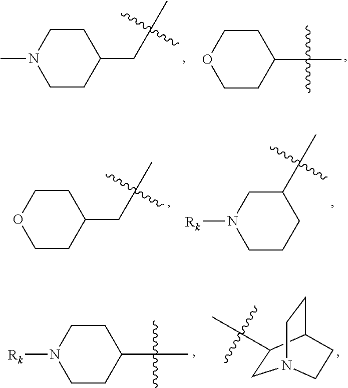
C(O)C₍₁₋₄₎alkyl;
or A¹ and A² are taken together with their attached nitrogen to form a ring selected from the group consisting of: 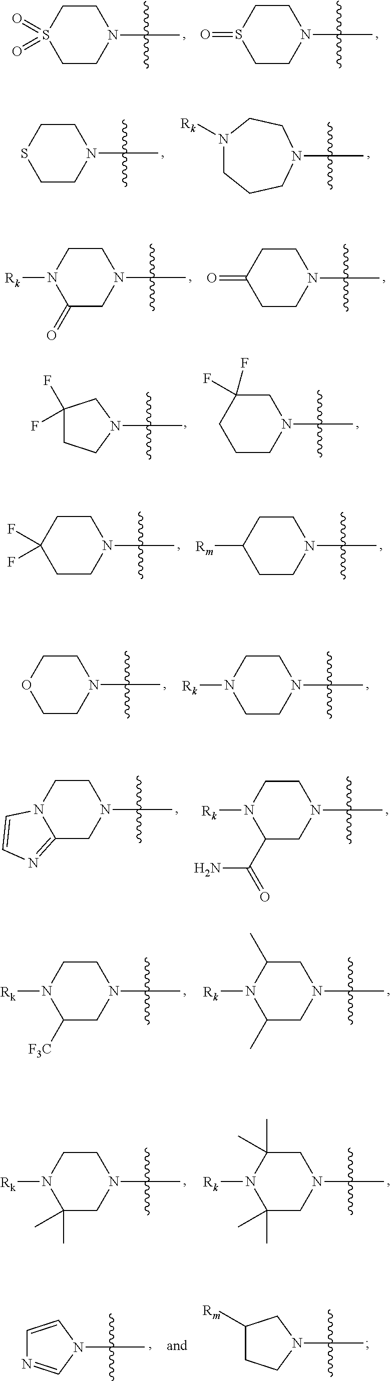
Rₖ is selected from the group consisting of H, CO₂C(CH₃)₃, C₍₁₋₃₎alkyl, COC₍₁₋₄₎alkyl, SO₂C₍₁₋₄₎ alkyl, CH₂CH₂CF₃, CH₂-cyclopropyl, and C₍₃₋₆₎cycloalkyl;
Rₘ is H, OCH₃, CH₂OH, NH(CH₃), N(CH₃)₂, NH₂, CH₃, F, or OH; and
R_{z} is independently selected from the group consisting of H, C₍₁₋₄₎alkyl, and Br;
and solvates, hydrates, tautomers, and pharmaceutically acceptable salts thereof.

4. A compound of claim 3, wherein:
R¹ is OC₍₁₋₄₎alkyl, SC₍₁₋₄₎alkyl, C₍₁₋₄₎alkyl, OCH₂C₍₃₋₅₎cycloalkyl, OC₍₃₋₅₎cycloalkyl, or OCF₃;
R² is H; or R¹ and R² may be taken together with their attached ring to form the fused bicycle 2-methyl benzofuran-7-yl;
R³ is SO₂NH₂, SO₂CH₃, CO₂H, CONH₂, CH₃, —CN, or H;
R⁴ is —CN, —CONH₂, —CO₂H, SO₂C₍₁₋₃₎alkyl, —SO₂NH₂, —NO₂, CH₂CONH₂, or R⁴ is selected from the group consisting of: pyrazolyl, and oxazolyl, wherein said pyrazolyl, and oxazolyl are optionally substituted with one Rd; provided that R⁴ may be H, if R³ is SO₂NH₂, SO₂CH₃, CO₂H, or CONH₂; or R³ and R⁴ may both be H, provided that the ring to which they are attached is pyridyl; or R⁴ may also be H provided that R¹ and R² are taken together with the ring to which they are attached, to form a fused ring system;
R⁵ is H;
R⁶ is H, C₍₁₋₆₎alkyl, C₍₃₋₆₎alkenylOC₍₁₋₆₎alkyl, C₍₂₋₆₎alkylOC₍₁₋₆₎alkyl, OCH₃, F, Cl, Br, —CN, CH₂OH, CF₃ 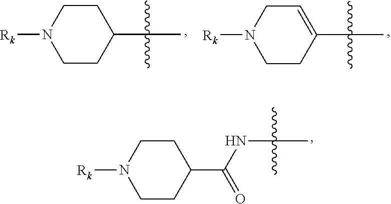
CO₂H, CO₂C₍₁₋₄₎alkyl, NA¹A², C(O)NA¹A², SO₂NA¹A², SONA¹A², C(O)N(CH₃)C₍₂₋₃₎alkylNA¹A², C(O)NHC₍₂₋₃₎alkylNA¹A², NHC(O)C₍₁₋₃₎alkylNA¹A², N(CH₃)C(O)C₍₁₋₃₎alkylNA¹A², CH₂OC₍₂₋₃₎alkylNA¹A², CH₂NHC₍₂₋₃₎alkylNA¹A², CH₂N(CH₃)C₍₂₋₃₎alkylNA¹A², NHC₍₂₋₃₎alkylNA¹A², N(CH₃)C₍₂₋₃₎alkylNA¹A², OC₍₂₋₃₎alkyl, OC₍₂₋₃₎alkylNA¹A², or CH₂NA¹A²;
A¹ is H, or C₍₁₋₃₎alkyl;
A² is H, C₍₁₋₅₎alkyl, CH₂-cyclopropyl, C₍₂₋₃₎alkylOCH₃, CH₂CH₂CO₂CH₂CH₃, CH₂CH₂OH, 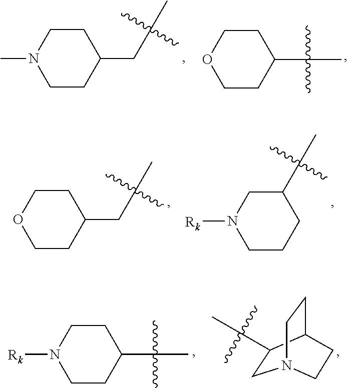
C(O)CO₍₁₋₄₎alkyl;
or A¹ and A² are taken together with their attached nitrogen to form a ring selected from the group consisting of: 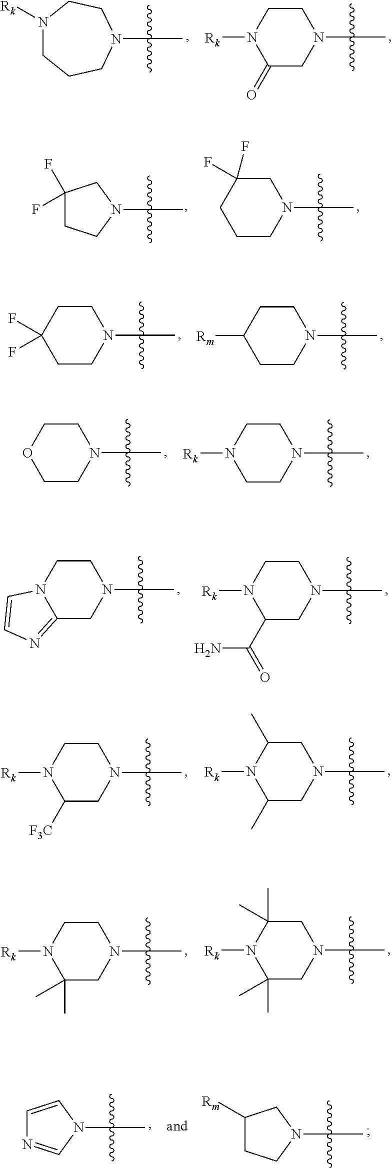
and
Rₖ is selected from the group consisting of H, CO₂C(CH₃)₃, C₍₁₋₃₎alkyl, COC₍₁₋₄₎alkyl, SO₂C₍₁₋₄₎alkyl, CH₂CH₂CF₃, CH₂-cyclopropyl, and C₍₃₋₆₎cycloalkyl;
and solvates, hydrates, tautomers, and pharmaceutically acceptable salts thereof.

5. A compound of claim 4, wherein:
R¹ is OC₍₁₋₃₎alkyl, isobutyl, or OCF₃;
Q is C—R²;
R³ is H, CH₃, or CONH₂;
J is C—R⁴;
R⁴ is CONH₂, SO₂CH₃, SO₂CH₂CH₃, NO₂, oxazolyl, CO₂H, CH₂CONH₂, —CN, or SO₂NH₂; or R₄ is H, provided that R₃ is CONH₂; or R⁴ may also be H provided that R¹ and R² are taken together with the ring to which they are attached, to form a fused ring system;
R⁶ is H, —CH═CH—CH₂OCH₃, CH₂CH₂CH₂OCH₃, NA¹A², Cl, C(O)NA¹A², CO₂H, N(CH₃)CH₂CH₂N(CH₃)₂, C(O)N(CH₃)CH₂CH₂N(CH₃)₂, NHC₍₂₋₃₎alkylNA¹A², C(O)NHCH₂CH₂N(CH₃)₂, CH₂OH, CH₃, Br, OCH₃, 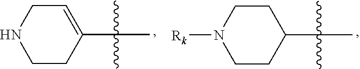
or CF₃;
A¹ is CH₃, or H;
A² is H, C₍₁₋₅₎alkyl, CH₂-cyclopropyl, C₍₂₋₃₎alkylOCH₃, CH₂CH₂CO₂CH₂CH₃, CH₂CH₂OH, 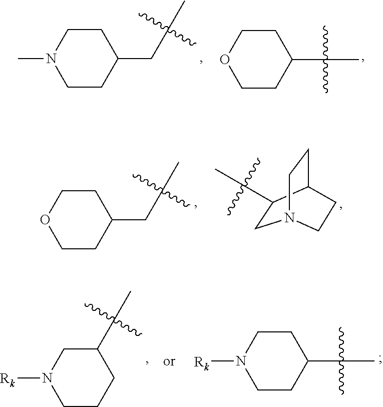
or A¹ and A² may be taken together with their attached nitrogen to form a ring selected from the group consisting of: 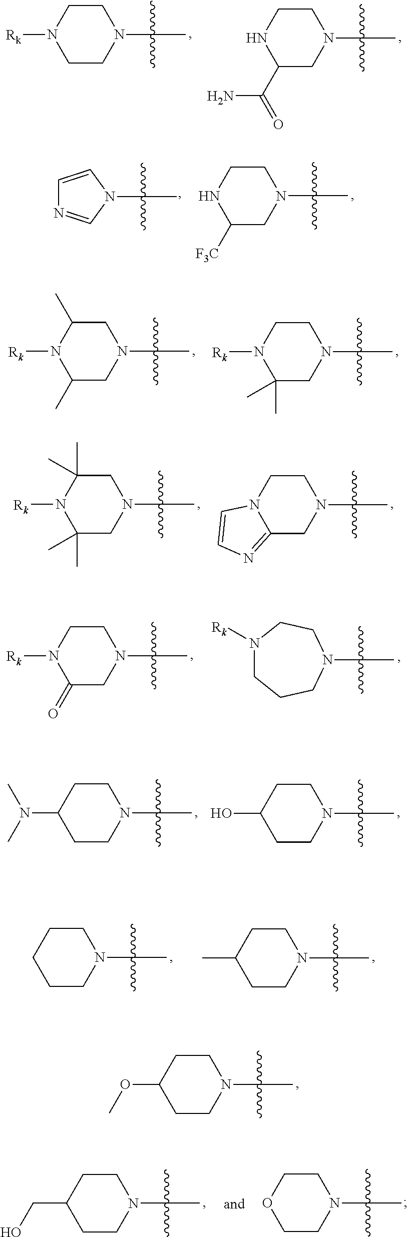
Rₖ is H, CO₂C(CH₃)₃, COCH₃, cyclopropyl, CH₂-cyclopropyl, CH₂CH₂CF₃, SO₂CH₃, or C₍₁₋₃₎alkyl; and
R_{z} is independently selected from the group consisting of H, CH₃, isopropyl, and Br;
and solvates, hydrates, tautomers, and pharmaceutically acceptable salts thereof.

6. A compound of claim 1, wherein:
R¹ is OCH(CH₃)₂;
Q is C—R²;
R² is H;
R³ is H;
J is C—R⁴;
R⁴ is —CONH₂, —CO₂H, or —SO₂NH₂; and
R⁵ is H;
and solvates, hydrates, tautomers, and pharmaceutically available salts thereof.

7. A compound selected from the group consisting of: 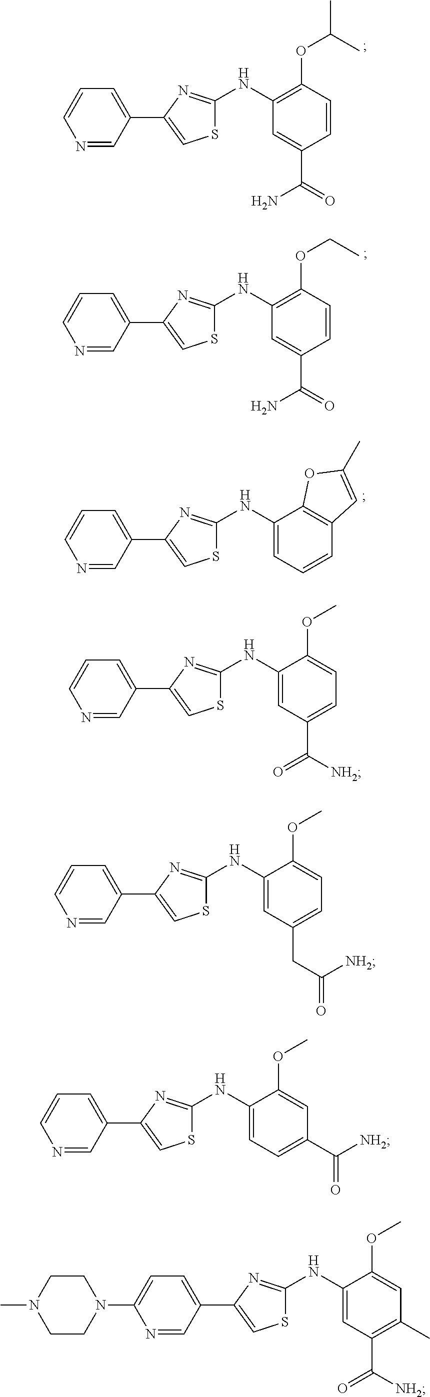 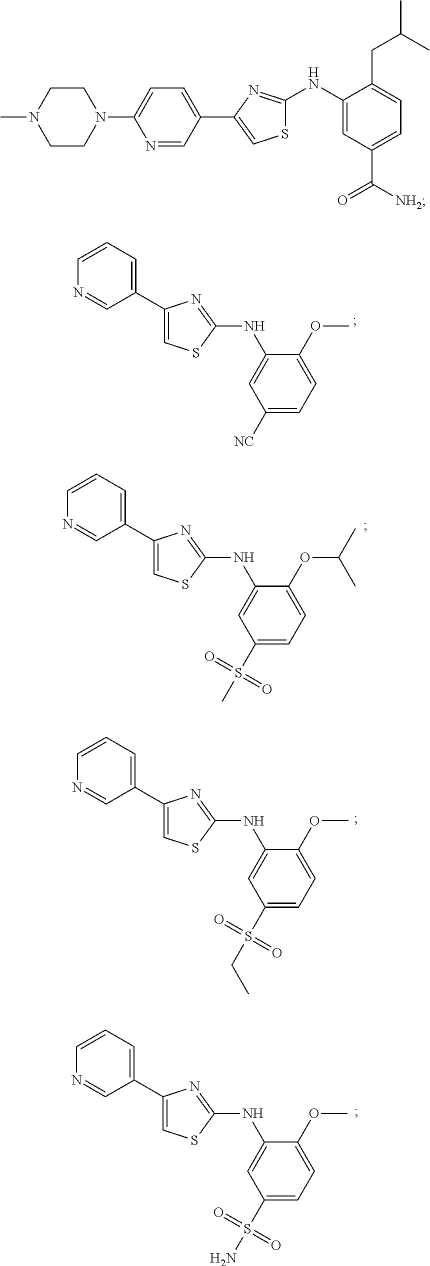 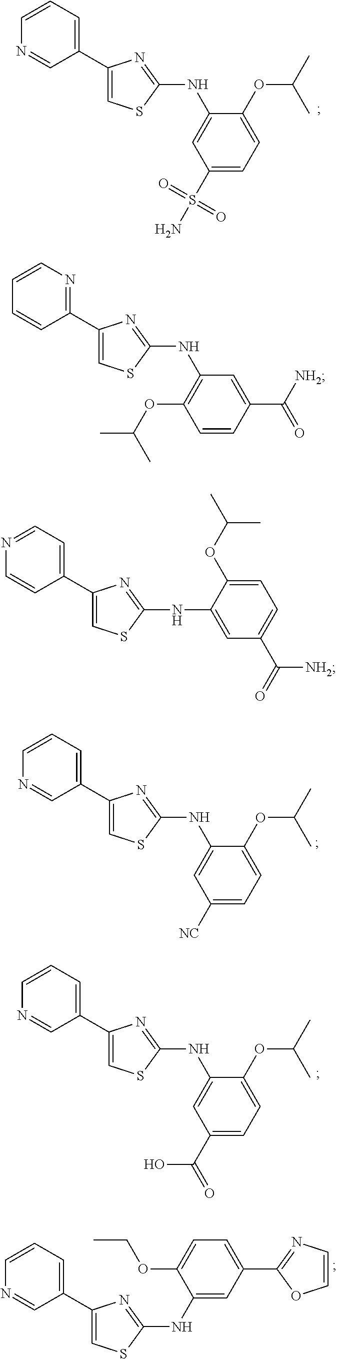 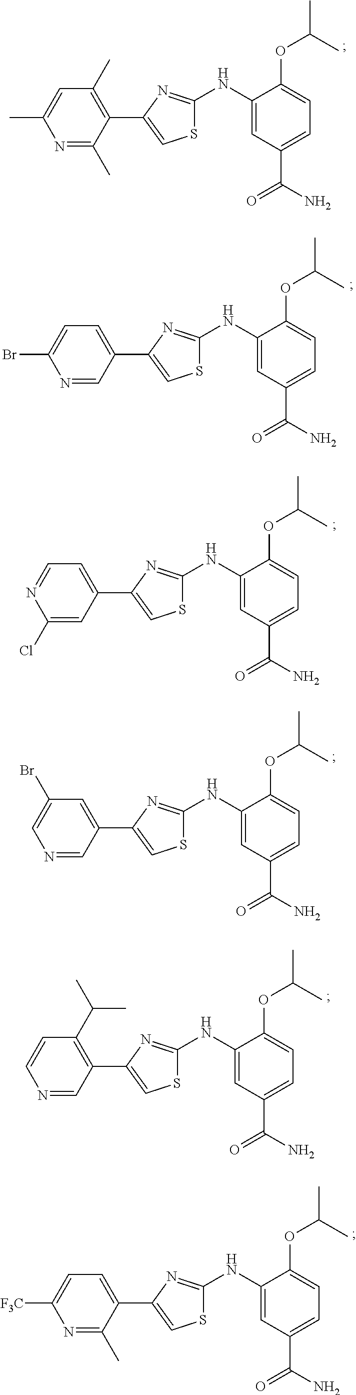 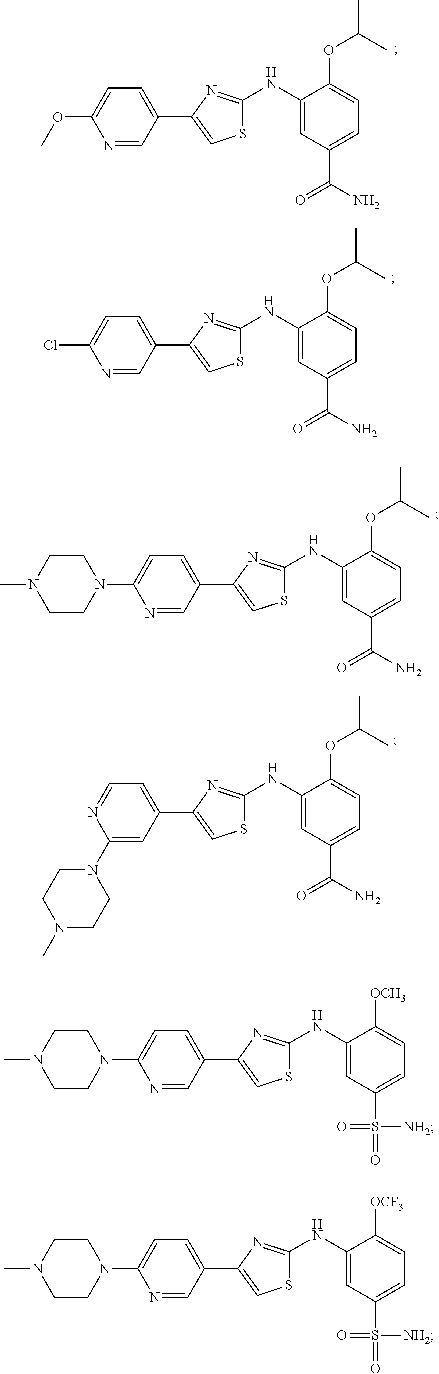 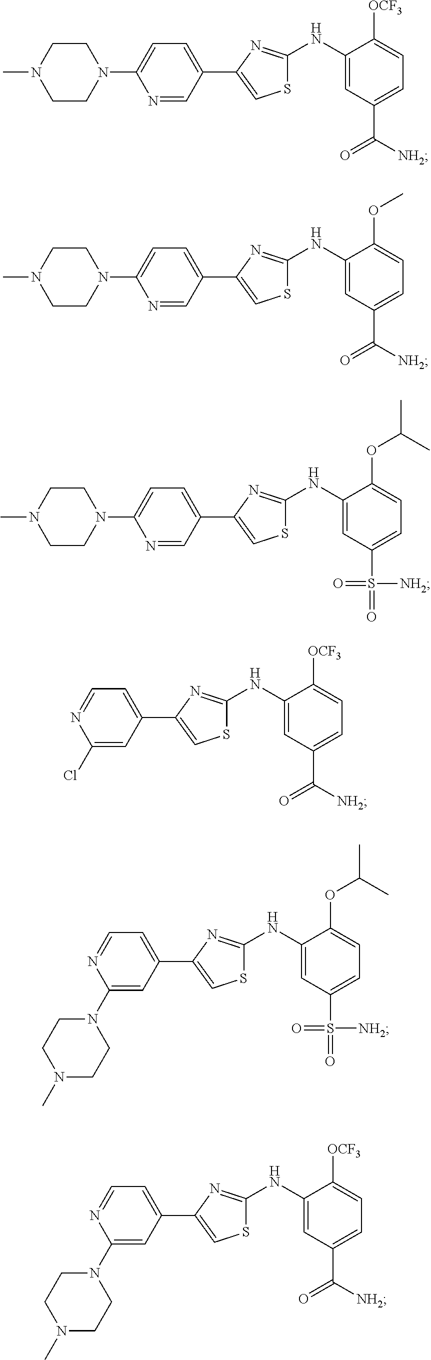 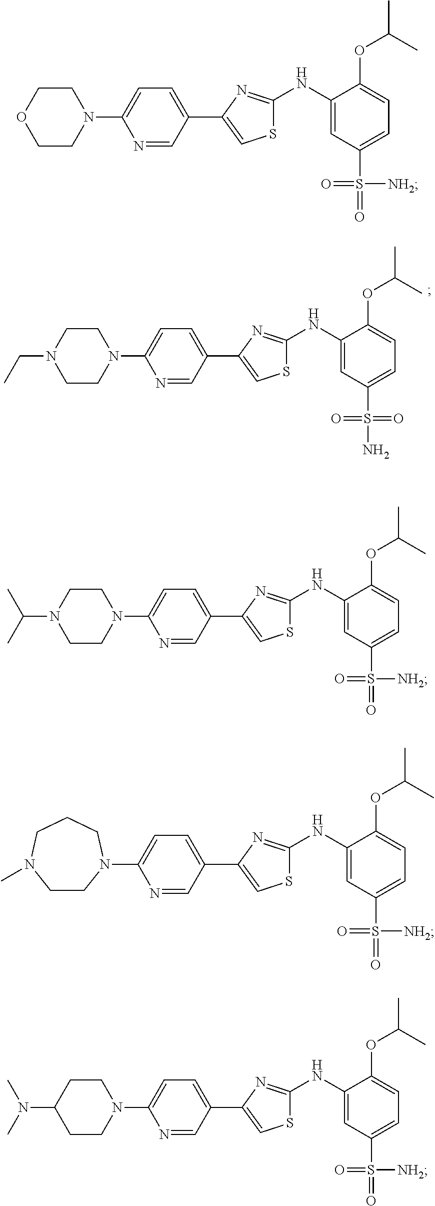 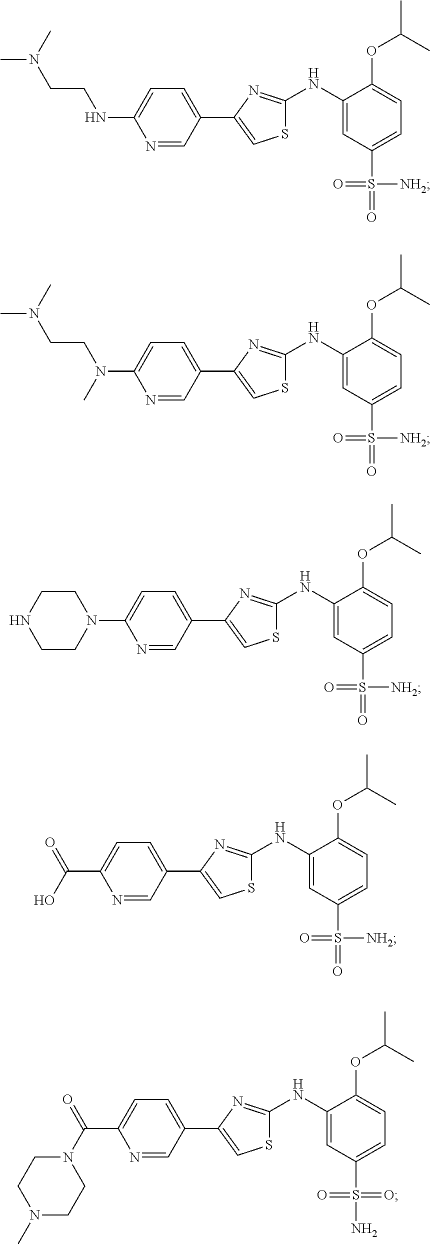 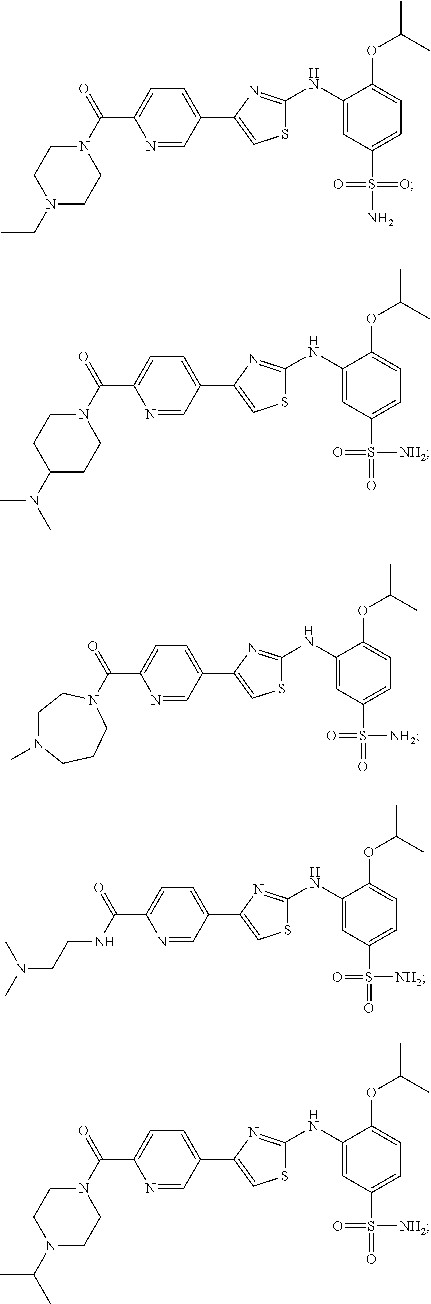 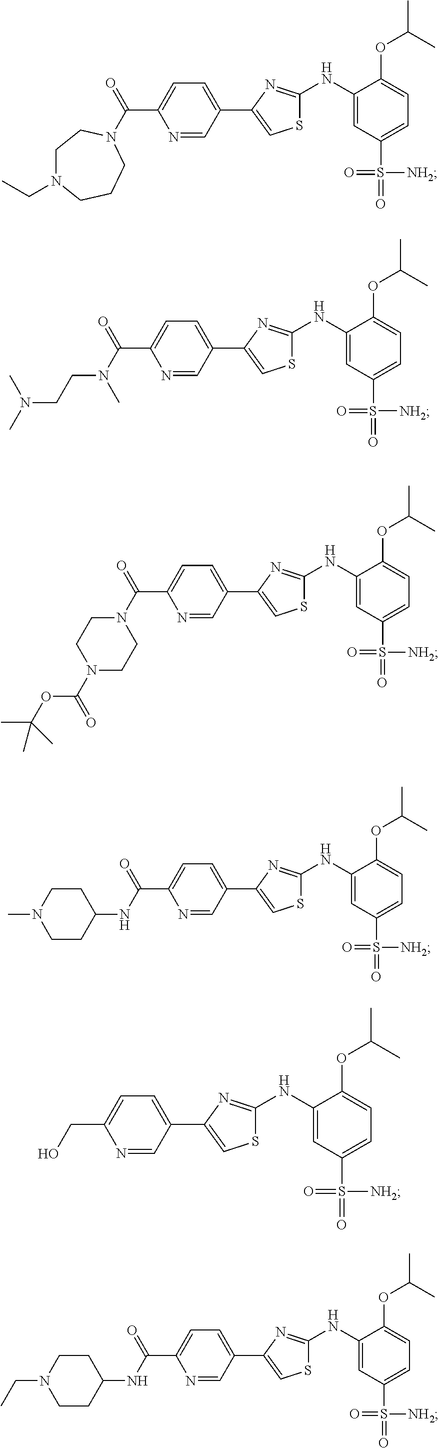 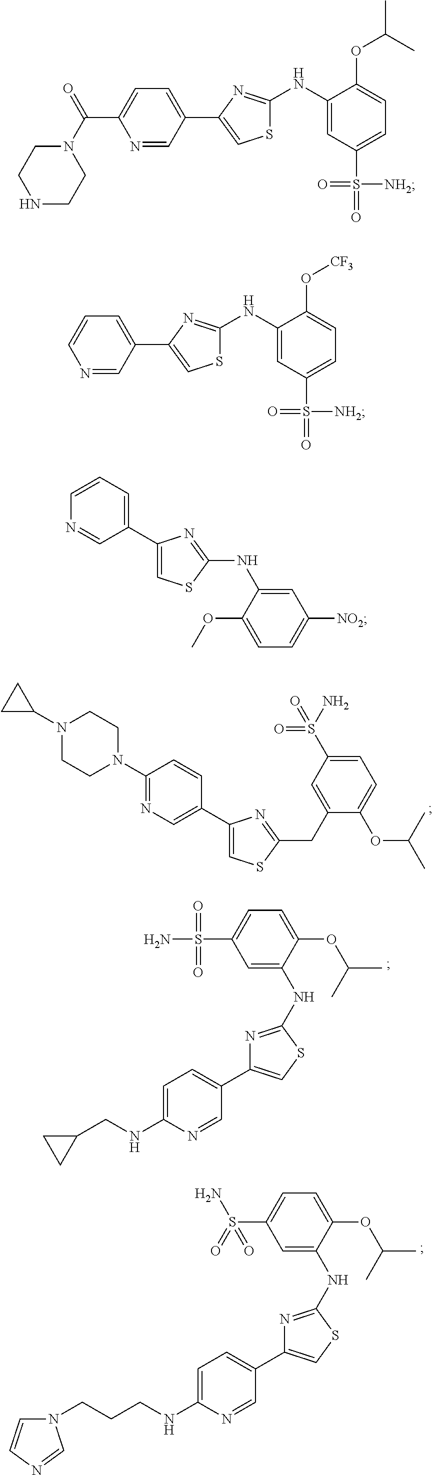 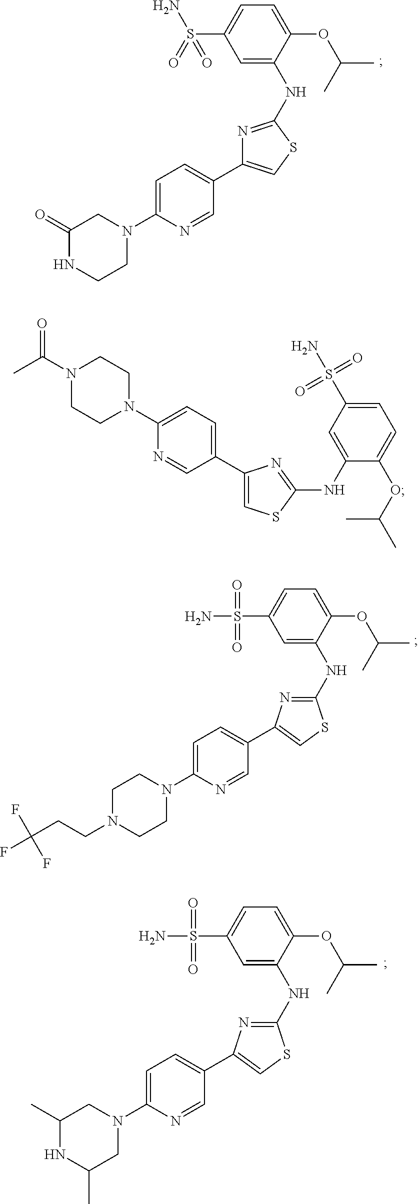 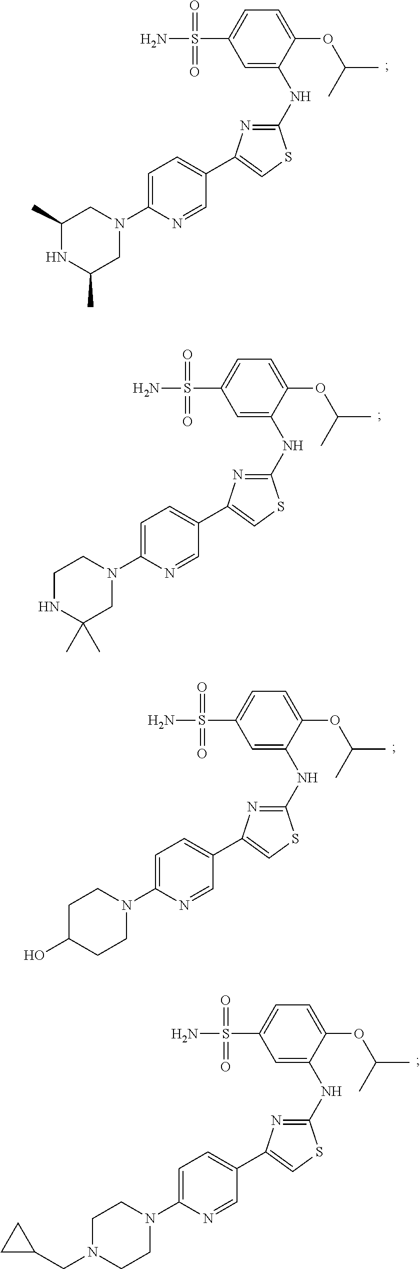 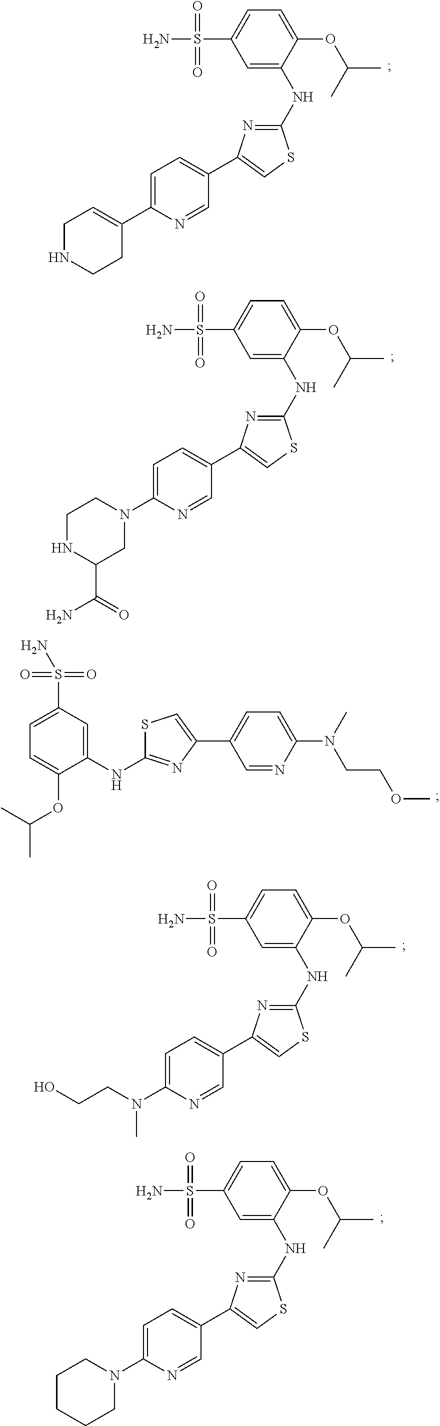 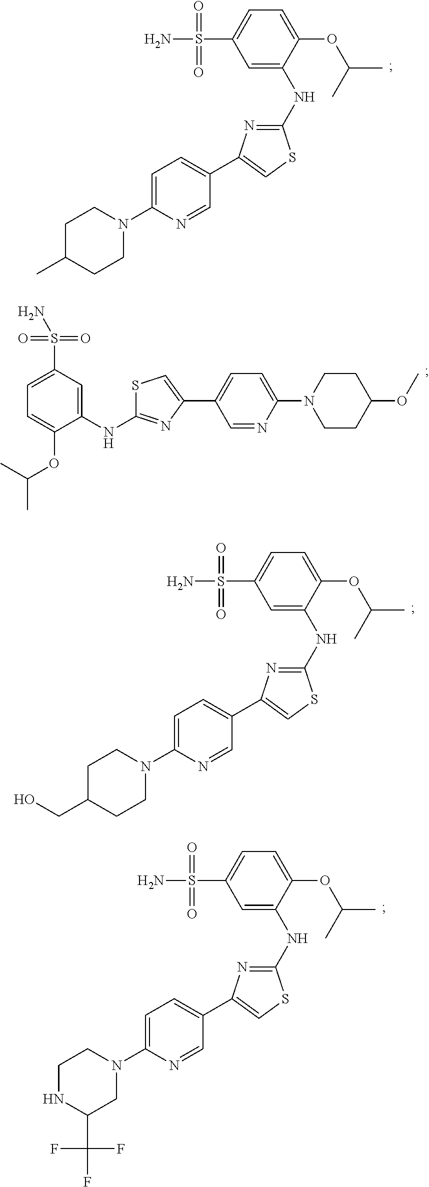 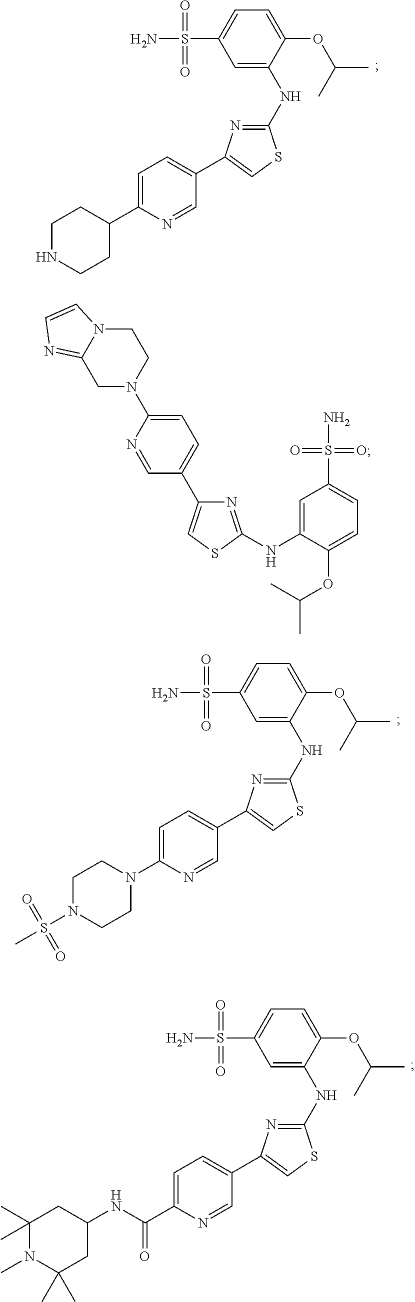 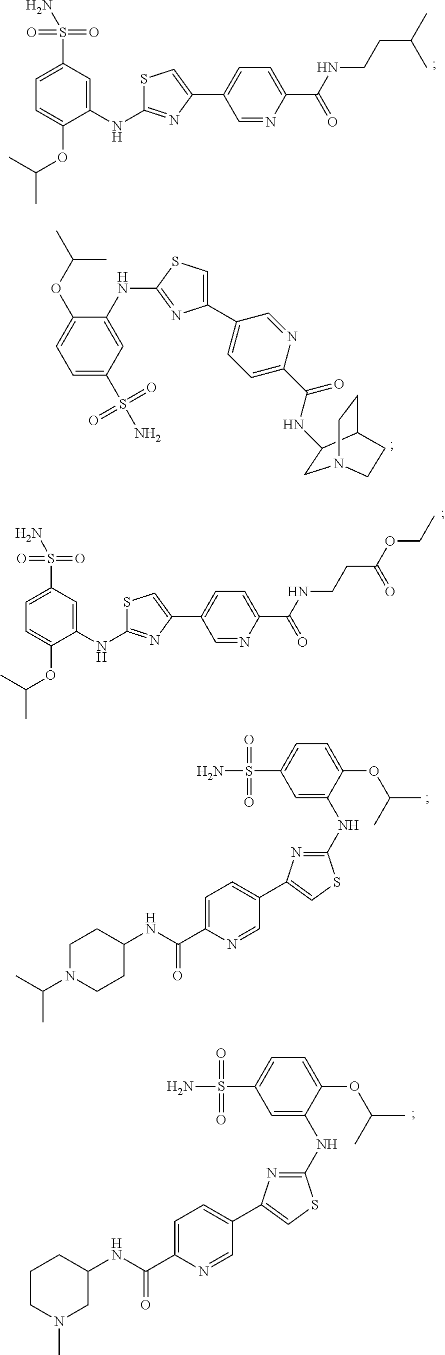 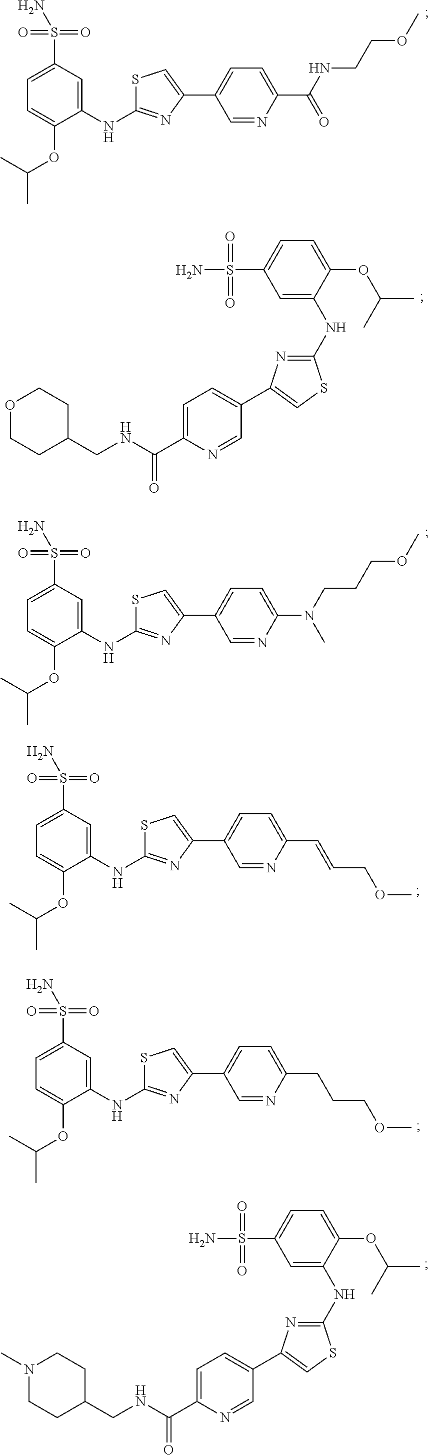 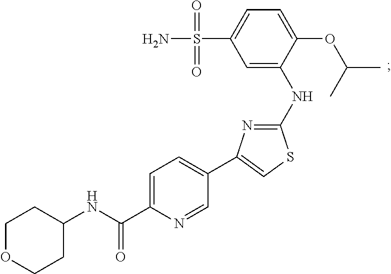
and solvates, hydrates, tautomers, and pharmaceutically acceptable salts thereof.

8. A pharmaceutical composition, comprising a compound of claim 1 and a pharmaceutically acceptable carrier.

9. A pharmaceutical composition, comprising a compound listed in the Examples section of this specification and a pharmaceutically acceptable carrier.

10. A method for preventing, treating or ameliorating an MMP9 mediated syndrome, disorder or disease comprising administering to a subject in need thereof an effective amount of a compound of claim 1 or a form, composition or medicament thereof.

11. A method for preventing, treating or ameliorating an MMP9 mediated syndrome, disorder or disease wherein said syndrome, disorder or disease is associated with elevated MMP9 expression or MMP9 overexpression, or is a condition that accompanies syndromes, disorders or diseases associated with elevated MMP9 expression or MMP9 overexpression comprising administering to a subject in need thereof an effective amount of a compound of Formula I or a form, composition or medicament thereof.

12. A method of preventing, treating or ameliorating a syndrome, disorder or disease, wherein said syndrome, disorder or disease is selected from the group consisting of: neoplastic disorders, osteoarthritis, rheumatoid arthritis, cardiovascular diseases, gastric ulcer, pulmonary hypertension, chronic obstructive pulmonary disease, inflammatory bowel syndrome, periodontal disease, skin ulcers, liver fibrosis, emphysema, Marfan syndrome, stroke, multiple sclerosis, asthma, abdominal aortic aneurysm, coronary artery disease, idiopathic pulmonary fibrosis, renal fibrosis, and migraine, comprising administering to a subject in need thereof an effective amount of a compound of Formula I or a form, composition or medicament thereof.

13. The method of claim 12, wherein said syndrome, disorder or disease is a neoplastic disorder, which is ovarian cancer.

14. The method of claim 12, wherein said syndrome, disorder or disease is a cardiovascular disease, wherein said cardiovascular disease is selected from the group consisting of: atherosclerotic plaque rupture, aneurysm, vascular tissue morphogenesis, coronary artery disease, and myocardial tissue morphogenesis.

15. The method of claim 14, wherein said cardiovascular disease is atherosclerotic plaque rupture.

16. The method of claim 12, wherein said syndrome, disorder or disease is rheumatoid arthritis.

17. The method of claim 12, wherein said syndrome, disorder or disease is asthma.

18. The method of claim 12, wherein said syndrome, disorder or disease is chronic obstructive pulmonary disease.

19. The method of claim 12, wherein said syndrome, disorder or disease is inflammatory bowel syndrome.

20. The method of claim 12, wherein said syndrome, disorder or disease is abdominal aortic aneurism.

21. The method of claim 12, wherein said syndrome, disorder or disease is osteoarthritis.

22. The method of claim 12, wherein said syndrome, disorder or disease is idiopathic pulmonary fibrosis.

23. A method of inhibiting MMP9 activity in a mammal by administration of an effective amount of at least one compound of claim 1.

24. A method for preventing, treating or ameliorating an MMP13 mediated syndrome, disorder or disease comprising administering to a subject in need thereof an effective amount of a compound of claim 1 or a form, composition or medicament thereof.

25. A method for preventing, treating or ameliorating an MMP13 mediated syndrome, disorder or disease wherein said syndrome, disorder or disease is associated with elevated MMP13 expression or MMP13 overexpression, or is a condition that accompanies syndromes, disorders or diseases associated with elevated MMP13 expression or MMP13 overexpression comprising administering to a subject in need thereof an effective amount of a compound of Formula I or a form, composition or medicament thereof.

26. A method of inhibiting MMP13 activity in a mammal by administration of an effective amount of at least one compound of claim 1.
